(19) United States
(12) Patent Application Publication
(10) Pub. No.: US 2024/0068032 A1
(43) Pub. Date: Feb. 29, 2024

(54) DISEASE MARKER

(71) Applicant: OXFORD BIODYNAMICS PLC, Oxford (GB)

(72) Inventors: Aroul Selvam RAMADASS, Oxford Oxfordshire (GB); Ewan HUNTER, Oxford Oxfordshire (GB); Alexandre AKOULITCHEV, Oxford Oxfordshire (GB)

(73) Assignee: OXFORD BIODYNAMICS PLC, Oxford, GB (GB)

(21) Appl. No.: 18/247,133

(22) PCT Filed: Oct. 11, 2021
(86) PCT No.: PCT/GB2021/052616

§ 371 Date: Mar. 29, 2023

(30) Foreign Application Priority Data
Oct. 12, 2020 (GB) 2016176.6

(51) Int. Cl. C12Q 1/6883

(52) U.S. Cl. CPC C12Q 1/6883; C12Q 2600/156; C12Q 2600/112

(57) ABSTRACT

A process for analysing chromosome interactions relating to muscular atrophy.

### CROSS-REFERENCE

This application is a 371 National Stage filing and claims the benefit under 35 U.S.C. § 120 of International Application No. PCT/GB2021/052616, filed 11 Oct. 2021, which claims priority to Great Britain Application No. GB2016176.6, filed 12 Oct. 2020, each of which is incorporated herein by reference in its entirety.

### SEQUENCE LISTING INCORPORATION BY REFERENCE

The application herein incorporates by reference in its entirety the sequence listing material in the ASCII text file named “Sequence Listing”, created Mar. 6, 2023, and having the size of 176 kilobytes, filed with this application.

### FIELD OF THE INVENTION

The invention relates to disease processes.

### BACKGROUND OF THE INVENTION

Muscular atrophy (MD) is a group of muscle diseases that typically results in increasing weakening and breakdown of skeletal muscles over time. The disorders differ in which muscles are primarily affected, the degree of weakness, how fast they worsen, and when symptoms begin. Many people will eventually become unable to walk. Some types are also associated with problems in other organs.

### SUMMARY OF THE INVENTION

The inventors have identified chromosome conformation signatures relevant to muscular atrophy. Accordingly the invention provides a method of detecting the muscular atrophy status in an individual, comprising determining the presence or absence of one or more chromosome interactions represented by the probes shown in Table 1, to thereby detect muscular atrophy in the individual.

Preferably the method is carried out to select an individual for receiving therapy or a treatment for muscular atrophy. The method may be carried out on individual that has been preselected based on a physical characteristic, risk factor or the presence of a symptom. The method is typically carried out to diagnose muscular atrophy or to determine prognosis for muscular atrophy, and preferably to determine severity of muscular atrophy.

### BRIEF DESCRIPTION OF THE DRAWINGS

FIG. 1 shows a description of the spinal and bulbar muscular atrophy (SBMA) samples used in the experimental work.

FIG. 2 shows the PCA of all SBMA arrays. PCA of more than 900k EpiSwitch CCSs. Sample SBMA0007 shows an issue. Squares show pHC and circles shows SMBA in all the figures.

FIG. 3 shows the PCA of all SBMA arrays with SBMA0007 removed. The data then separates out disease and control samples.

FIG. 4 shows a PCA with the samples labelled up by the FRS scope. There are no obvious clusters due to FRS score.

FIG. 5 shows a PCA with samples labelled up by the CAG repeat. There are no obvious clusters by CAG repeat.

FIG. 6 shows a PCA with samples labelled up by disease duration. There are no obvious clusters dues to disease duration.

FIG. 7 shows top significant chromosome interactions (also termed ‘CCS’, chromosome conformation signature) which are present and absent in SBMA. The top 200 interactions for each set were below <=0.05 FDR identified. The two tables show the top 40 interactions per cohort, ranked by interaction frequency.

FIG. 8 shows genetic location mapping for SBMA present interactions. Although the drawing is not included here, but PD-L1 expression and PD-1 checkpoint pathways in cancer were looked at and interactions associated with them were identified as present in SBMA patients.

FIG. 9 and FIG. 10 show a pathway analysis of the top SBMA present interaction associated genes.

FIG. 11 shows RET signalling associated pathways.

FIG. 12 shows genetic location mapping for SBMA absent interactions.

FIG. 13 shows a pathway analysis of top absent SBMA interaction associated genes.

FIG. 14 shows disease analysis of top SBMA absent interaction associated genes, going from the left box to the right box. The figure shows disease enrichment analysis of the genes associated to the top CCSs absent in SBMA.

FIG. 15 shows a preferred method for carrying out the marker detection step of the invention.

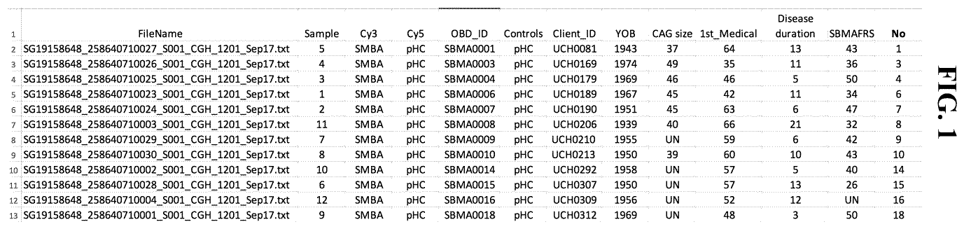

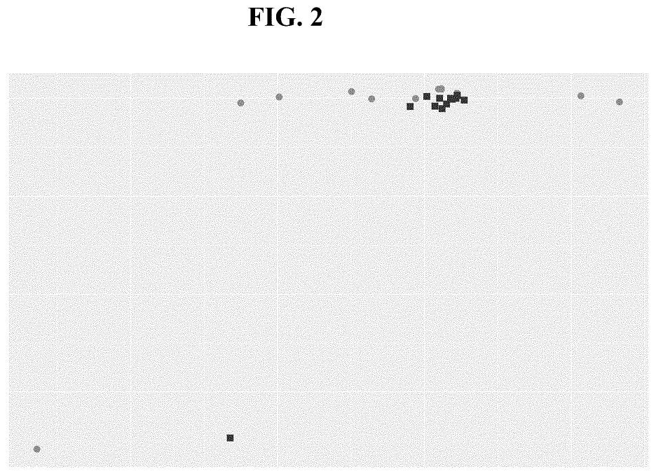

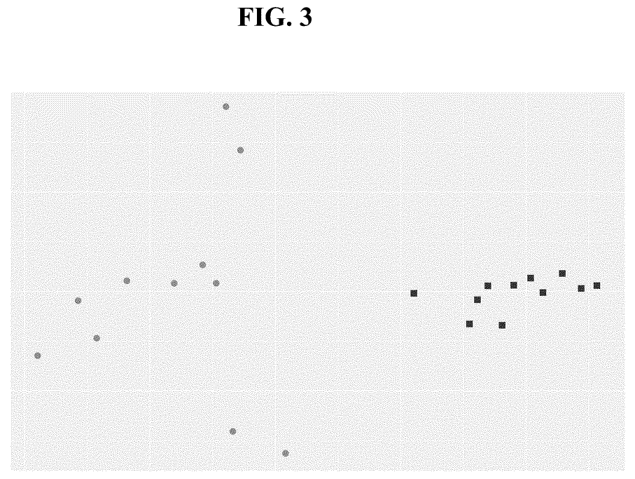

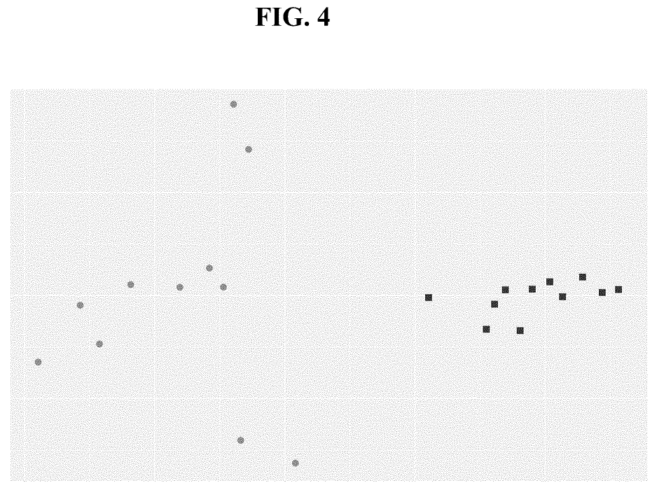

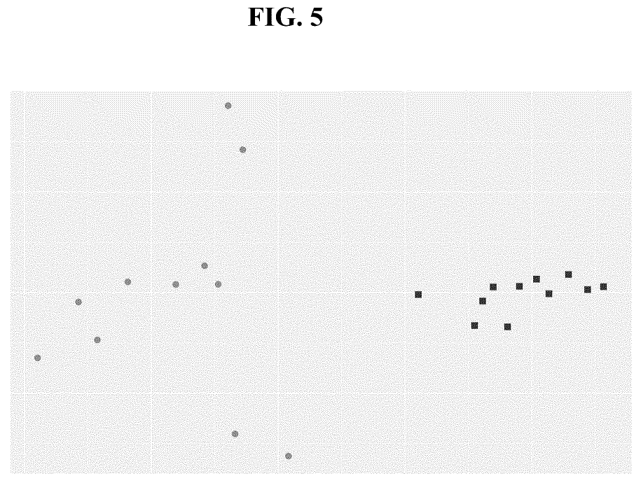

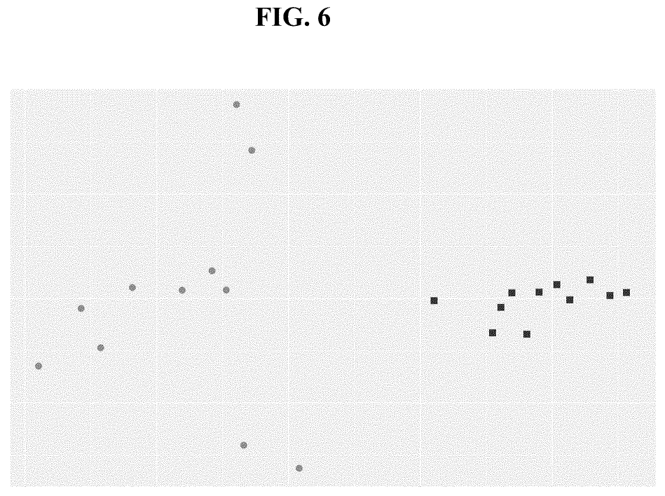

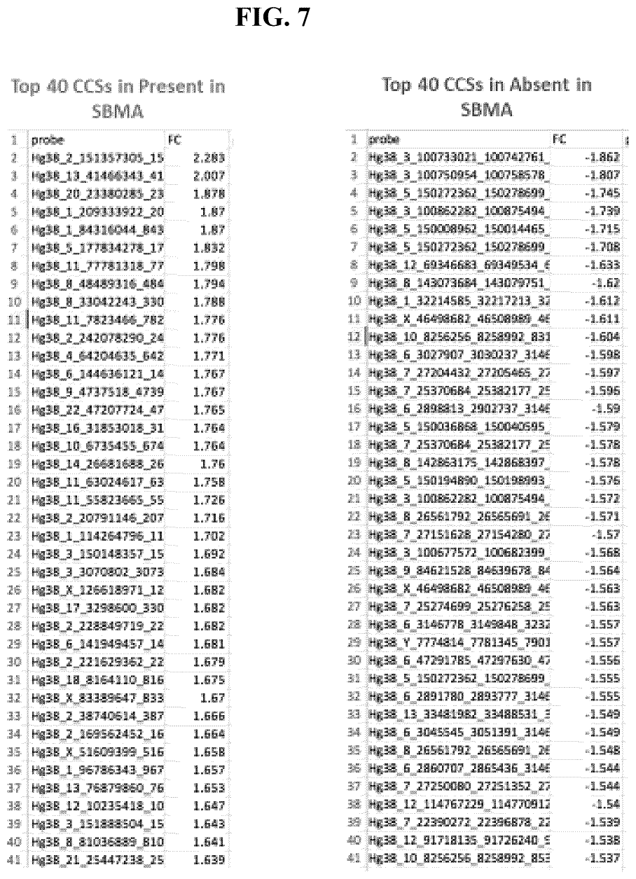

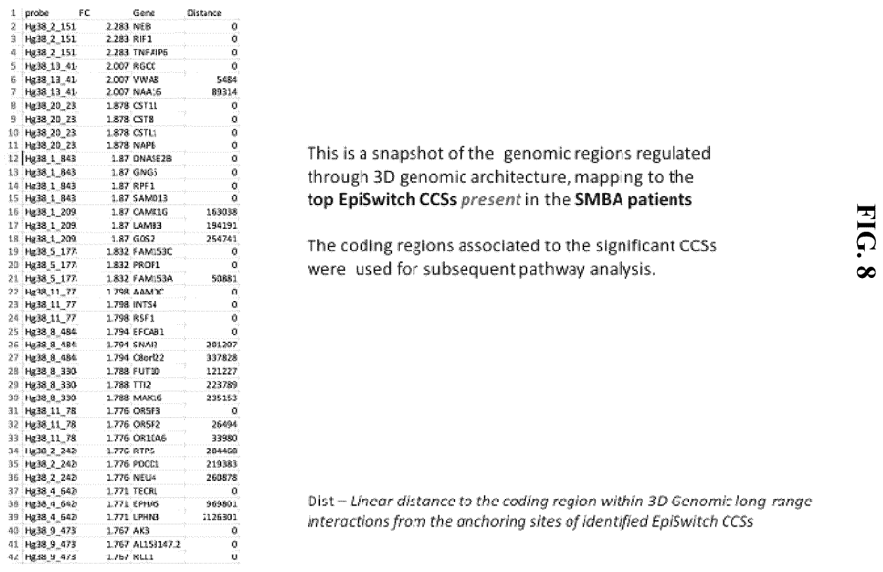

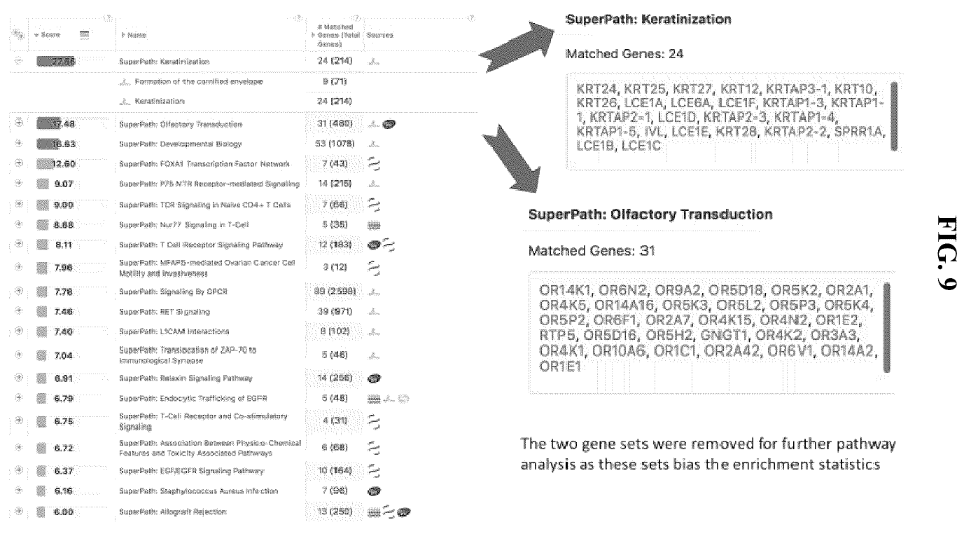

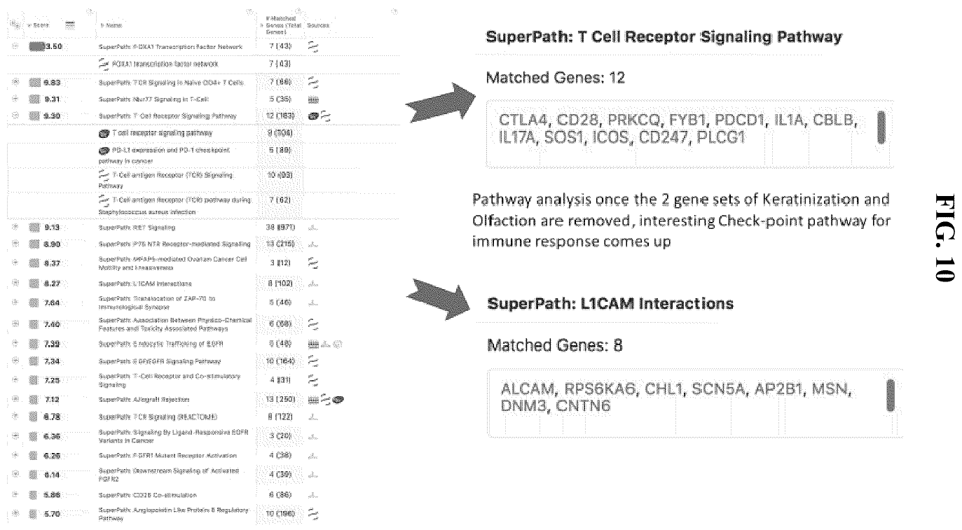

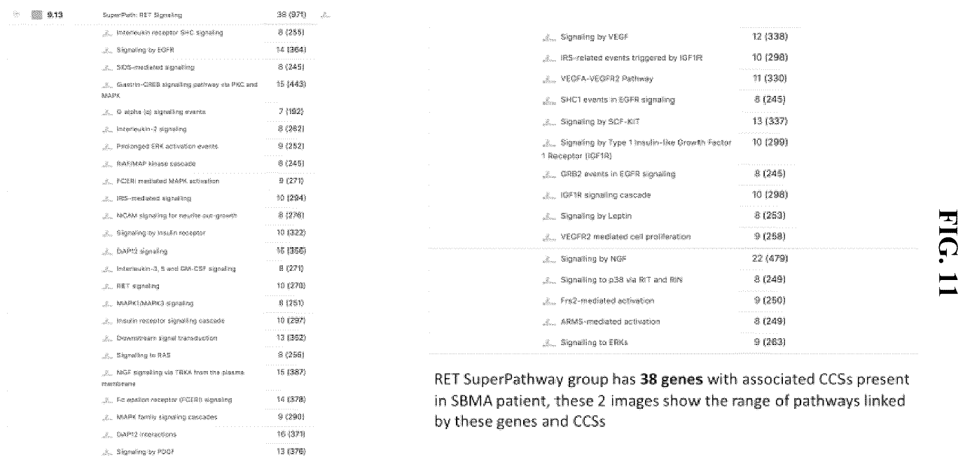

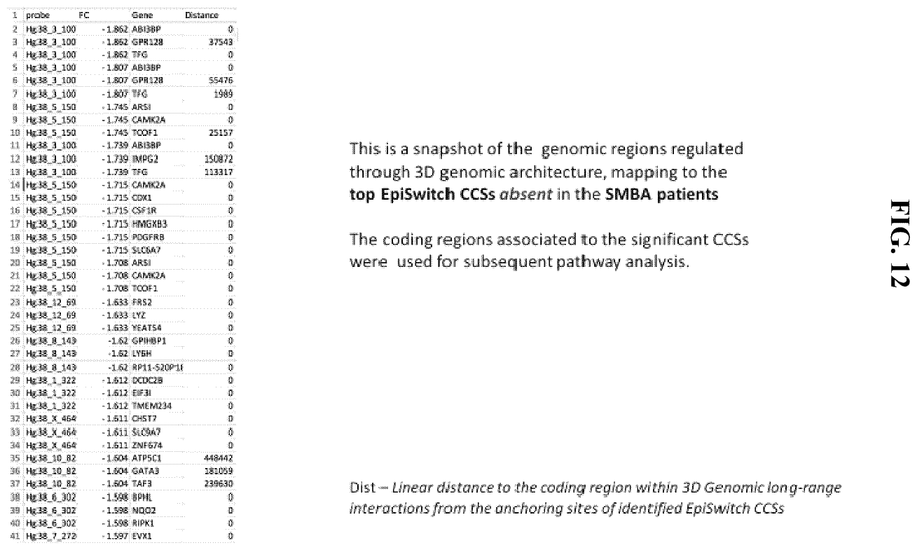

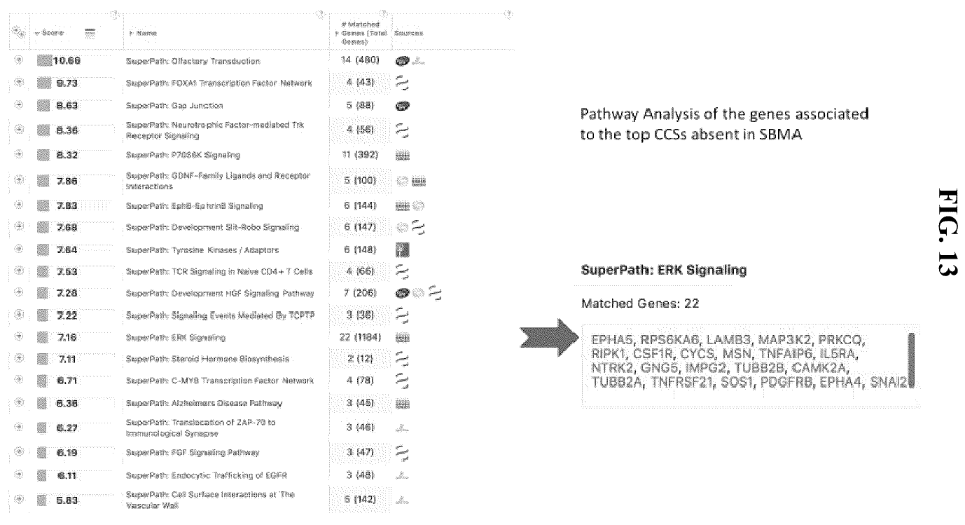

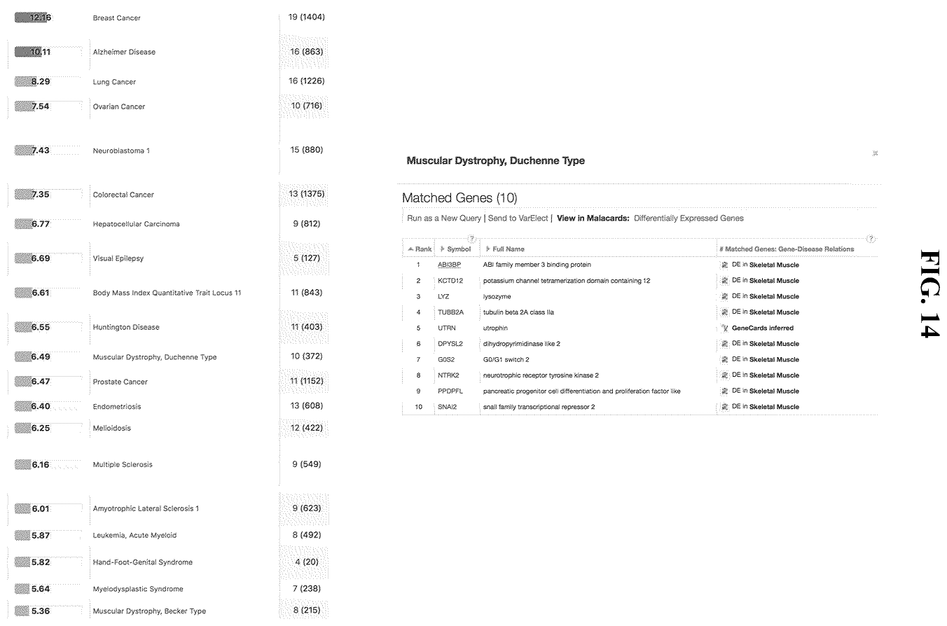

### DETAILED DESCRIPTION OF THE INVENTION

### Terms Used Herein

The method of the invention may be referred to as the ‘process’ of the invention herein.

The chromosome interactions which are typed may be referred to as ‘markers’, ‘CCS’, ‘chromosome conformation signature’ or ‘epigenetic interaction’ herein. Such interactions are recognised in the art as regions of the chromosome coming together in a stable manner and this represents a distinct mode of regulation. A chromosome interaction can also be referred to as a ‘juxtaposition’ of chromosomes, chromosome ‘folding’ or ‘chromatin interaction’. Such interactions can be detected, for example, using the 3C (chromosome conformation capture) method.

The word ‘type’ will be interpreted as per the context, but will usually refer to detection of whether a specific chromosome interaction is present or absent.

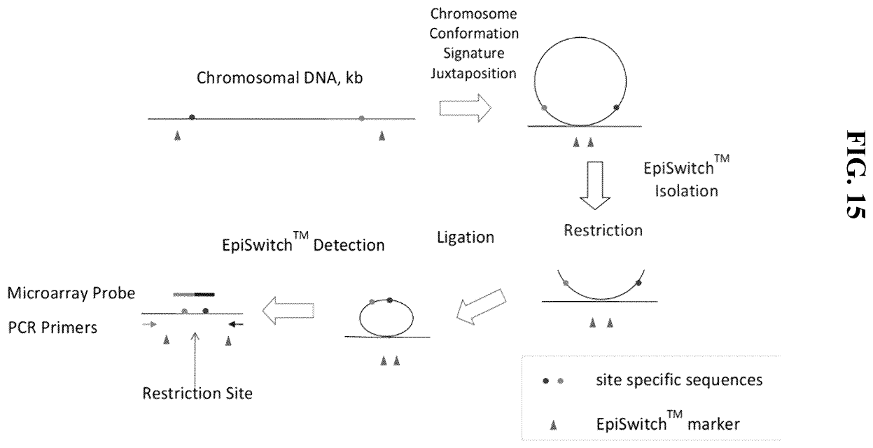

The chromosome interactions which are typed in the method of the invention are defined in Table 1. They are defined by means of the probe sequences which detect the ligated product made by an EpiSwitch method (see FIG. 15). They are also defined by the position numbers of the interaction which are included within the probe name and they are also defined by the primer sequences which allow detection of the ligated sequence. The chromosome interaction can be defined by the ‘probe location’ given in the tables with reference to the chromosome number and the ‘Start’ and ‘End’ positions given for the chromosome regions which come together to form the interaction.

### Aspects of the Invention

The invention relates to determining different aspects of muscular atrophy, including in respect to the presence or stage of muscular atrophy. This determining is by typing any of the relevant markers disclosed herein, for example in Table 1, or preferred combinations of markers, or markers in defined specific regions disclosed herein.

Specific number of markers may be chosen from any group of markers which is specifically disclosed herein. Preferred numbers of markers are at least 3, 5, 8, 10, 15 and at least 20. Preferred groups of markers are those shown in each table, or each part of a table (for example “Table 1A (part 1a)”), or all the markers associated with a distinct characteristic of muscular atrophy.

The invention includes a process of typing a patient to identify whether they have muscular atrophy and/or the stage of muscular atrophy. The invention includes diagnosis of an individual for any condition or stage of disease as defined herein (i.e. prognosis), which can be thought of as determining the subgroup they belong to.

The invention also concerns a panel of epigenetic markers which relates to muscular atrophy. The panel may have been optimised in some way, for example by GLMNET analysis.

The invention therefore allows personalised therapy to be given to the patient which accurately reflects the patient's needs.

Any therapy, for example drug, which is mentioned herein may be administered to an individual based on the result of the process.

Marker sets are disclosed in the Tables and Figures. In one embodiment at least 10 markers from any disclosed marker set are used in the invention. In another embodiment at least 20% of the markers from any disclosed marker set are used in the invention.

### The Epigenetic Interactions Relevant to the Invention

The chromosome interactions which are typed in the invention are typically interactions between distal regions of a chromosome, said interactions being dynamic and altering, forming or breaking depending upon the state of the region of the chromosome. That state will reflect different aspects of muscular atrophy and therefore the invention can be carried out to the presence, type, severity or stage of muscular atrophy.

The chromosome interaction may, for example, reflect if it is being transcribed or repressed. Chromosome interactions which are specific to muscular atrophy subgroups as defined herein have been found to be stable, thus providing a reliable means of measuring the differences between the two subgroups (for example a muscular atrophy group and a healthy group that does not have muscular atrophy).

Chromosome interactions specific to muscular atrophy will normally occur early in the disease process, for example compared to other epigenetic markers such as methylation or changes to binding of histone proteins. Thus the process of the invention is able to detect disease at an early stage. This allows early intervention (for example treatment) which as a consequence will be more effective. Chromosome interactions also reflect the current state of the individual and therefore can be used to assess changes to disease status. Furthermore there is little variation in the relevant chromosome interactions between individuals within the same subgroup. Detecting chromosome interactions is highly informative with up to 50 different possible interactions per gene, and so processes of the invention can for example interrogate 500,000 possible different interactions.

Chromosomal interactions may overlap and include the regions of chromosomes shown to encode relevant or undescribed genes, but equally may be in intergenic regions. It should further be noted that the inventors have discovered that chromosome interactions in all regions are equally important in determining the status of a chromosomal locus.

The chromosome interactions which are detected in the invention could be impacted by changes to the underlying DNA sequence, by environmental factors, DNA methylation, non-coding antisense RNA transcripts, non-mutagenic carcinogens, histone modifications, chromatin remodelling and specific local DNA interactions. However it must be borne in mind that chromosome interactions as defined herein are a regulatory modality in their own right and do not have a one to one correspondence with any genetic marker (DNA sequence change) or any other epigenetic marker.

The changes which lead to the chromosome interactions may be impacted by changes to the underlying nucleic acid sequence which themselves do not directly affect a gene product or the mode of gene expression. Such changes may be for example, SNPs within and/or outside of the genes, gene fusions and/or deletions of intergenic DNA, microRNA, and non-coding RNA. For example, it is known that roughly 20% of SNPs are in non-coding regions, and therefore the process as described is also informative in non-coding situation. In one aspect the regions of the chromosome which come together to form the interaction are less than 5 kb, 3 kb, 1 kb, 500 base pairs or 200 base pairs apart on the same chromosome.

The chromosome interaction which is detected may be within a gene, such as any gene mentioned herein. However it may also be upstream or downstream of the gene, for example up to 50,000, up to 30,000, up to 20,000, up to 10,000 or up to 5000 bases upstream or downstream from the gene or from the coding sequence.

The Process of the Invention

The process of the invention comprises a typing system for detecting chromosome interactions relevant to muscular atrophy. Any suitable typing method can be used, for example a method in which the proximity of the chromosomes in the interaction is detected. The typing method may be performed using the EpiSwitch™ system mentioned herein which for example may be carried out by a method comprising the following steps (for example on a sample from the subject):
(i) cross-linking regions of chromosome which have come together in a chromosome interaction,
(ii) optionally isolating the cross-linked DNA from said chromosomal locus
(iii) subjecting the cross-linked DNA to cleavage, and
(iv) ligating the nucleic acids present in the cross-linked entity to derive a ligated nucleic acid with sequence from both the regions which formed a chromosomal interaction.

Detection of this ligated nucleic acid allows determination of the presence or absence of a particular chromosome interaction. The ligated nucleic acid therefore acts as a marker for the presence of the chromosome interaction. Preferably the ligated nucleic acid is detected by PCR or a probe based method, including a qPCR method.

In the method the chromosomes can be cross-linked by any suitable means, for example by a cross-linking agent, which is typically a chemical compound. In a preferred aspect, the interactions are cross-linked using formaldehyde, but may also be cross-linked by any aldehyde, or D-Biotinoyl-e-aminocaproic acid-N-hydroxysuccinimide ester or Digoxigenin-3-O-methylcarbonyl-e-aminocaproic acid-N-hydroxysuccinimide ester. Para-formaldehyde can cross link DNA chains which are 4 Angstroms apart. Preferably the chromosome interactions are on the same chromosome. Typically the chromosome interactions are 2 to 10 Angstroms apart.

The cross-linking is preferably in vitro. The cleaving is preferably by restriction digestion with an enzyme, such as Taql. The ligating may form DNA loops.

Where PCR (polymerase chain reaction) is used to detect or identify the ligated nucleic acid, the size of the PCR product produced may be indicative of the specific chromosome interaction which is present, and may therefore be used to identify the status of the locus. In preferred aspects the primers shown in any table herein are used, for example the primer pairs shown in Table 1 are used (corresponding to the chromosome interaction which is being detected). Homologues of such primers or primer pairs may also be used, which can have at least 70% identity to the original sequence.

Where a probe is used to detect or identify the ligated nucleic acid, this is generally by Watson-Crick based base-pairing between the probe and ligated nucleic acid. Probe sequences as shown in any table herein may be used, for example the probe sequences shown in Table 1 (corresponding to the chromosome interaction which is being detected). Homologues of such probe sequences may also be used, which can have at least 70% identity to the original sequence.

Typing according to the process of the invention may be carried out at multiple time points, for example to monitor the progression of the disease. This may be at one or more defined time points, for example at at least 1, 2, 5, 8 or 10 different time points. The durations between at least 1, 2, 5 or 8 of the time points may be at least 5, 10, 20, 50, 80 or 100 days. Typically there are 3 time points at least 50 days apart.

### Subgroups and Personalised Treatment

As used herein, a “subgroup” preferably refers to a population subgroup, more preferably a subgroup in the population of a particular animal such as a particular eukaryote, or mammal. Most preferably, a “subgroup” refers to a subgroup in the human population. Therefore the process of the invention is preferably carried out to detect the presence of muscular atrophy in a human. The process of the invention may be carried out for diagnostic or prognostic purposes.

The invention includes detecting and treating particular subgroups in a population. The inventors have discovered that chromosome interactions differ between subsets (for example at least two subsets) in the relevant population. Identifying these differences will allow physicians to categorize their patients as a part of one subset of the population. The invention therefore provides physicians with a process of personalizing medicine for the patient based on their epigenetic chromosome interactions. Such testing may be used to select how to subsequently treat the patient, for example the type of drug and/or its dose and/or its frequency of administration.

The individual that is tested in the process of the invention may have been selected in some way. The individual may be susceptible to any condition mentioned herein and/or may be in need of any therapy mentioned in. The individual may be receiving any therapy mentioned herein. In particular, the individual may have, or be suspected of having, muscular atrophy. Thus the invention includes a process of typing a patient to diagnose muscular atrophy, which is equivalent to determining the subgroup they belong to. The muscular atrophy may be any of the following conditions: spinal bulbar muscle atrophy (SBMA), a polyglutamine disease, dentatorubral-pallidoluysian atrophy, spinocerebellar ataxia, sarcopenia or cachexia. Therefore the process of the invention may comprise detecting (or diagnosing) any of these conditions. The process of the invention may comprise determining prognosis of any of these conditions, such as determining the severity.

The individual may be receiving any of the following or may have received any of these in the previous 365 days: physiotherapy, rehabilitation, therapeutic agents against tremor and muscle cramps, hormone therapy, surgical treatment of gynecomastia, tube feeding or ventilatory support. The individual may have cancer. The individual may be a human male of age 30 to 60, for example of age 40 to 50. The individual may have gynecomastia, testicular atrophy, reduced fertility or androgen insensitivity. The individual may have reduced fertility due to androgen insensitivity.

### Tables Provided Herein

Table 1 shows 400 specific markers which can be used to detect muscular atrophy, i.e. their presence or absence can be used in such a detection (i.e. they are ‘disseminating’ markers). Table 1A shows 200 markers which are only present in muscular atrophy. Table 13 shows 200 markers which are present only healthy controls, i.e. they are absent in muscular atrophy. The process of the invention can therefore be carried out using markers from Table 1A or from Table 1B, or from a selection of markers from both Table 1A and Table 13.

The markers are defined using probe sequences (which detect a ligated product as defined herein). The first two sets of Start-End positions show probe positions, and the second two sets of Start-End positions show the relevant 4 kb region.

The following information is provided in the probe data table:
RP—Rsum the Rank Product statistics evaluated per each chromosome interaction.
FC—Interaction frequency (positive or negative).
Pfp—estimated percentage of false positive predictions (pfp), both considering positive and negative chromosome interactions.
Pval—estimated pvalues per each CCSs being positive and negative.
Adj.P.value(FDR)—False discovery rate adjusted p.value.
Loop Detected—which state the loop is found in.

Simple permutation-based estimation is used to determine how likely a given RP value or better is observed in a random experiment. This has the following steps:
1. Generate p permutations of k rank lists of length n.
2. Calculate the rank products of the n CCS in the p permutations.
3. Count (c) how many times the rank products of the CCS in the permutations are smaller or equal to the observed rank product. Set c to this value.
4. Calculate the average expected value for the rank product by: Erp(g)=c/p.
5. Calculate the percentage of false positives as: pfp (g)=Erp(g)/rank (g) where rank(g) is the rank of CCS g in a list of all n CCSs sorted by increasing RP.

The rank product statistic ranks chromosome interactions according to intensities within each microarray and calculates the product of these ranks across multiple microarrays. This technique can identify chromosome interactions that are consistently detected among the most differential chromosome interactions in a number of replicated microarrays. Where the p-value is 0 this indicates that there is very little variation in the Rank Product of the CCS across the samples, this is a good example of the signal to noise and effect size of CCS. Where p value is 0 and pfp is 0 this means that permutated Rank Product doesn't differ from the actual observed Rank Product. These methods are described Breitling R and Herzyk P (2005) Rank-based methods as a non-parametric alternative of the t-test for the analysis of biological microarray data. J Bioinf Comp Biol 3, 1171-1189.

The FC indicates prevalence of marker in each comparison, 2 means twice over average test, 1.5 means 1.5 over the average test, etc., and so FC indicates the weight of a marker to phenotype/group. The FC value can be used to give an indication of how many markers are needed for a highly effective test. Typically 5 to 10 markers will give a highly effective test, though even smaller numbers of markers will give a functional test for detection of muscular atrophy.

The probes are designed to be 30 bp away from the Taq1 site. In case of PCR, PCR primers are typically designed to detect ligated product but their locations from the Taq1 site vary. Probe locations:
Start 1−30 bases upstream of Taql site on fragment 1
End 1—Taql restriction site on fragment 1
Start 2—Taql restriction site on fragment 2
End 2—30 bases downstream of Taql site on fragment 2

4 kb Sequence Location:
Start 1—4000 bases upstream of Taql site on fragment 1
End 1—Taql restriction site on fragment 1
Start 2—Taql restriction site on fragment 2
End 2—4000 bases downstream of Taql site on fragment 2

### Preferred Marker Sets

The invention relates to detecting the presence of muscular atrophy by typing chromosome interaction markers, such as any of the specific markers disclosed herein, for example in Table 1, or preferred combinations of markers, or markers in defined specific regions disclosed herein. Markers present in genes and regions mentioned in the tables may be typed. Specific markers are defined herein by location or by probe and/or primer sequences. Therefore preferred markers are those which are represented by the probes and/or primer pairs disclosed in tables herein.

Combinations of markers can be defined in different ways, such as:
by ranking by any parameter defined herein, or
by any ‘part’ of Table 1,
by reference to the ‘number’ of the marker which is listed in the left hand column of Table 1.

In a preferred aspect at least 10 markers are typed from the top 40 markers for any parameter mentioned in Table 1, such as FC.

In one aspect one or more markers are typed which:
(i) are present in any one of the regions listed in Table 1; and/or
(ii) corresponds to any one of the chromosome interactions represented by any probe shown in Table 1; and/or
(iii) is present in a 4,000 base region which comprises or which flanks (i) or (ii).

In a preferred aspect:
at least 5 chromosome interactions are typed from Table 1A, and/or
at least 5 chromosome interactions are typed from Table 1B.

In another preferred aspect at least 5 chromosome interactions are typed selected from:
interaction numbers 1 to 40 from Table 1A, or
interaction numbers 1 to 40 from Table 1B.

### Preferred Numbers of Markers to be Typed

Typing a very low number of the markers disclosed herein will result in an effective test due to the nature of regulation by chromosome interaction, including their network-like properties. The different numbers and combination of markers give rise to different performance properties. Further as will be appreciated the markers can be selected from Table 1 as a whole or from the parts of the table defined by Table 1A and Table 1B, or from parts defined by a number and letter (reflecting certain marker numbers).

In one aspect the process comprising typing at least 3, 5, 8, 10, 15 or 20 of the chromosome interactions represented by the probes in Table 1. In one embodiment at least 10 chromosome interactions represented by the probes in Table 1 are typed.

In one aspect the process comprising typing at least 50, 80, 100, 150, 200, 250, 300, 350 or all of the chromosome interactions represented by the probes in Table 1.

In one aspect the process comprising typing at least 3, 5, 8, 10, 15 or 20 of the chromosome interactions represented by the probes in Table 1A. In one embodiment at least 10 chromosome interactions represented by the probes in Table 1A are typed.

In one aspect the process comprising typing at least 30, 50, 80, 100, 150 or all of the chromosome interactions represented by the probes in Table 1A.

In one aspect the process comprising typing at least 3, 5, 8, 10, 15 or 20 of the chromosome interactions represented by the probes in Table 1B. In one embodiment at least 10 chromosome interactions represented by the probes in Table 1B are typed.

In one aspect the process comprising typing at least 30, 50, 80, 100, 150 or all of the chromosome interactions represented by the probes in Table 1B.

In one aspect at least 3, 5, 8, 10, 15 or 20 chromosome interactions are typed from the markers listed as numbers 1 to 40 in Table 1A and/or at least 3, 5, 8, 10, 15 or 20 chromosome interactions are typed from the markers listed as numbers 1 to 40 in Table 1B.

In one aspect at least 10 chromosome interactions are typed from the markers listed as numbers 1 to 40 in Table 1A and/or at least 10 chromosome interactions are typed from the markers listed as numbers 1 to 40 in Table 1B.

### Types of Chromosome Interaction

In one aspect the locus (including the gene and/or place where the chromosome interaction is detected) may comprise a CTCF binding site. This is any sequence capable of binding transcription repressor CTCF. That sequence may consist of or comprise the sequence CCCTC which may be present in 1, 2 or 3 copies at the locus. The CTCF binding site sequence may comprise the sequence CCGCGNGGNGGCAG (in IUPAC notation). The CTCF binding site may be within at least 100, 500, 1000 or 4000 bases of the chromosome interaction or within any of the chromosome regions shown Table 1.

When detection is performed using a probe, typically sequence from both regions of the probe (i.e. from both sites of the chromosome interaction) could be detected. In preferred aspects probes are used in the process which comprise or consist of the same or complementary sequence to a probe shown in any table. In some aspects probes are used which comprise sequence which is homologous to any of the probe sequences shown in the tables.

### The Approach Taken to Identify Markers and Panels of Markers

The invention described herein relates to chromosome conformation profile and 3D architecture as a regulatory modality in its own right, closely linked to the phenotype. The discovery of biomarkers was based on annotations through pattern recognition and screening on representative cohorts of clinical samples representing the differences in phenotypes. We annotated and screened significant parts of the genome, across coding and non-coding parts and over large sways of non-coding 5′ and 3′ of known genes for identification of statistically disseminating consistent conditional disseminating chromosome conformations, which for example anchor in the non-coding sites within (intronic) or outside of open reading frames.

In selection of the best markers we are driven by statistical data and p values for the marker leads. Selected and validated chromosome conformations within the signature are disseminating stratifying entities in their own right, irrespective of the expression profiles of the genes used in the reference. Further work may be done on relevant regulatory modalities, such as SNPs at the anchoring sites, changes in gene transcription profiles, changes at the level of H3K27ac.

We are taking the question of clinical phenotype differences and their stratification from the basis of fundamental biology and epigenetic controls over phenotype—including for example from the framework of network of regulation. As such, to assist stratification, one can capture changes in the network and it is preferably done through signatures of several biomarkers, for example through following a machine learning algorithm for marker reduction which includes evaluating the optimal number of markers to stratify the testing cohort with minimal noise. This may end with 3-20 markers.

Selection of markers for panels may be done by cross-validation statistical performance (and not for example by the functional relevance of the neighbouring genes, used for the reference name).

A panel of markers (with names of adjacent genes) is a product of clustered selection from the screening across significant parts of the genome, in non-biased way analysing statistical disseminating powers over 14,000-60,000 annotated EpiSwitch sites across significant parts of the genome. It should not be perceived as a tailored capture of a chromosome conformation on the gene of know functional value for the question of stratification. The total number of sites for chromosome interaction are 1.2 million, and so the potential number of combinations is 1.2 million to the power 1.2 million. The approach that we have followed nevertheless allows the identifying of the relevant chromosome interactions.

The specific markers that are provided by this application have passed selection, being statistically (significantly) associated with the condition or subgroup. This is what the data in the relevant table demonstrates. Each marker can be seen as representing an event of biological epigenetic as part of network deregulation that is manifested in the relevant condition. In practical terms it means that these markers are prevalent across groups of patients when compared to controls. On average, as an example, an individual marker may typically be present in 80% of patients tested and in 10% of controls tested.

Simple addition of all markers would not directly represent the network interrelationships between some of the deregulations. This is where the standard multivariate biomarker analysis GLMNET (R package) can be brought in. GLMNET package helps to identify interdependence between some of the markers, that reflect their joint role in achieving deregulations leading to disease phenotype. Modelling and then testing markers with highest GLMNET scores offers not only identify the minimal number of markers that accurately identifies the patient cohort, but also the minimal number that offers the least false positive results in the control group of patients, due to background statistical noise of low prevalence in the control group. Typically a group (combination) of selected markers (such as 3 to 10) offers the best balance between both sensitivity and specificity of detection, emerging in the context of multivariate analysis from individual properties of all the selected statistical significant markers for the condition.

The tables herein show the reference names for the array probes (60-mer) for array analysis that overlaps the juncture between the long range interaction sites, the chromosome number and the start and end of two chromosomal fragments that come into juxtaposition.

### Samples and Sample Treatment

The process of the invention will normally be carried out on a sample. The sample may be obtained at a defined time point, for example at any time point defined herein. The sample will normally contain DNA from the individual. It will normally contain cells. In one aspect a sample is obtained by minimally invasive means, and may for example be a blood sample. DNA may be extracted and cut up with a standard restriction enzyme. This can pre-determine which chromosome conformations are retained and will be detected with the EpiSwitch™ platforms. Due to the synchronisation of chromosome interactions between tissues and blood, including horizontal transfer, a blood sample can be used to detect the chromosome interactions in tissues, such as tissues relevant to disease.

### Preferred Aspects for Sample Preparation and Chromosome Interaction Detection

Methods of preparing samples and detecting chromosome conformations are described herein. Optimised (non-conventional) versions of these processes can be used, for example as described in this section.

Typically the sample will contain at least 2×10⁵ cells. The sample may contain up to 5×10⁵ cells. In one aspect, the sample will contain 2×10⁵ to 5.5×10⁵ cells

Crosslinking of epigenetic chromosomal interactions present at the chromosomal locus is described herein. This may be performed before cell lysis takes place. Cell lysis may be performed for 3 to 7 minutes, such as 4 to 6 or about 5 minutes. In some aspects, cell lysis is performed for at least 5 minutes and for less than 10 minutes.

Digesting DNA with a restriction enzyme is described herein. Typically, DNA restriction is performed at about 55° C. to about 70° C., such as for about 65° C., for a period of about 10 to 30 minutes, such as about 20 minutes.

Preferably a frequent cutter restriction enzyme is used which results in fragments of ligated DNA with an average fragment size up to 4000 base pair. Optionally the restriction enzyme results in fragments of ligated DNA have an average fragment size of about 200 to 300 base pairs, such as about 256 base pairs. In one aspect, the typical fragment size is from 200 base pairs to 4,000 base pairs, such as 400 to 2,000 or 500 to 1,000 base pairs.

In one aspect of the EpiSwitch process a DNA precipitation step is not performed between the DNA restriction digest step and the DNA ligation step.

DNA ligation is described herein. Typically the DNA ligation is performed for 5 to 30 minutes, such as about 10 minutes.

The protein in the sample may be digested enzymatically, for example using a proteinase, optionally Proteinase K. The protein may be enzymatically digested for a period of about 30 minutes to 1 hour, for example for about 45 minutes. In one aspect after digestion of the protein, for example Proteinase K digestion, there is no cross-link reversal or phenol DNA extraction step.

In one aspect PCR detection is capable of detecting a single copy of the ligated nucleic acid, preferably with a binary read-out for presence/absence of the ligated nucleic acid.

FIG. 15 shows a preferred process of detecting chromosome interactions.

### Processes and Uses of the Invention

The process of the invention can be described in different ways. It can be described as a process of making a ligated nucleic acid comprising (i) in vitro cross-linking of chromosome regions which have come together in a chromosome interaction; (ii) subjecting said cross-linked DNA to cutting or restriction digestion cleavage; and (iii) ligating said cross-linked cleaved DNA ends to form a ligated nucleic acid, wherein detection of the ligated nucleic acid may be used to determine the chromosome state at a locus, and wherein preferably:
the locus may be any of the loci or regions mentioned in Table 1, and/or
wherein the chromosomal interaction may be any of the chromosome interactions mentioned herein or corresponding to any of the probes disclosed in Table 1, and/or
wherein the ligated product may have or comprise (i) sequence which is the same as or homologous to any of the probe sequences disclosed in Table 1; or (ii) sequence which is complementary to (ii).

The process of the invention can be described as a process for detecting chromosome states which represent different subgroups in a population comprising determining whether a chromosome interaction is present or absent within a defined epigenetically active region of the genome, wherein preferably:
the subgroup is defined by presence or stage of muscular atrophy, and/or
the chromosome state may be at any locus or region mentioned in Table 1; and/or
the chromosome interaction may be any of those mentioned in Table 1 or corresponding to any of the probes disclosed in those tables.

### Use of the Process of the Invention to Identify New Treatments

Knowledge of chromosome interactions can be used to identify new treatments for conditions. The invention provides processes and uses of chromosome interactions defined herein to identify or design new therapeutic agents, for example relating to therapy of muscular atrophy or related sub-conditions.

### Homologues

Homologues of polynucleotide/nucleic acid (e.g. DNA) sequences are referred to herein. Such homologues typically have at least 70% homology, preferably at least 80%, at least 85%, at least 90%, at least 95%, at least 97%, at least 98% or at least 99% homology, for example over a region of at least 10, 15, 20, 30, 100 or more contiguous nucleotides, or across the portion of the nucleic acid which is from the region of the chromosome involved in the chromosome interaction. The homology may be calculated on the basis of nucleotide identity (sometimes referred to as “hard homology”).

Therefore, in a particular aspect, homologues of polynucleotide/nucleic acid (e.g. DNA) sequences are referred to herein by reference to percentage sequence identity. Typically such homologues have at least 70% sequence identity, preferably at least 80%, at least 85%, at least 90%, at least 95%, at least 97%, at least 98% or at least 99% sequence identity, for example over a region of at least 10, 15, 20, 30, 100 or more contiguous nucleotides, or across the portion of the nucleic acid which is from the region of the chromosome involved in the chromosome interaction. The homologues may have at least 70% sequence identity, preferably at least 80%, at least 85%, at least 90%, at least 95%, at least 97%, at least 98% or at least 99% sequence identity across the entire probe, primer or primer pair.

For example the UWGCG Package provides the BESTFIT program which can be used to calculate homology and/or % sequence identity (for example used on its default settings) (Devereux et al (1984) Nucleic Acids Research 12, p387-395). The PILEUP and BLAST algorithms can be used to calculate homology and/or % sequence identity and/or line up sequences (such as identifying equivalent or corresponding sequences (typically on their default settings)), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W5 T and X determine the sensitivity and speed of the alignment. The BLAST program uses as defaults a word length (W) of 11 , the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by 1, 2, 3, 4 or more bases, such as less than 10, 15 or 20 bases (which may be substitutions, deletions or insertions of nucleotides). These changes may be measured across any of the regions mentioned above in relation to calculating homology and/or % percentage sequence identity.

Homology of a ‘pair of primers’ can be calculated, for example, by considering the two sequences as a single sequence (as if the two sequences are joined together) for the purpose of then comparing against the another primer pair which again is considered as a single sequence.

### EpiSwitch™ Technology

The EpiSwitch™ Technology also relates to the use of microarray EpiSwitch™ marker data in the detection of epigenetic chromosome conformation signatures specific for phenotypes. Aspects such as EpiSwitch™ which utilise ligated nucleic acids in the manner described herein have several advantages. They have a low level of stochastic noise, for example because the nucleic acid sequences from the first set of nucleic acids of the present invention either hybridise or fail to hybridise with the second set of nucleic acids. This provides a binary result permitting a relatively simple way to measure a complex mechanism at the epigenetic level. EpiSwitch™ technology also has fast processing time and low cost. In one aspect the processing time is 3 hours to 6 hours.

### Arrays

All nucleic acids disclosed herein may be bound to an array, and in one aspect there are at least 15,000, 45,000, 100,000 or 250,000 different nucleic acids bound to the array, which preferably represent at least 300, 900, 2000 or 5000 loci. In one aspect one, or more, or all of the different populations of nucleic acids are bound to more than one distinct region of the array, in effect repeated on the array allowing for error detection. The array may be based on an Agilent SurePrint G3 Custom CGH microarray platform. Detection of binding of first nucleic acids to the array may be performed by a dual colour system.

### The Threshold of Detection

The markers which are disclosed herein have been found to be ‘disseminating markers’ capable of determining muscular atrophy status or subgroup. In practical terms it means that these markers are prevalent across groups of patients when compared to controls (as is shown by the FC value, for example). On average, as an example, an individual marker may typically be present in 80% of patients tested and in 10% of controls tested. Thus in one aspect of the method an individual is deemed to be part of the relevant muscular atrophy subgroup if least 80% of the markers that are tested for that subgroup are present in the individual and/or if at least 80% of the markers that are tested which are related to the control (non-muscular atrophy group) are absent from the individual. Typically presence/absence of at least 8 markers out of 10 compared to the ‘ideal’ result shown in the table can be used to assign the individual to a subgroup.

### Therapeutic Agents and Treatments

This section is relevant both to:
therapeutic agents which are given to individuals selected by the process of the invention, and
therapeutic agents which are selected based on the results of the process of the invention.

The invention provides therapeutic agents for use in preventing or treating a disease condition in certain individuals, for example those identified by a process of the invention. This may comprise administering to an individual in need a therapeutically effective amount of the agent. The invention provides use of the agent in the manufacture of a medicament to prevent or treat a condition in certain individuals. The disease or condition may be muscular atrophy, any type of muscular atrophy sub-condition or a stage of muscular atrophy.

The formulation of the agent will depend upon the nature of the agent. The agent will be provided in the form of a pharmaceutical composition containing the agent and a pharmaceutically acceptable carrier or diluent. Suitable carriers and diluents include isotonic saline solutions, for example phosphate-buffered saline. Typical oral dosage compositions include tablets, capsules, liquid solutions and liquid suspensions. The agent may be formulated for parenteral, intravenous, intramuscular, subcutaneous, transdermal or oral administration.

The dose of an agent may be determined according to various parameters, especially according to the substance used; the age, weight and condition of the individual to be treated; the route of administration; and the required regimen. A physician will be able to determine the required route of administration and dosage for any particular agent. A suitable dose may however be from 0.1 to 100 mg/kg body weight such as 1 to 40 mg/kg body weight, for example, to be taken from 1 to 3 times daily.

The therapeutic agent may be any such agent disclosed herein, or may target any ‘target’ disclosed herein, including any protein or gene disclosed herein in any table. It is understood that any agent that is disclosed in a combination should be seen as also disclosed for administration individually.

Therapeutic agents and treatments which can be used in the invention include physiotherapy, rehabilitation, agents that treat muscle tremors, agents that treat muscle cramps, hormone therapy, anti-testosterone leuprorelin.

### Properties of Nucleic Acids of the Invention

The invention relates to certain nucleic acids, such as the ligated nucleic acids which are described herein as being used or generated in the process of the invention. These may be the same as, or have any of the properties of, the first and second nucleic acids mentioned herein. The nucleic acids of the invention typically comprise two portions each comprising sequence from one of the two regions of the chromosome which come together in the chromosome interaction. Typically each portion is at least 8, 10, 15, 20, 30 or 40 nucleotides in length, for example 10 to 40 nucleotides in length. Preferred nucleic acids comprise sequence from any of the genes mentioned in any of the tables. Typically preferred nucleic acids comprise the specific probe sequences mentioned in Table 1; or fragments and/or homologues of such sequences.

Preferably the nucleic acids are DNA. It is understood that where a specific sequence is provided the invention may use the complementary sequence as required in the particular aspect. Preferably the nucleic acids are DNA. It is understood that where a specific sequence is provided the invention may use the complementary sequence as required in the particular aspect.

The primers shown in Table 1 may also be used in the invention as mentioned herein. In one aspect primers are used which comprise any of: the sequences shown in Table 1; or fragments and/or homologues of any sequence shown in Table 1.

### Screening to Identify Relevant Chromosome Interactions

In one aspect one or more of the chromosome interactions which are typed have been identified by a process of determining which chromosomal interactions are relevant to a chromosome state corresponding to a muscular atrophy subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to the subgroup.

The second set of nucleic acid sequences has the function of being a set of index sequences, and is essentially a set of nucleic acid sequences which are suitable for identifying subgroup specific sequence. They can represents the ‘background’ chromosomal interactions and might be selected in some way or be unselected. They are in general a subset of all possible chromosomal interactions.

The second set of nucleic acids may be derived by any suitable process. They can be derived computationally or they may be based on chromosome interaction in individuals. They typically represent a larger population group than the first set of nucleic acids. In one particular aspect, the second set of nucleic acids represents all possible epigenetic chromosomal interactions in a specific set of genes. In another particular aspect, the second set of nucleic acids represents a large proportion of all possible epigenetic chromosomal interactions present in a population described herein. In one particular aspect, the second set of nucleic acids represents at least 50% or at least 80% of epigenetic chromosomal interactions in at least 20, 50, 100 or 500 genes, for example in 20 to 100 or 50 to 500 genes.

The second set of nucleic acids typically represents at least 100 possible epigenetic chromosome interactions which modify, regulate or in any way mediate a phenotype in population. The second set of nucleic acids may represent chromosome interactions that affect a disease state (typically relevant to diagnosis or prognosis) in a species. The second set of nucleic acids typically comprises sequences representing epigenetic interactions both relevant and not relevant to a prognosis subgroup.

In one particular aspect the second set of nucleic acids derive at least partially from naturally occurring sequences in a population, and are typically obtained by in silico processes. Said nucleic acids may further comprise single or multiple mutations in comparison to a corresponding portion of nucleic acids present in the naturally occurring nucleic acids. Mutations include deletions, substitutions and/or additions of one or more nucleotide base pairs. In one particular aspect, the second set of nucleic acids may comprise sequence representing a homologue and/or orthologue with at least 70% sequence identity to the corresponding portion of nucleic acids present in the naturally occurring species. In another particular aspect, at least 80% sequence identity or at least 90% sequence identity to the corresponding portion of nucleic acids present in the naturally occurring species is provided.

### Properties of the Second Set of Nucleic Acids

In one particular aspect, there are at least 100 different nucleic acid sequences in the second set of nucleic acids, preferably at least 1000, 2000 or 5000 different nucleic acids sequences, with up to 100,000, 1,000,000 or 10,000,000 different nucleic acid sequences. A typical number would be 100 to 1,000,000, such as 1,000 to 100,000 different nucleic acids sequences. All or at least 90% or at least 50% or these would correspond to different chromosomal interactions.

In one particular aspect, the second set of nucleic acids represent chromosome interactions in at least 20 different loci or genes, preferably at least 40 different loci or genes, and more preferably at least 100, at least 500, at least 1000 or at least 5000 different loci or genes, such as 100 to 10,000 different loci or genes. The lengths of the second set of nucleic acids are suitable for them to specifically hybridise according to Watson Crick base pairing to the first set of nucleic acids to allow identification of chromosome interactions specific to subgroups. Typically the second set of nucleic acids will comprise two portions corresponding in sequence to the two chromosome regions which come together in the chromosome interaction. The second set of nucleic acids typically comprise nucleic acid sequences which are at least 10, preferably 20, and preferably still 30 bases (nucleotides) in length. In another aspect, the nucleic acid sequences may be at the most 500, preferably at most 100, and preferably still at most 50 base pairs in length. In a preferred aspect, the second set of nucleic acids comprises nucleic acid sequences of between 17 and 25 base pairs. In one aspect at least 100, 80% or 50% of the second set of nucleic acid sequences have lengths as described above. Preferably the different nucleic acids do not have any overlapping sequences, for example at least 100%, 90%, 80% or 50% of the nucleic acids do not have the same sequence over at least 5 contiguous nucleotides.

Given that the second set of nucleic acids acts as an ‘index’ then the same set of second nucleic acids may be used with different sets of first nucleic acids which represent subgroups for different characteristics, i.e. the second set of nucleic acids may represent a ‘universal’ collection of nucleic acids which can be used to identify chromosome interactions relevant to different characteristics.

### The First Set of Nucleic Acids (Screening for Relevant Chromosome Interactions)

The first set of nucleic acids are typically from subgroups relevant to muscular atrophy. The first nucleic acids may have any of the characteristics and properties of the second set of nucleic acids mentioned herein. The first set of nucleic acids is normally derived from samples from the individuals which have undergone treatment and processing as described herein, particularly the EpiSwitch™ cross-linking and cleaving steps. Typically the first set of nucleic acids represents all or at least 80% or 50% of the chromosome interactions present in the samples taken from the individuals.

Typically, the first set of nucleic acids represents a smaller population of chromosome interactions across the loci or genes represented by the second set of nucleic acids in comparison to the chromosome interactions represented by second set of nucleic acids, i.e. the second set of nucleic acids is representing a background or index set of interactions in a defined set of loci or genes.

### Library of Nucleic Acids

Any of the types of nucleic acid populations mentioned herein may be present in the form of a library comprising at least 200, at least 500, at least 1000, at least 5000 or at least 10000 different nucleic acids of that type, such as ‘first’ or ‘second’ nucleic acids. Such a library may be in the form of being bound to an array. The library may comprise some or all of the probes or primer pairs shown in Table 1A or 1B. The library may comprise all of the probe sequence from any of the tables disclosed herein.

### Hybridisation

The invention typically requires a means for allowing wholly or partially complementary nucleic acid sequences to hybridise, for example in the method of the invention or between the first set of nucleic acids and the second set of nucleic acids to hybridise. In one aspect all of the first set of nucleic acids is contacted with all of the second set of nucleic acids in a single assay, i.e. in a single hybridisation step. However any suitable assay can be used.

### Labelled Nucleic Acids and Pattern of Hybridisation

The nucleic acids mentioned herein may be labelled, preferably using an independent label such as a fluorophore (fluorescent molecule) or radioactive label which assists detection of successful hybridisation. Certain labels can be detected under UV light. The pattern of hybridisation, for example on an array described herein, represents differences in epigenetic chromosome interactions between the two subgroups, and thus provides a process of comparing epigenetic chromosome interactions and determination of which epigenetic chromosome interactions are specific to a subgroup in the population of the present invention.

The term ‘pattern of hybridisation’ broadly covers the presence and absence of hybridisation, for example between the first and second set of nucleic acids, i.e. which specific nucleic acids from the first set hybridise to which specific nucleic acids from the second set, and so it not limited to any particular assay or technique, or the need to have a surface or array on which a ‘pattern’ can be detected.

### Forms of the Substance Mentioned Herein

Any of the substances, such as nucleic acids or therapeutic agents, mentioned herein may be in purified or isolated form. They may be in a form which is different from that found in nature, for example they may be present in combination with other substance with which they do not occur in nature. The nucleic acids (including portions of sequences defined herein) may have sequences which are different to those found in nature, for example having at least 1, 2, 3, 4 or more nucleotide changes in the sequence as described in the section on homology. The nucleic acids may have heterologous sequence at the 5′ or 3′ end. The nucleic acids may be chemically different from those found in nature, for example they may be modified in some way, but preferably are still capable of Watson-Crick base pairing. Where appropriate the nucleic acids will be provided in double stranded or single stranded form. The invention provides all of the specific nucleic acid sequences mentioned herein in single or double stranded form, and thus includes the complementary strand to any sequence which is disclosed.

The invention provides a kit for carrying out any process of the invention, including detection of a chromosomal interaction relating to prognosis. Such a kit can include a specific binding agent capable of detecting the relevant chromosomal interaction, such as agents capable of detecting a ligated nucleic acid generated by processes of the invention. Preferred agents present in the kit include probes capable of hybridising to the ligated nucleic acid or primer pairs, for example as described herein, capable of amplifying the ligated nucleic acid in a PCR reaction. A kit of the invention may comprise means to detect a panel of markers, such as any number of combination of markers disclosed herein.

The invention provides use of a reagent for preparing kit for carrying out the process of the invention. Such a reagent may be any suitable substance mentioned herein, such as the agents which are capable of detection of products of detection processes, including reagents which are any of the probes or primers mentioned herein. The invention provides use of the reagent in the process of the invention. The invention provides use of the reagent in the preparing of a means for carrying out the invention.

The invention provides a device that is capable of detecting the relevant chromosome interactions. The device preferably comprises any specific binding agents, probe or primer pair capable of detecting the chromosome interaction, such as any such agent, probe or primer pair described herein.

The invention provides use of detection of chromosome interactions as defined herein (for example by number or specific combination) to detect muscular atrophy or any characteristic of muscular atrophy, for example as defined herein. The invention provides use of a reagent (for example a probe, primer, label, device or array) in any method of the invention.

### Detection Process

In one aspect quantitative detection of the ligated sequence which is relevant to a chromosome interaction is carried out using a probe which is detectable upon activation during a PCR reaction, wherein said ligated sequence comprises sequences from two chromosome regions that come together in an epigenetic chromosome interaction, wherein said process comprises contacting the ligated sequence with the probe during a PCR reaction, and detecting the extent of activation of the probe, and wherein said probe binds the ligation site. The process typically allows particular interactions to be detected in a MIQE compliant manner using a dual labelled fluorescent hydrolysis probe.

The probe is generally labelled with a detectable label which has an inactive and active state, so that it is only detected when activated. The extent of activation will be related to the extent of template (ligation product) present in the PCR reaction. Detection may be carried out during all or some of the PCR, for example for at least 50% or 80% of the cycles of the PCR.

The probe can comprise a fluorophore covalently attached to one end of the oligonucleotide, and a quencher attached to the other end of the nucleotide, so that the fluorescence of the fluorophore is quenched by the quencher. In one aspect the fluorophore is attached to the 5′end of the oligonucleotide, and the quencher is covalently attached to the 3′ end of the oligonucleotide. Fluorophores that can be used in the process of the invention include FAM, TET, JOE, Yakima Yellow, HEX, Cyanine3, ATTO 550, TAMRA, ROX, Texas Red, Cyanine 3.5, LC610, LC 640, ATTO 647N, Cyanine 5, Cyanine 5.5 and ATTO 680. Quenchers that can be used with the appropriate fluorophore include TAM, BHQ1, DAB, Eclip, BHQ2 and BBQ650, optionally wherein said fluorophore is selected from HEX, Texas Red and FAM. Preferred combinations of fluorophore and quencher include FAM with BHQ1 and Texas Red with BHQ2.

### Use of the Probe in a qPCR Assay

Hydrolysis probes of the invention are typically temperature gradient optimised with concentration matched negative controls. Preferably single-step PCR reactions are optimized. More preferably a standard curve is calculated. An advantage of using a specific probe that binds across the junction of the ligated sequence is that specificity for the ligated sequence can be achieved without using a nested PCR approach. The processes described herein allow accurate and precise quantification of low copy number targets. The target ligated sequence can be purified, for example gel-purified, prior to temperature gradient optimization. The target ligated sequence can be sequenced. Preferably PCR reactions are performed using about 10 ng, or 5 to 15 ng, or 10 to 20 ng, or 10 to 50 ng, or 10 to 200 ng template DNA. Forward and reverse primers are designed such that one primer binds to the sequence of one of the chromosome regions represented in the ligated DNA sequence, and the other primer binds to other chromosome region represented in the ligated DNA sequence, for example, by being complementary to the sequence.

### Choice of Ligated DNA Target

The invention includes selecting primers and a probe for use in a PCR process as defined herein comprising selecting primers based on their ability to bind and amplify the ligated sequence and selecting the probe sequence based properties of the target sequence to which it will bind, in particular the curvature of the target sequence.

Probes are typically designed/chosen to bind to ligated sequences which are juxtaposed restriction fragments spanning the restriction site. In one aspect of the invention, the predicted curvature of possible ligated sequences relevant to a particular chromosome interaction is calculated, for example using a specific algorithm referenced herein. The curvature can be expressed as degrees per helical turn, e.g. 10.5° per helical turn. Ligated sequences are selected for targeting where the ligated sequence has a curvature propensity peak score of at least 5° per helical turn, typically at least 10°, 15° or 20° per helical turn, for example 5° to 20° per helical turn. Preferably the curvature propensity score per helical turn is calculated for at least 20, 50, 100, 200 or 400 bases, such as for 20 to 400 bases upstream and/or downstream of the ligation site. Thus in one aspect the target sequence in the ligated product has any of these levels of curvature. Target sequences can also be chosen based on lowest thermodynamic structure free energy.

### Particular Aspects

In one aspect only intrachromosomal interactions are typed/detected, and no extrachromosomal interactions (between different chromosomes) are typed/detected.

In particular aspects certain chromosome interactions are not typed, for example any specific interaction mentioned herein (for example as defined by any probe or primer pair mentioned herein). In some aspects chromosome interactions are not typed in any of the genes relevant to chromosome interactions mentioned herein.

The data provided herein shows that the markers are ‘disseminating’ ones able to differentiate cases and non-cases for the relevant disease situation. Therefore when carrying out the invention the skilled person will be able to determine by detection of the interactions which subgroup the individual is in. In one embodiment a threshold value of detection of at least 70% of the tested markers in the form they are associated with the relevant disease situation (either by absence or presence) may be used to determine whether the individual is in the relevant subgroup.

In one embodiment the process of the invention does not detect the presence of Huntington's disease, and is not carried out for the purpose of detection of Huntington's disease. In one embodiment the process of the invention is not carried out on an individual who is suspected of having Huntington's disease or who has symptoms of Huntington's disease.

### Use of a Classifier

The method of the invention may include analysis of the chromosome interactions identified in the individual, for example using a classifier, which may increase performance, such as sensitivity or specificity. The classifier is typically one that has been ‘trained’ on samples from the population and such training may assist the classifier to detect any subgroup mentioned herein.

### Screening Process

The invention provides a process of determining which chromosomal interactions are relevant to a chromosome state corresponding to an prognosis subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to an prognosis subgroup. The subgroup may be any of the specific subgroups defined herein, for example with reference to particular conditions or therapies.

### Publications

The contents of all publications mentioned herein are incorporated by reference into the present specification and may be used to further define the features relevant to the invention. The contents of the priority document, UK Patent Application No. 2016176.6 filed 12 Oct. 2021, is also incorporated herein by reference.

### Techniques Used to Identify the Specific Relevant Chromosome Interactions

The EpiSwitch™ platform technology detects epigenetic regulatory signatures of regulatory changes between normal and abnormal conditions at loci. The EpiSwitch™ platform identifies and monitors the fundamental epigenetic level of gene regulation associated with regulatory high order structures of human chromosomes also known as chromosome conformation signatures. Chromosome signatures are a distinct primary step in a cascade of gene deregulation. They are high order biomarkers with a unique set of advantages against biomarker platforms that utilize late epigenetic and gene expression biomarkers, such as DNA methylation and RNA profiling.

### EpiSwitch™ Array Assay

The custom EpiSwitch™ array-screening platforms come in 4 densities of, 15K, 45K, 100K, and 250K unique chromosome conformations, each chimeric fragment is repeated on the arrays 4 times, making the effective densities 60K, 180K, 400K and 1 Million respectively.

### Custom Designed EpiSwitch™ Arrays

The 15K EpiSwitch™ array can screen the whole genome including around 300 loci interrogated with the EpiSwitch™ Biomarker discovery technology. The EpiSwitch™ array is built on the Agilent SurePrint G3 Custom CGH microarray platform; this technology offers 4 densities, 60K, 180K, 400K and 1 Million probes. The density per array is reduced to 15K, 45K, 100K and 250K as each EpiSwitch™ probe is presented as a quadruplicate, thus allowing for statistical evaluation of the reproducibility. The average number of potential EpiSwitch™ markers interrogated per genetic loci is 50, as such the numbers of loci that can be investigated are 300, 900, 2000, and 5000.

### EpiSwitch™ Custom Array Pipeline

The EpiSwitch™ array is a dual colour system with one set of samples, after EpiSwitch™ library generation, labelled in Cy5 and the other of sample (controls) to be compared/analyzed labelled in Cy3. The arrays are scanned using the Agilent SureScan Scanner and the resultant features extracted using the Agilent Feature Extraction software. The data is then processed using the EpiSwitch™ array processing scripts in R. The arrays are processed using standard dual colour packages in Bioconductor in R: Limma*. The normalisation of the arrays is done using the normalisedWithinArrays function in Limma* and this is done to the on chip Agilent positive controls and EpiSwitch™ positive controls. The data is filtered based on the Agilent Flag calls, the Agilent control probes are removed and the technical replicate probes are averaged, in order for them to be analysed using Limma*. The probes are modelled based on their difference between the 2 scenarios being compared and then corrected by using False Discovery Rate. Probes with Coefficient of Variation (CV)<=30% that are <=−1.1 or =>1.1 and pass the p<=0.1 FDR p-value are used for further screening. To reduce the probe set further Multiple Factor Analysis is performed using the FactorMineR package in R.

* Note: LIMMA is Linear Models and Empirical Bayes Processes for Assessing Differential Expression in Microarray Experiments. Limma is an R package for the analysis of gene expression data arising from microarray or RNA-Seq.

The pool of probes is initially selected based on adjusted p-value, FC and CV<30% (arbitrary cut off point) parameters for final picking. Further analyses and the final list are drawn based only on the first two parameters (adj. p-value; FC).

### Statistical Pipeline

EpiSwitch™ screening arrays are processed using the EpiSwitch™ Analytical Package in R in order to select high value EpiSwitch™ markers for translation on to the EpiSwitch™ PCR platform.

Step 1

Probes are selected based on their corrected p-value (False Discovery Rate, FDR), which is the product of a modified linear regression model. Probes below p-value<=0.1 are selected and then further reduced by their Epigenetic ratio (ER), probes ER have to be <=−1.1 or =>1.1 in order to be selected for further analysis. The last filter is a coefficient of variation (CV), probes have to be below <=0.3.

Step 2

The top 40 markers from the statistical lists are selected based on their ER for selection as markers for PCR translation. The top 20 markers with the highest negative ER load and the top 20 markers with the highest positive ER load form the list.

Step 3

The resultant markers from step 1, the statistically significant probes form the bases of enrichment analysis using hypergeometric enrichment (HE). This analysis enables marker reduction from the significant probe list, and along with the markers from step 2 forms the list of probes translated on to the EpiSwitch™ PCR platform.

The statistical probes are processed by HE to determine which genetic locations have an enrichment of statistically significant probes, indicating which genetic locations are hubs of epigenetic difference.

The most significant enriched loci based on a corrected p-value are selected for probe list generation. Genetic locations below p-value of 0.3 or 0.2 are selected. The statistical probes mapping to these genetic locations, with the markers from step 2, form the high value markers for EpiSwitch™ PCR translation.

### Array Design and Processing

Array Design

Genetic loci are processed using the SII software (currently v3.2) to:
Pull out the sequence of the genome at these specific genetic loci (gene sequence with 50 kb upstream and 20 kb downstream)
Define the probability that a sequence within this region is involved in CCs
Cut the sequence using a specific RE
Determine which restriction fragments are likely to interact in a certain orientation
Rank the likelihood of different CCs interacting together.
Determine array size and therefore number of probe positions available (x)
Pull out x/4 interactions.
For each interaction define sequence of 30 bp to restriction site from part 1 and 30 bp to restriction site of part 2. Check those regions aren't repeats, if so exclude and take next interaction down on the list. Join both 30 bp to define probe.
Create list of x/4 probes plus defined control probes and replicate 4 times to create list to be created on array
Upload list of probes onto Agilent Sure design website for custom CGH array.
Use probe group to design Agilent custom CGH array.

Array Processing
Process samples using EpiSwitch™ Standard Operating Procedure (SOP) for template production.
Clean up with ethanol precipitation by array processing laboratory.
Process samples as per Agilent SureTag complete DNA labelling kit—Agilent Oligonucleotide Array-based CGH for Genomic DNA Analysis Enzymatic labelling for Blood, Cells or Tissues
Scan using Agilent C Scanner using Agilent feature extraction software.

EpiSwitch™ biomarker signatures demonstrate high robustness, sensitivity and specificity in the stratification of complex disease phenotypes. This technology takes advantage of the latest breakthroughs in the science of epigenetics, monitoring and evaluation of chromosome conformation signatures as a highly informative class of epigenetic biomarkers. Current research methods deployed in academic environment require from 3 to 7 days for biochemical processing of cellular material in order to detect CCSs. Those procedures have limited sensitivity, and reproducibility; and furthermore, do not have the benefit of the targeted insight provided by the EpiSwitch™ Analytical Package at the design stage.

EpiSwitch™ Array in Silico Marker Identification

CCS sites across the genome are directly evaluated by the EpiSwitch™ Array on clinical samples from testing cohorts for identification of all relevant stratifying lead biomarkers. The EpiSwitch™ Array platform is used for marker identification due to its high-throughput capacity, and its ability to screen large numbers of loci rapidly. The array used was the Agilent custom-CGH array, which allows markers identified through the in silico software to be interrogated.

EpiSwitch™ PCR

Potential markers identified by EpiSwitch™ Array are then validated either by EpiSwitch™ PCR or DNA sequencers (i.e. Roche 454, Nanopore MinION, etc.). The top PCR markers which are statistically significant and display the best reproducibility are selected for further reduction into the final EpiSwitch™ Signature Set, and validated on an independent cohort of samples. EpiSwitch™ PCR can be performed by a trained technician following a standardised operating procedure protocol established. All protocols and manufacture of reagents are performed under ISO 13485 and 9001 accreditation to ensure the quality of the work and the ability to transfer the protocols. EpiSwitch™ PCR and EpiSwitch™ Array biomarker platforms are compatible with analysis of both whole blood and cell lines. The tests are sensitive enough to detect abnormalities in very low copy numbers using small volumes of blood.

The invention is illustrated by the following:

### Example 1

Spinal and bulbar muscular atrophy (SBMA) is an exemplifying model muscle atrophy disease that can be used to identify the chromosome interactions relevant to muscle atrophy. SBMA is a rare disease with predominant manifestation in males (2.6:100,000) associated with genetic mutations in androgen receptor (AR) gene and inherited in X-linked recessive manner. Modulation of endocrine, neurological and muscular regulatory networks associated with impairment of AR gene lead to significant pathophysiology and extreme disability. Interestingly, in homozygous females with AR impairment the symptoms are very mild, indicating the significance of the regulatory context in compensating the defect. As part of its pathophysiology, SBMA leads to muscle atrophy, largely due to lower motor neuron degeneration and lack of adequate stimulation. The disease has no treatment and there are limited prognostic insights.

Spinal muscular atrophy is a recognized model disease for the better understanding of spinal sarcopenia, motor neuron loss in sarcopenia, muscle atrophy, muscle wasting phenomenon as a result of ageing (sarcopenia) and as a result of underlying pathological signalling (cachexia, as particularly often observed in cancer) and for understanding shared mechanisms of muscle wasting with cancer.

Here we have analysed chromosome interactions of SBMA patients in comparison to healthy cohorts. We have done whole genome screening across over 900,000 chromosome conformations covering all annotated genes and ncRNA across the genome.

A whole blood sample was provided from a United Kingdom cohort consisting of 12 SBMA patients and 7 age matched controls. The 12 SBMA patients were split between early and late disease onset patients, and are described in the table below.

| | | | Age 1st | | |
|---|---|---|---|---|---|
| | | | medical | Disease | |
| Code | YOB | CAG size | encounter | duration | SBMAFRS |
| UCH0206 | 1939 | 40 | 66 | 21 | 32 |
| UCH0081 | 1943 | 37 | 64 | 13 | 43 |
| UCH0307 | 1950 | | 57 | 13 | 26 |
| UCH0213 | 1950 | 39 | 60 | 10 | 43 |
| UCH0190 | 1951 | 45 | 63 | 6 | 47 |
| UCH0210 | 1955 | | 59 | 6 | 42 |
| UCH0309 | 1956 | | 52 | 12 | |
| UCH0292 | 1958 | | 57 | 5 | 40 |
| UCH0189 | 1967 | 45 | 42 | 11 | 34 |
| UCH0179 | 1969 | 46 | 46 | 5 | 50 |
| UCH0312 | 1969 | | 48 | 3 | 50 |
| UCH0169 | 1974 | 49 | 35 | 11 | 36 |

In case of bulbar and spinal muscular atrophy genetic confirmation of CAG repeats in AR gene acts a accepted diagnostic readout. One challenge is to have a readout that confirms the severity of disease manifestation, especially as genetic disorder in many patients is compensated by epigenetic network. That is why the present work is based on samples representing both early and late stages of muscular atrophy allowing prognosis to be determined, in particular in respect of severity in clinical manifestation of the disease, as well as a general diagnosis.

We have identified top 200 disseminating SBMA-specific CCS, with a False Discovery Rate FDR<0.05. Analysing genetic loci affected by conditional CCS, we performed pathway analysis and demonstrated heavy involvement in SBMA specific profiling of Keratinization (24 genetic loci affected) and Olfactory Transduction (31 genetic loci affected). Importantly, the same analysis demonstrated heavy involvement of T Cell Receptor Signalling Pathway (12 genes affected) and L1CAM Interactions (8 genes affected, L1CAM is a neuronal cell adhesion molecule with a strong implication in cell migration, adhesion, neurite outgrowth, myelination and neuronal differentiation). Most importantly, this analysis reveals an overlap between sites implicated in SBMA regulation and PD-1 checkpoint network implicated in cancer immuno-therapy. This is a first molecular evidence for SBMA and cancer overlap, in the context of muscular atrophy being observed under both conditions. The results of the work are shown in the Figures and tables herein. The identified markers are suitable for detection on nested PCR platform.

Tables 1A and Table 1B show the markers that were identified by this work. They represent part of the 3D genomic regulatory control. There were distinct CCSs in the early phenotype compared to the late showing the CCSs change as the disease progresses and varies between phenotypes. The CCSs can be linked to each clinically defined subgroup to be used as a biomarker tool to predict outcome and progression in patients. The present work therefore provides both diagnostic and prognostic markers.

TABLE 1A

| No. | Probe | RP/Rsum | Rprank | FC:(class1/class2) |
|---|---|---|---|---|
| 1 | Hg38_2_151357305_151361853_151612320_151619007_RF | 87.63840883 | 1 | 2.283 |
| 2 | Hg38_13_41466343_41476518_41555919_41561354_RF | 288.7780393 | 2 | 2.007 |
| 3 | Hg38_20_23380285_23385557_23532159_23546814_RR | 534.5889718 | 4 | 1.878 |
| 4 | Hg38_1_84316044_84322127_84529225_84540074_RF | 663.6694959 | 10 | 1.87 |
| 5 | Hg38_1_209333922_209340142_209404224_209420680_FF | 446.7372857 | 3 | 1.87 |
| 6 | Hg38_5_177834278_177839098_178009945_178017869_FR | 662.5483965 | 9 | 1.832 |
| 7 | Hg38_11_77781318_77787851_77893153_77899469_FR | 732.8885954 | 12 | 1.798 |
| 8 | Hg38_8_48489316_48497835_48710460_48716484_RR | 755.6786775 | 13 | 1.794 |
| 9 | Hg38_8_33042243_33047675_33243637_33249598_FR | 930.5017332 | 18 | 1.788 |
| 10 | Hg38_11_7823466_7827013_7890344_7893739_FR | 1064.533822 | 22 | 1.776 |
| 11 | Hg38_2_242078290_242088090_242115056_242117195_RR | 626.2882701 | 6 | 1.776 |
| 12 | Hg38_4_64204635_64210880_64341978_64349763_RF | 1183.854495 | 27 | 1.771 |
| 13 | Hg38_9_4737518_4739216_4871409_4877222_FF | 855.3083509 | 15 | 1.767 |
| 14 | Hg38_6_144636121_144641243_144691336_144697440_RF | 578.9188967 | 5 | 1.767 |
| 15 | Hg38_22_47207724_47217624_47356169_47358600_FF | 665.6838613 | 11 | 1.765 |
| 16 | Hg38_16_31853018_31860663_31871565_31877473_FR | 836.8314653 | 14 | 1.764 |
| 17 | Hg38_10_6735455_6749237_6766912_6769302_RF | 648.2374782 | 8 | 1.764 |
| 18 | Hg38_14_26681688_26687550_26802034_26810557_FR | 1342.435576 | 33 | 1.76 |
| 19 | Hg38_11_63024617_63034264_63124362_63131307_RR | 639.5975048 | 7 | 1.758 |
| 20 | Hg38_11_55823665_55829626_55865366_55868750_FR | 866.3041714 | 17 | 1.726 |
| 21 | Hg38_2_20791146_20796987_20975471_20980938_RR | 857.2109811 | 16 | 1.716 |
| 22 | Hg38_1_114264796_114271415_114388190_114393574_FF | 1043.317342 | 20 | 1.702 |
| 23 | Hg38_3_150148357_150152202_150276483_150282205_RF | 1018.4725 | 19 | 1.692 |
| 24 | Hg38_3_3070802_3073954_3162690_3172869_FF | 2146.093548 | 53 | 1.684 |
| 25 | Hg38_2_228849719_228856896_228936338_228946445_RR | 2144.540397 | 52 | 1.682 |
| 26 | Hg38_X_126618971_126632078_126813931_126821389_RR | 1596.946758 | 38 | 1.682 |
| 27 | Hg38_17_3298600_3300356_3435567_3441497_RF | 1286.046191 | 32 | 1.682 |
| 28 | Hg38_6_141949457_141959891_142112622_142115111_FF | 1073.365868 | 23 | 1.681 |
| 29 | Hg38_2_221629362_221633765_221703220_221711044_FF | 1177.041857 | 26 | 1.679 |
| 30 | Hg38_18_8164110_8169146_8228637_8240170_RF | 1187.605744 | 28 | 1.675 |
| 31 | Hg38_X_83389647_83396643_83505067_83507528_RR | 1123.648127 | 25 | 1.67 |
| 32 | Hg38_2_38740614_38746415_38982199_38993639_FF | 1062.516677 | 21 | 1.666 |
| 33 | Hg38_2_169562452_169564303_169638099_169647209_FF | 1122.728889 | 24 | 1.664 |
| 34 | Hg38_X_51609399_51612166_51861901_51874841_RF | 1207.025498 | 29 | 1.658 |
| 35 | Hg38_1_96786343_96792340_96953170_96960029_RR | 1260.746357 | 30 | 1.657 |
| 36 | Hg38_13_76879860_76881474_77089881_77098142_FF | 1281.56965 | 31 | 1.653 |
| 37 | Hg38_12_10235418_10237590_10417499_10425564_RF | 1635.909028 | 39 | 1.647 |
| 38 | Hg38_3_151888504_151893566_151931687_151943298_RR | 1384.989976 | 35 | 1.643 |
| 39 | Hg38_8_81036889_81041061_81244260_81253029_RF | 2113.213401 | 50 | 1.641 |
| 40 | Hg38_21_25447238_25454612_25514175_25521235_RF | 2472.139306 | 68 | 1.639 |
| 41 | Hg38_12_39724269_39737447_39830409_39841867_RF | 2034.859484 | 48 | 1.637 |
| 42 | Hg38_2_2911175_2916426_3008403_3014033_FR | 1376.720339 | 34 | 1.635 |
| 43 | Hg38_5_39037737_39046956_39197630_39209276_FR | 2497.876062 | 69 | 1.63 |
| 44 | Hg38_2_127388555_127395831_127412534_127416129_RR | 3444.281703 | 126 | 1.623 |
| 45 | Hg38_21_14583254_14600479_14842273_14847749_RF | 1987.875145 | 45 | 1.622 |
| 46 | Hg38_22_32455270_32461039_32549159_32556587_FR | 1392.694528 | 36 | 1.621 |
| 47 | Hg38_13_77416226_77433379_77635636_77642621_RR | 2244.157946 | 57 | 1.617 |
| 48 | Hg38_X_66017310_66021076_66266598_66279138_RR | 1554.424806 | 37 | 1.616 |
| 49 | Hg38_9_119814503_119824066_119942887_119944563_RR | 2152.405439 | 54 | 1.61 |
| 50 | Hg38_15_96378302_96385292_96582969_96587891_FR | 1671.300538 | 40 | 1.607 |

TABLE 1A (part 1b)

| | | | | Probe sequence |
|---|---|---|---|---|
| No. | pfp | P.value | Type | 60 mer |
| 1 | 0 | 0 | SBMA | AATGTTGATAAGTATTAGACGACTGGTTTCGATTTTCTTTTAATCACTGCTAAATCTGCA |
| 2 | 0 | 0 | SBMA | TACTTACACTCAACAGGAGGGATATATTTCGAATCATTTTGGGGGCATAAACATAAAACC |
| 3 | 0 | 0 | SBMA | GTAAGAGAAACTGTTCTTTCTGTCAGCTTCGAACTACATTGAAAATCTCTTGTTTGTGTA |
| 4 | 0 | 0 | SBMA | TTTGCATTTATTTGATATATTCCCATTCTCGACCATTTTTTCAATTGAGTTATTAGTTTT |
| 5 | 0 | 0 | SBMA | CAAACATGTCTAACTCATGCAATTTTCTTCGAGTCTCCATTTGAAAAAAGAGGTTTTTTG |
| 6 | 0 | 0 | SBMA | GACTGAAAAAGTTAAGAAAACTTTCTACTCGATTAACACCTATTTTATTTGTGCTGCATT |
| 7 | 0 | 0 | SBMA | CTTCAGAACAGTAAAAAAAAAAAAAAGTTCGACTCCTATTTCTTCATCAAGCTTTCCATT |
| 8 | 0 | 0 | SBMA | GCTGACTTGATTTTGCTGGATAAGCTTTTCGACTTCCTTATACTAGTAAGAGACTTAAGC |
| 9 | 0 | 0 | SBMA | CAGAGAGGGACATTATATAATGAAATAGTCGAGGTCTGATAGCAACCATTTACCAAACTT |
| 10 | 0 | 0 | SBMA | GCTTCAAAAGGTCCTAGAAATAATCCACTCGAATCCTTGGCTTTTATTTTTCTTCAGGGA |
| 11 | 0 | 0 | SBMA | AGGAAAGAAACATAAGAATATTATTATTTCGAAGTCATTGAAGATTCACAGCAGGAGACA |
| 12 | 0 | 0 | SBMA | CCAGGCCTTGAAGAGATAATACAGGAATTCGAACCAATGTACATGTTACACATATTGATT |
| 13 | 0 | 0 | SBMA | ACAATCTCTGTTCTTTCTTTCTTTTTTTTCGAGAATCATGGCTTTAAAGACACAGAGATG |
| 14 | 0 | 0 | SBMA | AAATTTAAAGTTTCTTCTTACAAAGCATTCGAAGTGTAAGCCGTCATGATTTGTTTCATA |
| 15 | 0 | 0 | SBMA | AGTACTGAAATATTGTATTGCTACTATTTCGACACACACTTGTATTGAATAAAAGGCGTA |
| 16 | 0 | 0 | SBMA | GTCTTTTTAGAGATGAACAAAAATAAATTCGATCTGTCTGGTCAACATTCTGGGACAATG |
| 17 | 0 | 0 | SBMA | AAGTGTTAGAGAAAGAAGATTAAGCAATTCGACATTCCCACCAATAAGGTATAAGTATTT |
| 18 | 0 | 0 | SBMA | TCAAGTCACTGGCAGGAAGAACAAGCTCTCGAAAAAATACAGTGACATCTTTATTTGTCT |
| 19 | 0 | 0 | SBMA | TATCCATTTCCAGAAATTTTTCATTATCTCGATGTATGGATTAACTGTGTGATATTAACA |
| 20 | 0 | 0 | SBMA | ATATTGGGATTACTATGCAGATTACATTTCGAGTGTTTTAGGAAGTCATATAATGAACAA |
| 21 | 0 | 0 | SBMA | ATAAAAATGGAGTAGAAATCAGTGAAATTCGAATCATTCATGAAAGGAAAATCTGAGAAG |
| 22 | 0 | 0 | SBMA | GTGTCTGTTACTGATCCTTATAGCTTCTTCGAAGAAACAAAACTATCTTCAAAAATAAGT |
| 23 | 0 | 0 | SBMA | AGTTTGGTAAAGTTTCAGGATAAAAAAATCGAATTAGGAGTCTTGTAATTCCATGTTCAT |
| 24 | 0 | 0 | SBMA | TGTATTGGCATAAAGATAGACAAATAGATCGAGAAGAACAGGATTTTGGAATTGAGATAG |
| 25 | 0 | 0 | SBMA | TGTGAAACAAAGCCCGCTATTCACAGTCTCGACTTTGTTCAGAGTTCAGAATCAATATCT |
| 26 | 0 | 0 | SBMA | CAAATCTTTCCCAAAGTTAAAGTCCAAGTCGAACACAGCTACTACATTAAATATAACAAT |
| 27 | 0 | 0 | SBMA | CTCATCAGAATTGTTATAATAAAAAAGATCGAACAAGACTCTCTTTCTCTAGTAAGTCCT |
| 28 | 0 | 0 | SBMA | AGAAATAAAAGAAGGAGTCAAAATACCTTCGATTTGAGCAGAAAGTCCTGAAGGCACACA |
| 29 | 0 | 0 | SBMA | GATTGTATTACTTCAAAATAAAACTTGATCGAAGAAGTACAAGTCAAAGGCAGACAGCAC |
| 30 | 0 | 0 | SBMA | GAAAAAGAGAGAAACAAACTATTACTTATCGAGTTTTAGATTCTAAAATGTTGTTTAGTA |
| 31 | 0 | 0 | SBMA | AATCAAATGATAGAAACATAGACCAAAGTCGAGTTTAAGGCAAAATGCAGTCTATGAAAA |
| 32 | 0 | 0 | SBMA | TAATCTTTTACATTCTGCTTTAAGCACATCGATTTTTAAGATTCTCTCAGTTGGAAGAAA |
| 33 | 0 | 0 | SBMA | TTGAACACAGACAAGGTACTAGGTAATATCGATTTTAGCTTTTTTTTGACTTTTTCTCCA |
| 34 | 0 | 0 | SBMA | TTCTTGGTATGCCTGGTTTTTTTTTAATTCGATTTTAGCCCTAATCCCATTGTGCTGTAA |
| 35 | 0 | 0 | SBMA | TGACTTCTGCTAGGATACACCAGAAAATTCGAGTATGACAAAGAGCTTAAGCCAAGGTAG |
| 36 | 0 | 0 | SBMA | TACATGGCTTTATTGAAATAACATTCCATCGAGTTTTCTTTTCAAAGGAGATGGATCCAA |
| 37 | 0 | 0 | SBMA | CGTGACTGTATATCATGTCCTTTGTTAATCGAGACTGGTTAATTTAAAAAGAAAAGAGGT |
| 38 | 0 | 0 | SBMA | TAGAAACAAAAGTTACAGAAATAATCCCTCGATAACATTGTTTTAATTTTCTGAGAAAAA |
| 39 | 0 | 0 | SBMA | CAAGCAAAACAATGGCTTAAATTGTTTCTCGAACAGCTGAAATCCCTGATGTATGCAAAG |
| 40 | 0 | 0 | SBMA | TCCTACAAGAAATGCTAAAGAGAGTTATTCGATGAATTAATACATGCATTTTGAAATTGA |
| 41 | 0 | 0 | SBMA | TCTTAGGTTTGCAAACAGACCAAGTAAGTCGATATTGGTTGGGAACTGGTTAGTAATTAA |
| 42 | 0 | 0 | SBMA | CTAAAAGAAAACATAGGGGAAAAAGTCCTCGATTTTCATAACATTTATCTTAGCCTCAAA |
| 43 | 0 | 0 | SBMA | TTACCCTTTAAGTCAATGCCTCAAAAGTTCGAAAACTGCCTTTGATAATACCAGTGTCTT |
| 44 | 0 | 0 | SBMA | AATGTACACAACCAGTCATAACCAAGTATCGACTTTTCTCTTCCCAAGGGAGAGTTTACT |
| 45 | 0 | 0 | SBMA | TATAAACTGGAACTTTCAAATTTCATGCTCGATTACTTAAGGCATGCTATTATAAATAAA |
| 46 | 0 | 0 | SBMA | TTGTGTACTAGTAAACTGCAGGCATCCATCGAATGCTTTGCAGTCAAGAGAAGGCAATGA |
| 47 | 0 | 0 | SBMA | TAAACACGAGCTTTCTCAGCTGTGCTGCTCGAGATTTTTTAATTAATAGGATAGATGGAT |
| 48 | 0 | 0 | SBMA | CATGGTCACCAAATATGGCATGACAGAATCGACAGAGAAAGCTACGACGCAATAGGAGGT |
| 49 | 0 | 0 | SBMA | ATATTATTCTTTTGATTTTTCCAACCATTCGACGCTGTCCAGTGAAAATATAACACGAGC |
| 50 | 0 | 0 | SBMA | ATCTGTGAGATTTGCAGCTTCTTTTTGCTCGAATTGAGTTTATTTCAAAGATATTGATAA |

TABLE 1A
(part 1c)

| | Probe Location | | | | |
|---|---|---|---|---|---|
| No. | Chr | Start1 | End1 | Start2 | End2 |
| 1 | 2 | 151357307 | 151357336 | 151618976 | 151619005 |
| 2 | 13 | 41466345 | 41466374 | 41561323 | 41561352 |
| 3 | 20 | 23380287 | 23380316 | 23532161 | 23532190 |
| 4 | 1 | 84316046 | 84316075 | 84540043 | 84540072 |
| 5 | 1 | 209340111 | 209340140 | 209420649 | 209420678 |
| 6 | 5 | 177839067 | 177839096 | 178009947 | 178009976 |
| 7 | 11 | 77787820 | 77787849 | 77893155 | 77893184 |
| 8 | 8 | 48489318 | 48489347 | 48710462 | 48710491 |
| 9 | 8 | 33047644 | 33047673 | 33243639 | 33243668 |
| 10 | 11 | 7826982 | 7827011 | 7890346 | 7890375 |
| 11 | 2 | 242078292 | 242078321 | 242115058 | 242115087 |
| 12 | 4 | 64204637 | 64204666 | 64349732 | 64349761 |
| 13 | 9 | 4739185 | 4739214 | 4877191 | 4877220 |
| 14 | 6 | 144636123 | 144636152 | 144697409 | 144697438 |
| 15 | 22 | 47217593 | 47217622 | 47358569 | 47358598 |
| 16 | 16 | 31860632 | 31860661 | 31871567 | 31871596 |
| 17 | 10 | 6735457 | 6735486 | 6769271 | 6769300 |
| 18 | 14 | 26687519 | 26687548 | 26802036 | 26802065 |
| 19 | 11 | 63024619 | 63024648 | 63124364 | 63124393 |
| 20 | 11 | 55829595 | 55829624 | 55865368 | 55865397 |
| 21 | 2 | 20791148 | 20791177 | 20975473 | 20975502 |
| 22 | 1 | 114271384 | 114271413 | 114393543 | 114393572 |
| 23 | 3 | 150148359 | 150148388 | 150282174 | 150282203 |
| 24 | 3 | 3073923 | 3073952 | 3172838 | 3172867 |
| 25 | 2 | 228849721 | 228849750 | 228936340 | 228936369 |
| 26 | X | 126618973 | 126619002 | 126813933 | 126813962 |
| 27 | 17 | 3298602 | 3298631 | 3441466 | 3441495 |
| 28 | 6 | 141959860 | 141959889 | 142115080 | 142115109 |
| 29 | 2 | 221633734 | 221633763 | 221711013 | 221711042 |
| 30 | 18 | 8164112 | 8164141 | 8240139 | 8240168 |
| 31 | X | 83389649 | 83389678 | 83505069 | 83505098 |
| 32 | 2 | 38746384 | 38746413 | 38993608 | 38993637 |
| 33 | 2 | 169564272 | 169564301 | 169647178 | 169647207 |
| 34 | X | 51609401 | 51609430 | 51874810 | 51874839 |
| 35 | 1 | 96786345 | 96786374 | 96953172 | 96953201 |
| 36 | 13 | 76881443 | 76881472 | 77098111 | 77098140 |
| 37 | 12 | 10235420 | 10235449 | 10425533 | 10425562 |
| 38 | 3 | 151888506 | 151888535 | 151931689 | 151931718 |
| 39 | 8 | 81036891 | 81036920 | 81252998 | 81253027 |
| 40 | 21 | 25447240 | 25447269 | 25521204 | 25521233 |
| 41 | 12 | 39724271 | 39724300 | 39841836 | 39841865 |
| 42 | 2 | 2916395 | 2916424 | 3008405 | 3008434 |
| 43 | 5 | 39046925 | 39046954 | 39197632 | 39197661 |
| 44 | 2 | 127388557 | 127388586 | 127412536 | 127412565 |
| 45 | 21 | 14583256 | 14583285 | 14847718 | 14847747 |
| 46 | 22 | 32461008 | 32461037 | 32549161 | 32549190 |
| 47 | 13 | 77416228 | 77416257 | 77635638 | 77635667 |
| 48 | X | 66017312 | 66017341 | 66266600 | 66266629 |
| 49 | 9 | 119814505 | 119814534 | 119942889 | 119942918 |
| 50 | 15 | 96385261 | 96385290 | 96582971 | 96583000 |

TABLE 1A
(part 1d)

| | 4 kb Sequence Location | | | | |
|---|---|---|---|---|---|
| No. | Chr | Start1 | End1 | Start2 | End2 |
| 1 | 2 | 151357307 | 151361306 | 151615006 | 151619005 |
| 2 | 13 | 41466345 | 41470344 | 41557353 | 41561352 |
| 3 | 20 | 23380287 | 23384286 | 23532161 | 23536160 |
| 4 | 1 | 84316046 | 84320045 | 84536073 | 84540072 |
| 5 | 1 | 209336141 | 209340140 | 209416679 | 209420678 |
| 6 | 5 | 177835097 | 177839096 | 178009947 | 178013946 |
| 7 | 11 | 77783850 | 77787849 | 77893155 | 77897154 |
| 8 | 8 | 48489318 | 48493317 | 48710462 | 48714461 |
| 9 | 8 | 33043674 | 33047673 | 33243639 | 33247638 |
| 10 | 11 | 7823012 | 7827011 | 7890346 | 7894345 |
| 11 | 2 | 242078292 | 242082291 | 242115058 | 242119057 |
| 12 | 4 | 64204637 | 64208636 | 64345762 | 64349761 |
| 13 | 9 | 4735215 | 4739214 | 4873221 | 4877220 |
| 14 | 6 | 144636123 | 144640122 | 144693439 | 144697438 |
| 15 | 22 | 47213623 | 47217622 | 47354599 | 47358598 |
| 16 | 16 | 31856662 | 31860661 | 31871567 | 31875566 |
| 17 | 10 | 6735457 | 6739456 | 6765301 | 6769300 |
| 18 | 14 | 26683549 | 26687548 | 26802036 | 26806035 |
| 19 | 11 | 63024619 | 63028618 | 63124364 | 63128363 |
| 20 | 11 | 55825625 | 55829624 | 55865368 | 55869367 |
| 21 | 2 | 20791148 | 20795147 | 20975473 | 20979472 |
| 22 | 1 | 114267414 | 114271413 | 114389573 | 114393572 |
| 23 | 3 | 150148359 | 150152358 | 150278204 | 150282203 |
| 24 | 3 | 3069953 | 3073952 | 3168868 | 3172867 |
| 25 | 2 | 228849721 | 228853720 | 228936340 | 228940339 |
| 26 | X | 126618973 | 126622972 | 126813933 | 126817932 |
| 27 | 17 | 3298602 | 3302601 | 3437496 | 3441495 |
| 28 | 6 | 141955890 | 141959889 | 142111110 | 142115109 |
| 29 | 2 | 221629764 | 221633763 | 221707043 | 221711042 |
| 30 | 18 | 8164112 | 8168111 | 8236169 | 8240168 |
| 31 | X | 83389649 | 83393648 | 83505069 | 83509068 |
| 32 | 2 | 38742414 | 38746413 | 38989638 | 38993637 |
| 33 | 2 | 169560302 | 169564301 | 169643208 | 169647207 |
| 34 | X | 51609401 | 51613400 | 51870840 | 51874839 |
| 35 | 1 | 96786345 | 96790344 | 96953172 | 96957171 |
| 36 | 13 | 76877473 | 76881472 | 77094141 | 77098140 |
| 37 | 12 | 10235420 | 10239419 | 10421563 | 10425562 |
| 38 | 3 | 151888506 | 151892505 | 151931689 | 151935688 |
| 39 | 8 | 81036891 | 81040890 | 81249028 | 81253027 |
| 40 | 21 | 25447240 | 25451239 | 25517234 | 25521233 |
| 41 | 12 | 39724271 | 39728270 | 39837866 | 39841865 |
| 42 | 2 | 2912425 | 2916424 | 3008405 | 3012404 |
| 43 | 5 | 39042955 | 39046954 | 39197632 | 39201631 |
| 44 | 2 | 127388557 | 127392556 | 127412536 | 127416535 |
| 45 | 21 | 14583256 | 14587255 | 14843748 | 14847747 |
| 46 | 22 | 32457038 | 32461037 | 32549161 | 32553160 |
| 47 | 13 | 77416228 | 77420227 | 77635638 | 77639637 |
| 48 | X | 66017312 | 66021311 | 66266600 | 66270599 |
| 49 | 9 | 119814505 | 119818504 | 119942889 | 119946888 |
| 50 | 15 | 96381291 | 96385290 | 96582971 | 96586970 |

TABLE 1A
(part 1e)

| No. | probe | Primer ID |
|---|---|---|
| 1 | Hg38_2_151357305_151361853_151612320_151619007_RF | OBD160.001 |
| 2 | Hg38_13_41466343_41476518_41555919_41561354_RF | OBD160.005 |
| 3 | Hg38_20_23380285_23385557_23532159_23546814_RR | OBD160.009 |
| 4 | Hg38_1_84316044_84322127_84529225_84540074_RF | OBD160.013 |
| 5 | Hg38_1_209333922_209340142_209404224_209420680_FF | OBD160.017 |
| 6 | Hg38_5_177834278_177839098_178009945_178017869_FR | OBD160.021 |
| 7 | Hg38_11_77781318_77787851_77893153_77899469_FR | OBD160.025 |
| 8 | Hg38_8_48489316_48497835_48710460_48716484_RR | OBD160.029 |
| 9 | Hg38_8_33042243_33047675_33243637_33249598_FR | OBD160.033 |
| 10 | Hg38_11_7823466_7827013_7890344_7893739_FR | OBD160.037 |
| 11 | Hg38_2_242078290_242088090_242115056_242117195_RR | OBD160.041 |
| 12 | Hg38_4_64204635_64210880_64341978_64349763_RF | OBD160.045 |
| 13 | Hg38_9_4737518_4739216_4871409_4877222_FF | OBD160.049 |
| 14 | Hg38_6_144636121_144641243_144691336_144697440_RF | OBD160.053 |
| 15 | Hg38_22_47207724_47217624_47356169_47358600_FF | OBD160.057 |
| 16 | Hg38_16_31853018_31860663_31871565_31877473_FR | OBD160.061 |
| 17 | Hg38_10_6735455_6749237_6766912_6769302_RF | OBD160.065 |
| 18 | Hg38_14_26681688_26687550_26802034_26810557_FR | OBD160.069 |
| 19 | Hg38_11_63024617_63034264_63124362_63131307_RR | OBD160.073 |
| 20 | Hg38_11_55823665_55829626_55865366_55868750_FR | OBD160.077 |
| 21 | Hg38_2_20791146_20796987_20975471_20980938_RR | OBD160.081 |
| 22 | Hg38_1_114264796_114271415_114388190_114393574_FF | OBD160.085 |
| 23 | Hg38_3_150148357_150152202_150276483_150282205_RF | OBD160.089 |
| 24 | Hg38_3_3070802_3073954_3162690_3172869_FF | OBD160.093 |
| 25 | Hg38_2_228849719_228856896_228936338_228946445_RR | OBD160.097 |
| 26 | Hg38_X_126618971_126632078_126813931_126821389_RR | OBD160.101 |
| 27 | Hg38_17_3298600_3300356_3435567_3441497_RF | OBD160.105 |
| 28 | Hg38_6_141949457_141959891_142112622_142115111 FF | OBD160.109 |
| 29 | Hg38_2_221629362_221633765_221703220_221711044_FF | OBD160.113 |
| 30 | Hg38_18_8164110_8169146_8228637_8240170_RF | OBD160.117 |
| 31 | Hg38_X_83389647_83396643_83505067_83507528_RR | OBD160.121 |
| 32 | Hg38_2_38740614_38746415_38982199_38993639_FF | OBD160.125 |
| 33 | Hg38_2_169562452_169564303_169638099_169647209_FF | OBD160.129 |
| 34 | Hg38_X_51609399_51612166_51861901_51874841_RF | OBD160.133 |
| 35 | Hg38_1_96786343_96792340_96953170_96960029_RR | OBD160.137 |
| 36 | Hg38_13_76879860_76881474_77089881_77098142_FF | OBD160.141 |
| 37 | Hg38_12_10235418_10237590_10417499_10425564_RF | OBD160.145 |
| 38 | Hg38_3_151888504_151893566_151931687_151943298_RR | OBD160.149 |
| 39 | Hg38_8_81036889_81041061_81244260_81253029_RF | OBD160.153 |
| 40 | Hg38_21_25447238_25454612_25514175_25521235_RF | OBD160.157 |
| 41 | Hg38_12_39724269_39737447_39830409_39841867_RF | OBD160.161 |
| 42 | Hg38_2_2911175_2916426_3008403_3014033_FR | OBD160.165 |
| 43 | Hg38_5_39037737_39046956_39197630_39209276_FR | OBD160.169 |
| 44 | Hg38_2_127388555_127395831_127412534_127416129_RR | OBD160.173 |
| 45 | Hg38_21_14583254_14600479_14842273_14847749_RF | OBD160.177 |
| 46 | Hg38_22_32455270_32461039_32549159_32556587_FR | OBD160.181 |
| 47 | Hg38_13_77416226_77433379_77635636_77642621_RR | OBD160.185 |
| 48 | Hg38_X_66017310_66021076_66266598_66279138_RR | OBD160.189 |
| 49 | Hg38_9_119814503_119824066_119942887_119944563_RR | OBD160.193 |
| 50 | Hg38_15_96378302_96385292_96582969_96587891_FR | OBD160.197 |

TABLE 1A (part 1f)

| No. | Sequence | Primer ID | Sequence |
|---|---|---|---|
| 1 | CACACTGAGACTGATGGTGCGTGAGT | OBD160.003 | CAGTGTCTGAAGCAAATCCTCTGAC |
| 2 | AAACTTCTTAGTGGAGGAGGCTC | OBD160.007 | AGCCTGGGTGACAGAGTGAGACT |
| 3 | AGTGTCTGGAGTTGGAAGAGCAG | OBD160.011 | ACAGCAAGTTATCCCGAAGATGC |
| 4 | CACCTCCTGTGGTTTCTGCTTTATGTGT | OBD160.015 | GCTTCAAGACATTAGTTTTGGCAATAGT |
| 5 | CCCTGTGAGATAGGTGAAGTGGGTGT | OBD160.019 | ATGGCTGAGTCTGAAAATGGAATGGG |
| 6 | GCAAAAGCAATAGCCAGCACCATAGA | OBD160.023 | ACCAACCTCTCTTCCTTCTCAGCCTG |
| 7 | GAGCCCACAAAGCCTAAAATACG | OBD160.027 | GCCCACACTGTTCTTACACTTGC |
| 8 | TGCGTATGTTGAACCAGTCTTGC | OBD160.031 | GCCTGGAAATGAGGAACCCTGGT |
| 9 | GATAATGCCATCGTCAGGTTGAAATC | OBD160.035 | GCCAAAGCGGAACCAGGTAACTA |
| 10 | GCCCTCCTCCATACCAACTCGCT | OBD160.039 | CCCTTTGCCACCACAGACTTCAG |
| 11 | AGGCAATGTGGATGCTGTAGTCAACT | OBD160.043 | CTTCCCCTGCTGCCATCTAACATCCT |
| 12 | CCCTGAGTGAAAATCCAAAATCCTTG | OBD160.047 | CATTTGCCCAGGATGGTCGGTGGTA |
| 13 | ACCCCAACCAGTAGCAGTATCTG | OBD160.051 | GTTGGCTATCCACTCTTTCTCATCT |
| 14 | CTAAAGGAGTGAACTGAAGCAGC | OBD160.055 | GTTTGCTGAGAATGATGGTTTCCAG |
| 15 | CGTTCAGACTTTCCATCAATGTAACA | OBD160.059 | GCTGTGGTTTACCTCTCTGCTATCTT |
| 16 | GGGCTGGAAGAAAATGAAGAGTGACTAC | OBD160.063 | GCTATTTGCCAAGACCACAGTCAGACCA |
| 17 | CATAACTGACTTTACTTTTGGGATGATG | OBD160.067 | GTAGACAGCATCCAGATTCCTTGAGTGT |
| 18 | TTGATGCCCAGGTGCTTGTTTGATGC | OBD160.071 | GCTGAGAGCATTGTGGAAGAATCT |
| 19 | GCAGCCCTTAGGTAGCAAGATGC | OBD160.075 | GAAGTTATCAAGTTCAACCTCCTATC |
| 20 | TGACATCAGGAAGAGACTCAGTGTAT | OBD160.079 | CTGACTGATGTATCCTACCAATAAGC |
| 21 | ACACAGTTGGTAAGTGGCAAAGCC | OBD160.083 | AACTATTCTAAGGGTCTTGGAAAT |
| 22 | TCTGTGATTTTCCTTCTCAAGTTGTG | OBD160.087 | CATTTTGGTGCCTTCCCACACATAAA |
| 23 | GCCAAATAGGACAGACCCACAGC | OBD160.091 | GTCATTCGTTCCCCAGTCACCTGCTTCA |
| 24 | AACACAATCTCACCCCAAATCCCAGC | OBD160.095 | CTTACCCATCTGACTGTGTTCTTAGC |
| 25 | CTACTCTCAATGTGACGACTGGAAGG | OBD160.099 | GTAGGGAGGCAGAAATGGTGTGTTTG |
| 26 | TATGAGTCCCCAGAGGATAGAAGAAT | OBD160.103 | TGGCACTACATAAAATCACCCATTGT |
| 27 | CAGCAAGATGCCTGAACAGACTTTAC | OBD160.107 | TATCCACAACGAGGTGAGGACTCTCC |
| 28 | CCAGAAAAGCAGCAGTTACACCTACAGG | OBD160.111 | CTTGAGAGTAAAGGTGGTAACATAACAC |
| 29 | ATGAAGGGCACCAAAAGATGCTACCT | OBD160.115 | TGGCAGCAAACTCTCACCTTCTGGAG |
| 30 | CGGGAGGCTCAAAAGAGGGAGAAAGA | OBD160.119 | TTGGCTAACACAGTCTCATCCAGTGA |
| 31 | GAGGTGTTTCAGTGAGAGTAGAGAGG | OBD160.123 | AGAGCCACATTCAATAGACGACCCGT |
| 32 | TGTAGTCACCAATACCGTAAAACCCC | OBD160.127 | CAAAGACTACTGGGTAACTACAGACG |
| 33 | TTTCCTTATCTTGGTGCTGTTACATAGC | OBD160.131 | GGCTTTTCTGTGATGCTCTGGTTGGCAA |
| 34 | TTTCTCCTCAAGATGGGTCCTATTTT | OBD160.135 | CTGCCACCTCCTCTGTATGGTTCTTA |
| 35 | GACACACACAGAGGTAGTAGAGATTC | OBD160.139 | GCTCTCTCAGCCCAGGTGCTTAGTTT |
| 36 | CGAAGGATTGACTCTGATAGGCTTAC | OBD160.143 | TGCTTGGCTGGCTCTAAAGGCTTGGA |
| 37 | ACTTTTATTCCACTGGCAGCAAGGTAAT | OBD160.147 | GAAGACAGATGACAGAAGACAGGTGGCT |
| 38 | AAGGAGTTGTTCTCAAGGTATGTTAC | OBD160.151 | CTGACTGATTGCTCCTATTGGTAAAT |
| 39 | CAGCCCAAAGCGAGGGTGGAAAACAT | OBD160.155 | GTGAACTACTCTTTCTTGCTGCTTCC |
| 40 | ACCTGTCCTACAAGAAATGCT | OBD160.159 | ACATGCATTTTGAAATTGATAGTT |
| 41 | CAGTAACTAATCTTCTCTGGTGACAA | OBD160.163 | ACTCTACAGATTCCCAGGACCTACTG |
| 42 | CAAGACCACCAAAATACAGAATGGAG | OBD160.167 | GAGAAACGATTCCTAACCCCTACCAT |
| 43 | CCTTTTCCTCAGGTTTCTCTTTTCCC | OBD160.171 | GAAGCCAAGCGAAGGCATCTGACTCA |
| 44 | GAGGAGCGAAAGAAAGGGTTTTAGACTC | OBD160.175 | CACTAAAAGTCCTAAAGAAGAAGCCAAA |
| 45 | GGAATCAGAAGAACTTGTGTGACCCA | OBD160.179 | CAGTGAGAGAGAGCAATGGAGGAAGC |
| 46 | GTTGGTAGAATGAATCAATGGTAGAATG | OBD160.183 | GCCTGGGTGACAGAGGGAGACTC |
| 47 | GCTTCAAACAGACAAAGGTATTTCCT | OBD160.187 | GCCCAGAGACTATGCTTTGAGAAT |
| 48 | CCACCCAGCAGCAAAAGCAGCAG | OBD160.191 | CCTTCTGCTTCTCCACCACCCAT |
| 49 | CCCTTGTAGTTGGAAAGCAGCCGCAGCC | OBD160.195 | AATGTAGCCAGTGTCACAGGCAGAAGTG |
| 50 | GTGCCTCTCCTTACACTCATCTCTCA | OBD160.199 | GCGAAAGACTCTGTAACTGTGTTGGC |

TABLE 1A
(part 1g)

| No. | Probe | Marker |
|---|---|---|
| 1 | Hg38_2_151357305_151361853_151612320_151619007_RF | OBD160.001.003 |
| 2 | Hg38_13_41466343_41476518_41555919_41561354_RF | OBD160.005.009 |
| 3 | Hg38_20_23380285_23385557_23532159_23546814_RR | OBD160.011.013 |
| 4 | Hg38_1_84316044_84322127_84529225_84540074_RF | OBD160.015.017 |
| 5 | Hg38_1_209333922_209340142_209404224_209420680_FF | OBD160.019.021 |
| 6 | Hg38_5_177834278_177839098_178009945_178017869_FR | OBD160.023.025 |
| 7 | Hg38_11_77781318_77787851_77893153_77899469_FR | OBD160.025.027 |
| 8 | Hg38_8_48489316_48497835_48710460_48716484_RR | OBD160.029.031 |
| 9 | Hg38_8_33042243_33047675_33243637_33249598_FR | OBD160.033.035 |
| 10 | Hg38_11_7823466_7827013_7890344_7893739_FR | OBD160.037.039 |
| 11 | Hg38_2_242078290_242088090_242115056_242117195_RR | OBD160.041.043 |
| 12 | Hg38_4_64204635_64210880_64341978_64349763_RF | OBD160.045.047 |
| 13 | Hg38_9_4737518_4739216_4871409_4877222_FF | OBD160.049.051 |
| 14 | Hg38_6_144636121_144641243_144691336_144697440_RF | OBD160.053.055 |
| 15 | Hg38_22_47207724_47217624_47356169_47358600_FF | OBD160.057.059 |
| 16 | Hg38_16_31853018_31860663_31871565_31877473_FR | OBD160.061.063 |
| 17 | Hg38_10_6735455_6749237_6766912_6769302_RF | OBD160.065.067 |
| 18 | Hg38_14_26681688_26687550_26802034_26810557_FR | OBD160.069.071 |
| 19 | Hg38_11_63024617_63034264_63124362_63131307_RR | OBD160.073.075 |
| 20 | Hg38_11_55823665_55829626_55865366_55868750_FR | OBD160.077.079 |
| 21 | Hg38_2_20791146_20796987_20975471_20980938_RR | OBD160.081.083 |
| 22 | Hg38_1_114264796_114271415_114388190_114393574_FF | OBD160.085.087 |
| 23 | Hg38_3_150148357_150152202_150276483_150282205_RF | OBD160.089.091 |
| 24 | Hg38_3_3070802_3073954_3162690_3172869_FF | OBD160.093.095 |
| 25 | Hg38_2_228849719_228856896_228936338_228946445_RR | OBD160.097.099 |
| 26 | Hg38_X_126618971_126632078_126813931_126821389_RR | OBD160.101.103 |
| 27 | Hg38_17_3298600_3300356_3435567_3441497_RF | OBD160.105.107 |
| 28 | Hg38_6_141949457_141959891_142112622_142115111_FF | OBD160.109.111 |
| 29 | Hg38_2_221629362_221633765_221703220_221711044_FF | OBD160.113.115 |
| 30 | Hg38_18_8164110_8169146_8228637_8240170_RF | OBD160.117.119 |
| 31 | Hg38_X_83389647_83396643_83505067_83507528_RR | OBD160.121.123 |
| 32 | Hg38_2_38740614_38746415_38982199_38993639_FF | OBD160.125.127 |
| 33 | Hg38_2_169562452_169564303_169638099_169647209_FF | OBD160.129.131 |
| 34 | Hg38_X_51609399_51612166_51861901_51874841_RF | OBD160.133.135 |
| 35 | Hg38_1_96786343_96792340_96953170_96960029_RR | OBD160.137.139 |
| 36 | Hg38_13_76879860_76881474_77089881_77098142_FF | OBD160.141.143 |
| 37 | Hg38_12_10235418_10237590_10417499_10425564_RF | OBD160.145.147 |
| 38 | Hg38_3_151888504_151893566_151931687_151943298_RR | OBD160.149.151 |
| 39 | Hg38_8_81036889_81041061_81244260_81253029_RF | OBD160.153.155 |
| 40 | Hg38_21_25447238_25454612_25514175_25521235_RF | OBD160.157.159 |
| 41 | Hg38_12_39724269_39737447_39830409_39841867_RF | OBD160.161.163 |
| 42 | Hg38_2_2911175_2916426_3008403_3014033_FR | OBD160.165.167 |
| 43 | Hg38_5_39037737_39046956_39197630_39209276_FR | OBD160.169.171 |
| 44 | Hg38_2_127388555_127395831_127412534_127416129_RR | OBD160.173.175 |
| 45 | Hg38_21_14583254_14600479_14842273_14847749_RF | OBD160.177.179 |
| 46 | Hg38_22_32455270_32461039_32549159_32556587_FR | OBD160.181.183 |
| 47 | Hg38_13_77416226_77433379_77635636_77642621_RR | OBD160.185.187 |
| 48 | Hg38_X_66017310_66021076_66266598_66279138_RR | OBD160.189.191 |
| 49 | Hg38_9_119814503_119824066_119942887_119944563_RR | OBD160.193.195 |
| 50 | Hg38_15_96378302_96385292_96582969_96587891_FR | OBD160.197.199 |

TABLE 1A
(part 2a)

| No. | probe | RP/Rsum | Rprank | FC:(class1/class2) |
|---|---|---|---|---|
| 51 | Hg38_4_37200238_37206260_37300406_37312473_FF | 2932.387886 | 93 | 1.603 |
| 52 | Hg38_3_98352533_98365728_98383505_98386616_FR | 1732.032487 | 41 | 1.602 |
| 53 | Hg38_21_16639172_16644423_16670368_16680104_FF | 3320.296898 | 119 | 1.601 |
| 54 | Hg38_2_42656271_42661181_42695432_42702668_RF | 2433.098065 | 66 | 1.599 |
| 55 | Hg38_10_25043685_25049585_25100349_25104868_RR | 2030.307559 | 47 | 1.598 |
| 56 | Hg38_1_229090863_229096006_229196083_229202342_FR | 2179.775328 | 55 | 1.597 |
| 57 | Hg38_13_105283036_105288043_105320265_105325556_RR | 1894.786347 | 43 | 1.597 |
| 58 | Hg38_12_103957683_103963295_104120354_104125638_RR | 2134.567802 | 51 | 1.592 |
| 59 | Hg38_6_114008656_114013787_114232226_114238214_RF | 1834.397344 | 42 | 1.588 |
| 60 | Hg38_8_140877455_140883144_140932435_140938098_RF | 2969.154809 | 97 | 1.585 |
| 61 | Hg38_4_136781505_136792883_136902701_136917925_FR | 1926.209278 | 44 | 1.584 |
| 62 | Hg38_17_44726208_44728331_44900687_44908527_FR | 3766.109078 | 142 | 1.583 |
| 63 | Hg38_8_116664671_116672423_116798829_116802256_RR | 2372.310095 | 62 | 1.582 |
| 64 | Hg38_3_105989527_105998279_106059965_106070080_FR | 150657.5165 | 69345 | 1.581 |
| 65 | Hg38_2_112740760_112742979_112765786_112772711_RF | 2216.789753 | 56 | 1.58 |
| 66 | Hg38_18_9337930_9343491_9573987_9583586_RR | 2016.059075 | 46 | 1.58 |
| 67 | Hg38_11_19074376_19084676_19339671_19348554_FR | 2412.878673 | 65 | 1.579 |
| 68 | Hg38_11_57219000_57228121_57426633_57429019_FF | 52224.03779 | 6959 | 1.576 |
| 69 | Hg38_11_12078291_12088535_12276885_12282796_FF | 2992.859117 | 99 | 1.575 |
| 70 | Hg38_4_152885427_152900293_153068111_153076707_FR | 2107.102525 | 49 | 1.574 |
| 71 | Hg38_1_47112037_47119435_47237146_47242526_FF | 3119.590551 | 104 | 1.572 |
| 72 | Hg38_13_27255589_27257567_27412662_27420769_FR | 2412.337745 | 64 | 1.572 |
| 73 | Hg38_8_31019968_31023828_31152507_31157947_FR | 3265.30875 | 113 | 1.571 |
| 74 | Hg38_7_30496219_30498858_30628404_30631817_FF | 2885.244353 | 89 | 1.571 |
| 75 | Hg38_X_41694532_41696489_41776262_41781759_FF | 2614.17827 | 75 | 1.57 |
| 76 | Hg38_6_51827509_51843341_51906482_51917695_FF | 24590.08703 | 1846 | 1.568 |
| 77 | Hg38_4_52550935_52553766_52741887_52744874_RF | 2398.639908 | 63 | 1.568 |
| 78 | Hg38_4_143208146_143211670_143254394_143265663_FR | 117290.0204 | 40943 | 1.567 |
| 79 | Hg38_2_79143668_79146500_79293195_79300892_FR | 2822.115975 | 86 | 1.567 |
| 80 | Hg38_3_386460_393029_417553_428199_RF | 2371.626586 | 61 | 1.567 |
| 81 | Hg38_8_105746180_105747310_105814873_105824107_FR | 2281.090504 | 58 | 1.567 |
| 82 | Hg38_20_47934845_47942853_47960362_47966156_RF | 2812.677411 | 84 | 1.566 |
| 83 | Hg38_1_193673926_193687017_193767073_193774723_FR | 2511.193509 | 70 | 1.566 |
| 84 | Hg38_X_55562087_55565924_55755522_55768422_RF | 2890.107857 | 90 | 1.565 |
| 85 | Hg38_12_22051055_22053335_22293062_22296971_FR | 3103.749832 | 103 | 1.564 |
| 86 | Hg38_5_168367648_168371678_168496703_168499633_RF | 2705.325572 | 80 | 1.564 |
| 87 | Hg38_9_13378184_13386623_13453370_13455410_RR | 2293.493641 | 59 | 1.564 |
| 88 | Hg38_4_13607623_13610565_13677034_13689726_RR | 3789.680583 | 144 | 1.563 |
| 89 | Hg38_1_158836157_158842400_158905246_158912243_FF | 2663.851596 | 76 | 1.563 |
| 90 | Hg38_2_59529259_59533399_59556418_59563869_RF | 3642.187614 | 135 | 1.56 |
| 91 | Hg38_1_172365073_172375924_172561646_172570053_RR | 3195.829634 | 108 | 1.558 |
| 92 | Hg38_1_167272741_167278581_167541602_167544034_FF | 2582.676531 | 72 | 1.558 |
| 93 | Hg38_1_27783032_27786599_27834284_27836433_FF | 2679.371207 | 78 | 1.557 |
| 94 | Hg38_2_144038117_144048675_144274589_144280493_FF | 2464.995797 | 67 | 1.557 |
| 95 | Hg38_17_41002849_41006661_41059440_41062968_RF | 2959.335905 | 96 | 1.556 |
| 96 | Hg38_11_90904893_90913850_91035518_91039190_FF | 2939.352384 | 94 | 1.554 |
| 97 | Hg38_1_247706861_247710350_247812273_247815652_FR | 2718.801607 | 81 | 1.554 |
| 98 | Hg38_12_23014365_23020377_23180775_23184338_RR | 2861.202321 | 88 | 1.552 |
| 99 | Hg38_4_28348510_28364344_28414655_28418162_FF | 2688.720003 | 79 | 1.551 |
| 100 | Hg38_17_35578150_35580887_35656691_35659920_FF | 3346.702466 | 121 | 1.55 |

TABLE 1A (part 2b)

| | | P. | | Probe sequence |
|---|---|---|---|---|
| No | pfp | value | Type | 60 mer |
| 51 | 0 | 0 | SBMA | ATACAGGTATCAAATCATCTAGTTGTACTCGATGTATAAAAATCACAAGCATTCCTATAC |
| 52 | 0 | 0 | SBMA | TTTAGGGTCTTTTCTTTATCCTTGATCTTCGAGAAGATGTTGAGTTTTATCAGATGCTTT |
| 53 | 0 | 0 | SBMA | TATTTGTTATCTATTACTTCCTAACAAATCGAGGACCTGAATTACTTATCACCCTTCTCA |
| 54 | 0 | 0 | SBMA | TCTGTTTAGCAAATAAACACAAAAAGTATCGACAGATTGACCAGTTTTTAGTTGTAGCAC |
| 55 | 0 | 0 | SBMA | ACTGAAGTCCCAGTAGAACAGAAGAGAATCGAAAGATTCTGAATGGGGAAAAACTTACTC |
| 56 | 0 | 0 | SBMA | CACTTAAGAGCCAGCTATTTGACTGAATTCGAAAAGCTTCTACCAAAAAAATCCTTAAAA |
| 57 | 0 | 0 | SBMA | TTTCCACTGTTTTCTTACAACTTTCCCTTCGATTTGTCTTAGCAGACATTTTAAGTTACA |
| 58 | 0 | 0 | SBMA | AATGGAAAAATTCCTGGATTAAGTTATGTCGATACAATCAAATTTTTTAAGCATTTAAAA |
| 59 | 0 | 0 | SBMA | CCCATGAGTAAATGTAGACCTATGTCTATCGACGTATACCCTATGGAGAGGATATTCCCT |
| 60 | 0 | 0 | SBMA | CACATCTAACACAATTCACAGTACAAAATCGAATCAAAGCTGTGTCACAAACTATGTAAC |
| 61 | 0 | 0 | SBMA | TAAAAATGTAAACGGCCTGTTTTTAAACTCGATGTTTGATCATTTAAATGCATTATGTCT |
| 62 | 0 | 0 | SBMA | ATAATTTCCACATGTTCTATTGGTCCAATCGACTGAAGCACCAAAATTAAAAATATTTTT |
| 63 | 0 | 0 | SBMA | CTTGATCTACTGAACATATCTTGAGTTTTCGACTGGTTTTTCTCCTCATTATATTTTCCT |
| 64 | 0.087873867 | 0. | SBMA | AGCCTGTGATGGCAAGACTTGCCAAAACTCGAGTTGCCCAGGTATGCATCTTTAGAAAGG |
| | | 006561035 | | |
| 65 | 0 | 0 | SBMA | AAAATCTTTTCCAGAAGTTGTTTCCAGATCGAGACTTGGACTAGAATTAAGTCTGTGAAC |
| 66 | 0 | 0 | SBMA | TGCCTTGATGATTGGAAGAGATATAACTTCGAAAGTATAAATTCTCTAAATTCTAATTTT |
| 67 | 0 | 0 | SBMA | CTGCCTTTTAATCCTATGTGGAAATTGGTCGAGTTATTTTTGACAATTGAACTTTTTGGT |
| 68 | 0.000182018 | 0.0000 | SBMA | ACCCAGAAGAAAGACTGCCAAATCTTCCTCGAACCAAAGAACATGGGAATGGTTCAGAGG |
| | | 013638 | | |
| | | 285394 | | |
| | | 7602 | | |
| 69 | 0 | 0 | SBMA | TAGTCTATTTCTGTTCTAGTATTTATTGTCGAACACATTTCTCAATATCATCTATTTGGT |
| 70 | 0 | 0 | SBMA | CACTCAGAATGTTAATGTATGTAATTATTCGATCATGTCTCTTTCCACATGTCTTCTTCT |
| 71 | 0 | 0 | SBMA | AACAAACTAGACATTGAAGAAATGTACCTCGATACTATTCTAATCTCCCATTTAGTAGGC |
| 72 | 0 | 0 | SBMA | AGATCCCTTGTACACTGTATACTGTTTGTCGAAATACCTACGAGTCCTAAAATATAGTTC |
| 73 | 0 | 0 | SBMA | GATATTGGACAGGATCAACAAAAAGGAATCGACTCGTGTATATGTTTGATATATCCCACA |
| 74 | 0 | 0 | SBMA | CACTTTCGTAATTTGTCATCAGATAACTTCGATGAACTTGGCACCAAGGTCAGTATGAGA |
| 75 | 0 | 0 | SBMA | CTATATGTTTTGTTCCCTATCATGCCTTTCGATTACTCTAAGATTTAAAGTCATGTGGAG |
| 76 | 0 | 0 | SBMA | GGAATCACATGGGCAAGAGAGCTGAGACTCGAATGTCCCCCACCATCTTACACATCATTG |
| 77 | 0 | 0 | SBMA | CATTACCTGACCAGGTCAATCCAGAATTTCGACAAAAACATTGTAGGATGAAAAGAGGAG |
| 78 | 0.024207964 | 0. | SBMA | TCCAAAGTTTCAACTTACAATCACATCTTCGACTCCTTTGCATGTTTTCTTGCATCCACT |
| | | 001067174 | | |
| 79 | 0 | 0 | SBMA | CCTTTTCAAAAAAAAACAACTCTCATTCTCGATGACATCAATGGTTTTGCAGTATTGAGC |
| 80 | 0 | 0 | SBMA | GGAATCTTCAAAGAATAAGTGTTTTTCATCGAACATCATAATGTTCTACTTGCAAAACAG |
| 81 | 0 | 0 | SBMA | TTAACTCCAAATTTCCAAGTCAGTTAACTCGACCTTTTTCTGTTTCAGTAACCAACCCAG |
| 82 | 0 | 0 | SBMA | ACTGCAGATCTGTACCCAAGTGATATGATCGACATTCCAGTAAGACTTGTCTTTTATTGG |
| 83 | 0 | 0 | SBMA | GGAACACTTCTCTAAGAACCTGAATTATTCGAGCTTCAAATACAAAACATTTGGGCTCAA |
| 84 | 0 | 0 | SBMA | TAATCCCTTTCAATTTCGTTAAGTTTCCTCGATTAAGCCATGCATTGATACCTGACCCAA |
| 85 | 0 | 0 | SBMA | ATGCAGTTCATCTTTCCCTCAGTAGATATCGAGACTAAGAAACAAACACTTGTCAAAATA |
| 86 | 0 | 0 | SBMA | TTATAAACATCTTTTGGAGTTCTGTACTTCGAGTTTTGGAATCTGGCAGTGGAACTTCAG |
| 87 | 0 | 0 | SBMA | TTTTCTTCCTAATTTTGGTTAGCCAGTATCGAGTCTTAGAAGTATTCAATTTGCAATTTG |
| 88 | 0 | 0 | SBMA | AAATTGAAATGTAACAAGTCTGCATTTGTCGAAACAAATATTTGTCTACTGCATTGTTAA |
| 89 | 0 | 0 | SBMA | CATCACTTAAAAACCTTGTTAACAATCTTCGAGGTTACCATAACCCATTATTTTAAGCTG |
| 90 | 0 | 0 | SBMA | TTTTAAAGAATGCAGAAAAAAAACAAAATCGACAAACAGTTCACACAATAACACTATGTG |
| 91 | 0 | 0 | SBMA | TTTTTAAGGTTATGTGTTTGAGTTTGAATCGAAAAGAAGTATTACACTATTTTGCTCCCC |
| 92 | 0 | 0 | SBMA | ACGATGAAGATAATGTTACATTTATCATTCGAAGTTAAAGTTTACTTTCACAGGACATAA |
| 93 | 0 | 0 | SBMA | TTGTCTATCTCTTTTCCTTAGAAGACTCTCGAAAACAAAAACAAACAAAAAAATAAAAAT |
| 94 | 0 | 0 | SBMA | CTATGATTTAACTTTACTTCATTAGGTATCGAATTACAACAAAAAAACCCCCAAAATCCT |
| 95 | 0 | 0 | SBMA | CTTGGAAGGTTCAATGTGAAAGAAAGAGTCGATTTTGAAATATTATATAGATTCAGGAGA |
| 96 | 0 | 0 | SBMA | AATTTATCTCATTTCAGCATTGTATCAGTCGAATAATCCCACAGGAAAACCAAGTTGGCC |
| 97 | 0 | 0 | SBMA | CATCATAGGTACCTTAAGAGGTAGACTTTCGAAATACATAGTCAGTTCCCAGTTTGTATA |
| 98 | 0 | 0 | SBMA | TTTGTATATGGGCTCAGTAAAATGTGAATCGAACTTCTGCTTTTAAAATACTCTACTCTC |
| 99 | 0 | 0 | SBMA | CAAAGAAATGTTGTTCATGAAACTTATATCGACATAAAGAGCGTTAGGGCAAATTATAAG |
| 100 | 0 | 0 | SBMA | AAAAAAAAAGAAAACAGAAATTTGCAACTCGAAAAAGGAACCTTGATTCTCTGAGTGAAT |

TABLE 1A
(part 2c)

| | Probe Location | | | | |
|---|---|---|---|---|---|
| No. | Chr | Start1 | End1 | Start2 | End2 |
| 51 | 4 | 37206229 | 37206258 | 37312442 | 37312471 |
| 52 | 3 | 98365697 | 98365726 | 98383507 | 98383536 |
| 53 | 21 | 16644392 | 16644421 | 16680073 | 16680102 |
| 54 | 2 | 42656273 | 42656302 | 42702637 | 42702666 |
| 55 | 10 | 25043687 | 25043716 | 25100351 | 25100380 |
| 56 | 1 | 229095975 | 229096004 | 229196085 | 229196114 |
| 57 | 13 | 105283038 | 105283067 | 105320267 | 105320296 |
| 58 | 12 | 103957685 | 103957714 | 104120356 | 104120385 |
| 59 | 6 | 114008658 | 114008687 | 114238183 | 114238212 |
| 60 | 8 | 140877457 | 140877486 | 140938067 | 140938096 |
| 61 | 4 | 136792852 | 136792881 | 136902703 | 136902732 |
| 62 | 17 | 44728300 | 44728329 | 44900689 | 44900718 |
| 63 | 8 | 116664673 | 116664702 | 116798831 | 116798860 |
| 64 | 3 | 105998248 | 105998277 | 106059967 | 106059996 |
| 65 | 2 | 112740762 | 112740791 | 112772680 | 112772709 |
| 66 | 18 | 9337932 | 9337961 | 9573989 | 9574018 |
| 67 | 11 | 19084645 | 19084674 | 19339673 | 19339702 |
| 68 | 11 | 57228090 | 57228119 | 57428988 | 57429017 |
| 69 | 11 | 12088504 | 12088533 | 12282765 | 12282794 |
| 70 | 4 | 152900262 | 152900291 | 153068113 | 153068142 |
| 71 | 1 | 47119404 | 47119433 | 47242495 | 47242524 |
| 72 | 13 | 27257536 | 27257565 | 27412664 | 27412693 |
| 73 | 8 | 31023797 | 31023826 | 31152509 | 31152538 |
| 74 | 7 | 30498827 | 30498856 | 30631786 | 30631815 |
| 75 | X | 41696458 | 41696487 | 41781728 | 41781757 |
| 76 | 6 | 51843310 | 51843339 | 51917664 | 51917693 |
| 77 | 4 | 52550937 | 52550966 | 52744843 | 52744872 |
| 78 | 4 | 143211639 | 143211668 | 143254396 | 143254425 |
| 79 | 2 | 79146469 | 79146498 | 79293197 | 79293226 |
| 80 | 3 | 386462 | 386491 | 428168 | 428197 |
| 81 | 8 | 105747279 | 105747308 | 105814875 | 105814904 |
| 82 | 20 | 47934847 | 47934876 | 47966125 | 47966154 |
| 83 | 1 | 193686986 | 193687015 | 193767075 | 193767104 |
| 84 | X | 55562089 | 55562118 | 55768391 | 55768420 |
| 85 | 12 | 22053304 | 22053333 | 22293064 | 22293093 |
| 86 | 5 | 168367650 | 168367679 | 168499602 | 168499631 |
| 87 | 9 | 13378186 | 13378215 | 13453372 | 13453401 |
| 88 | 4 | 13607625 | 13607654 | 13677036 | 13677065 |
| 89 | 1 | 158842369 | 158842398 | 158912212 | 158912241 |
| 90 | 2 | 59529261 | 59529290 | 59563838 | 59563867 |
| 91 | 1 | 172365075 | 172365104 | 172561648 | 172561677 |
| 92 | 1 | 167278550 | 167278579 | 167544003 | 167544032 |
| 93 | 1 | 27786568 | 27786597 | 27836402 | 27836431 |
| 94 | 2 | 144048644 | 144048673 | 144280462 | 144280491 |
| 95 | 17 | 41002851 | 41002880 | 41062937 | 41062966 |
| 96 | 11 | 90913819 | 90913848 | 91039159 | 91039188 |
| 97 | 1 | 247710319 | 247710348 | 247812275 | 247812304 |
| 98 | 12 | 23014367 | 23014396 | 23180777 | 23180806 |
| 99 | 4 | 28364313 | 28364342 | 28418131 | 28418160 |
| 100 | 17 | 35580856 | 35580885 | 35659889 | 35659918 |

TABLE 1A
(part 2d)

| | 4 kb Sequence Location | | | | |
|---|---|---|---|---|---|
| No. | Chr | Start1 | End1 | Start2 | End2 |
| 51 | 4 | 37202259 | 37206258 | 37308472 | 37312471 |
| 52 | 3 | 98361727 | 98365726 | 98383507 | 98387506 |
| 53 | 21 | 16640422 | 16644421 | 16676103 | 16680102 |
| 54 | 2 | 42656273 | 42660272 | 42698667 | 42702666 |
| 55 | 10 | 25043687 | 25047686 | 25100351 | 25104350 |
| 56 | 1 | 229092005 | 229096004 | 229196085 | 229200084 |
| 57 | 13 | 105283038 | 105287037 | 105320267 | 105324266 |
| 58 | 12 | 103957685 | 103961684 | 104120356 | 104124355 |
| 59 | 6 | 114008658 | 114012657 | 114234213 | 114238212 |
| 60 | 8 | 140877457 | 140881456 | 140934097 | 140938096 |
| 61 | 4 | 136788882 | 136792881 | 136902703 | 136906702 |
| 62 | 17 | 44724330 | 44728329 | 44900689 | 44904688 |
| 63 | 8 | 116664673 | 116668672 | 116798831 | 116802830 |
| 64 | 3 | 105994278 | 105998277 | 106059967 | 106063966 |
| 65 | 2 | 112740762 | 112744761 | 112768710 | 112772709 |
| 66 | 18 | 9337932 | 9341931 | 9573989 | 9577988 |
| 67 | 11 | 19080675 | 19084674 | 19339673 | 19343672 |
| 68 | 11 | 57224120 | 57228119 | 57425018 | 57429017 |
| 69 | 11 | 12084534 | 12088533 | 12278795 | 12282794 |
| 70 | 4 | 152896292 | 152900291 | 153068113 | 153072112 |
| 71 | 1 | 47115434 | 47119433 | 47238525 | 47242524 |
| 72 | 13 | 27253566 | 27257565 | 27412664 | 27416663 |
| 73 | 8 | 31019827 | 31023826 | 31152509 | 31156508 |
| 74 | 7 | 30494857 | 30498856 | 30627816 | 30631815 |
| 75 | X | 41692488 | 41696487 | 41777758 | 41781757 |
| 76 | 6 | 51839340 | 51843339 | 51913694 | 51917693 |
| 77 | 4 | 52550937 | 52554936 | 52740873 | 52744872 |
| 78 | 4 | 143207669 | 143211668 | 143254396 | 143258395 |
| 79 | 2 | 79142499 | 79146498 | 79293197 | 79297196 |
| 80 | 3 | 386462 | 390461 | 424198 | 428197 |
| 81 | 8 | 105743309 | 105747308 | 105814875 | 105818874 |
| 82 | 20 | 47934847 | 47938846 | 47962155 | 47966154 |
| 83 | 1 | 193683016 | 193687015 | 193767075 | 193771074 |
| 84 | X | 55562089 | 55566088 | 55764421 | 55768420 |
| 85 | 12 | 22049334 | 22053333 | 22293064 | 22297063 |
| 86 | 5 | 168367650 | 168371649 | 168495632 | 168499631 |
| 87 | 9 | 13378186 | 13382185 | 13453372 | 13457371 |
| 88 | 4 | 13607625 | 13611624 | 13677036 | 13681035 |
| 89 | 1 | 158838399 | 158842398 | 158908242 | 158912241 |
| 90 | 2 | 59529261 | 59533260 | 59559868 | 59563867 |
| 91 | 1 | 172365075 | 172369074 | 172561648 | 172565647 |
| 92 | 1 | 167274580 | 167278579 | 167540033 | 167544032 |
| 93 | 1 | 27782598 | 27786597 | 27832432 | 27836431 |
| 94 | 2 | 144044674 | 144048673 | 144276492 | 144280491 |
| 95 | 17 | 41002851 | 41006850 | 41058967 | 41062966 |
| 96 | 11 | 90909849 | 90913848 | 91035189 | 91039188 |
| 97 | 1 | 247706349 | 247710348 | 247812275 | 247816274 |
| 98 | 12 | 23014367 | 23018366 | 23180777 | 23184776 |
| 99 | 4 | 28360343 | 28364342 | 28414161 | 28418160 |
| 100 | 17 | 35576886 | 35580885 | 35655919 | 35659918 |

TABLE 1A
(part 2e)

| No. | probe | Primer ID |
|---|---|---|
| 51 | Hg38_4_37200238_37206260_37300406_37312473_FF | OBD160.201 |
| 52 | Hg38_3_98352533_98365728_98383505_98386616_FR | OBD160.205 |
| 53 | Hg38_21_16639172_16644423_16670368_16680104_FF | OBD160.209 |
| 54 | Hg38_2_42656271_42661181_42695432_42702668_RF | OBD160.213 |
| 55 | Hg38_10_25043685_25049585_25100349_25104868_RR | OBD160.217 |
| 56 | Hg38_1_229090863_229096006_229196083_229202342_FR | OBD160.221 |
| 57 | Hg38_13_105283036_105288043_105320265_105325556_RR | OBD160.225 |
| 58 | Hg38_12_103957683_103963295_104120354_104125638_RR | OBD160.229 |
| 59 | Hg38_6_114008656_114013787 114232226_114238214_RF | OBD160.233 |
| 60 | Hg38_8_140877455_140883144 140932435_140938098_RF | OBD160.237 |
| 61 | Hg38_4_136781505_136792883_136902701_136917925_FR | OBD160.241 |
| 62 | Hg38_17_44726208_44728331_44900687_44908527_FR | OBD160.245 |
| 63 | Hg38_8_116664671_116672423_116798829_116802256_RR | OBD160.249 |
| 64 | Hg38_3_105989527_105998279_106059965_106070080_FR | OBD160.253 |
| 65 | Hg38_2_112740760_112742979_112765786_112772711_RF | OBD160.257 |
| 66 | Hg38_18_9337930_9343491_9573987_9583586_RR | OBD160.261 |
| 67 | Hg38_11_19074376_19084676_19339671_19348554_FR | OBD160.265 |
| 68 | Hg38_11_57219000_57228121_57426633_57429019_FF | OBD160.269 |
| 69 | Hg38_11_12078291_12088535_12276885_12282796_FF | OBD160.273 |
| 70 | Hg38_4_152885427_152900293_153068111_153076707_FR | OBD160.277 |
| 71 | Hg38_1_47112037_47119435_47237146_47242526_FF | OBD160.281 |
| 72 | Hg38_13_27255589_27257567_27412662_27420769_FR | OBD160.285 |
| 73 | Hg38_8_31019968_31023828_31152507_31157947_FR | OBD160.289 |
| 74 | Hg38_7_30496219_30498858_30628404_30631817_FF | OBD160.293 |
| 75 | Hg38_X_41694532_41696489_41776262_41781759_FF | OBD160.297 |
| 76 | Hg38_6_51827509_51843341_51906482_51917695_FF | OBD160.301 |
| 77 | Hg38_4_52550935_52553766_52741887_52744874_RF | OBD160.305 |
| 78 | Hg38_4_143208146_143211670_143254394_143265663_FR | OBD160.309 |
| 79 | Hg38_2_79143668_79146500_79293195_79300892_FR | OBD160.313 |
| 80 | Hg38_3_386460_393029_417553_428199_RF | OBD160.317 |
| 81 | Hg38_8_105746180_105747310_105814873_105824107_FR | OBD160.321 |
| 82 | Hg38_20_47934845_47942853_47960362_47966156_RF | OBD160.325 |
| 83 | Hg38_1_193673926_193687017_193767073 193774723_FR | OBD160.329 |
| 84 | Hg38_X_55562087_55565924_55755522_55768422_RF | OBD160.333 |
| 85 | Hg38_12_22051055_22053335_22293062_22296971_FR | OBD160.337 |
| 86 | Hg38_5_168367648_168371678_168496703_168499633_RF | OBD160.341 |
| 87 | Hg38_9_13378184_13386623_13453370_13455410_RR | OBD160.345 |
| 88 | Hg38_4_13607623_13610565_13677034_13689726_RR | OBD160.349 |
| 89 | Hg38_1_158836157_158842400_158905246_158912243_FF | OBD160.353 |
| 90 | Hg38_2_59529259_59533399_59556418_59563869_RF | OBD160.357 |
| 91 | Hg38_1_172365073_172375924_172561646_172570053_RR | OBD160.361 |
| 92 | Hg38_1_167272741_167278581_167541602_167544034_FF | OBD160.365 |
| 93 | Hg38_1_27783032_27786599_27834284_27836433_FF | OBD160.369 |
| 94 | Hg38_2_144038117_144048675_144274589_144280493_FF | OBD160.373 |
| 95 | Hg38_17_41002849_41006661_41059440_41062968_RF | OBD160.377 |
| 96 | Hg38_11_90904893_90913850_91035518_91039190_FF | OBD160.381 |
| 97 | Hg38_1_247706861_247710350_247812273_247815652_FR | OBD160.385 |
| 98 | Hg38_12_23014365_23020377_23180775_23184338_RR | OBD160.389 |
| 99 | Hg38_4_28348510_28364344_28414655_28418162_FF | OBD160.393 |
| 100 | Hg38_17_35578150_35580887_35656691_35659920_FF | OBD160.397 |

TABLE 1A (part 2f)

| No. | Sequence | Primer ID | Sequence |
|---|---|---|---|
| 51 | GGAATAGAGGGAAGAAAACCCATAAA | OBD160.203 | CTCTCTGTTTGTCTGTTATTGGTGTAT |
| 52 | AGTCTGTAAGGTTTCCTATGAGAAGC | OBD160.207 | GAAACGAATACCCCTGATGAAGATAG |
| 53 | CTCAGATTTCTCACCTGTAGAGCAGT | OBD160.211 | GGAAAAGTGAAGAATAAGCACCCATC |
| 54 | TTCTGGTAGCAGTGGTGGGAGTGTCA | OBD160.215 | AATGCCATACTCATACTCACCGTGCT |
| 55 | GCCTTGAGAACCTTAGAACAATGCCA | OBD160.219 | GGAGAAATCCCAGTTACACAGTTGGC |
| 56 | TAAGTCCAGGAGATTGAGGCTGC | OBD160.223 | AGTAACTAAGTCTCTACCTTCTTTGC |
| 57 | CAATGTGTCTAAGTAACGCCTGTGTG | OBD160.227 | GAAGAACATAACTGCCCCTGCTCCAG |
| 58 | TGCTGGCTGGGATTACAGGCGTA | OBD160.231 | CCACAGACGGTCAACAACAAAATGT |
| 59 | GATTGGTAGAGGAAAGAGAAGCAGAGAA | OBD160.235 | GTGCCAGAAAGGGTTATGCTGTTAGTGA |
| 60 | CTAAGAAGACAACACAACCTCCTC | OBD160.239 | GTATGCTGCTTACACCAGATAACTTA |
| 61 | GAGGAACTCAGAAGACTGGCTTTAGG | OBD160.243 | CACTGGAAAAGAGGCAGGTTTGAGAC |
| 62 | GCCACCTTCCCAGAAAGTGAGGC | OBD160.247 | GACCCCAAAGCACTCTGGACATC |
| 63 | GCTCTACTCTTTCCCTACAGGCTG | OBD160.251 | AGGAATAGAAAAGTGTAGCCACCCTA |
| 64 | TTGTGCTGTCTGGAGTTGTAGGAGGG | OBD160.255 | CCCTACTTCATCTCCTCTACACACAA |
| 65 | TCATTGTGTGGCTCTGACTTCCCAGT | OBD160.259 | CCGAATAGCCAAGGCAGAACAGCATA |
| 66 | TACTTCCTCTTACTTTTCTCCCCAGA | OBD160.263 | GTCCTCTGGTGCTCTTGATACCTACG |
| 67 | CCATCTTTCATCCACTCATCCCTCCA | OBD160.267 | GAATCCATCATAATGCTGACTCTGAA |
| 68 | CCCTGGGTCCCTCTCTTATCATC | OBD160.271 | GCCTCCACTCCACATCTCCCATC |
| 69 | AGAGCCGAGTTGTCCCAGCAGAG | OBD160.275 | GCAGGTCTCTGAGTAGGCACAAC |
| 70 | GAAAATGAGAGAGGGAGAAGGGAGGG | OBD160.279 | CCAATACGACTGGTGTTCCTTGTAAG |
| 71 | CTCAGTAGACGCAGAAAACGCTT | OBD160.283 | ATCTCTCTATTCCTATTTCCAGCCTA |
| 72 | GCTGGCAGGTTCGGTGTCTTGTG | OBD160.287 | GGAAAAGTCTGGCTGGGAGATGC |
| 73 | CAGTTCACAGGTCAGTATCCTTTCTG | OBD160.291 | CTTTGCCAACTTTTGTCCTTATTGCC |
| 74 | GTAATCACCACCAGTCTGTAAATAGC | OBD160.295 | GGTAGGACCCAAGAGTCAGATTTA |
| 75 | CTATTGAGGATTTCTAAAAGGAGGTC | OBD160.299 | TGAGCAATGAGCCCCTAAATACCAGG |
| 76 | GTGTCAGCAGATGTAGCCAGTAGCCA | OBD160.303 | TGCCAGATGACAAAATGGGAAGCGTA |
| 77 | GATTGGAACCTGTGAGCCACCAG | OBD160.307 | CTCACAGGCAAGATACTTCCACGGG |
| 78 | GGCTAAACTTCCTTCCTTCAGAATGC | OBD160.311 | CTCAAGTTGGGAGGGCTGATGGAAAC |
| 79 | TGGGTCTTCTATTTTCTTAGTCTGGC | OBD160.315 | ACACCCACAGAAACTAAGCAGCCAGT |
| 80 | CTTGTTTCTCTGCTCACTGGCTACTC | OBD160.319 | GGCTTTTGGTTTTGCTTTTGGTGTCC |
| 81 | GCACTTCACTGCTCCCCTTGCCAGC | OBD160.323 | CTGTTCTCCCAAGTCTCTAACTGTAGGC |
| 82 | CCCCAGCCTCGTGAGAGAGTCTA | OBD160.327 | CGAAGAGCCCCAACCCAACACAT |
| 83 | CCTGTGCTTCTGAAATGTGCTCTGCT | OBD160.331 | GCTATTTACCCTCAGAGTAGTATCAT |
| 84 | TAGCCGACTCTTGGCATTTATGAGGG | OBD160.335 | GCACCCTTGTATTACTTATCTATGGC |
| 85 | GCCTGGGTGACAGAGCAAGACTC | OBD160.339 | CAGCAAATACCCCAAATCCTGGA |
| 86 | TTCCAACCTCAGTGACAGAGTGA | OBD160.343 | ACACAGTTAGTAAACACCACAGGTAT |
| 87 | GGAAGTTCAGGAATGTGGTGTGCTCT | OBD160.347 | CAACTCTGGAAAATGCTTCTTGTGC |
| 88 | GGGATTGTGCTCTCCTTTGTTGACCA | OBD160.351 | CAGAACTTTCACTTGAGGTCCCATCA |
| 89 | CCTGGGACACTCACTCACAAAAGCATA | OBD160.355 | GTAGAAAGACTAAAAGACAAACCTCTCA |
| 90 | GCTGGAACAATGTATCACTTTAGGGA | OBD160.359 | TATTGAAGTTTCCCTGTCCGCCCAGC |
| 91 | CCTTAGAAAACATAAACACAATAC | OBD160.363 | GACAAGAGTAGAGGTGAGTGGGTA |
| 92 | GGGACCTTGTCTGCTTTACTCACTGC | OBD160.367 | CAGTGTGGAGACTTATGGGAAAACAG |
| 93 | TATCCTCTGCCCACCACTGTCACTT | OBD160.371 | GAGAGTTATTCCTCAAGTGCTCCCAC |
| 94 | GCAGAGGTAAAACAGGTAGAAAGGTC | OBD160.375 | GTCTCCTAAAGCCAAAACACCACCAT |
| 95 | CTAAAGTCAGGGAAGGCAAAGTAATC | OBD160.379 | CAGTATGAACGAGGTCACAGAGGGTA |
| 96 | TTTCTTAGAGATACAATCATTCTGGCAG | OBD160.383 | CCTCATAGGCTGCTTCTTTTGTTTCTCC |
| 97 | GCCTCGGTCAGATGTTCAATGGCAGGAA | OBD160.387 | TACTGGCTGAGGGATTTTCTTCACCAT |
| 98 | TCAGTTTCAGAGACCTCACCACATTA | OBD160.391 | GTCCTCAGAGAAAATCGCAGGGAGTC |
| 99 | GGGACTTACACAGCCCTATGCTTCCTTG | OBD160.395 | GCCGAGTGGGCTCTTCTGCCTACACAAC |
| 100 | GGCAGGAGAATCACTTGAACCCG | OBD160.399 | CTTGGTGTTACAGCCCCAGAAAT |

TABLE 1A
(part 2g)

| No. | probe | Marker |
|---|---|---|
| 51 | Hg38_4_37200238_37206260_37300406_37312473_FF | OBD160.201.203 |
| 52 | Hg38_3_98352533_98365728_98383505_98386616_FR | OBD160.205.207 |
| 53 | Hg38_21_16639172_16644423_16670368_16680104_FF | OBD160.209.211 |
| 54 | Hg38_2_42656271_42661181_42695432_42702668_RF | OBD160.213.215 |
| 55 | Hg38_10_25043685_25049585_25100349_25104868_RR | OBD160.217.219 |
| 56 | Hg38_1_229090863_229096006_229196083_229202342_FR | OBD160.221.223 |
| 57 | Hg38_13_105283036_105288043_105320265_105325556_RR | OBD160.225.227 |
| 58 | Hg38_12_103957683_103963295_104120354_104125638_RR | OBD160.229.231 |
| 59 | Hg38_6_114008656_114013787_114232226_114238214_RF | OBD160.233.235 |
| 60 | Hg38_8_140877455_140883144_140932435_140938098_RF | OBD160.237.239 |
| 61 | Hg38_4_136781505_136792883_136902701_136917925_FR | OBD160.241.243 |
| 62 | Hg38_17_44726208_44728331_44900687_44908527_FR | OBD160.245.247 |
| 63 | Hg38_8_116664671_116672423_116798829_116802256_RR | OBD160.249.251 |
| 64 | Hg38_3_105989527_105998279_106059965_106070080_FR | OBD160.253.255 |
| 65 | Hg38_2_112740760_112742979_112765786_112772711_RF | OBD160.257.259 |
| 66 | Hg38_18_9337930_9343491_9573987_9583586_RR | OBD160.261.263 |
| 67 | Hg38_11_19074376_19084676_19339671_19348554_FR | OBD160.265.267 |
| 68 | Hg38_11_57219000_57228121_57426633_57429019_FF | OBD160.269.271 |
| 69 | Hg38_11_12078291_12088535_12276885_12282796_FF | OBD160.273.275 |
| 70 | Hg38_4_152885427_152900293 153068111 153076707_FR | OBD160.277.279 |
| 71 | Hg38_1_47112037_47119435_47237146_47242526_FF | OBD160.281.283 |
| 72 | Hg38_13_27255589_27257567_27412662_27420769_FR | OBD160.285.287 |
| 73 | Hg38_8_31019968_31023828_31152507_31157947_FR | OBD160.289.291 |
| 74 | Hg38_7_30496219_30498858_30628404 30631817 FF | OBD160.293.295 |
| 75 | Hg38_X_41694532_41696489_41776262_41781759_FF | OBD160.297.299 |
| 76 | Hg38_6_51827509_51843341 51906482 51917695_FF | OBD160.301.303 |
| 77 | Hg38_4_52550935_52553766_52741887_52744874_RF | OBD160.305.307 |
| 78 | Hg38_4_143208146_143211670_143254394 143265663 FR | OBD160.309.311 |
| 79 | Hg38_2_79143668_79146500_79293195_79300892_FR | OBD160.313.315 |
| 80 | Hg38_3_386460_393029_417553_428199_RF | OBD160.317.319 |
| 81 | Hg38_8_105746180_105747310_105814873_105824107_FR | OBD160.321.323 |
| 82 | Hg38_20_47934845_47942853_47960362_47966156_RF | OBD160.325.327 |
| 83 | Hg38_1_193673926_193687017_193767073_193774723_FR | OBD160.329.331 |
| 84 | Hg38_X_55562087_55565924 55755522 55768422_RF | OBD160.333.335 |
| 85 | Hg38_12_22051055_22053335_22293062_22296971_FR | OBD160.337.339 |
| 86 | Hg38_5_168367648_168371678_168496703_168499633_RF | OBD160.341.343 |
| 87 | Hg38_9_13378184_13386623_13453370_13455410_RR | OBD160.345.347 |
| 88 | Hg38_4_13607623_13610565_13677034_13689726_RR | OBD160.349.351 |
| 89 | Hg38_1_158836157_158842400_158905246_158912243_FF | OBD160.353.355 |
| 90 | Hg38_2_59529259_59533399 59556418_59563869_RF | OBD160.357.359 |
| 91 | Hg38_1_172365073_172375924_172561646_172570053_RR | OBD160.361.363 |
| 92 | Hg38_1 167272741_167278581_167541602_167544034_FF | OBD160.365.367 |
| 93 | Hg38_1_27783032_27786599_27834284_27836433_FF | OBD160.369.371 |
| 94 | Hg38_2_144038117_144048675_144274589_144280493_FF | OBD160.373.375 |
| 95 | Hg38_17_41002849_41006661_41059440_41062968_RF | OBD160.377.379 |
| 96 | Hg38_11_90904893_90913850_91035518_91039190_FF | OBD160.381.383 |
| 97 | Hg38_1_247706861_247710350_247812273_247815652_FR | OBD160.385.387 |
| 98 | Hg38_12_23014365_23020377_23180775_23184338_RR | OBD160.389.391 |
| 99 | Hg38_4_28348510_28364344_28414655_28418162_FF | OBD160.393.395 |
| 100 | Hg38_17_35578150_35580887_35656691_35659920_FF | OBD160.397.399 |

TABLE 1A
(part 3a)

| | | | | FC: |
|---|---|---|---|---|
| No. | probe | RP/Rsum | Rprank | (class1/class2) |
| 101 | Hg38_13_28438004_28449201_28551336_28558541_RR | 5473.459527 | 216 | 1.548 |
| 102 | Hg38_8_132637112_132642401_132681711 132690255_FR | 3591.973084 | 130 | 1.545 |
| 103 | Hg38_20_33827938_33830467_34035296_34047038_FR | 3359.632084 | 122 | 1.545 |
| 104 | Hg38_4_55932433_55939189_55969220_55974802_FF | 2903.160734 | 91 | 1.545 |
| 105 | Hg38_7_142995810_142998826_143057496_143064818_RR | 3759.403871 | 140 | 1.544 |
| 106 | Hg38_18_75676630_75682929_75700545_75706785_RR | 2791.763006 | 83 | 1.544 |
| 107 | Hg38_5_12486934_12496757_12590668_12597008_FF | 2558.874957 | 71 | 1.544 |
| 108 | Hg38_7_113374693_113380998_113411335_113420030_FR | 2582.839821 | 73 | 1.542 |
| 109 | Hg38_3_98493912_98497028_98612320_98617232_RF | 2676.998144 | 77 | 1.54 |
| 110 | Hg38_6_71312879_71323494_71524385_71527337_FR | 2845.485946 | 87 | 1.539 |
| 111 | Hg38_4_71700748_71714803_71741385_71749255_FF | 3490.546135 | 128 | 1.537 |
| 112 | Hg38_13_62231930_62237495_62330216_62339040_FR | 2587.198527 | 74 | 1.537 |
| 113 | Hg38_12_90700058_90702509_90796185_90802319_RF | 3605.32438 | 131 | 1.536 |
| 114 | Hg38_4_67686135_67693477_67782747_67789927_RR | 2955.347004 | 95 | 1.536 |
| 115 | Hg38_6_39611834_39623490_39813403_39823235_FF | 3385.094489 | 123 | 1.535 |
| 116 | Hg38_6_125238508_125249295_125290548_125297233_FF | 3558.225301 | 129 | 1.534 |
| 117 | Hg38_4_126064916_126070028_126094394_126106891_RR | 3138.820724 | 105 | 1.534 |
| 118 | Hg38_10_12235815_12238464_12383561_12388246_FR | 2359.06621 | 60 | 1.534 |
| 119 | Hg38_7_88599386_88615125_88740721_88745628_FF | 2728.216 | 82 | 1.533 |
| 120 | Hg38_2_52892382_52905124_52921980_52933672_RR | 3219.804543 | 110 | 1.532 |
| 121 | Hg38_1_74603597_74607998_74778978_74800362_FR | 3742.651266 | 139 | 1.531 |
| 122 | Hg38_Y_22721647_22725457_22859118_22870072_RF | 3212.534981 | 109 | 1.53 |
| 123 | Hg38_4_97463862_97470977_97576564_97590463_RR | 3052.561735 | 101 | 1.53 |
| 124 | Hg38_1_96357496_96364474_96392998_96404426_RF | 2982.624056 | 98 | 1.53 |
| 125 | Hg38_1_158905246_158912243_159126541_159136350_FF | 2817.776717 | 85 | 1.53 |
| 126 | Hg38_11_106098496_106106725_106275961_106290738_RR | 3268.3717 | 114 | 1.529 |
| 127 | Hg38_11_72859306_72866489_72925691_72936492_RF | 3458.227037 | 127 | 1.525 |
| 128 | Hg38_14_49020009_49028815_49076509_49078072_FR | 4442.91003 | 165 | 1.524 |
| 129 | Hg38_4_173125717_173130524_173207477_173215489_FR | 3607.202568 | 132 | 1.524 |
| 130 | Hg38_14_19825779_19829413_19968132_19975589_FF | 3185.904497 | 107 | 1.524 |
| 131 | Hg38_15_56425187_56434244_56691312_56696438_FR | 2916.936995 | 92 | 1.524 |
| 132 | Hg38_7_30612223_30619592_30804622_30809524_FR | 4045.43792 | 152 | 1.523 |
| 133 | Hg38_20_46372997_46377307_46622048_46631132_FF | 3314.955421 | 118 | 1.523 |
| 134 | Hg38_14_24423148_24425179_24517993_24525921_RR | 5033.707838 | 189 | 1.522 |
| 135 | Hg38_7_93821969_93826969_93960806_93964392_FR | 3286.118138 | 115 | 1.522 |
| 136 | Hg38_2_36912180_36914993_37085573_37089692_RR | 3310.62397 | 117 | 1.521 |
| 137 | Hg38_4_77717993_77727364_77982270_77985832_RR | 3083.119716 | 102 | 1.521 |
| 138 | Hg38_3_87991842_87995289_88126188_88133417_FF | 3239.086494 | 111 | 1.518 |
| 139 | Hg38_4_121310394_121315169_121339971_121354127_RF | 3150.912042 | 106 | 1.518 |
| 140 | Hg38_2_15677778_15684445_15733618_15739157_RR | 4181.003667 | 156 | 1.517 |
| 141 | Hg38_12_29701302_29708431_29828391_29843699_FF | 3425.857677 | 124 | 1.517 |
| 142 | Hg38_3_64067438_64075904_64228797_64236262_FR | 3767.332371 | 143 | 1.516 |
| 143 | Hg38_17_40700822_40703790_40868811_40875117_FR | 3434.139591 | 125 | 1.516 |
| 144 | Hg38_9_120795804_120798748_120831347_120833895_RR | 3002.794717 | 100 | 1.515 |
| 145 | Hg38_7_144246203_144258031_144391768_144394587_FR | 5823.554282 | 235 | 1.513 |
| 146 | Hg38_12_71685734_71687005_71771992_71780701_FR | 4285.485291 | 160 | 1.513 |
| 147 | Hg38_11_82067024_82073102_82131728_82146376_RF | 3887.012897 | 147 | 1.513 |
| 148 | Hg38_7_144153639_144162181_144246203_144258031_RF | 6897.235002 | 308 | 1.512 |
| 149 | Hg38_2_21769905_21774394_21960934_21972734_FR | 5044.788457 | 190 | 1.512 |
| 150 | Hg38_4_38385027_38395353_38450896_38455314_RF | 3309.983405 | 116 | 1.512 |

TABLE 1A (part 3b)

| | | | | Probe sequence |
|---|---|---|---|---|
| No. | pfp | P.value | Type | 60 mer |
| 101 | 0 | 0 | SBMA | AATAAAATGATGGTGTCTTAAGCATAAATCGATTGATGTAGGAGTATTAGTAGTAATTGA |
| 102 | 0 | 0 | SBMA | TCATGCTATTTCTCATGGAGAGATGGCATCGAAGATGAAATTAATTTTAAATGATTGTCT |
| 103 | 0 | 0 | SBMA | ATTTATTTTCTCAGTTCTGGAGACTCAGTCGAAGGCTAGAGTTATCAGAAAATGTTAATT |
| 104 | 0 | 0 | SBMA | AGTTAATATAGAAACAACATTCTTCTTTTCGACGTTTTATTCCATGTTCTAGTCTCATCC |
| 105 | 0 | 0 | SBMA | TTGTTTGTTTACTTTGCTATTGAGTTCTTCGATTTCCAGACATTTCCATACGTCCTCTGA |
| 106 | 0 | 0 | SBMA | ACATCTGATTTAACCAAATCTTTGCCAATCGATTGTATCTTCTATAATTAGCAATAGCTA |
| 107 | 0 | 0 | SBMA | ATATATTTAATGAAATAAAATTACTATATCGATTACACTCTGGGCTAGAGGTTCCTAAAT |
| 108 | 0 | 0 | SBMA | CCATAATTTTTAGCACATAAAACTTTTCTCGACAGAAGTGAAGAAACAATCCTGAAAAAA |
| 109 | 0 | 0 | SBMA | TGACATGATCTAAGATAATCTCCTTATCTCGAGGTGAGAATTTAACACATTATTAAATTA |
| 110 | 0 | 0 | SBMA | ATGTCAATAAAATTGTACAGGATTACATTCGAAAAAACCAAAAACCAAACAAACAAAAAA |
| 111 | 0 | 0 | SBMA | ATAAAACAGATGAATAGACTGTAAATAATCGACAGTAAATTTATAGCAGCTGAGTAGCCT |
| 112 | 0 | 0 | SBMA | ACTGAGTTTATACAACCTTTTAAAATTTTCGAGAATAACTCAGGATAAAATAGATACAAG |
| 113 | 0 | 0 | SBMA | TATTGTGGGGTTTTTTTATTGTTTGTTTTCGAGTAAAAAACAGTTTTTGCAATATCAAGT |
| 114 | 0 | 0 | SBMA | GTTCTTTCATAAAAGGGTGAATTTTTAATCGATGTTTACCATTTTGACTGAAGATATAAT |
| 115 | 0 | 0 | SBMA | AAAATGTACTATCTTGTTCATAAAGTTCTCGATGTAGCCTTTCTTAATCTTTGGGCCTTT |
| 116 | 0 | 0 | SBMA | ATTTTTCCCTCTATTCTTTAAAAACTTTTCGAGATTCTTAGTTGCAGAAAGGAATTAGCA |
| 117 | 0 | 0 | SBMA | TCTGCATTTAATGTTGCAACATGAGGAATCGATGTATAATGTTGTTCAGTTAAACTATGT |
| 118 | 0 | 0 | SBMA | TGACTTTTTTTCTCCTTTACAGTTGTGTTCGAGCTTGTTATTTTCTCTTTCTTACCTAGT |
| 119 | 0 | 0 | SBMA | ACACAACTATGATGCATAAGAAAAAAAATCGAATTTAAAAACAAAAAAAGCCCCTAATAT |
| 120 | 0 | 0 | SBMA | AATAACTGCAGAATATACATTTTTCCCATCGAGTCTGATTGATTCAGACTCTGAAATCAA |
| 121 | 0 | 0 | SBMA | TATCATCACCACTTTACAAATGAGAAAATCGAAAATAGAAACTTACGTTATCAGGTTCAA |
| 122 | 0 | 0 | SBMA | ACTATATTATGACCTGTATCAAGATGTATCGAAATACAAGAATCTAGTCTGATTAGATTA |
| 123 | 0 | 0 | SBMA | ATGGGATCTCAGAATAGAAAAAGAACATTCGACCTCTACATATCATCTAAAGAAATTAAC |
| 124 | 0 | 0 | SBMA | ATTTTTAAAATGAAAAAAATGACATAATTCGACCTCTCTTTTCTTGCTGGAAAATATGCT |
| 125 | 0 | 0 | SBMA | CAGCTTAAAATAATGGGTTATGGTAACCTCGAGGTAGATACATAGGAGGTGCAAACACAA |
| 126 | 0 | 0 | SBMA | AAACATTCAAATTTTTTTCTTTTACTAATCGAGGGATATGATGTTCTCAGTTGGATTTTT |
| 127 | 0 | 0 | SBMA | CAGTGTGATTTTATCAAACTCAAGAATCTCGAGTATATGCAAGCTATGTAAGCTACTTGT |
| 128 | 0 | 0 | SBMA | ATTTTATAAAGCCAGAATAACCCTGATATCGAACCCTATAAATACTGTTTTTTCCTATAA |
| 129 | 0 | 0 | SBMA | AATGTTATGAAATGAAGACATATGTGACTCGAAACCATCTTTCATACCATTAAATATTTT |
| 130 | 0 | 0 | SBMA | GCTATTTTAAACAAAACATCATTTAATATCGATTTTGGAAGGGACTTAATTTCAGCTACC |
| 131 | 0 | 0 | SBMA | TTTGTTTCTGAGCATTCATTAATTCTATTCGATTACTGATAGATATACTGTAGGTACATT |
| 132 | 0 | 0 | SBMA | GGTTAAGAACATAAGTATACTTAACCAGTCGATATCTGTTGAGTCCTCACTATCTGACTG |
| 133 | 0 | 0 | SBMA | TTTATTACTTGTTGAAATGGTAACGTTTTCGAGAACCACTGGATTAAATGATCTCAGCTT |
| 134 | 0 | 0 | SBMA | AAATATTGTAGTACAAATGTCTACACTCTCGATTTGTGGTTGTTGCCAGGAATGGAGGCA |
| 135 | 0 | 0 | SBMA | ACAGAAATGAGAAAAACAATAGAGAATATCGACTCTAAAGTCTATGTAGAGAAGCAAAGG |
| 136 | 0 | 0 | SBMA | ACCAATAAAAGGGAAAGAGTAGAGATACTCGAGAAAATGAAGGAAATGATTGCCATAAAA |
| 137 | 0 | 0 | SBMA | AAACAGCTTAAGTTTATTTTGTGTATGATCGATAAGCTACCCTTCCTTAGGATAACAAAT |
| 138 | 0 | 0 | SBMA | CCGGATAATTTTTTTTATTTCTTACTTTTCGATCCTACCTATTTCCCCAACCATTAATAT |
| 139 | 0 | 0 | SBMA | TTTGATTCCTATAGTTTTGCAGTATAATTCGAAGATCAAAACATGCAAATACTAGCAGTC |
| 140 | 0 | 0 | SBMA | CAGGACTGTAAAAGAGCCTTTTCTTCTTTCGATTGGAACCTTTATGTCTGGACTCTGAAA |
| 141 | 0 | 0 | SBMA | ACCAAAAGTCAGAGGAACAAATAATTTGTCGATTCTTCAGAACTTCCTTGATATTTTAAA |
| 142 | 0 | 0 | SBMA | ATTCTCTATCTCCATGAATTTAATTGTTTCGACCTTCAGAACTTTCTGTTTAGGTAGAAT |
| 143 | 0 | 0 | SBMA | CTGCCTTTTTGGCCTTTATATACACACTTCGAAATCCTTTGTTGGATTCAGATTTCTAAA |
| 144 | 0 | 0 | SBMA | AAACATCATTTCTACCAGCAAGTTTATTTCGAACAATGTCATTTGTTTTCATTACACTTT |
| 145 | 0 | 0 | SBMA | AAACAGAACCCGGATTTTTACAGACTGATCGAAGTTAACTGCACATCCTTTTTTTTATCT |
| 146 | 0 | 0 | SBMA | GCCTTTAATAAATGCTTGTTGTGATAATTCGAGTCATTTAGGTTGAAAGGGACCAGTTGT |
| 147 | 0 | 0 | SBMA | GAATGCACAATGTAAAACTATTAACCTTTCGAACTTATCTCTATTTTTACTGTACTGTGG |
| 148 | 0 | 0 | SBMA | AAACAGAACCCGGATTTTTACAGACTGATCGACTCAAAAATTACTGTATGTGATATGGGA |
| 149 | 0 | 0 | SBMA | TGTCCCAGTAAATCGCTGGGGAATAGAATCGATAAGAACTATGAAAAATTTTAATTTAAA |
| 150 | 0 | 0 | SBMA | ATCACTTTCAGAAAAGAATAGAGGTTTTTCGATATTCAAGCTCACAAAATAAGCTCAAAC |

TABLE 1A
(part 3c)

| | Probe Location | | | | |
|---|---|---|---|---|---|
| No. | Chr | Start1 | End1 | Start2 | End2 |
| 101 | 13 | 28438006 | 28438035 | 28551338 | 28551367 |
| 102 | 8 | 132642370 | 132642399 | 132681713 | 132681742 |
| 103 | 20 | 33830436 | 33830465 | 34035298 | 34035327 |
| 104 | 4 | 55939158 | 55939187 | 55974771 | 55974800 |
| 105 | 7 | 142995812 | 142995841 | 143057498 | 143057527 |
| 106 | 18 | 75676632 | 75676661 | 75700547 | 75700576 |
| 107 | 5 | 12496726 | 12496755 | 12596977 | 12597006 |
| 108 | 7 | 113380967 | 113380996 | 113411337 | 113411366 |
| 109 | 3 | 98493914 | 98493943 | 98617201 | 98617230 |
| 110 | 6 | 71323463 | 71323492 | 71524387 | 71524416 |
| 111 | 4 | 71714772 | 71714801 | 71749224 | 71749253 |
| 112 | 13 | 62237464 | 62237493 | 62330218 | 62330247 |
| 113 | 12 | 90700060 | 90700089 | 90802288 | 90802317 |
| 114 | 4 | 67686137 | 67686166 | 67782749 | 67782778 |
| 115 | 6 | 39623459 | 39623488 | 39823204 | 39823233 |
| 116 | 6 | 125249264 | 125249293 | 125297202 | 125297231 |
| 117 | 4 | 126064918 | 126064947 | 126094396 | 126094425 |
| 118 | 10 | 12238433 | 12238462 | 12383563 | 12383592 |
| 119 | 7 | 88615094 | 88615123 | 88745597 | 88745626 |
| 120 | 2 | 52892384 | 52892413 | 52921982 | 52922011 |
| 121 | 1 | 74607967 | 74607996 | 74778980 | 74779009 |
| 122 | Y | 22721649 | 22721678 | 22870041 | 22870070 |
| 123 | 4 | 97463864 | 97463893 | 97576566 | 97576595 |
| 124 | 1 | 96357498 | 96357527 | 96404395 | 96404424 |
| 125 | 1 | 158912212 | 158912241 | 159136319 | 159136348 |
| 126 | 11 | 106098498 | 106098527 | 106275963 | 106275992 |
| 127 | 11 | 72859308 | 72859337 | 72936461 | 72936490 |
| 128 | 14 | 49028784 | 49028813 | 49076511 | 49076540 |
| 129 | 4 | 173130493 | 173130522 | 173207479 | 173207508 |
| 130 | 14 | 19829382 | 19829411 | 19975558 | 19975587 |
| 131 | 15 | 56434213 | 56434242 | 56691314 | 56691343 |
| 132 | 7 | 30619561 | 30619590 | 30804624 | 30804653 |
| 133 | 20 | 46377276 | 46377305 | 46631101 | 46631130 |
| 134 | 14 | 24423150 | 24423179 | 24517995 | 24518024 |
| 135 | 7 | 93826938 | 93826967 | 93960808 | 93960837 |
| 136 | 2 | 36912182 | 36912211 | 37085575 | 37085604 |
| 137 | 4 | 77717995 | 77718024 | 77982272 | 77982301 |
| 138 | 3 | 87995258 | 87995287 | 88133386 | 88133415 |
| 139 | 4 | 121310396 | 121310425 | 121354096 | 121354125 |
| 140 | 2 | 15677780 | 15677809 | 15733620 | 15733649 |
| 141 | 12 | 29708400 | 29708429 | 29843668 | 29843697 |
| 142 | 3 | 64075873 | 64075902 | 64228799 | 64228828 |
| 143 | 17 | 40703759 | 40703788 | 40868813 | 40868842 |
| 144 | 9 | 120795806 | 120795835 | 120831349 | 120831378 |
| 145 | 7 | 144258000 | 144258029 | 144391770 | 144391799 |
| 146 | 12 | 71686974 | 71687003 | 71771994 | 71772023 |
| 147 | 11 | 82067026 | 82067055 | 82146345 | 82146374 |
| 148 | 7 | 144153641 | 144153670 | 144258000 | 144258029 |
| 149 | 2 | 21774363 | 21774392 | 21960936 | 21960965 |
| 150 | 4 | 38385029 | 38385058 | 38455283 | 38455312 |

TABLE 1A
(part 3d)

| | 4 kb Sequence Location | | | | |
|---|---|---|---|---|---|
| No. | Chr | Start1 | End1 | Start2 | End2 |
| 101 | 13 | 28438006 | 28442005 | 28551338 | 28555337 |
| 102 | 8 | 132638400 | 132642399 | 132681713 | 132685712 |
| 103 | 20 | 33826466 | 33830465 | 34035298 | 34039297 |
| 104 | 4 | 55935188 | 55939187 | 55970801 | 55974800 |
| 105 | 7 | 142995812 | 142999811 | 143057498 | 143061497 |
| 106 | 18 | 75676632 | 75680631 | 75700547 | 75704546 |
| 107 | 5 | 12492756 | 12496755 | 12593007 | 12597006 |
| 108 | 7 | 113376997 | 113380996 | 113411337 | 113415336 |
| 109 | 3 | 98493914 | 98497913 | 98613231 | 98617230 |
| 110 | 6 | 71319493 | 71323492 | 71524387 | 71528386 |
| 111 | 4 | 71710802 | 71714801 | 71745254 | 71749253 |
| 112 | 13 | 62233494 | 62237493 | 62330218 | 62334217 |
| 113 | 12 | 90700060 | 90704059 | 90798318 | 90802317 |
| 114 | 4 | 67686137 | 67690136 | 67782749 | 67786748 |
| 115 | 6 | 39619489 | 39623488 | 39819234 | 39823233 |
| 116 | 6 | 125245294 | 125249293 | 125293232 | 125297231 |
| 117 | 4 | 126064918 | 126068917 | 126094396 | 126098395 |
| 118 | 10 | 12234463 | 12238462 | 12383563 | 12387562 |
| 119 | 7 | 88611124 | 88615123 | 88741627 | 88745626 |
| 120 | 2 | 52892384 | 52896383 | 52921982 | 52925981 |
| 121 | 1 | 74603997 | 74607996 | 74778980 | 74782979 |
| 122 | Y | 22721649 | 22725648 | 22866071 | 22870070 |
| 123 | 4 | 97463864 | 97467863 | 97576566 | 97580565 |
| 124 | 1 | 96357498 | 96361497 | 96400425 | 96404424 |
| 125 | 1 | 158908242 | 158912241 | 159132349 | 159136348 |
| 126 | 11 | 106098498 | 106102497 | 106275963 | 106279962 |
| 127 | 11 | 72859308 | 72863307 | 72932491 | 72936490 |
| 128 | 14 | 49024814 | 49028813 | 49076511 | 49080510 |
| 129 | 4 | 173126523 | 173130522 | 173207479 | 173211478 |
| 130 | 14 | 19825412 | 19829411 | 19971588 | 19975587 |
| 131 | 15 | 56430243 | 56434242 | 56691314 | 56695313 |
| 132 | 7 | 30615591 | 30619590 | 30804624 | 30808623 |
| 133 | 20 | 46373306 | 46377305 | 46627131 | 46631130 |
| 134 | 14 | 24423150 | 24427149 | 24517995 | 24521994 |
| 135 | 7 | 93822968 | 93826967 | 93960808 | 93964807 |
| 136 | 2 | 36912182 | 36916181 | 37085575 | 37089574 |
| 137 | 4 | 77717995 | 77721994 | 77982272 | 77986271 |
| 138 | 3 | 87991288 | 87995287 | 88129416 | 88133415 |
| 139 | 4 | 121310396 | 121314395 | 121350126 | 121354125 |
| 140 | 2 | 15677780 | 15681779 | 15733620 | 15737619 |
| 141 | 12 | 29704430 | 29708429 | 29839698 | 29843697 |
| 142 | 3 | 64071903 | 64075902 | 64228799 | 64232798 |
| 143 | 17 | 40699789 | 40703788 | 40868813 | 40872812 |
| 144 | 9 | 120795806 | 120799805 | 120831349 | 120835348 |
| 145 | 7 | 144254030 | 144258029 | 144391770 | 144395769 |
| 146 | 12 | 71683004 | 71687003 | 71771994 | 71775993 |
| 147 | 11 | 82067026 | 82071025 | 82142375 | 82146374 |
| 148 | 7 | 144153641 | 144157640 | 144254030 | 144258029 |
| 149 | 2 | 21770393 | 21774392 | 21960936 | 21964935 |
| 150 | 4 | 38385029 | 38389028 | 38451313 | 38455312 |

TABLE 1A
(part 3e)

| No. | probe | Primer ID |
|---|---|---|
| 101 | Hg38_13_28438004_28449201_28551336_28558541_RR | OBD160.401 |
| 102 | Hg38_8_132637112_132642401_132681711 132690255_FR | OBD160.405 |
| 103 | Hg38_20_33827938_33830467_34035296_34047038_FR | OBD160.409 |
| 104 | Hg38_4_55932433_55939189_55969220_55974802_FF | OBD160.413 |
| 105 | Hg38_7_142995810_142998826_143057496_143064818_RR | OBD160.417 |
| 106 | Hg38_18_75676630_75682929_75700545_75706785_RR | OBD160.421 |
| 107 | Hg38_5_12486934_12496757_12590668_12597008_FF | OBD160.425 |
| 108 | Hg38_7_113374693_113380998_113411335_113420030_FR | OBD160.429 |
| 109 | Hg38_3_98493912_98497028_98612320_98617232_RF | OBD160.433 |
| 110 | Hg38_6_71312879_71323494_71524385_71527337_FR | OBD160.437 |
| 111 | Hg38_4_71700748_71714803_71741385_71749255_FF | OBD160.441 |
| 112 | Hg38_13_62231930_62237495_62330216_62339040_FR | OBD160.445 |
| 113 | Hg38_12_90700058_90702509_90796185_90802319_RF | OBD160.449 |
| 114 | Hg38_4_67686135_67693477_67782747_67789927_RR | OBD160.453 |
| 115 | Hg38_6_39611834_39623490_39813403_39823235_FF | OBD160.457 |
| 116 | Hg38_6_125238508_125249295_125290548_125297233_FF | OBD160.461 |
| 117 | Hg38_4_126064916_126070028_126094394_126106891_RR | OBD160.465 |
| 118 | Hg38_10_12235815_12238464_12383561_12388246_FR | OBD160.469 |
| 119 | Hg38_7_88599386_88615125_88740721_88745628_FF | OBD160.473 |
| 120 | Hg38_2_52892382_52905124_52921980_52933672_RR | OBD160.477 |
| 121 | Hg38_1_74603597_74607998_74778978_74800362_FR | OBD160.481 |
| 122 | Hg38_Y_22721647_22725457_22859118_22870072_RF | OBD160.485 |
| 123 | Hg38_4_97463862_97470977_97576564_97590463_RR | OBD160.489 |
| 124 | Hg38_1_96357496_96364474_96392998_96404426_RF | OBD160.493 |
| 125 | Hg38_1_158905246_158912243_159126541_159136350_FF | OBD160.497 |
| 126 | Hg38_11_106098496_106106725_106275961_106290738_RR | OBD160.501 |
| 127 | Hg38_11_72859306_72866489_72925691 72936492_RF | OBD160.505 |
| 128 | Hg38_14_49020009_49028815_49076509_49078072_FR | OBD160.509 |
| 129 | Hg38_4_173125717_173130524_173207477_173215489_FR | OBD160.513 |
| 130 | Hg38_14_19825779_19829413_19968132_19975589_FF | OBD160.517 |
| 131 | Hg38_15_56425187_56434244_56691312_56696438_FR | OBD160.521 |
| 132 | Hg38_7_30612223_30619592_30804622_30809524_FR | OBD160.525 |
| 133 | Hg38_20_46372997_46377307_46622048_46631132_FF | OBD160.529 |
| 134 | Hg38_14_24423148_24425179_24517993_24525921_RR | OBD160.533 |
| 135 | Hg38_7_93821969_93826969_93960806_93964392_FR | OBD160.537 |
| 136 | Hg38_2_36912180_36914993_37085573_37089692_RR | OBD160.541 |
| 137 | Hg38_4_77717993_77727364_77982270_77985832_RR | OBD160.545 |
| 138 | Hg38_3_87991842_87995289_88126188_88133417_FF | OBD160.549 |
| 139 | Hg38_4_121310394_121315169_121339971 121354127_RF | OBD160.553 |
| 140 | Hg38_2_15677778_15684445_15733618_15739157_RR | OBD160.557 |
| 141 | Hg38_12_29701302_29708431_29828391_29843699_FF | OBD160.561 |
| 142 | Hg38_3_64067438_64075904_64228797_64236262_FR | OBD160.565 |
| 143 | Hg38_17_40700822_40703790_40868811_40875117_FR | OBD160.569 |
| 144 | Hg38_9_120795804_120798748_120831347_120833895_RR | OBD160.573 |
| 145 | Hg38_7_144246203_144258031_144391768_144394587_FR | OBD160.577 |
| 146 | Hg38_12_71685734_71687005_71771992_71780701_FR | OBD160.581 |
| 147 | Hg38_11_82067024_82073102_82131728_82146376_RF | OBD160.585 |
| 148 | Hg38_7_144153639_144162181_144246203_144258031_RF | OBD160.589 |
| 149 | Hg38_2_21769905_21774394_21960934_21972734_FR | OBD160.593 |
| 150 | Hg38_4_38385027_38395353_38450896_38455314_RF | OBD160.597 |

TABLE 1A (part 3f)

| No. | Sequence | Primer ID | Sequence |
|---|---|---|---|
| 101 | GTGGACTTTCCTGTAACATCCTTTTA | OBD160.403 | GGCTGGGTTCTAAAGATGAAGACTT |
| 102 | GTGGGCACTTGGCTCTGAGATGC | OBD160.407 | GCCCTCAACGGTGGAGTAGGATA |
| 103 | GGTGGGAGAGACACTGAGGCTGT | OBD160.411 | CACCACCTACCTGCCAAGTTGCT |
| 104 | GATGTCCCACTACCTTGCCTGAGG | OBD160.415 | GCAGGGAGACTTGGACCTATTGG |
| 105 | CCAGCAGTGGATGAGGGCTCGCCTGTCT | OBD160.419 | CACAGCAGCCTCTTTCATCACAGCCCAG |
| 106 | GGAATGATAGAGATAGGGAGAAATGGAA | OBD160.423 | GAGAAGCAGACGGAAACTGTGACTGACA |
| 107 | CTCAAGCAACTCAGCAGCATAAAACACA | OBD160.427 | CTTCCCCACTCATTTCTTGTGATTCTAT |
| 108 | TTTATTTGTCAAGTGCCAAGGAATGC | OBD160.431 | TAGGAAACACCAGTGAGTCCAAGGCT |
| 109 | CACAGTCCCCTCTTACTACAGCGTGAAA | OBD160.435 | ACACTATTTCATTGTAGAGGTTACCACG |
| 110 | CTTGGAAATGTCAGACCCTTGCCCAT | OBD160.439 | ATTGAGCCGTGTGTTTGGTGCTTGC |
| 111 | GGCTGGATGGCAGTAGAATGCTGTTC | OBD160.443 | CTCCATTCATCGCACTCTACTTTAGC |
| 112 | CCTGCCTCAACCTCCCAAGTAGC | OBD160.447 | CTGAGGTAAACGGAGTAAAGGGTA |
| 113 | CTGTTGGTAGTTCCTAACCTCATA | OBD160.451 | CAATAAGAAGTAAACAAAGCATAAC |
| 114 | GCCCCAAGGGAATAAAACTAAGACTA | OBD160.455 | CTTCTTCTCTCTTCATCACAACAGTC |
| 115 | CTTCTTGGTTGTCTATCCTTTCAGAT | OBD160.459 | CAAGTTGGCACCGAGTGGTTTCCTAA |
| 116 | ACCAAAAGTTCTCACTGGAGGCACCA | OBD160.463 | CTCGTCCACAATGTCCAGATTTACCC |
| 117 | CCATCCCCAGGACCCCACTGTGC | OBD160.467 | CAGACAAAGGTGACTTCAGAGCAAGAAC |
| 118 | GTGACTCCCATCTTTCCCATAGAACCAG | OBD160.471 | CACTCTTGTGTCGCTCCTGAGGCTA |
| 119 | CGGGATTCATAAAGGAGAAAGCAAGA | OBD160.475 | GTGCCAACCCCACAGAGCAACTTATG |
| 120 | GGAAAAGGAAGGACGCAAATGGTGTCAC | OBD160.479 | GTCTTGTGCCAGTTTTCAAAGGGAGAGC |
| 121 | TAAGATTATTGAGCACTCTCTCTGTG | OBD160.483 | GGGTGTAGGGCACAGATTCTTAC |
| 122 | TCCAGCAAAAGTATCCTTCAACAGG | OBD160.487 | CTGCTGTATGCTCAGGCTTTGCTCC |
| 123 | TACAGCAATAGAAAGGAGTGCGGTAT | OBD160.491 | GTCAAAGCCAGTGTCCAGAAGGGTAT |
| 124 | GCCTCATTAGGAAAGTGTAGAAGAGA | OBD160.495 | GACTATCCTGGAGACTCTGGAAAACT |
| 125 | GTAGAAAGACTAAAAGACAAACCTCTC | OBD160.499 | CACGCACAGCCTCCAAGAAACTA |
| 126 | CTTGACCTTTATTTACTATGCTTGGC | OBD160.503 | CAAGGGACCTCTCAAAGCAGAAGCAA |
| 127 | ATTTTGGTGACAGACTGGGCTTCT | OBD160.507 | CATCTCTCTCTTCTTCATTACAGTCCCC |
| 128 | AAGGATTGGTGAACTCAAAGACAGGTTA | OBD160.511 | GGTATTTTGAGAGAGAGTCCACATTGAC |
| 129 | CGGAACTTTAGCATCTGGAAGAAGAA | OBD160.515 | CAACATCAGAGAGGTGAGAAACAACC |
| 130 | CAGTCTTTTGGTAGAGCAATCAGGAT | OBD160.519 | GAACATCTCCTTGACAACCCTCCCCT |
| 131 | GTTGGCGACGCTCTTCAATAAGTTTT | OBD160.523 | GGAGCAACACAAGCCAAACACAGCAG |
| 132 | CCATCCAGAGAAGTAACCACTATCCC | OBD160.527 | GACTGCTTGGTCATTTTGTGCCTCAG |
| 133 | CCTGTGAGGTCCAGTTTGGTAGCCCC | OBD160.531 | CCTTCAGTTTCCTTGTGTGCTGTAGACA |
| 134 | ACTCACTCCTTCCCTTCAGCAGC | OBD160.535 | ACTGCCTCAGGAGTTGGCTGCCT |
| 135 | ATGAAACAGAAATGAGAAAAACA | OBD160.539 | TCTATGTAGAGAAGCAAAGGGCCAA |
| 136 | GTGATGTGTGTCTTAGAGGAGTAACC | OBD160.543 | AGCCCTCTTTGTCCCCTATCCAGTTG |
| 137 | TTGGGTGGGTTTTCTGCTCTGGAGGTAT | OBD160.547 | ATCAGGAGAAGAGCAAGAGTTGAAATCA |
| 138 | CTCACCTCAGTCTCCCAAGTAGC | OBD160.551 | GTGTGCCACTCCAATCACTGTAT |
| 139 | TTATTGAAGAGACTGTCCTTTCCCTA | OBD160.555 | GTGAATAGTGCCGCAATAAACATACG |
| 140 | ATTATGCCTCCTACATTCCCTCCCCG | OBD160.559 | CCTCCCTGTCTTCCCTGTTGATAAAA |
| 141 | GAATGAGTGAATACAGCCAGGGAATG | OBD160.563 | TGGCTATGGAGATGTCACCTGAAGGG |
| 142 | ACCCATCCCCAAGCATTTATCCTTTGTG | OBD160.567 | GTTGCTGCTGTTTTGCTGTCTTGTGTCC |
| 143 | GGCGAATAGGAGAGGTGATGACG | OBD160.571 | GCCCTGTTTCCCACGAGGTAGGA |
| 144 | GTCATCTAAACTACCTCCCCAGGCAAGT | OBD160.575 | GGTTTAGAATGGATGATTTGCGTAATGG |
| 145 | CAGCGGCAAACACAGGCAGATGT | OBD160.579 | CCCCAGAGCCCCATCCTGTCAAT |
| 146 | CCTCTGAATGGTTATGAATGGTTGTG | OBD160.583 | CCTGCCTGGTCAACCCACAGAATCAT |
| 147 | ATTCAGTGTAATCCCTATCAAAAT | OBD160.587 | TCTGCCATAAAGTCATTCTCAAAA |
| 148 | CCATTGAACAGTAGAGACAGGGTCCC | OBD160.591 | GGCACTCTTTCTGCTCTATTTTGTGG |
| 149 | GTTATTACTGGTTGATGACTCCACAT | OBD160.595 | GCAATCTCAGAAATCAACACTAAAGAAC |
| 150 | GATGTCTGAGAATGTGGAGTGTGCTA | OBD160.599 | TTTACCGCCTTTTCCTCCCACCTCTC |

TABLE 1A
(part 3g)

| No. | probe | Marker |
|---|---|---|
| 101 | Hg38_13_28438004_28449201_28551336_28558541_RR | OBD160.401.403 |
| 102 | Hg38_8_132637112_132642401_132681711_132690255_FR | OBD160.405.407 |
| 103 | Hg38_20_33827938_33830467_34035296_34047038_FR | OBD160.409.411 |
| 104 | Hg38_4_55932433_55939189_55969220_55974802_FF | OBD160.413.415 |
| 105 | Hg38_7_142995810_142998826_143057496_143064818_RR | OBD160.417.419 |
| 106 | Hg38_18_75676630_75682929_75700545_75706785_RR | OBD160.421.423 |
| 107 | Hg38_5_12486934_12496757_12590668_12597008_FF | OBD160.425.427 |
| 108 | Hg38_7_113374693_113380998_113411335_113420030_FR | OBD160.429.431 |
| 109 | Hg38_3_98493912_98497028_98612320_98617232_RF | OBD160.433.435 |
| 110 | Hg38_6_71312879_71323494_71524385_71527337_FR | OBD160.437.439 |
| 111 | Hg38_4_71700748_71714803_71741385_71749255_FF | OBD160.441.443 |
| 112 | Hg38_13_62231930_62237495_62330216_62339040_FR | OBD160.445.447 |
| 113 | Hg38_12_90700058_90702509_90796185_90802319_RF | OBD160.449.451 |
| 114 | Hg38_4_67686135_67693477_67782747_67789927_RR | OBD160.453.455 |
| 115 | Hg38_6_39611834_39623490_39813403_39823235_FF | OBD160.457.459 |
| 116 | Hg38_6_125238508_125249295_125290548_125297233_FF | OBD160.461.463 |
| 117 | Hg38_4_126064916_126070028_126094394_126106891_RR | OBD160.465.467 |
| 118 | Hg38_10_12235815_12238464_12383561_12388246_FR | OBD160.469.471 |
| 119 | Hg38_7_88599386_88615125_88740721_88745628_FF | OBD160.473.475 |
| 120 | Hg38_2_52892382_52905124_52921980_52933672_RR | OBD160.477.479 |
| 121 | Hg38_1_74603597_74607998_74778978_74800362_FR | OBD160.481.483 |
| 122 | Hg38_Y_22721647_22725457_22859118_22870072_RF | OBD160.485.487 |
| 123 | Hg38_4_97463862_97470977_97576564_97590463_RR | OBD160.489.491 |
| 124 | Hg38_1_96357496_96364474_96392998_96404426_RF | OBD160.493.495 |
| 125 | Hg38_1_158905246_158912243_159126541_159136350_FF | OBD160.497.499 |
| 126 | Hg38_11_106098496_106106725_106275961_106290738_RR | OBD160.501.503 |
| 127 | Hg38_11_72859306_72866489_72925691_72936492_RF | OBD160.505.507 |
| 128 | Hg38_14_49020009_49028815_49076509_49078072_FR | OBD160.509.511 |
| 129 | Hg38_4_173125717_173130524_173207477_173215489_FR | OBD160.513.515 |
| 130 | Hg38_14_19825779_19829413_19968132_19975589_FF | OBD160.517.519 |
| 131 | Hg38_15_56425187_56434244_56691312_56696438_FR | OBD160.521.523 |
| 132 | Hg38_7_30612223_30619592_30804622_30809524_FR | OBD160.525.527 |
| 133 | Hg38_20_46372997_46377307_46622048_46631132_FF | OBD160.529.531 |
| 134 | Hg38_14_24423148_24425179_24517993_24525921_RR | OBD160.533.535 |
| 135 | Hg38_7_93821969_93826969_93960806_93964392_FR | OBD160.537.539 |
| 136 | Hg38_2_36912180_36914993_37085573_37089692_RR | OBD160.541.543 |
| 137 | Hg38_4_77717993_77727364_77982270_77985832_RR | OBD160.545.547 |
| 138 | Hg38_3_87991842_87995289_88126188_88133417_FF | OBD160.549.551 |
| 139 | Hg38_4_121310394_121315169_121339971_121354127_RF | OBD160.553.555 |
| 140 | Hg38_2_15677778_15684445_15733618_15739157_RR | OBD160.557.559 |
| 141 | Hg38_12_29701302_29708431_29828391_29843699_FF | OBD160.561.563 |
| 142 | Hg38_3_64067438_64075904_64228797_64236262_FR | OBD160.565.567 |
| 143 | Hg38_17_40700822_40703790_40868811_40875117_FR | OBD160.569.571 |
| 144 | Hg38_9_120795804_120798748_120831347_120833895_RR | OBD160.573.575 |
| 145 | Hg38_7_144246203_144258031_144391768_144394587_FR | OBD160.577.579 |
| 146 | Hg38_12_71685734_71687005_71771992_71780701_FR | OBD160.581.583 |
| 147 | Hg38_11_82067024_82073102_82131728_82146376_RF | OBD160.585.587 |
| 148 | Hg38_7_144153639_144162181_144246203_144258031_RF | OBD160.589.591 |
| 149 | Hg38_2_21769905_21774394_21960934_21972734_FR | OBD160.593.595 |
| 150 | Hg38_4_38385027_38395353_38450896_38455314_RF | OBD160.597.599 |

TABLE 1A
(part 4a)

| No. | probe | RP/Rsum | Rprank | FC:(class1/class2) |
|---|---|---|---|---|
| 151 | Hg38_17_36023309_36028028_36272442_36275457_FF | 181419.9448 | 102901 | 1.51 |
| 152 | Hg38_12_78435225_78440781_78459801_78476525_RF | 5179.767063 | 196 | 1.51 |
| 153 | Hg38_X_40477200_40482652_40589768_40592788_RF | 3846.655135 | 146 | 1.51 |
| 154 | Hg38_Y_3584113_3585230_3719457_3726617_FF | 5222.745707 | 200 | 1.508 |
| 155 | Hg38_7_103380032_103385339_103627677_103636524_RR | 3342.65815 | 120 | 1.508 |
| 156 | Hg38_1_152724530_152728363_152991207_152992512_FR | 3244.654375 | 112 | 1.508 |
| 157 | Hg38_10_103825506_103831138_103895197_103900793_FR | 4497.05848 | 167 | 1.504 |
| 158 | Hg38_Y_2866835_2871971_2958559_2972902_RF | 3649.087311 | 136 | 1.502 |
| 159 | Hg38_4_142475549_142484776_142514277_142521702_RR | 4031.321645 | 150 | 1.501 |
| 160 | Hg38_4_124396134_124401011_124507033_124522866_FR | 3626.70075 | 134 | 1.501 |
| 161 | Hg38_12_31992399_31994148_32218403_32222271_RF | 4824.003128 | 175 | 1.499 |
| 162 | Hg38_X_2551528_2553681_2721164_2724566_FR | 4920.501174 | 180 | 1.498 |
| 163 | Hg38_4_12250997_12254184_12322283_12330696_FF | 4300.114047 | 161 | 1.498 |
| 164 | Hg38_2_203737190_203742853_203950615_203958558_RR | 4856.527848 | 177 | 1.497 |
| 165 | Hg38_4_171028618_171040398_171297049_171298480_FR | 4401.334551 | 164 | 1.497 |
| 166 | Hg38_18_74451499_74454993_74653959_74660559_RR | 4203.559159 | 157 | 1.497 |
| 167 | Hg38_7_10927538_10936617_11059573_11063361_FF | 4045.05349 | 151 | 1.497 |
| 168 | Hg38_2_199918581_199920573_200063524_200066499_FF | 3765.522187 | 141 | 1.497 |
| 169 | Hg38_20_40684507_40687277_40697330_40701314_RF | 3823.485364 | 145 | 1.495 |
| 170 | Hg38_1_69381686_69386805_69431002_69439637_FF | 5021.561295 | 188 | 1.494 |
| 171 | Hg38_X_80882210_80890038_81125945_81129443_RF | 3947.387733 | 148 | 1.494 |
| 172 | Hg38_X_81004388_81008183_81249768_81260862_RR | 3665.225818 | 137 | 1.494 |
| 173 | Hg38_2_43218506_43222954_43338993_43342008_RR | 4532.348136 | 168 | 1.493 |
| 174 | Hg38_12_78001793_78008398_78104543_78106544_FF | 4479.616333 | 166 | 1.493 |
| 175 | Hg38_8_138470025_138485193_138738624_138748013_FR | 3670.508224 | 138 | 1.493 |
| 176 | Hg38_12_85366513_85378853_85614124_85622896_FF | 4696.514771 | 172 | 1.492 |
| 177 | Hg38_7_112896017_112908366_113061946_113067988_RR | 3616.071678 | 133 | 1.492 |
| 178 | Hg38_2_200576039_200582576_200625910_200631991_FF | 4070.103419 | 153 | 1.491 |
| 179 | Hg38_15_30512275_30516087_30637530_30644724_RF | 5643.964375 | 225 | 1.49 |
| 180 | Hg38_7_25392695_25398339_25542599_25548176_FR | 4947.457038 | 183 | 1.49 |
| 181 | Hg38_6_56692561_56704365_56788190_56791583_FR | 4933.771069 | 182 | 1.49 |
| 182 | Hg38_9_63901466_63907814_64080352_64086066_RF | 4265.343025 | 158 | 1.49 |
| 183 | Hg38_13_21325011_21327390_21575413_21582625_RF | 46100.56928 | 5384 | 1.488 |
| 184 | Hg38_4_164713694_164729294_164967949_164972230_RF | 5809.93205 | 233 | 1.488 |
| 185 | Hg38_1_63238122_63243108_63375818_63382270_FR | 4098.295424 | 154 | 1.488 |
| 186 | Hg38_11_106045994_106053482_106098496_106106725_FR | 5214.04095 | 199 | 1.486 |
| 187 | Hg38_2_191681747_191683098_191825543_191836084_RF | 5765.279979 | 232 | 1.485 |
| 188 | Hg38_14_70326561_70339130_70362648_70369731_RR | 5337.378645 | 208 | 1.485 |
| 189 | Hg38_5_3428156_3436940_3502869_3506924_RF | 97200.98548 | 27570 | 1.484 |
| 190 | Hg38_9_93834125_93843642_94033256_94043963_RR | 5264.953933 | 204 | 1.484 |
| 191 | Hg38_4_166975421_166983791_167173650_167191011_FF | 4931.651686 | 181 | 1.484 |
| 192 | Hg38_12_114559051_114568056_114591211_114602162_FF | 5641.122845 | 223 | 1.483 |
| 193 | Hg38_13_91314238_91325788_91448901_91460521_RR | 5289.858609 | 205 | 1.483 |
| 194 | Hg38_X_107971578_107974327_108029689_108034634_RR | 4958.888197 | 185 | 1.483 |
| 195 | Hg38_8_24835942_24839566_24905842_24907666_FR | 4823.525297 | 174 | 1.483 |
| 196 | Hg38_8_115234769_115239118_115394021_115408137_FR | 4268.313107 | 159 | 1.483 |
| 197 | Hg38_12_126756498_126769103_126960572_126965968_RF | 4607.573373 | 170 | 1.482 |
| 198 | Hg38_11_93302415_93309584_93391985_93400931_RF | 5161.045697 | 194 | 1.481 |
| 199 | Hg38_3_81259865_81272497_81351596_81353868_RR | 4609.489918 | 171 | 1.481 |
| 200 | Hg38_3_38500849_38506870_38670653_38683212_FR | 4308.252287 | 162 | 1.479 |

TABLE 1A (part 4b)

| | | P. | | Probe sequence |
|---|---|---|---|---|
| No. | pfp | value | Type | 60 mer |
| 151 | 0. | 0. | SBMA | TGAGCTAATAAACTATTTCTGGTTTTGCTCGAGTTCCACATTCTATACCATGTTTCTTTT |
| | 201236982 | 02229589 | | |
| 152 | 0 | 0 | SBMA | TGTCTTCTGGATTTTATTATTTTTTCTGTCGAAATGATCATATAATTTTACTCCTTCAGT |
| 153 | 0 | 0 | SBMA | TTTTTTTTTTTTTCAGACTTTCCTGTGGTCGACAAGGAGGTAAGATTAGATACATACATC |
| 154 | 0 | 0 | SBMA | TACATTCGGTCACTGAAAACATTAGTTTTCGATGAGGGTGTCTTTTCTCCAGTTTATGTT |
| 155 | 0 | 0 | SBMA | TTTGAGTGTTCTTTTCTTCTGTGGAATATCGATTGCTCACATGTGGTTGTTAATAAAACT |
| 156 | 0 | 0 | SBMA | GCATGTCAGGTAATGAAAACAGACATTATCGAAAGACATTTATACACGACTTTAACCGTT |
| 157 | 0 | 0 | SBMA | TGAATAATTTTTAATTTACATAAATGGATCGATTCTTTTTAAACATATAGCACCAGGCAG |
| 158 | 0 | 0 | SBMA | AAACTTCAGTTTGCTTTACTGTAAAATGTCGACTTACTGTTGCTGAAGAGAGAGATAAGA |
| 159 | 0 | 0 | SBMA | GAATTGGGTTTCTTCTTTCTTTTGAATGTCGACTTTAATTTATCCTTTCTTTGAAAGCCA |
| 160 | 0 | 0 | SBMA | TTACACTCATTGATTTTCAGATATTAAATCGAATTCTTGTACATGTAAGTGGCAGATAAG |
| 161 | 0 | 0 | SBMA | CAGAAGATTGGAGTTATCAAATGAATTATCGAAGAGAAGCTTCAGTGCAGATGGATTTGA |
| 162 | 0 | 0 | SBMA | GCCAAGGCAAAACTCCAATTATTAGTTTTCGAAAGTTTATATGTTTAGGTTTTTTTGTTT |
| 163 | 0 | 0 | SBMA | AAGGGACTAGAAGAAGAAATCATTAAGTTCGACAATTTTTAAATAACTGCCTGAAGATAA |
| 164 | 0 | 0 | SBMA | CTTAAAAATCAACCAAAGTACAGTATTTTCGAACTGCAGACTTAGAGCTGTCAAGCGGTT |
| 165 | 0 | 0 | SBMA | TAGCTTTGCATGAACTAAATGTCAAATTTCGAAAAATTATACTGCGAGAAAATTCATACC |
| 166 | 0 | 0 | SBMA | GGCAAATCTCGTATAGATTTGTATTTCATCGATATAATTTTAATGAGTGCTCAATTACAG |
| 167 | 0 | 0 | SBMA | TTAGAGGCAAAATATAACACAGATGAGATCGACATATTGCAAAATTTATACTGTGGTCAG |
| 168 | 0 | 0 | SBMA | AACTATAAAAAGGATTTAGATGAGCCCTTCGAGATTTAAAACACACACACACACATATTT |
| 169 | 0 | 0 | SBMA | TCTAAACTTGGAGTCAGAAGAACTGAGTTCGAGCTAGAATTCCAAGCTTCTCTCCTATTG |
| 170 | 0 | 0 | SBMA | TTGCTATTCTTTCTTCATTCATCACTCTTCGATTTGTAAACTTACAAAACCTAAACATTC |
| 171 | 0 | 0 | SBMA | CCATTTGTATTTCCTGTCTGTGAATTTTTCGAAATGTATGAATCAGAATAATTCAGAGGA |
| 172 | 0 | 0 | SBMA | TTCACTCAAAGCCTGAATGATTTTAGGGTCGAAAGAAACCAGAAAGTAACTATGCAAGTA |
| 173 | 0 | 0 | SBMA | CTGAAATAGAAGGAAAGATATTAAGGACTCGAGTGAAAAAAGGAAGGAAGCTATAACTTA |
| 174 | 0 | 0 | SBMA | TTGCTTGTTGTTCTGTGATTTGTTTTAATCGACAGAACATCTTCTGGTGTTCTGTTCACC |
| 175 | 0 | 0 | SBMA | ATTTCAGCAGAGTTTACAGACTGTAATTTCGAGGCTCAGACCTCATGTCTCTCTTGTTGC |
| 176 | 0 | 0 | SBMA | AGTAAGGCAGAAGTGATATTGTATGACTTCGAACTTTTTTTTTTCTACTGCATACATATA |
| 177 | 0 | 0 | SBMA | TATGTAAGCTTTGCTGGAGAAGTTTTAGTCGAGTTTGAGGCACTAAGGTAAATAAAACAT |
| 178 | 0 | 0 | SBMA | GCAGATTATAGCTGGAATTCACAATATTTCGAAAATCTGAGTTTAAAAAAAGAGCACAGA |
| 179 | 0 | 0 | SBMA | ATTTTCAGGTAGGATCATAAAGGACTTATCGAAAAGAAAAAAAAGAATTATTTAACTTAA |
| 180 | 0 | 0 | SBMA | CTTCAAGTAGTCACAGAGAATGACAGAATCGAAGTACTGTTTTGTTTATTGCTGTATCCC |
| 181 | 0 | 0 | SBMA | AGTCTTCATAGAGATCATTCACATGTTTTCGAAAGTATTAAGTTCTTCTTTTTTTTTTTT |
| 182 | 0 | 0 | SBMA | CCATCTGATTACAGTTACATGTATCATATCGAATGACCTCCCTTCTATTCCTGTTAATAA |
| 183 | 0. | 0. | SBMA | ACTTAACTTCCTTCTAAAACAACCTTTTTCGACTTTAGTTGGTGACATCATCATCCCCCT |
| | 0000854 | 0000004 | | |
| | 3833 | 9528 | | |
| | 5809 | 5101 | | |
| | 8068 | 178133 | | |
| 184 | 0 | 0 | SBMA | CAGCTATTGACTGTGTATCTTTAGAAAATCGATCTCTAAATAAACTGCAGAGGAGGAGGT |
| 185 | 0 | 0 | SBMA | CCTTAAAAGATGTGAAACAGAGAACAGCTCGAATTATTTTTGTAATTTATATTTTTCTAG |
| 186 | 0 | 0 | SBMA | AGTTTTTCCACCTATGACAAATAATCTATCGATTAGTAAAAGAAAAAAATTTGAATGTTT |
| 187 | 0 | 0 | SBMA | CTCCTCTTCATCTTCCACAATAGTCAAGTCGACAGAATTTTTGTATTCTTTTGTAAATAG |
| 188 | 0 | 0 | SBMA | GTAGTTGATTTATTTGCTACAGTTGTAATCGAAAGATTAGTTACTTCATGAATCTTTGTT |
| 189 | 0. | 0. | SBMA | AATATTAGAATGGGCTACTCGCCTTTCCTCGAGACGTGGGCAAATGTATTTAAAAGTCAT |
| | 008319429 | 000246961 | | |
| 190 | 0 | 0 | SBMA | AAGTTCAACACAGTTGCAGAATATAGGATCGAAATGTTTCTTCAGAATCATGTTTTATAT |
| 191 | 0 | 0 | SBMA | CCCCTATGTGGAATTATGTAGTTATCTCTCGAAACAGTATGTTCTGGCATAAAAACAGAC |
| 192 | 0 | 0 | SBMA | CTGTACTTAGGGTATCATAAGGCTGAAATCGATTTCCTGTCTTACCCACACATTCAGGCT |
| 193 | 0 | 0 | SBMA | GAATATTTTATTTGCCATTTCAACAAAATCGAAATGCGGCTGCTTTTCAAGGTAAGTGGA |
| 194 | 0 | 0 | SBMA | CCACCACCACCAGTGGTCCTCCCAGTATTCGATGGAATAGTGAAAACCTTCACCAATTGC |
| 195 | 0 | 0 | SBMA | CTTCAACTCAATATAAATAAGATCTCTATCGAGGTAACTAGGTCTTTACTTGCCTGAATC |
| 196 | 0 | 0 | SBMA | GTGTCCTTTACATCCTAAGTACAGAGATTCGATGGAGAAAGTGGTGTGCACACCAGTACA |
| 197 | 0 | 0 | SBMA | AGAAACAAGTAAGAGATTCTAAAAGTTCTCGATTTTGAAGGTAATTTGCACTTTTCTACT |
| 198 | 0 | 0 | SBMA | ATTTCACCACTTTTATTCAACATAATACTCGACAGAATGAAGAACAAAAACGATTTGATC |
| 199 | 0 | 0 | SBMA | TTAAAATGATTTAGCAGGGCACAAAATGTCGATAAATAATACGACATTTGCATCACTACC |
| 200 | 0 | 0 | SBMA | ATTTGTTTTTCAGAATAGAAATGTACTGTCGACCTATCTTCAGACTCACTGTTTCTTTCC |

TABLE 1A
(part 4c)

| | Probe Location | | | | |
|---|---|---|---|---|---|
| No. | Chr | Start1 | End1 | Start2 | End2 |
| 151 | 17 | 36027997 | 36028026 | 36275426 | 36275455 |
| 152 | 12 | 78435227 | 78435256 | 78476494 | 78476523 |
| 153 | X | 40477202 | 40477231 | 40592757 | 40592786 |
| 154 | Y | 3585199 | 3585228 | 3726586 | 3726615 |
| 155 | 7 | 103380034 | 103380063 | 103627679 | 103627708 |
| 156 | 1 | 152728332 | 152728361 | 152991209 | 152991238 |
| 157 | 10 | 103831107 | 103831136 | 103895199 | 103895228 |
| 158 | Y | 2866837 | 2866866 | 2972871 | 2972900 |
| 159 | 4 | 142475551 | 142475580 | 142514279 | 142514308 |
| 160 | 4 | 124400980 | 124401009 | 124507035 | 124507064 |
| 161 | 12 | 31992401 | 31992430 | 32222240 | 32222269 |
| 162 | X | 2553650 | 2553679 | 2721166 | 2721195 |
| 163 | 4 | 12254153 | 12254182 | 12330665 | 12330694 |
| 164 | 2 | 203737192 | 203737221 | 203950617 | 203950646 |
| 165 | 4 | 171040367 | 171040396 | 171297051 | 171297080 |
| 166 | 18 | 74451501 | 74451530 | 74653961 | 74653990 |
| 167 | 7 | 10936586 | 10936615 | 11063330 | 11063359 |
| 168 | 2 | 199920542 | 199920571 | 200066468 | 200066497 |
| 169 | 20 | 40684509 | 40684538 | 40701283 | 40701312 |
| 170 | 1 | 69386774 | 69386803 | 69439606 | 69439635 |
| 171 | X | 80882212 | 80882241 | 81129412 | 81129441 |
| 172 | X | 81004390 | 81004419 | 81249770 | 81249799 |
| 173 | 2 | 43218508 | 43218537 | 43338995 | 43339024 |
| 174 | 12 | 78008367 | 78008396 | 78106513 | 78106542 |
| 175 | 8 | 138485162 | 138485191 | 138738626 | 138738655 |
| 176 | 12 | 85378822 | 85378851 | 85622865 | 85622894 |
| 177 | 7 | 112896019 | 112896048 | 113061948 | 113061977 |
| 178 | 2 | 200582545 | 200582574 | 200631960 | 200631989 |
| 179 | 15 | 30512277 | 30512306 | 30644693 | 30644722 |
| 180 | 7 | 25398308 | 25398337 | 25542601 | 25542630 |
| 181 | 6 | 56704334 | 56704363 | 56788192 | 56788221 |
| 182 | 9 | 63901468 | 63901497 | 64086035 | 64086064 |
| 183 | 13 | 21325013 | 21325042 | 21582594 | 21582623 |
| 184 | 4 | 164713696 | 164713725 | 164972199 | 164972228 |
| 185 | 1 | 63243077 | 63243106 | 63375820 | 63375849 |
| 186 | 11 | 106053451 | 106053480 | 106098498 | 106098527 |
| 187 | 2 | 191681749 | 191681778 | 191836053 | 191836082 |
| 188 | 14 | 70326563 | 70326592 | 70362650 | 70362679 |
| 189 | 5 | 3428158 | 3428187 | 3506893 | 3506922 |
| 190 | 9 | 93834127 | 93834156 | 94033258 | 94033287 |
| 191 | 4 | 166983760 | 166983789 | 167190980 | 167191009 |
| 192 | 12 | 114568025 | 114568054 | 114602131 | 114602160 |
| 193 | 13 | 91314240 | 91314269 | 91448903 | 91448932 |
| 194 | X | 107971580 | 107971609 | 108029691 | 108029720 |
| 195 | 8 | 24839535 | 24839564 | 24905844 | 24905873 |
| 196 | 8 | 115239087 | 115239116 | 115394023 | 115394052 |
| 197 | 12 | 126756500 | 126756529 | 126965937 | 126965966 |
| 198 | 11 | 93302417 | 93302446 | 93400900 | 93400929 |
| 199 | 3 | 81259867 | 81259896 | 81351598 | 81351627 |
| 200 | 3 | 38506839 | 38506868 | 38670655 | 38670684 |

TABLE 1A
(part 4d)

| | 4 kb Sequence Location | | | | |
|---|---|---|---|---|---|
| No. | Chr | Start1 | End1 | Start2 | End2 |
| 151 | 17 | 36024027 | 36028026 | 36271456 | 36275455 |
| 152 | 12 | 78435227 | 78439226 | 78472524 | 78476523 |
| 153 | X | 40477202 | 40481201 | 40588787 | 40592786 |
| 154 | Y | 3581229 | 3585228 | 3722616 | 3726615 |
| 155 | 7 | 103380034 | 103384033 | 103627679 | 103631678 |
| 156 | 1 | 152724362 | 152728361 | 152991209 | 152995208 |
| 157 | 10 | 103827137 | 103831136 | 103895199 | 103899198 |
| 158 | Y | 2866837 | 2870836 | 2968901 | 2972900 |
| 159 | 4 | 142475551 | 142479550 | 142514279 | 142518278 |
| 160 | 4 | 124397010 | 124401009 | 124507035 | 124511034 |
| 161 | 12 | 31992401 | 31996400 | 32218270 | 32222269 |
| 162 | X | 2549680 | 2553679 | 2721166 | 2725165 |
| 163 | 4 | 12250183 | 12254182 | 12326695 | 12330694 |
| 164 | 2 | 203737192 | 203741191 | 203950617 | 203954616 |
| 165 | 4 | 171036397 | 171040396 | 171297051 | 171301050 |
| 166 | 18 | 74451501 | 74455500 | 74653961 | 74657960 |
| 167 | 7 | 10932616 | 10936615 | 11059360 | 11063359 |
| 168 | 2 | 199916572 | 199920571 | 200062498 | 200066497 |
| 169 | 20 | 40684509 | 40688508 | 40697313 | 40701312 |
| 170 | 1 | 69382804 | 69386803 | 69435636 | 69439635 |
| 171 | X | 80882212 | 80886211 | 81125442 | 81129441 |
| 172 | X | 81004390 | 81008389 | 81249770 | 81253769 |
| 173 | 2 | 43218508 | 43222507 | 43338995 | 43342994 |
| 174 | 12 | 78004397 | 78008396 | 78102543 | 78106542 |
| 175 | 8 | 138481192 | 138485191 | 138738626 | 138742625 |
| 176 | 12 | 85374852 | 85378851 | 85618895 | 85622894 |
| 177 | 7 | 112896019 | 112900018 | 113061948 | 113065947 |
| 178 | 2 | 200578575 | 200582574 | 200627990 | 200631989 |
| 179 | 15 | 30512277 | 30516276 | 30640723 | 30644722 |
| 180 | 7 | 25394338 | 25398337 | 25542601 | 25546600 |
| 181 | 6 | 56700364 | 56704363 | 56788192 | 56792191 |
| 182 | 9 | 63901468 | 63905467 | 64082065 | 64086064 |
| 183 | 13 | 21325013 | 21329012 | 21578624 | 21582623 |
| 184 | 4 | 164713696 | 164717695 | 164968229 | 164972228 |
| 185 | 1 | 63239107 | 63243106 | 63375820 | 63379819 |
| 186 | 11 | 106049481 | 106053480 | 106098498 | 106102497 |
| 187 | 2 | 191681749 | 191685748 | 191832083 | 191836082 |
| 188 | 14 | 70326563 | 70330562 | 70362650 | 70366649 |
| 189 | 5 | 3428158 | 3432157 | 3502923 | 3506922 |
| 190 | 9 | 93834127 | 93838126 | 94033258 | 94037257 |
| 191 | 4 | 166979790 | 166983789 | 167187010 | 167191009 |
| 192 | 12 | 114564055 | 114568054 | 114598161 | 114602160 |
| 193 | 13 | 91314240 | 91318239 | 91448903 | 91452902 |
| 194 | X | 107971580 | 107975579 | 108029691 | 108033690 |
| 195 | 8 | 24835565 | 24839564 | 24905844 | 24909843 |
| 196 | 8 | 115235117 | 115239116 | 115394023 | 115398022 |
| 197 | 12 | 126756500 | 126760499 | 126961967 | 126965966 |
| 198 | 11 | 93302417 | 93306416 | 93396930 | 93400929 |
| 199 | 3 | 81259867 | 81263866 | 81351598 | 81355597 |
| 200 | 3 | 38502869 | 38506868 | 38670655 | 38674654 |

Table 1A
(part 4e)

| No. | probe | Primer ID |
|---|---|---|
| 151 | Hg38_17_36023309_36028028_36272442_36275457_FF | OBD160.601 |
| 152 | Hg38_12_78435225_78440781_78459801_78476525_RF | OBD160.605 |
| 153 | Hg38_X_40477200_40482652_40589768_40592788_RF | OBD160.609 |
| 154 | Hg38_Y_3584113_3585230_3719457_3726617_FF | OBD160.613 |
| 155 | Hg38_7_103380032_103385339_103627677_103636524_RR | OBD160.617 |
| 156 | Hg38_1_152724530_152728363_152991207_152992512_FR | OBD160.621 |
| 157 | Hg38_10_103825506_103831138_103895197_103900793_FR | OBD160.625 |
| 158 | Hg38_Y_2866835_2871971_2958559_2972902_RF | OBD160.629 |
| 159 | Hg38_4_142475549_142484776_142514277_142521702_RR | OBD160.633 |
| 160 | Hg38_4_124396134_124401011_124507033_124522866_FR | OBD160.637 |
| 161 | Hg38_12_31992399_31994148_32218403_32222271_RF | OBD160.641 |
| 162 | Hg38_X_2551528_2553681_2721164_2724566_FR | OBD160.645 |
| 163 | Hg38_4_12250997_12254184_12322283_12330696_FF | OBD160.649 |
| 164 | Hg38_2_203737190_203742853_203950615_203958558_RR | OBD160.653 |
| 165 | Hg38_4_171028618_171040398_171297049_171298480_FR | OBD160.657 |
| 166 | Hg38_18_74451499_74454993_74653959_74660559_RR | OBD160.661 |
| 167 | Hg38_7_10927538_10936617_11059573_11063361_FF | OBD160.665 |
| 168 | Hg38_2_199918581_199920573_200063524_200066499_FF | OBD160.669 |
| 169 | Hg38_20_40684507_40687277_40697330_40701314_RF | OBD160.673 |
| 170 | Hg38_1_69381686_69386805_69431002_69439637_FF | OBD160.677 |
| 171 | Hg38_X_80882210_80890038_81125945_81129443_RF | OBD160.681 |
| 172 | Hg38_X_81004388_81008183_81249768_81260862_RR | OBD160.685 |
| 173 | Hg38_2_43218506_43222954_43338993_43342008_RR | OBD160.689 |
| 174 | Hg38_12_78001793_78008398_78104543_78106544_FF | OBD160.693 |
| 175 | Hg38_8_138470025_138485193_138738624_138748013_FR | OBD160.697 |
| 176 | Hg38_12_85366513_85378853_85614124_85622896_FF | OBD160.701 |
| 177 | Hg38_7_112896017_112908366_113061946_113067988_RR | OBD160.705 |
| 178 | Hg38_2_200576039_200582576_200625910_200631991_FF | OBD160.709 |
| 179 | Hg38_15_30512275_30516087_30637530_30644724_RF | OBD160.713 |
| 180 | Hg38_7_25392695_25398339_25542599_25548176_FR | OBD160.717 |
| 181 | Hg38_6_56692561_56704365_56788190_56791583_FR | OBD160.721 |
| 182 | Hg38_9_63901466_63907814_64080352_64086066_RF | OBD160.725 |
| 183 | Hg38_13_21325011_21327390_21575413_21582625_RF | OBD160.729 |
| 184 | Hg38_4_164713694_164729294_164967949_164972230_RF | OBD160.733 |
| 185 | Hg38_1_63238122_63243108_63375818_63382270_FR | OBD160.737 |
| 186 | Hg38_11_106045994_106053482_106098496_106106725_FR | OBD160.741 |
| 187 | Hg38_2_191681747_191683098_191825543_191836084_RF | OBD160.745 |
| 188 | Hg38_14_70326561_70339130_70362648_70369731_RR | OBD160.749 |
| 189 | Hg38_5_3428156_3436940_3502869_3506924_RF | OBD160.753 |
| 190 | Hg38_9_93834125_93843642_94033256_94043963_RR | OBD160.757 |
| 191 | Hg38_4_166975421_166983791_167173650_167191011_FF | OBD160.761 |
| 192 | Hg38_12_114559051_114568056_114591211_114602162_FF | OBD160.765 |
| 193 | Hg38_13_91314238_91325788_91448901_91460521_RR | OBD160.769 |
| 194 | Hg38_X_107971578_107974327_108029689_108034634_RR | OBD160.773 |
| 195 | Hg38_8_24835942_24839566_24905842_24907666_FR | OBD160.777 |
| 196 | Hg38_8_115234769_115239118_115394021_115408137_FR | OBD160.781 |
| 197 | Hg38_12_126756498_126769103_126960572_126965968_RF | OBD160.785 |
| 198 | Hg38_11_93302415_93309584_93391985_93400931_RF | OBD160.789 |
| 199 | Hg38_3_81259865_81272497_81351596_81353868_RR | OBD160.793 |
| 200 | Hg38_3_38500849_38506870_38670653_38683212_FR | OBD160.797 |

TABLE 1A (part 4f)

| No. | Sequence | Primer ID | Sequence |
|---|---|---|---|
| 151 | TACGCCTGCCATCCACTCTGAAT | OBD160.603 | GGATTACAGATGTGAGCCACCAC |
| 152 | ATGTGTTGCTCTACTGGTTTGTAAGTTA | OBD160.607 | AGACACATCCAATAAAGAAACTGCGGGC |
| 153 | GGTCTATGTGATGCTGAAGTTTTGGG | OBD160.611 | GCCCTGGTTTTCTGTAGGCAATCAGC |
| 154 | ATGAAGGGAAAACATTGGAGTGGAAT | OBD160.615 | CACACACCTACAGTGAACTCATTTTC |
| 155 | ATAGTGGTCAAAGTGAAAAGTCTGAGC | OBD160.619 | GCACCTTGAGCAGCACCATAGGAGTTT |
| 156 | GCTCACAGTCTCCTGGCTGAACC | OBD160.623 | GAGGGTTCTGCGGTATCTGCCTA |
| 157 | AAGCATCCTTCCAGTCTGTGTTTGC | OBD160.627 | TAATCATTTAGAGGCGGCTCAAAGC |
| 158 | CCAGAGGTTGATGGTGGTTTCTTGTG | OBD160.631 | GGTTTATTTACTGGAGGGTCAGAGGA |
| 159 | CCTCCTCTTGGTTGATTTTGCTGTTA | OBD160.635 | CACCTTTGGTTGGAAGTTTAGGGAGG |
| 160 | CTACAACCAGTGAAAGATGGCTCCAA | OBD160.639 | CTGCCATCCCCACTAATGTCACCTCT |
| 161 | GAATGCAGAAGATTGGAGTT | OBD160.643 | TGCAGATGGATTTGAGATGC |
| 162 | GCACACTCTGAGGATTTTAGACTTC | OBD160.647 | CACAAATAGCCATCAAAATGTCAACAAG |
| 163 | GAACAACTACCTGTGCCTCAAAAGAC | OBD160.651 | CAACTCTGAAGCCTACTGTGTTTATC |
| 164 | CAACAGTTCTTCAGCACCCAAGT | OBD160.655 | GAAGGCTGACTTAGGTTTCCACATA |
| 165 | GGGTGTCACTGTTCTTTCCAGATAGC | OBD160.659 | TCTTCTCATTCTGGAACAACCGATTG |
| 166 | TCACAACTCCTGTTTGGAATCGCCCT | OBD160.663 | CTTCCTCAAATGGTCCTTCAATAACA |
| 167 | ACCCATAGATTCCAATACGCTCTTTG | OBD160.667 | GTAGTTCCAAGTTCAGGAGATACACC |
| 168 | ACTGTAGTTTCCAGGACCTCAGG | OBD160.671 | AGCATTGACTTTTGTCCCTCTTGGGC |
| 169 | CCTACCCCTCTCTTAGCCTCACT | OBD160.675 | CCTGGAGGTAGTGATGGCTGAGG |
| 170 | CGGTTTCTCCAATGCCCTTTATTCTA | OBD160.679 | GTGAGAAGGGTAAAATCTTAGGCAGC |
| 171 | CTGGCACTGGAGAGCAGCACCAA | OBD160.683 | GCGAACACTGCCTTGAATCTGCC |
| 172 | TTGTAAGGCATTGACCTGTTCTACCATA | OBD160.687 | AAAAGCAAGTCCATAACTCCAGAACA |
| 173 | CCTTGGTGCCGCTGGCTGTCTGC | OBD160.691 | CCGTTTTCCCCTTCATTGTTGTGTGC |
| 174 | TTCCCACAGCAAGGCATAGGTTTCTA | OBD160.695 | GTGAAGAGATACTCATTTGTGGATTG |
| 175 | TCAACGAGGTTTCACCAAAGCCACCAAT | OBD160.699 | CAGAGTTGCTGAATGAGAGGAGAGGACG |
| 176 | GCTGGCAGGAAAAGTGTAGACTGTTT | OBD160.703 | GAGGCAAAGTAATAAGAGAGGAAAAC |
| 177 | CGCACACTCACCCTGACTTTGTTATC | OBD160.707 | CCATTTGTAAACTGCTGACTTCATTG |
| 178 | TTGCTCAGCCCCAGGAAATAACTTGG | OBD160.711 | GTGGATAGGCAGAATCAATAATACTG |
| 179 | CAGATAATGAAACAACCACCATCGGT | OBD160.715 | GACTTCCTATTCCTGCTGCCTATGGT |
| 180 | TCATAACACCTCCCTCCTCTGTGGAACA | OBD160.719 | CCTTCGGGAGATGTATTTAGGAGTTGCG |
| 181 | ACCAAGGTATCTCCTGAAAGAACC | OBD160.723 | TAATACTTAGGCTGGGAGCGATGGCT |
| 182 | GGAATGAAGCCCAGTTGAGTTGC | OBD160.727 | GCCCAAACCCGTGTGGTCCAACA |
| 183 | TTTCCTTTTGACTTTTCCCCTAAT | OBD160.731 | ATTGGCAGGAGGAATAAGGAAGAG |
| 184 | ATTGAGAGGACACCAGGTTTGAATCC | OBD160.735 | GGTGATTAGGTTATGAGGGCTCCGTC |
| 185 | ATTTGAGTTACTTGTTTAGGGCTCCT | OBD160.739 | CAATGGTTGAACAATGACTAAACTGCCA |
| 186 | TATGCCTCTGGGTAATGGGCTAACAG | OBD160.743 | CTTGACCTTTATTTACTATGCTTGGC |
| 187 | AGGATGGCACGGTGGCTCTAAGC | OBD160.747 | ATCAAGCCCTCTCTATCCACCATA |
| 188 | CAGCCTTTTCCTTGTCCTCTCAA | OBD160.751 | GCCCAGGAGTTCAAGACCAGCCT |
| 189 | CAGAGACGGTTTCCACGGTTGTC | OBD160.755 | ACACCTGGGACTGGCTCCAACTC |
| 190 | CACTTCCGTTCAACTTTTACTGGAGG | OBD160.759 | ATGTTGCCTCCACAGAAACCAGAAAA |
| 191 | TAATGTCCTCTGGGCTCATCCAT | OBD160.763 | CTGCTCTATTGGTCTAAGTGTCTG |
| 192 | GTTAGAAATCTGGTATGAAATGACTGG | OBD160.767 | TTTATTAGGAATGCTGGAGAACTCAG |
| 193 | GCCTAAACTCCTCAAATAATGCTAATGA | OBD160.771 | AATGTTGGCAGACCTGGAAGAACCCTAT |
| 194 | GCTCAACTCTGCCATCTTACTGTGAA | OBD160.775 | CTCACCTTCCTCTCACCTTTTGACCC |
| 195 | GGCTCAAAGAGAATGTGTAAGAGAGAAA | OBD160.779 | GTCCCTGAACTTGATTGCCTGTGGTCTA |
| 196 | CTGTTTACTCCCTTGGCATCTGCTGC | OBD160.783 | GGCTATTTGCTCTTGCTACTTCTTTC |
| 197 | GCTGCCTTCCTGTTTTGATACCCTGC | OBD160.787 | AATACTACCAAATGACAGCCCAGCAT |
| 198 | GACTTCCAGTTTCAAAATGGTGGC | OBD160.791 | ATTGTGTTGAGGTATGTTCCTTCCAT |
| 199 | GACATCTCTATCTTTACAGACTGCCCCT | OBD160.795 | GCCTCCCAAAGCACTGAGATTATGACAT |
| 200 | TTTCTTGGTATCTTTCATAGGAAT | OBD160.799 | AATAACAGCAGTGAAGAATACCTT |

TABLE 1A
(part 4g)

| No. | probe | Marker |
|---|---|---|
| 151 | Hg38_17_36023309_36028028_36272442_36275457_FF | OBD160.601.603 |
| 152 | Hg38_12_78435225_78440781_78459801_78476525_RF | OBD160.605.607 |
| 153 | Hg38_X_40477200_40482652_40589768_40592788_RF | OBD160.609.611 |
| 154 | Hg38_Y_3584113_3585230_3719457_3726617_FF | OBD160.613.615 |
| 155 | Hg38_7_103380032_103385339_103627677_103636524_RR | OBD160.617.619 |
| 156 | Hg38_1_152724530_152728363_152991207_152992512_FR | OBD160.621.623 |
| 157 | Hg38_10_103825506_103831138_103895197_103900793_FR | OBD160.625.627 |
| 158 | Hg38_Y_2866835_2871971_2958559_2972902_RF | OBD160.629.631 |
| 159 | Hg38_4_142475549_142484776_142514277_142521702_RR | OBD160.633.635 |
| 160 | Hg38_4_124396134_124401011_124507033_124522866_FR | OBD160.637.639 |
| 161 | Hg38_12_31992399_31994148_32218403_32222271_RF | OBD160.641.643 |
| 162 | Hg38_X_2551528_2553681_2721164_2724566_FR | OBD160.645.647 |
| 163 | Hg38_4_12250997_12254184_12322283_12330696_FF | OBD160.649.651 |
| 164 | Hg38_2_203737190_203742853_203950615_203958558_RR | OBD160.653.655 |
| 165 | Hg38_4_171028618_171040398_171297049_171298480_FR | OBD160.657.659 |
| 166 | Hg38_18_74451499_74454993_74653959_74660559_RR | OBD160.661.663 |
| 167 | Hg38_7_10927538_10936617_11059573_11063361_FF | OBD160.665.667 |
| 168 | Hg38_2_199918581_199920573_200063524_200066499_FF | OBD160.669.671 |
| 169 | Hg38_20_40684507_40687277_40697330_40701314_RF | OBD160.673.675 |
| 170 | Hg38_1_69381686_69386805_69431002_69439637_FF | OBD160.677.679 |
| 171 | Hg38_X_80882210_80890038_81125945_81129443_RF | OBD160.681.683 |
| 172 | Hg38_X_81004388_81008183_81249768_81260862_RR | OBD160.685.687 |
| 173 | Hg38_2_43218506_43222954_43338993_43342008_RR | OBD160.689.691 |
| 174 | Hg38_12_78001793_78008398_78104543_78106544_FF | OBD160.693.695 |
| 175 | Hg38_8_138470025_138485193_138738624_138748013_FR | OBD160.697.699 |
| 176 | Hg38_12_85366513_85378853_85614124_85622896_FF | OBD160.701.703 |
| 177 | Hg38_7_112896017_112908366_113061946_113067988_RR | OBD160.705.707 |
| 178 | Hg38_2_200576039_200582576_200625910_200631991_FF | OBD160.709.711 |
| 179 | Hg38_15_30512275_30516087_30637530_30644724_RF | OBD160.713.715 |
| 180 | Hg38_7_25392695_25398339_25542599_25548176_FR | OBD160.717.719 |
| 181 | Hg38_6_56692561_56704365_56788190_56791583_FR | OBD160.721.723 |
| 182 | Hg38_9_63901466_63907814_64080352_64086066_RF | OBD160.725.727 |
| 183 | Hg38_13_21325011_21327390_21575413_21582625_RF | OBD160.729.731 |
| 184 | Hg38_4_164713694_164729294_164967949_164972230_RF | OBD160.733.735 |
| 185 | Hg38_1_63238122_63243108_63375818_63382270_FR | OBD160.737.739 |
| 186 | Hg38_11_106045994_106053482_106098496_106106725_FR | OBD160.741.743 |
| 187 | Hg38_2_191681747_191683098_191825543_191836084_RF | OBD160.745.747 |
| 188 | Hg38_14_70326561_70339130_70362648_70369731_RR | OBD160.749.751 |
| 189 | Hg38_5_3428156_3436940_3502869_3506924_RF | OBD160.753.755 |
| 190 | Hg38_9_93834125_93843642_94033256_94043963_RR | OBD160.757.759 |
| 191 | Hg38_4_166975421_166983791_167173650_167191011_FF | OBD160.761.763 |
| 192 | Hg38_12_114559051_114568056_114591211_114602162_FF | OBD160.765.767 |
| 193 | Hg38_13_91314238_91325788_91448901_91460521_RR | OBD160.769.771 |
| 194 | Hg38_X_107971578_107974327_108029689_108034634_RR | OBD160.773.775 |
| 195 | Hg38_8_24835942_24839566_24905842_24907666_FR | OBD160.777.779 |
| 196 | Hg38_8_115234769_115239118_115394021_115408137_FR | OBD160.781.783 |
| 197 | Hg38_12_126756498_126769103_126960572_126965968_RF | OBD160.785.787 |
| 198 | Hg38_11_93302415_93309584_93391985_93400931_RF | OBD160.789.791 |
| 199 | Hg38_3_81259865_81272497_81351596_81353868_RR | OBD160.793.795 |
| 200 | Hg38_3_38500849_38506870_38670653_38683212_FR | OBD160.797.799 |

TABLE 1B
(part 1a)

| | | | | FC:(class | |
|---|---|---|---|---|---|
| No | probe | RP/Rsum | Rprank | 1/class2) | pfp |
| 1 | Hg38_3_100733021_100742761_100862282_100875494_RF | 1276.982875 | 9 | −1.862 | 0 |
| 2 | Hg38_3_100750954_100758578_100862282_100875494_RF | 1853.765048 | 19 | −1.807 | 0 |
| 3 | Hg38_5_150272362_150278699_150330165_150332483_FR | 1170.349245 | 3 | −1.745 | 0 |
| 4 | Hg38_3_100862282_100875494_101052914_101071675_FF | 3484.363128 | 62 | −1.739 | 0 |
| 5 | Hg38_5_150008962_150014465_150272362_150278699_RF | 1191.048051 | 5 | −1.715 | 0 |
| 6 | Hg38_5_150272362_150278699_150382773_150387928_FF | 1176.134465 | 4 | −1.708 | 0 |
| 7 | Hg38_12_69346683_69349534_69539751_69542024_FF | 1240.559047 | 8 | −1.633 | 0 |
| 8 | Hg38_8_143073684_143079751_143219138_143224471_RF | 891.0362935 | 1 | −1.62 | 0 |
| 9 | Hg38_1_32214585_32217213_32237144_32241139_RF | 1916.433227 | 20 | −1.612 | 0 |
| 10 | Hg38_X_46498682_46508989_46616810_46620218_FR | 1191.059693 | 6 | −1.611 | 0 |
| 11 | Hg38_10_8256256_8258992_8314718_8322822_FF | 1997.333433 | 23 | −1.604 | 0 |
| 12 | Hg38_6_3027907_3030237_3146778_3149848_FR | 1332.376903 | 10 | −1.598 | 0 |
| 13 | Hg38_7_27204432_27205465_27302548_27312509_FF | 2411.122208 | 36 | −1.597 | 0 |
| 14 | Hg38_7_25370684_25382177_25542599_25548176_FF | 2022.117984 | 24 | −1.596 | 0 |
| 15 | Hg38_6_2898813_2902737_3146778_3149848_RR | 1435.327398 | 11 | −1.59 | 0 |
| 16 | Hg38_5_150036868_150040595_150272362_150278699_RF | 2126.214981 | 28 | −1.579 | 0 |
| 17 | Hg38_8_142863175_142868397_143073684_143079751_RR | 1054.173681 | 2 | −1.578 | 0 |
| 18 | Hg38_5_150194890_150198993_150272362_150278699_RF | 2149.76038 | 31 | −1.576 | 0 |
| 19 | Hg38_3_100862282_100875494_101027240_101038115_FF | 3687.763264 | 73 | −1.572 | 0 |
| 20 | Hg38_8_26561792_26565691_26638318_26644530_RR | 2165.336533 | 32 | −1.571 | 0 |
| 21 | Hg38_7_27151628_27154280_27302548_27312509_FF | 2361.083379 | 34 | −1.57 | 0 |
| 22 | Hg38_3_100677572_100682399_100862282_100875494_RF | 3589.606187 | 67 | −1.568 | 0 |
| 23 | Hg38_9_84621528_84639678_84804816_84814325_RF | 1828.923149 | 17 | −1.564 | 0 |
| 24 | Hg38_X_46498682_46508989_46571235_46574591_FR | 1785.89045 | 15 | −1.563 | 0 |
| 25 | Hg38_7_25274699_25276258_25370684_25382177_RF | 2139.963434 | 30 | −1.563 | 0 |
| 26 | Hg38_6_3146778_3149848_3232412_3234356_RF | 1805.502041 | 16 | −1.557 | 0 |
| 27 | Hg38_Y_7774814_7781345_7901883_7918222_RR | 2848.762302 | 43 | −1.557 | 0 |
| 28 | Hg38_6_47291785_47297630_47538465_47543792_RF | 1946.170671 | 22 | −1.556 | 0 |
| 29 | Hg38_5_150272362_150278699_150390262_150397231_FR | 2615.809608 | 40 | −1.555 | 0 |
| 30 | Hg38_6_2891780_2893777_3146778_3149848_FR | 1661.21676 | 12 | −1.555 | 0 |
| 31 | Hg38_13_33481982_33488531_33704999_33717490_RF | 1752.746474 | 13 | −1.549 | 0 |
| 32 | Hg38_6_3045545_3051391_3146778_3149848_FR | 1924.507183 | 21 | −1.549 | 0 |
| 33 | Hg38_8_26561792_26565691_26776216_26787349_RR | 3150.83329 | 51 | −1.548 | 0 |
| 34 | Hg38_6_2860707_2865436_3146778_3149848_FR | 1839.184279 | 18 | −1.544 | 0 |
| 35 | Hg38_7_27250080_27251352_27302548_27312509_FF | 2509.589873 | 38 | −1.544 | 0 |
| 36 | Hg38_12_114767229_114770912_114977828_114985191_RF | 2775.735277 | 42 | −1.54 | 0 |
| 37 | Hg38_7_22390272_22396878_22624843_22634759_FF | 7861.738142 | 308 | −1.539 | 0 |
| 38 | Hg38_12_91718135_91726240_91895416_91906634_FR | 3218.63397 | 53 | −1.538 | 0 |
| 39 | Hg38_10_8256256_8258992_8533854_8544622_FF | 3243.075285 | 57 | −1.537 | 0 |
| 40 | Hg38_8_142835475_142844052_143073684_143079751_FR | 1213.884569 | 7 | −1.535 | 0 |
| 41 | Hg38_6_3146778_3149848_3207080_3212780_RF | 2066.688372 | 25 | −1.533 | 0 |
| 42 | Hg38_10_102798644_102804186_102874567_102879563_RR | 1775.993146 | 14 | −1.527 | 0 |
| 43 | Hg38_6_3146778_3149848_3196920_3199851_RF | 2136.395315 | 29 | −1.525 | 0 |
| 44 | Hg38_12_114977828_114985191_115073537_115080207_FR | 3454.341156 | 60 | −1.521 | 0 |
| 45 | Hg38_X_46498682_46508989_46621627_46624211_FF | 2395.87684 | 35 | −1.521 | 0 |
| 46 | Hg38_5_150272362_150278699_150335788_150341587_FF | 3220.31145 | 54 | −1.518 | 0 |
| 47 | Hg38_12_91718135_91726240_91888283_91891782_FR | 3918.107486 | 81 | −1.515 | 0 |
| 48 | Hg38_X_46498682_46508989_46624211_46629863_FF | 2684.932505 | 41 | −1.514 | 0 |
| 49 | Hg38_7_27201418_27203903_27302548_27312509_FF | 3018.091514 | 48 | −1.513 | 0 |
| 50 | Hg38_5_132650160_132653783_132755645_132763810_RR | 3592.88293 | 68 | −1.51 | 0 |

TABLE 1B (part 1b)

| | P. | | Probe sequence |
|---|---|---|---|
| No. | value | Type | 60 mer |
| 1 | 0 | Healthy Control | CCCGATTTGCTTAATCTCGGCATAGATTTCGAATTATATTTGATTTGTCTTATCTCCTCA |
| 2 | 0 | Healthy Control | CCCGATTTGCTTAATCTCGGCATAGATTTCGAATCAAATAGTTTATTTAGAGAACCTCCA |
| 3 | 0 | Healthy Control | AGTGCCTGTGAAAGTGTTTCTGAATTCCTCGATCAGCTTCTCAGAAAAATGAAATTGACT |
| 4 | 0 | Healthy Control | CCCGATTTGCTTAATCTCGGCATAGATTTCGAATTACAGATTTTATCCAAAATATCTGTC |
| 5 | 0 | Healthy Control | AGTGCCTGTGAAAGTGTTTCTGAATTCCTCGACTTCTTGCTTTTGATTTATTGAAGAATC |
| 6 | 0 | Healthy Control | AGTGCCTGTGAAAGTGTTTCTGAATTCCTCGACAAAAATCAGAGGGGGTTTAATCACCTT |
| 7 | 0 | Healthy Control | AGTTATTTTATAGTTTAGTACATAGTTGTCGAGTGTGTGAGGAAAAATTATTCTGAAAAT |
| 8 | 0 | Healthy Control | CATGTTTTGGAGTCTTTTATCTAGTTTATCGAGGAGACAGCCGTTGTCCCCAGGGCTCCT |
| 9 | 0 | Healthy Control | AAGGGCTCGGGAGCTCCCTCGGCACACCTCGAGGAGTGCCAGGCATCTACTGCTCTGTCC |
| 10 | 0 | Healthy Control | ACAGAGCCCTCAGAAATAATGCCGCATATCGATGAGAAGTCTGATTCTTGCTTTTTGGAG |
| 11 | 0 | Healthy Control | TGGGGTGATTGGGTTCATTTATGTATTATCGAGAGACATAACAATATTTATAAAGTTTGG |
| 12 | 0 | Healthy Control | AAACTTGGTATGTGTCTAATAAACAGCTTCGAGTGCTGGTTTGGGTCGGAGTGCTGGTTC |
| 13 | 0 | Healthy Control | CTTTAAGTCAACAAATACACTGAAGACTTCGAAGTAATGAAGAGGGAGGTGGTTTTCTTT |
| 14 | 0 | Healthy Control | AGCCAGCCATTGTTAACATAGTTCAACTTCGAAGGAATGATTCTTTTTTCTTAACCATGC |
| 15 | 0 | Healthy Control | CCTCCAGTCCTATTTCTTTTTTTTTTTTTCGAGTGCTGGTTTGGGTCGGAGTGCTGGTTC |
| 16 | 0 | Healthy Control | AGTGCCTGTGAAAGTGTTTCTGAATTCCTCGAATTATTATGAGTCTCCGTCCACGCAGTG |
| 17 | 0 | Healthy Control | TCGTCTATGCATGGAATATTCTCCAAAATCGAGGAGACAGCCGTTGTCCCCAGGGCTCCT |
| 18 | 0 | Healthy Control | AGTGCCTGTGAAAGTGTTTCTGAATTCCTCGAGCGTACACCAATGGAGGAGGTATGGGCC |
| 19 | 0 | Healthy Control | CCCGATTTGCTTAATCTCGGCATAGATTTCGAAAGGCCTGAGAAAAAAGGGAGCTGGTGG |
| 20 | 0 | Healthy Control | CAGCCAGCCAGTAGATCTTCATAGGAGCTCGAAATTTTGCTATAAATGTGAGCTTTGAAA |
| 21 | 0 | Healthy Control | ATGCTTTTTGGAATAAATATATTATTTTTCGAAGTAATGAAGAGGGAGGTGGTTTTCTTT |
| 22 | 0 | Healthy Control | CCCGATTTGCTTAATCTCGGCATAGATTTCGAAGAACTCCTTTTAGCATTTCTTGTAGGT |
| 23 | 0 | Healthy Control | CTCTTCAGGGGTGTGTGGAGTAAATAGCTCGAGGAAACCTAAAAGTGTCATGTTATCCAT |
| 24 | 0 | Healthy Control | ACAGAGCCCTCAGAAATAATGCCGCATATCGAGGATCTATCTCCTTTCTCACCTGTCTGG |
| 25 | 0 | Healthy Control | AGCCAGCCATTGTTAACATAGTTCAACTTCGATATTTTTACATCATAATTTTGTTTTATT |
| 26 | 0 | Healthy Control | GCCCCATTCTGAGAACTCCTGATTGGATTCGAGTGCTGGTTTGGGTCGGAGTGCTGGTTC |
| 27 | 0 | Healthy Control | TGCCCAGCCCATTATTGATTTCTTTATGTCGAACTTGCCAACGGAAATTGAGGAAAATTG |
| 28 | 0 | Healthy Control | TGGGTGTTAGGGTTTCAATATATGAATTTCGAAGGCCTCTTCCAATTCTGAAGTAAACAG |
| 29 | 0 | Healthy Control | AGTGCCTGTGAAAGTGTTTCTGAATTCCTCGAGTCCCTCATGAGATTTTCCAGCGTAGTG |
| 30 | 0 | Healthy Control | TGAGGATGGTGAAAGATACCTAAGTCACTCGAGTGCTGGTTTGGGTCGGAGTGCTGGTTC |
| 31 | 0 | Healthy Control | AAGGCGGGCAGATCACGAGGTCAGGCAATCGAAAAAAAAAAAAAAGAAAGAAAAAAAAA |
| | | | G |
| 32 | 0 | Healthy Control | TGATCACTCCAAACACCCAAAGGTGACTTCGAGTGCTGGTTTGGGTCGGAGTGCTGGTTC |
| 33 | 0 | Healthy Control | CAGCCAGCCAGTAGATCTTCATAGGAGCTCGAATTCCTATCCAGCACTGGTCATTAGGGG |
| 34 | 0 | Healthy Control | AGTGGTGTAACCTCAGTTGACTGCAGCCTCGAGTGCTGGTTTGGGTCGGAGTGCTGGTTC |
| 35 | 0 | Healthy Control | TTGTGAAAGTCATATCCTTGCGTCTTCATCGAAGTAATGAAGAGGGAGGTGGTTTTCTTT |
| 36 | 0 | Healthy Control | GGCAGGGGCAGTATTCTCAGGTGGTTCCTCGAAGAACGGGCCTGGCCCAAGGCACTTTGG |
| 37 | 0 | Healthy Control | TGTCTTGAGGCTTACTAGTGCACTCCCGTCGATGTATTCACTAATAAAAAAGGAAGATTC |
| 38 | 0 | Healthy Control | GTCAGGGTCAGATATTGATTTAACGAAGTCGAAGAACTAAAAACTTTTTTCTAGTTTCAC |
| 39 | 0 | Healthy Control | TGGGGTGATTGGGTTCATTTATGTATTATCGACTCATAGATCCAGGTACAACGTATAATT |
| 40 | 0 | Healthy Control | AAGCAGTCTTGGGGAAAAAGAAAAAAGCTCGAGGAGACAGCCGTTGTCCCCAGGGCTCCT |
| 41 | 0 | Healthy Control | TCAAAGTCCAAGTTAGAATTCTGAGAACTCGAGTGCTGGTTTGGGTCGGAGTGCTGGTTC |
| 42 | 0 | Healthy Control | AAGGCGGGCAGATCACGAGGTCAACAGATCGACATTTCATCTGTTCTCTTTTAGATCAAA |
| 43 | 0 | Healthy Control | ACTATGGTGGATTTAAAAATAAATGCCCTCGAGTGCTGGTTTGGGTCGGAGTGCTGGTTC |
| 44 | 0 | Healthy Control | GGCAGGGGCAGTATTCTCAGGTGGTTCCTCGATACACTTCTCATAAAAAATGGCACTGAA |
| 45 | 0 | Healthy Control | ACAGAGCCCTCAGAAATAATGCCGCATATCGATAATTTGCTAGAATGACTCACAGAACAC |
| 46 | 0 | Healthy Control | AGTGCCTGTGAAAGTGTTTCTGAATTCCTCGATGTGTTTTGGGGACTGAAAAGTCCTGCA |
| 47 | 0 | Healthy Control | GTCAGGGTCAGATATTGATTTAACGAAGTCGACATGCGATTTGAACAGGGACACAAATCC |
| 48 | 0 | Healthy Control | ACAGAGCCCTCAGAAATAATGCCGCATATCGAGGCTAATTTAGAGTATAACAGTTTGGGA |
| 49 | 0 | Healthy Control | AAAAGGAAAAAGGGAAACCAGGTTTTAATCGAAGTAATGAAGAGGGAGGTGGTTTTCTTT |
| 50 | 0 | Healthy Control | TCTTTTTATTGAAGAGCGTGTTTTAAAATCGAATATTAGTAGTCCCGTGGCAAGCCTTTG |

TABLE 1B
(part 1c)

| | Probe Location | | | | |
|---|---|---|---|---|---|
| No. | Chr | Start1 | End1 | Start2 | End2 |
| 1 | 3 | 100733023 | 100733052 | 100875463 | 100875492 |
| 2 | 3 | 100750956 | 100750985 | 100875463 | 100875492 |
| 3 | 5 | 150278668 | 150278697 | 150330167 | 150330196 |
| 4 | 3 | 100875463 | 100875492 | 101071644 | 101071673 |
| 5 | 5 | 150008964 | 150008993 | 150278668 | 150278697 |
| 6 | 5 | 150278668 | 150278697 | 150387897 | 150387926 |
| 7 | 12 | 69349503 | 69349532 | 69541993 | 69542022 |
| 8 | 8 | 143073686 | 143073715 | 143224440 | 143224469 |
| 9 | 1 | 32214587 | 32214616 | 32241108 | 32241137 |
| 10 | X | 46508958 | 46508987 | 46616812 | 46616841 |
| 11 | 10 | 8258961 | 8258990 | 8322791 | 8322820 |
| 12 | 6 | 3030206 | 3030235 | 3146780 | 3146809 |
| 13 | 7 | 27205434 | 27205463 | 27312478 | 27312507 |
| 14 | 7 | 25382146 | 25382175 | 25548145 | 25548174 |
| 15 | 6 | 2898815 | 2898844 | 3146780 | 3146809 |
| 16 | 5 | 150036870 | 150036899 | 150278668 | 150278697 |
| 17 | 8 | 142863177 | 142863206 | 143073686 | 143073715 |
| 18 | 5 | 150194892 | 150194921 | 150278668 | 150278697 |
| 19 | 3 | 100875463 | 100875492 | 101038084 | 101038113 |
| 20 | 8 | 26561794 | 26561823 | 26638320 | 26638349 |
| 21 | 7 | 27154249 | 27154278 | 27312478 | 27312507 |
| 22 | 3 | 100677574 | 100677603 | 100875463 | 100875492 |
| 23 | 9 | 84621530 | 84621559 | 84814294 | 84814323 |
| 24 | X | 46508958 | 46508987 | 46571237 | 46571266 |
| 25 | 7 | 25274701 | 25274730 | 25382146 | 25382175 |
| 26 | 6 | 3146780 | 3146809 | 3234325 | 3234354 |
| 27 | Y | 7774816 | 7774845 | 7901885 | 7901914 |
| 28 | 6 | 47291787 | 47291816 | 47543761 | 47543790 |
| 29 | 5 | 150278668 | 150278697 | 150390264 | 150390293 |
| 30 | 6 | 2893746 | 2893775 | 3146780 | 3146809 |
| 31 | 13 | 33481984 | 33482013 | 33717459 | 33717488 |
| 32 | 6 | 3051360 | 3051389 | 3146780 | 3146809 |
| 33 | 8 | 26561794 | 26561823 | 26776218 | 26776247 |
| 34 | 6 | 2865405 | 2865434 | 3146780 | 3146809 |
| 35 | 7 | 27251321 | 27251350 | 27312478 | 27312507 |
| 36 | 12 | 114767231 | 114767260 | 114985160 | 114985189 |
| 37 | 7 | 22396847 | 22396876 | 22634728 | 22634757 |
| 38 | 12 | 91726209 | 91726238 | 91895418 | 91895447 |
| 39 | 10 | 8258961 | 8258990 | 8544591 | 8544620 |
| 40 | 8 | 142844021 | 142844050 | 143073686 | 143073715 |
| 41 | 6 | 3146780 | 3146809 | 3212749 | 3212778 |
| 42 | 10 | 102798646 | 102798675 | 102874569 | 102874598 |
| 43 | 6 | 3146780 | 3146809 | 3199820 | 3199849 |
| 44 | 12 | 114985160 | 114985189 | 115073539 | 115073568 |
| 45 | X | 46508958 | 46508987 | 46624180 | 46624209 |
| 46 | 5 | 150278668 | 150278697 | 150341556 | 150341585 |
| 47 | 12 | 91726209 | 91726238 | 91888285 | 91888314 |
| 48 | X | 46508958 | 46508987 | 46629832 | 46629861 |
| 49 | 7 | 27203872 | 27203901 | 27312478 | 27312507 |
| 50 | 5 | 132650162 | 132650191 | 132755647 | 132755676 |

TABLE 1B
(part 1d)

| | 4 kb Sequence Location | | | | |
|---|---|---|---|---|---|
| No. | Chr | Start1 | End1 | Start2 | End2 |
| 1 | 3 | 100733023 | 100737022 | 100871493 | 100875492 |
| 2 | 3 | 100750956 | 100754955 | 100871493 | 100875492 |
| 3 | 5 | 150274698 | 150278697 | 150330167 | 150334166 |
| 4 | 3 | 100871493 | 100875492 | 101067674 | 101071673 |
| 5 | 5 | 150008964 | 150012963 | 150274698 | 150278697 |
| 6 | 5 | 150274698 | 150278697 | 150383927 | 150387926 |
| 7 | 12 | 69345533 | 69349532 | 69538023 | 69542022 |
| 8 | 8 | 143073686 | 143077685 | 143220470 | 143224469 |
| 9 | 1 | 32214587 | 32218586 | 32237138 | 32241137 |
| 10 | X | 46504988 | 46508987 | 46616812 | 46620811 |
| 11 | 10 | 8254991 | 8258990 | 8318821 | 8322820 |
| 12 | 6 | 3026236 | 3030235 | 3146780 | 3150779 |
| 13 | 7 | 27201464 | 27205463 | 27308508 | 27312507 |
| 14 | 7 | 25378176 | 25382175 | 25544175 | 25548174 |
| 15 | 6 | 2898815 | 2902814 | 3146780 | 3150779 |
| 16 | 5 | 150036870 | 150040869 | 150274698 | 150278697 |
| 17 | 8 | 142863177 | 142867176 | 143073686 | 143077685 |
| 18 | 5 | 150194892 | 150198891 | 150274698 | 150278697 |
| 19 | 3 | 100871493 | 100875492 | 101034114 | 101038113 |
| 20 | 8 | 26561794 | 26565793 | 26638320 | 26642319 |
| 21 | 7 | 27150279 | 27154278 | 27308508 | 27312507 |
| 22 | 3 | 100677574 | 100681573 | 100871493 | 100875492 |
| 23 | 9 | 84621530 | 84625529 | 84810324 | 84814323 |
| 24 | X | 46504988 | 46508987 | 46571237 | 46575236 |
| 25 | 7 | 25274701 | 25278700 | 25378176 | 25382175 |
| 26 | 6 | 3146780 | 3150779 | 3230355 | 3234354 |
| 27 | Y | 7774816 | 7778815 | 7901885 | 7905884 |
| 28 | 6 | 47291787 | 47295786 | 47539791 | 47543790 |
| 29 | 5 | 150274698 | 150278697 | 150390264 | 150394263 |
| 30 | 6 | 2889776 | 2893775 | 3146780 | 3150779 |
| 31 | 13 | 33481984 | 33485983 | 33713489 | 33717488 |
| 32 | 6 | 3047390 | 3051389 | 3146780 | 3150779 |
| 33 | 8 | 26561794 | 26565793 | 26776218 | 26780217 |
| 34 | 6 | 2861435 | 2865434 | 3146780 | 3150779 |
| 35 | 7 | 27247351 | 27251350 | 27308508 | 27312507 |
| 36 | 12 | 114767231 | 114771230 | 114981190 | 114985189 |
| 37 | 7 | 22392877 | 22396876 | 22630758 | 22634757 |
| 38 | 12 | 91722239 | 91726238 | 91895418 | 91899417 |
| 39 | 10 | 8254991 | 8258990 | 8540621 | 8544620 |
| 40 | 8 | 142840051 | 142844050 | 143073686 | 143077685 |
| 41 | 6 | 3146780 | 3150779 | 3208779 | 3212778 |
| 42 | 10 | 102798646 | 102802645 | 102874569 | 102878568 |
| 43 | 6 | 3146780 | 3150779 | 3195850 | 3199849 |
| 44 | 12 | 114981190 | 114985189 | 115073539 | 115077538 |
| 45 | X | 46504988 | 46508987 | 46620210 | 46624209 |
| 46 | 5 | 150274698 | 150278697 | 150337586 | 150341585 |
| 47 | 12 | 91722239 | 91726238 | 91888285 | 91892284 |
| 48 | X | 46504988 | 46508987 | 46625862 | 46629861 |
| 49 | 7 | 27199902 | 27203901 | 27308508 | 27312507 |
| 50 | 5 | 132650162 | 132654161 | 132755647 | 132759646 |

TABLE 1B
(part 1e)

| No. | probe | Primer ID |
|---|---|---|
| 1 | Hg38_3_100733021_100742761_100862282_100875494_RF | OBD160.801 |
| 2 | Hg38_3_100750954_100758578_100862282_100875494_RF | OBD160.805 |
| 3 | Hg38_5_150272362_150278699_150330165_150332483_FR | OBD160.809 |
| 4 | Hg38_3_100862282_100875494_101052914_101071675_FF | OBD160.813 |
| 5 | Hg38_5_150008962_150014465_150272362_150278699_RF | OBD160.817 |
| 6 | Hg38_5_150272362_150278699_150382773_150387928_FF | OBD160.821 |
| 7 | Hg38_12_69346683_69349534_69539751_69542024_FF | OBD160.825 |
| 8 | Hg38_8_143073684_143079751_143219138_143224471_RF | OBD160.829 |
| 9 | Hg38_1_32214585_32217213_32237144_32241139_RF | OBD160.833 |
| 10 | Hg38_X_46498682_46508989_46616810_46620218_FR | OBD160.837 |
| 11 | Hg38_10_8256256_8258992_8314718_8322822_FF | OBD160.841 |
| 12 | Hg38_6_3027907_3030237_3146778_3149848_FR | OBD160.845 |
| 13 | Hg38_7_27204432_27205465_27302548_27312509_FF | OBD160.849 |
| 14 | Hg38_7_25370684_25382177_25542599_25548176_FF | OBD160.853 |
| 15 | Hg38_6_2898813_2902737_3146778_3149848_RR | OBD160.857 |
| 16 | Hg38_5_150036868_150040595_150272362_150278699_RF | OBD160.861 |
| 17 | Hg38_8_142863175_142868397_143073684_143079751_RR | OBD160.865 |
| 18 | Hg38_5_150194890_150198993_150272362_150278699_RF | OBD160.869 |
| 19 | Hg38_3_100862282_100875494_101027240_101038115_FF | OBD160.873 |
| 20 | Hg38_8_26561792_26565691_26638318_26644530_RR | OBD160.877 |
| 21 | Hg38_7_27151628_27154280_27302548_27312509_FF | OBD160.881 |
| 22 | Hg38_3_100677572_100682399_100862282_100875494_RF | OBD160.885 |
| 23 | Hg38_9_84621528_84639678_84804816_84814325_RF | OBD160.889 |
| 24 | Hg38_X_46498682_46508989_46571235_46574591_FR | OBD160.893 |
| 25 | Hg38_7_25274699_25276258_25370684_25382177_RF | OBD160.897 |
| 26 | Hg38_6_3146778_3149848_3232412_3234356_RF | OBD160.901 |
| 27 | Hg38_Y_7774814_7781345_7901883_7918222_RR | OBD160.905 |
| 28 | Hg38_6_47291785_47297630_47538465_47543792_RF | OBD160.909 |
| 29 | Hg38_5_150272362_150278699_150390262_150397231_FR | OBD160.913 |
| 30 | Hg38_6_2891780_2893777_3146778_3149848_FR | OBD160.917 |
| 31 | Hg38_13_33481982_33488531_33704999_33717490_RF | OBD160.921 |
| 32 | Hg38_6_3045545_3051391_3146778_3149848_FR | OBD160.925 |
| 33 | Hg38_8_26561792_26565691_26776216_26787349_RR | OBD160.929 |
| 34 | Hg38_6_2860707_2865436_3146778_3149848_FR | OBD160.933 |
| 35 | Hg38_7_27250080_27251352_27302548_27312509_FF | OBD160.937 |
| 36 | Hg38_12_114767229_114770912_114977828_114985191_RF | OBD160.941 |
| 37 | Hg38_7_22390272_22396878_22624843_22634759_FF | OBD160.945 |
| 38 | Hg38_12_91718135_91726240_91895416_91906634_FR | OBD160.949 |
| 39 | Hg38_10_8256256_8258992_8533854_8544622_FF | OBD160.953 |
| 40 | Hg38_8_142835475_142844052_143073684_143079751_FR | OBD160.957 |
| 41 | Hg38_6_3146778_3149848_3207080_3212780_RF | OBD160.961 |
| 42 | Hg38_10_102798644_102804186_102874567_102879563_RR | OBD160.965 |
| 43 | Hg38_6_3146778_3149848_3196920_3199851_RF | OBD160.969 |
| 44 | Hg38_12_114977828_114985191_115073537_115080207_FR | OBD160.973 |
| 45 | Hg38_X_46498682_46508989_46621627_46624211_FF | OBD160.977 |
| 46 | Hg38_5_150272362_150278699_150335788_150341587_FF | OBD160.981 |
| 47 | Hg38_12_91718135_91726240_91888283_91891782_FR | OBD160.985 |
| 48 | Hg38_X_46498682_46508989_46624211_46629863_FF | OBD160.989 |
| 49 | Hg38_7_27201418_27203903_27302548_27312509_FF | OBD160.993 |
| 50 | Hg38_5_132650160_132653783_132755645_132763810_RR | OBD160.997 |

TABLE 1B (part 1f)

| No. | Primer Sequence | Primer ID | Primer Sequence |
|---|---|---|---|
| 1 | GCAGGTAGGACAGGTGCCATCATTCCC | OBD160.803 | CCCAACCTGAAGAACAGAGAGAAGACTG |
| 2 | GGACACGAGCCAGGATGCTAAACAGTGA | OBD160.807 | GCTGTGTAATGAGGACTGAATAAGACTG |
| 3 | GGGCAAGGGTGGGAAGCGATGGC | OBD160.811 | TTCCCTGCCACCATTCTCCAGACGGGCG |
| 4 | GCAGGTAGGACAGGTGCCATCATTCCC | OBD160.815 | CCTCCCACATCTGATACTCCTCACCCC |
| 5 | AGCCCAGAAACCTTGGCAAACAGAAGAG | OBD160.819 | GCACCCAATAAGGAAAACAGAAAAGTTC |
| 6 | GGGCAAGGGTGGGAAGCGATGGC | OBD160.823 | GAGGGTCAGGTGGGCTCTGGGAGTTGTG |
| 7 | TAGTTCCCTTCAGTCACTCTTTGTAT | OBD160.827 | TGAGCCCTGGAGGTGGAGGTTGC |
| 8 | CCAACCAACTGATTTATGCCAGG | OBD160.831 | GACGACGGCTGTCTCCTCAAACA |
| 9 | CGCCCTTTCCAGCCCCTCAGTCC | OBD160.835 | CTCCCTCCTTGCTGCCTCTTCCCACCAG |
| 10 | AGATTTTGGGCTGAGACAATGGG | OBD160.839 | GGGCATTCCATAACTACAAGAGAAAA |
| 11 | AGAGCAGAGGGAAGAAGGAAGTCT | OBD160.843 | TATTATTATCCATTCTGCCAAAGC |
| 12 | GAAGGTCTCTGTGTAATGATGAACCC | OBD160.847 | AACCAGCACTCGCCCAAACCAGCA |
| 13 | CAACAAAACAGGAAACCAGGACTG | OBD160.851 | CAACAGAGATGGGAGACTTGAAG |
| 14 | GCCCTATCCTTACTCCCTGAACCTTGGG | OBD160.855 | GAACTGGCATCTCACGAATCCTCCCTGC |
| 15 | ATCCATCAAACAGAGACAACCACCCTTG | OBD160.859 | CGCCCAAACCAGCACTCCGACCC |
| 16 | CAGCCAGCAGATGGGTGGGTGGGC | OBD160.863 | ATAATGGGCTGCTACAGAGAAGGCACTG |
| 17 | CAACACCTCATTGACACCACTGGACAG | OBD160.867 | CTCAAACAGGGACCACGAGGATGATGGC |
| 18 | GGGCAAGGGTGGGAAGCGATGGC | OBD160.871 | ACCCCAGACACCCTCCGACAGGACCTCA |
| 19 | GCAGGTAGGACAGGTGCCATCATTCCC | OBD160.875 | CACTCACTAAGGTGGGTGGAGGTAATCC |
| 20 | GAGCAGCAAGTGAGCCAGCATTACCGCC | OBD160.879 | GCGTGTCTCTCAGGGAAGGCAGGATGC |
| 21 | GCCAGAGAAGGAGGGATTGATTC | OBD160.883 | CAACAGAGATGGGAGACTTGAAG |
| 22 | GCAGGTAGGACAGGTGCCATCATTCCC | OBD160.887 | GGGATTTCATCAACACCAGCCCTGACCT |
| 23 | CATTAGTTAGATTCTCATAAGGATGGC | OBD160.891 | TCACGGCGAACCCAATGTCTATT |
| 24 | AGATTTTGGGCTGAGACAATGGG | OBD160.895 | AAACAAGCCTGCTGACCGCCAGA |
| 25 | GCCCTATCCTTACTCCCTGAACCTTGGG | OBD160.899 | GTCTGGAGAACACGGCAGTTTGGCAGG |
| 26 | GCATCCTGTGAGGGCACCTGTGTCATTT | OBD160.903 | CTCCGACCCAAACCAGCACTCCAGCCG |
| 27 | AGGTTACAGCCACTGAGCCTGGATGTG | OBD160.907 | GGGAAATGAGTAAGTCTGAGGGAAACAA |
| 28 | GGGAGGGCAATGGTGGGCTGATGTCTCT | OBD160.911 | AATCAGAGGGCTGCCACCAGGCGAGTG |
| 29 | ACAGGACAGGGCAGGAGAGGCAACGAGG | OBD160.915 | GCCTTGCTGAATGCTGGTCACATCGCAG |
| 30 | GTAGATGGCATTGACAAGAACCAGCCTG | OBD160.919 | CCGACCCAAACCAGCACTCCAGCCG |
| 31 | ATGCCCACTTTCACCACTGCTATTGGAC | OBD160.923 | CGTCCACCTACATCTCCCTTACCTAT |
| 32 | GGTGGGTGCTGAGGAGTGGGCTC | OBD160.927 | ACTCCGACCCAAACCAGCACTCCAGCCG |
| 33 | GAGCAGCAAGTGAGCCAGCATTACCGCC | OBD160.931 | TGGGTGAGGTGGAGCCGAGTTTGGGTC |
| 34 | CCTTCCTCCCTCCCTCCCCGCTT | OBD160.935 | ACTCCGACCCAAACCAGCACTCCAGCCG |
| 35 | TAGTTAGATTCTCATAAGGATGGC | OBD160.939 | TTTCGTCCTTCGTCACATTCACGG |
| 36 | CTGGCTCAGAGGAGGTGGTGATTATGGA | OBD160.943 | GCCGAGGTCTCCAGTAACACCCTTGC |
| 37 | CAGTCTTGTGATAGAGGTTGGTGAGTGT | OBD160.947 | GATTTCAGAAGCACCTCCCTCCCCAGC |
| 38 | CTTTGTCAGTGCCTGTTGCCACCTTGGT | OBD160.951 | CTGTGGAGGAAAATAGTTGAGCAGGAAC |
| 39 | GCTCTTGGGTAGGCAGCCAGCAGCC | OBD160.955 | CCCACTTACTCTCTGACCTATCACCCAG |
| 40 | GTGTTTTGACAAGTTGGTGCCGAATGCT | OBD160.959 | CTCAAACAGGGACCACGAGGATGATGGC |
| 41 | CAGGGAGGGAGAAACCCAGACAG | OBD160.963 | CAAACCAGCACTCCGACCCAAAC |
| 42 | AACAGCAAGCCAGCCAGGTGTGGTGG | OBD160.967 | CACTCTCCTCCTCACCTTCAGCACCCG |
| 43 | GTGAGCAGGAGGCTTGGGCAGGC | OBD160.971 | ACTCCGACCCAAACCAGCACTCCAGCCG |
| 44 | CTGGCTCAGAGGAGGTGGTGATTATGGA | OBD160.975 | CCAACCCCAGCCACACACAGCAGACCC |
| 45 | AGATTTTGGGCTGAGACAATGGG | OBD160.979 | GGCTATTTCCGAGTTGTATCCTTTAT |
| 46 | GGGCAAGGGTGGGAAGCGATGGC | OBD160.983 | AGGTTGGCTGGCTGCTCCTGCTCCCTTG |
| 47 | TTTGTCAGTGCCTGTTGCCACCTTGGT | OBD160.987 | GTTTTCTGAGGCTGATTTCTTGTTGAGA |
| 48 | AGATTTTGGGCTGAGACAATGGG | OBD160.991 | AGAGCCATTATCTTTCCTTTGGTGAA |
| 49 | CAGGCATTAGTTAGATTCTCATAAGG | OBD160.995 | TCACGGCGAACCCAATGTCTATT |
| 50 | CAACCACTTCACCAAGGAGCCAGAGTTC | OBD160.999 | GGGATGGAGGGAAGAGTCACAGGAAAGT |

TABLE 1B
(part 1g)

| No. | probe | Markers |
|---|---|---|
| 1 | Hg38_3_100733021_100742761_100862282_100875494_RF | OBD160.801.803 |
| 2 | Hg38_3_100750954_100758578_100862282_100875494_RF | OBD160.805.807 |
| 3 | Hg38_5_150272362_150278699_150330165_150332483_FR | OBD160.809.811 |
| 4 | Hg38_3_100862282_100875494_101052914_101071675_FF | OBD160.813.815 |
| 5 | Hg38_5_150008962_150014465_150272362_150278699_RF | OBD160.817.819 |
| 6 | Hg38_5_150272362_150278699_150382773_150387928_FF | OBD160.821.823 |
| 7 | Hg38_12_69346683_69349534_69539751_69542024_FF | OBD160.825.827 |
| 8 | Hg38_8_143073684_143079751_143219138_143224471_RF | OBD160.829.831 |
| 9 | Hg38_1_32214585_32217213_32237144_32241139_RF | OBD160.833.835 |
| 10 | Hg38_X_46498682_46508989_46616810_46620218_FR | OBD160.837.839 |
| 11 | Hg38_10_8256256_8258992_8314718_8322822_FF | OBD160.841.843 |
| 12 | Hg38_6_3027907_3030237_3146778_3149848_FR | OBD160.845.847 |
| 13 | Hg38_7_27204432_27205465_27302548_27312509_FF | OBD160.849.851 |
| 14 | Hg38_7_25370684_25382177_25542599_25548176_FF | OBD160.853.855 |
| 15 | Hg38_6_2898813_2902737_3146778_3149848_RR | OBD160.857.859 |
| 16 | Hg38_5_150036868_150040595_150272362_150278699_RF | OBD160.861.863 |
| 17 | Hg38_8_142863175_142868397_143073684_143079751_RR | OBD160.865.867 |
| 18 | Hg38_5_150194890_150198993_150272362_150278699_RF | OBD160.869.871 |
| 19 | Hg38_3_100862282_100875494_101027240_101038115_FF | OBD160.873.875 |
| 20 | Hg38_8_26561792_26565691_26638318_26644530_RR | OBD160.877.879 |
| 21 | Hg38_7_27151628_27154280_27302548_27312509_FF | OBD160.881.883 |
| 22 | Hg38_3_100677572_100682399_100862282_100875494_RF | OBD160.885.887 |
| 23 | Hg38_9_84621528_84639678_84804816_84814325_RF | OBD160.889.891 |
| 24 | Hg38_X_46498682_46508989_46571235_46574591_FR | OBD160.893.895 |
| 25 | Hg38_7_25274699_25276258_25370684_25382177_RF | OBD160.897.899 |
| 26 | Hg38_6_3146778_3149848_3232412_3234356_RF | OBD160.901.903 |
| 27 | Hg38_Y_7774814_7781345_7901883_7918222_RR | OBD160.905.907 |
| 28 | Hg38_6_47291785_47297630_47538465_47543792_RF | OBD160.909.911 |
| 29 | Hg38_5_150272362_150278699_150390262_150397231_FR | OBD160.913.915 |
| 30 | Hg38_6_2891780_2893777_3146778_3149848_FR | OBD160.917.919 |
| 31 | Hg38_13_33481982_33488531_33704999_33717490_RF | OBD160.921.923 |
| 32 | Hg38_6_3045545_3051391_3146778_3149848_FR | OBD160.925.927 |
| 33 | Hg38_8_26561792_26565691_26776216_26787349_RR | OBD160.929.931 |
| 34 | Hg38_6_2860707_2865436_3146778_3149848_FR | OBD160.933.935 |
| 35 | Hg38_7_27250080_27251352_27302548_27312509_FF | OBD160.937.939 |
| 36 | Hg38_12_114767229_114770912_114977828_114985191_RF | OBD160.941.943 |
| 37 | Hg38_7_22390272_22396878_22624843_22634759_FF | OBD160.945.947 |
| 38 | Hg38_12_91718135_91726240_91895416_91906634_FR | OBD160.949.951 |
| 39 | Hg38_10_8256256_8258992_8533854_8544622_FF | OBD160.953.955 |
| 40 | Hg38_8_142835475_142844052_143073684_143079751_FR | OBD160.957.959 |
| 41 | Hg38_6_3146778_3149848_3207080_3212780_RF | OBD160.961.963 |
| 42 | Hg38_10_102798644_102804186_102874567_102879563_RR | OBD160.965.967 |
| 43 | Hg38_6_3146778_3149848_3196920_3199851_RF | OBD160.969.971 |
| 44 | Hg38_12_114977828_114985191_115073537_115080207_FR | OBD160.973.975 |
| 45 | Hg38_X_46498682_46508989_46621627_46624211_FF | OBD160.977.979 |
| 46 | Hg38_5_150272362_150278699_150335788_150341587_FF | OBD160.981.983 |
| 47 | Hg38_12_91718135_91726240_91888283_91891782_FR | OBD160.985.987 |
| 48 | Hg38_X_46498682_46508989_46624211_46629863_FF | OBD160.989.991 |
| 49 | Hg38_7_27201418_27203903_27302548_27312509_FF | OBD160.993.995 |
| 50 | Hg38_5_132650160_132653783_132755645_132763810_RR | OBD160.997.999 |

TABLE 1B
(part 2a)

| No. | probe | RP/Rsum | Rprank | FC:(class1/class2) | pfp |
|---|---|---|---|---|---|
| 51 | Hg38_21_44826248_44830834_44873351_44875256_RF | 2956.424781 | 46 | −1.509 | 0 |
| 52 | Hg38_6_3114019_3116008_3146778_3149848_RR | 2593.745836 | 39 | −1.508 | 0 |
| 53 | Hg38_5_132755645_132763810_132924803_132929300_RF | 3865.979955 | 80 | −1.507 | 0 |
| 54 | Hg38_6_2882362_2886326_3146778_3149848_FR | 2479.56492 | 37 | −1.504 | 0 |
| 55 | Hg38_12_68747488_68752550_68814505_68818951_RR | 2290.703965 | 33 | −1.503 | 0 |
| 56 | Hg38_18_46958975_46962799_47078769_47089265_FR | 3240.639266 | 56 | −1.5 | 0 |
| 57 | Hg38_18_46958975_46962799_47114979_47119139_FR | 3161.351495 | 52 | −1.498 | 0 |
| 58 | Hg38_19_34277524_34285717_34302513_34306297_RF | 4267.515378 | 97 | −1.497 | 0 |
| 59 | Hg38_5_132755645_132763810_132929300_132935025_RF | 3744.91901 | 77 | −1.495 | 0 |
| 60 | Hg38_5_132755645_132763810_132845490_132849804_RF | 3637.844194 | 69 | −1.491 | 0 |
| 61 | Hg38_12_89512516_89520005_89567382_89573321_FF | 3120.819609 | 49 | −1.491 | 0 |
| 62 | Hg38_18_46958975_46962799_47181285_47187993_FF | 4071.28168 | 87 | −1.489 | 0 |
| 63 | Hg38_4_145441661_145450618_145639668_145646739_FR | 4351.192236 | 102 | −1.489 | 0 |
| 64 | Hg38_8_65429758_65438145_65603423_65608237_FR | 3669.316935 | 70 | −1.488 | 0 |
| 65 | Hg38_21_44873351_44875256_45009774_45011942_FR | 3284.39733 | 59 | −1.484 | 0 |
| 66 | Hg38_7_27176915_27178105_27302548_27312509_FF | 3462.395102 | 61 | −1.481 | 0 |
| 67 | Hg38_8_143073684_143079751_143187205_143191965_RF | 2069.623189 | 26 | −1.48 | 0 |
| 68 | Hg38_21_44873351_44875256_44975178_44979847_FF | 3715.369973 | 75 | −1.477 | 0 |
| 69 | Hg38_X_104167549_104169666_104260928_104265364_RF | 2992.791924 | 47 | −1.47 | 0 |
| 70 | Hg38_X_46474993_46482135_46498682_46508989_RF | 4532.171008 | 111 | −1.469 | 0 |
| 71 | Hg38_18_46958975_46962799_47152390_47157459_FR | 4325.866296 | 101 | −1.469 | 0 |
| 72 | Hg38_18_46958975_46962799_47218671_47222248_FF | 4552.140095 | 116 | −1.467 | 0 |
| 73 | Hg38_12_8943814_8945391_9061693_9073885_RR | 3991.953624 | 85 | −1.467 | 0 |
| 74 | Hg38_18_46958975_46962799_47102132_47108776_FF | 4425.666246 | 106 | −1.465 | 0 |
| 75 | Hg38_3_126843341_126854251_126975387_126980610_FR | 4230.256246 | 96 | −1.463 | 0 |
| 76 | Hg38_X_46451412_46455647_46498682_46508989_RF | 5850.564876 | 178 | −1.463 | 0 |
| 77 | Hg38_6_47538465_47543792_47623265_47631675_FR | 3781.879009 | 78 | −1.462 | 0 |
| 78 | Hg38_1_69357288_69362673_69431002_69439637_RF | 4224.385254 | 95 | −1.461 | 0 |
| 79 | Hg38_2_3461060_3464337_3620203_3628632_RR | 4223.660735 | 94 | −1.461 | 0 |
| 80 | Hg38_3_126715028_126724613_126843341_126854251_RF | 4187.552449 | 92 | −1.459 | 0 |
| 81 | Hg38_X_104123664_104126241_104260928_104265364_RF | 3137.27664 | 50 | −1.455 | 0 |
| 82 | Hg38_X_147757585_147771392_148009858_148011061_RF | 4078.660813 | 88 | −1.454 | 0 |
| 83 | Hg38_X_46402474_46404815_46498682_46508989_RF | 5728.942859 | 169 | −1.454 | 0 |
| 84 | Hg38_2_191868245_191877609_192052829_192065719_RR | 3706.781886 | 74 | −1.453 | 0 |
| 85 | Hg38_20_19501430_19515696_19750261_19752596_RF | 4044.106873 | 86 | −1.453 | 0 |
| 86 | Hg38_3_13325884_13327571_13490970_13499186_FF | 4953.040936 | 132 | −1.452 | 0 |
| 87 | Hg38_18_46958975_46962799_47093730_47099291_FF | 4534.435881 | 112 | −1.452 | 0 |
| 88 | Hg38_4_145441661_145450618_145487239_145494181_FR | 6098.1236 | 198 | −1.451 | 0 |
| 89 | Hg38_X_104171401_104174188_104260928_104265364_RF | 3522.63992 | 64 | −1.45 | 0 |
| 90 | Hg38_5_150272362_150278699_150355072_150357974_FR | 4630.538732 | 120 | −1.449 | 0 |
| 91 | Hg38_7_148757046_148761950_148843816_148853209_RF | 3272.699272 | 58 | −1.447 | 0 |
| 92 | Hg38_X_46462567_46466475_46498682_46508989_RF | 5873.726154 | 181 | −1.447 | 0 |
| 93 | Hg38_1_211900971_211908280_212023996_212027825_FF | 3673.530159 | 71 | −1.447 | 0 |
| 94 | Hg38_7_27236651_27239035_27302548_27312509_FF | 3973.626367 | 83 | −1.447 | 0 |
| 95 | Hg38_18_46917333_46922591_46958975_46962799_RF | 6372.471571 | 218 | −1.445 | 0 |
| 96 | Hg38_17_17926103_17934669_18063962_18066155_RF | 4609.709066 | 117 | −1.443 | 0 |
| 97 | Hg38_17_44261859_44271957_44545451_44548502_RF | 6733.043227 | 242 | −1.443 | 0 |
| 98 | Hg38_4_145441661_145450618_145529286_145534613_FF | 5808.960219 | 173 | −1.443 | 0 |
| 99 | Hg38_3_52542416_52548124_52787566_52791869_FR | 3941.729282 | 82 | −1.441 | 0 |
| 100 | Hg38_2_27492451_27495056_27719544_27726504_FF | 5608.079466 | 162 | −1.439 | 0 |

TABLE 1B (part 2b)

| | P. | | Probe sequence |
|---|---|---|---|
| No. | value | Type | 60 mer |
| 51 | 0 | Healthy Control | TTTGCAGGTGTGTGGTTAACACTATCCTTCGATGAGTTCTTTGAGAGTTCTGTATTGTTT |
| 52 | 0 | Healthy Control | AATATAAATTCAATTCATTAAAAAATAATCGAGTGCTGGTTTGGGTCGGAGTGCTGGTTC |
| 53 | 0 | Healthy Control | AAAATTTGATTTAATTCTCCAATTATCATCGAATATTAGTAGTCCCGTGGCAAGCCTTTG |
| 54 | 0 | Healthy Control | CAAATAAAGCCAGAACTCACTCATTACCTCGAGTGCTGGTTTGGGTCGGAGTGCTGGTTC |
| 55 | 0 | Healthy Control | AGGCGGGCAGATCACCTGAGGTCAGAGTTCGAAGGAAAAGCTAAATTCTAGTTAGAAGTT |
| 56 | 0 | Healthy Control | ATCGTCGGGAGAATCTTCCCTTGTAGTCTCGATTTTCCTAAGCCCAAATAAAATGGTTCA |
| 57 | 0 | Healthy Control | ATCGTCGGGAGAATCTTCCCTTGTAGTCTCGACAGGAAACCTCATTTTATAGGTAGCCAT |
| 58 | 0 | Healthy Control | AGGACTGTGGGTGAGAAAAGCTCTGGATTCGATTAGATTTTGATTCAGTATTTTAGGCAA |
| 59 | 0 | Healthy Control | ATATAATTCTGAACTAATTTTCCAAACTTCGAATATTAGTAGTCCCGTGGCAAGCCTTTG |
| 60 | 0 | Healthy Control | AGTTTTTAATGTCAAATATGATATACAGTCGAATATTAGTAGTCCCGTGGCAAGCCTTTG |
| 61 | 0 | Healthy Control | GCCAAAGAGAGCTTTGGTACCAATAAATTCGAGTTTGCGTGGGCAGGAAATTTATTCTTT |
| 62 | 0 | Healthy Control | ATCGTCGGGAGAATCTTCCCTTGTAGTCTCGATAGTTTAATTTCCTAAGAGGAAAGTACT |
| 63 | 0 | Healthy Control | TCCCAGCCCAGTGTACAGAGTAAAAACATCGAATGGCGACTTTTTTCACAATATTTGGAT |
| 64 | 0 | Healthy Control | ACAGAGCCCTCAGAAATAATGCCACATATCGAGCAATTCATGTTTTAAAGTTGGACTCTA |
| 65 | 0 | Healthy Control | TTTGCAGGTGTGTGGTTAACACTATCCTTCGAAAAAAAAGCCTTTTGAGACTTCAGTTGG |
| 66 | 0 | Healthy Control | CCTAATGAAAAGATACCAGGTCCTGAGATCGAAGTAATGAAGAGGGAGGTGGTTTTCTTT |
| 67 | 0 | Healthy Control | GATCGGTCAGACATTGCCCATCTGTTGATCGAGGAGACAGCCGTTGTCCCCAGGGCTCCT |
| 68 | 0 | Healthy Control | TTTGCAGGTGTGTGGTTAACACTATCCTTCGATTACCGAGGGATTCATGTTCCTGCTCCT |
| 69 | 0 | Healthy Control | GGCAGGCAGATCACTTGAGGTCAGTAGTTCGATTCTCTCTTTTTTTCCTCCTCCTTTATT |
| 70 | 0 | Healthy Control | ACAGAGCCCTCAGAAATAATGCCGCATATCGAAACCATGAAGCTAGCAAACACTGGAATG |
| 71 | 0 | Healthy Control | ATCGTCGGGAGAATCTTCCCTTGTAGTCTCGAATGTTTTCACAATATTTGACTTCTAATG |
| 72 | 0 | Healthy Control | ATCGTCGGGAGAATCTTCCCTTGTAGTCTCGACTTCCCAGGTGATTCTCCTACCTCAGCC |
| 73 | 0 | Healthy Control | GTCGTGCAGTGGTGATGATCTCCTGCAATCGATCATGATGGCAGGCAATCAAAGAATCTT |
| 74 | 0 | Healthy Control | ATCGTCGGGAGAATCTTCCCTTGTAGTCTCGAGCATCAGATGAAGAAAAAATTAATCCAC |
| 75 | 0 | Healthy Control | GCATATGCAGGTGTGGGCTTTTTAAAAATCGATTAAGTTTTTATTCTGATTATTGTATGT |
| 76 | 0 | Healthy Control | ACAGAGCCCTCAGAAATAATGCCGCATATCGATATATAAAGATTGTTTTAATTTCTATCT |
| 77 | 0 | Healthy Control | TGGGTGTTAGGGTTTCAATATATGAATTTCGAAGCTAGCCAAAGCATGTATTGTTTATGT |
| 78 | 0 | Healthy Control | GAATGTTTAGGTTTTGTAAGTTTACAAATCGAGTGCCCAGTTACAGAATTTTCTGGAGTT |
| 79 | 0 | Healthy Control | ATGGGTGTGGGAGTTAAGGTCGCTGTCTTCGATTGGAGATTTTTGACTACAGAATCAGTC |
| 80 | 0 | Healthy Control | GCATATGCAGGTGTGGGCTTTTTAAAAATCGACTAGTTTTTAAAGTAGGTTGTCTGTTTT |
| 81 | 0 | Healthy Control | GGCAGGCAGATCACTTGAGGTCAGTAGTTCGACCTGTCATCTACATTAGGTATTTCTGCT |
| 82 | 0 | Healthy Control | AAGGCGGGCAGATCACGAGGTCAGGAGTTCGATCTCATATAGATATAAACCAAGTCTGTG |
| 83 | 0 | Healthy Control | ACAGAGCCCTCAGAAATAATGCCGCATATCGACAAGCACTTTTCTGCTAGAATGTGATAT |
| 84 | 0 | Healthy Control | GGCGGGCAGATCACTTGAGGTCAGGGATTCGAAGTCCATCCTTAATGAAAGATACCTCTA |
| 85 | 0 | Healthy Control | AGAATGGGCGATGTTTCTCAGGGCTGCTTCGAAAAGCATTTATTAAACACCTACCTTGCT |
| 86 | 0 | Healthy Control | TGACTGAGGTCCCAGGTGTGGCCCCAACTCGAAAAAGAAAAAAAGAGCCTGGTGAAAAAG |
| 87 | 0 | Healthy Control | ATCGTCGGGAGAATCTTCCCTTGTAGTCTCGACCAGAATCCTCTTCTCCAGGCGGCGTAA |
| 88 | 0 | Healthy Control | TCCCAGCCCAGTGTACAGAGTAAAAACATCGAAAACAAATCACATTCCTAACCAAAAGCT |
| 89 | 0 | Healthy Control | GGCAGGCAGATCACTTGAGGTCAGTAGTTCGAAGTGTTAGGTAACTTTCTCTTAGTGGAA |
| 90 | 0 | Healthy Control | AGTGCCTGTGAAAGTGTTTCTGAATTCCTCGACAACTTCTCATCTTCATCTCTAGCCCAG |
| 91 | 0 | Healthy Control | GGCAGGCAGATCACTTGAGGTCAGGAATTCGATTTTCAAGCAAAGTGCTGAATGGAATTT |
| 92 | 0 | Healthy Control | ACAGAGCCCTCAGAAATAATGCCGCATATCGACTCCCCTCATTCCTGGCTGTGTGACCTG |
| 93 | 0 | Healthy Control | GGCAGGCAGATCACTTGAGGTCAGGAGCTCGATGCCAACCTTTTCAAAGTAGTTAAACAG |
| 94 | 0 | Healthy Control | CTCATCTACCTGCAAGTGTGAGATCCACTCGAAGTAATGAAGAGGGAGGTGGTTTTCTTT |
| 95 | 0 | Healthy Control | ATCGTCGGGAGAATCTTCCCTTGTAGTCTCGAAATCCGCCATTTTATACCACCCGCGGGC |
| 96 | 0 | Healthy Control | AGCAAATGTTGACTTAGTAATTTTTATATCGAGGCCGCAGTGAGTTGTGATCATACCACT |
| 97 | 0 | Healthy Control | GTTCTAGAAGGTTCTGGAAGACAAAATATCGAGGACACCCGGGATTCAGCCAGGGAGGCC |
| 98 | 0 | Healthy Control | TCCCAGCCCAGTGTACAGAGTAAAAACATCGAGTCTTTTAGACATTTAATTCCACTACAT |
| 99 | 0 | Healthy Control | TGTTGAGGGTGTCCCGGAGCATCAAATCTCGATAATATGTCCTTGTCTTCTACAGAGATG |
| 100 | 0 | Healthy Control | GGGCGCGGGTCTGCGGAGCCCCCAGGGCTCGAGAAGACTTATTGTTTTGTCTGCCCAGAA |

TABLE 1B
(part 2c)

| | Probe Location | | | | |
|---|---|---|---|---|---|
| No. | Chr | Start1 | End1 | Start2 | End2 |
| 51 | 21 | 44826250 | 44826279 | 44875225 | 44875254 |
| 52 | 6 | 3114021 | 3114050 | 3146780 | 3146809 |
| 53 | 5 | 132755647 | 132755676 | 132929269 | 132929298 |
| 54 | 6 | 2886295 | 2886324 | 3146780 | 3146809 |
| 55 | 12 | 68747490 | 68747519 | 68814507 | 68814536 |
| 56 | 18 | 46962768 | 46962797 | 47078771 | 47078800 |
| 57 | 18 | 46962768 | 46962797 | 47114981 | 47115010 |
| 58 | 19 | 34277526 | 34277555 | 34306266 | 34306295 |
| 59 | 5 | 132755647 | 132755676 | 132934994 | 132935023 |
| 60 | 5 | 132755647 | 132755676 | 132849773 | 132849802 |
| 61 | 12 | 89519974 | 89520003 | 89573290 | 89573319 |
| 62 | 18 | 46962768 | 46962797 | 47187962 | 47187991 |
| 63 | 4 | 145450587 | 145450616 | 145639670 | 145639699 |
| 64 | 8 | 65438114 | 65438143 | 65603425 | 65603454 |
| 65 | 21 | 44875225 | 44875254 | 45009776 | 45009805 |
| 66 | 7 | 27178074 | 27178103 | 27312478 | 27312507 |
| 67 | 8 | 143073686 | 143073715 | 143191934 | 143191963 |
| 68 | 21 | 44875225 | 44875254 | 44979816 | 44979845 |
| 69 | X | 104167551 | 104167580 | 104265333 | 104265362 |
| 70 | X | 46474995 | 46475024 | 46508958 | 46508987 |
| 71 | 18 | 46962768 | 46962797 | 47152392 | 47152421 |
| 72 | 18 | 46962768 | 46962797 | 47222217 | 47222246 |
| 73 | 12 | 8943816 | 8943845 | 9061695 | 9061724 |
| 74 | 18 | 46962768 | 46962797 | 47108745 | 47108774 |
| 75 | 3 | 126854220 | 126854249 | 126975389 | 126975418 |
| 76 | X | 46451414 | 46451443 | 46508958 | 46508987 |
| 77 | 6 | 47543761 | 47543790 | 47623267 | 47623296 |
| 78 | 1 | 69357290 | 69357319 | 69439606 | 69439635 |
| 79 | 2 | 3461062 | 3461091 | 3620205 | 3620234 |
| 80 | 3 | 126715030 | 126715059 | 126854220 | 126854249 |
| 81 | X | 104123666 | 104123695 | 104265333 | 104265362 |
| 82 | X | 147757587 | 147757616 | 148011030 | 148011059 |
| 83 | X | 46402476 | 46402505 | 46508958 | 46508987 |
| 84 | 2 | 191868247 | 191868276 | 192052831 | 192052860 |
| 85 | 20 | 19501432 | 19501461 | 19752565 | 19752594 |
| 86 | 3 | 13327540 | 13327569 | 13499155 | 13499184 |
| 87 | 18 | 46962768 | 46962797 | 47099260 | 47099289 |
| 88 | 4 | 145450587 | 145450616 | 145487241 | 145487270 |
| 89 | X | 104171403 | 104171432 | 104265333 | 104265362 |
| 90 | 5 | 150278668 | 150278697 | 150355074 | 150355103 |
| 91 | 7 | 148757048 | 148757077 | 148853178 | 148853207 |
| 92 | X | 46462569 | 46462598 | 46508958 | 46508987 |
| 93 | 1 | 211908249 | 211908278 | 212027794 | 212027823 |
| 94 | 7 | 27239004 | 27239033 | 27312478 | 27312507 |
| 95 | 18 | 46917335 | 46917364 | 46962768 | 46962797 |
| 96 | 17 | 17926105 | 17926134 | 18066124 | 18066153 |
| 97 | 17 | 44261861 | 44261890 | 44548471 | 44548500 |
| 98 | 4 | 145450587 | 145450616 | 145534582 | 145534611 |
| 99 | 3 | 52548093 | 52548122 | 52787568 | 52787597 |
| 100 | 2 | 27495025 | 27495054 | 27726473 | 27726502 |

TABLE 1B (part 2d)

| | 4 kb Sequence Location | | | | |
|---|---|---|---|---|---|
| No. | Chr | Start1 | End1 | Start2 | End2 |
| 51 | 21 | 44826250 | 44830249 | 44871255 | 44875254 |
| 52 | 6 | 3114021 | 3118020 | 3146780 | 3150779 |
| 53 | 5 | 132755647 | 132759646 | 132925299 | 132929298 |
| 54 | 6 | 2882325 | 2886324 | 3146780 | 3150779 |
| 55 | 12 | 68747490 | 68751489 | 68814507 | 68818506 |
| 56 | 18 | 46958798 | 46962797 | 47078771 | 47082770 |
| 57 | 18 | 46958798 | 46962797 | 47114981 | 47118980 |
| 58 | 19 | 34277526 | 34281525 | 34302296 | 34306295 |
| 59 | 5 | 132755647 | 132759646 | 132931024 | 132935023 |
| 60 | 5 | 132755647 | 132759646 | 132845803 | 132849802 |
| 61 | 12 | 89516004 | 89520003 | 89569320 | 89573319 |
| 62 | 18 | 46958798 | 46962797 | 47183992 | 47187991 |
| 63 | 4 | 145446617 | 145450616 | 145639670 | 145643669 |
| 64 | 8 | 65434144 | 65438143 | 65603425 | 65607424 |
| 65 | 21 | 44871255 | 44875254 | 45009776 | 45013775 |
| 66 | 7 | 27174104 | 27178103 | 27308508 | 27312507 |
| 67 | 8 | 143073686 | 143077685 | 143187964 | 143191963 |
| 68 | 21 | 44871255 | 44875254 | 44975846 | 44979845 |
| 69 | X | 104167551 | 104171550 | 104261363 | 104265362 |
| 70 | X | 46474995 | 46478994 | 46504988 | 46508987 |
| 71 | 18 | 46958798 | 46962797 | 47152392 | 47156391 |
| 72 | 18 | 46958798 | 46962797 | 47218247 | 47222246 |
| 73 | 12 | 8943816 | 8947815 | 9061695 | 9065694 |
| 74 | 18 | 46958798 | 46962797 | 47104775 | 47108774 |
| 75 | 3 | 126850250 | 126854249 | 126975389 | 126979388 |
| 76 | X | 46451414 | 46455413 | 46504988 | 46508987 |
| 77 | 6 | 47539791 | 47543790 | 47623267 | 47627266 |
| 78 | 1 | 69357290 | 69361289 | 69435636 | 69439635 |
| 79 | 2 | 3461062 | 3465061 | 3620205 | 3624204 |
| 80 | 3 | 126715030 | 126719029 | 126850250 | 126854249 |
| 81 | X | 104123666 | 104127665 | 104261363 | 104265362 |
| 82 | X | 147757587 | 147761586 | 148007060 | 148011059 |
| 83 | X | 46402476 | 46406475 | 46504988 | 46508987 |
| 84 | 2 | 191868247 | 191872246 | 192052831 | 192056830 |
| 85 | 20 | 19501432 | 19505431 | 19748595 | 19752594 |
| 86 | 3 | 13323570 | 13327569 | 13495185 | 13499184 |
| 87 | 18 | 46958798 | 46962797 | 47095290 | 47099289 |
| 88 | 4 | 145446617 | 145450616 | 145487241 | 145491240 |
| 89 | X | 104171403 | 104175402 | 104261363 | 104265362 |
| 90 | 5 | 150274698 | 150278697 | 150355074 | 150359073 |
| 91 | 7 | 148757048 | 148761047 | 148849208 | 148853207 |
| 92 | X | 46462569 | 46466568 | 46504988 | 46508987 |
| 93 | 1 | 211904279 | 211908278 | 212023824 | 212027823 |
| 94 | 7 | 27235034 | 27239033 | 27308508 | 27312507 |
| 95 | 18 | 46917335 | 46921334 | 46958798 | 46962797 |
| 96 | 17 | 17926105 | 17930104 | 18062154 | 18066153 |
| 97 | 17 | 44261861 | 44265860 | 44544501 | 44548500 |
| 98 | 4 | 145446617 | 145450616 | 145530612 | 145534611 |
| 99 | 3 | 52544123 | 52548122 | 52787568 | 52791567 |
| 100 | 2 | 27491055 | 27495054 | 27722503 | 27726502 |

TABLE 1B (part 2e)

| No. | probe | Primer ID |
|---|---|---|
| 51 | Hg38_21_44826248_44830834_44873351_44875256_RF | OBD160.1001 |
| 52 | Hg38_6_3114019_3116008_3146778_3149848_RR | OBD160.1005 |
| 53 | Hg38_5_132755645_132763810_132924803_132929300_RF | OBD160.1009 |
| 54 | Hg38_6_2882362_2886326_3146778_3149848_FR | OBD160.1013 |
| 55 | Hg38_12_68747488_68752550_68814505_68818951_RR | OBD160.1017 |
| 56 | Hg38_18_46958975_46962799_47078769_47089265_FR | OBD160.1021 |
| 57 | Hg38_18_46958975_46962799_47114979_47119139_FR | OBD160.1025 |
| 58 | Hg38_19_34277524_34285717_34302513_34306297_RF | OBD160.1029 |
| 59 | Hg38_5_132755645_132763810_132929300_132935025_RF | OBD160.1033 |
| 60 | Hg38_5_132755645_132763810_132845490_132849804_RF | OBD160.1037 |
| 61 | Hg38_12_89512516_89520005_89567382_89573321_FF | OBD160.1041 |
| 62 | Hg38_18_46958975_46962799_47181285_47187993_FF | OBD160.1045 |
| 63 | Hg38_4_145441661_145450618_145639668_145646739_FR | OBD160.1049 |
| 64 | Hg38_8_65429758_65438145_65603423_65608237_FR | OBD160.1053 |
| 65 | Hg38_21_44873351_44875256_45009774_45011942_FR | OBD160.1057 |
| 66 | Hg38_7_27176915_27178105_27302548_27312509_FF | OBD160.1061 |
| 67 | Hg38_8_143073684_143079751_143187205_143191965_RF | OBD160.1065 |
| 68 | Hg38_21_44873351_44875256_44975178_44979847_FF | OBD160.1069 |
| 69 | Hg38_X_104167549_104169666_104260928_104265364_RF | OBD160.1073 |
| 70 | Hg38_X_46474993_46482135_46498682_46508989_RF | OBD160.1077 |
| 71 | Hg38_18_46958975_46962799_47152390_47157459_FR | OBD160.1081 |
| 72 | Hg38_18_46958975_46962799_47218671_47222248_FF | OBD160.1085 |
| 73 | Hg38_12_8943814_8945391_9061693_9073885_RR | OBD160.1089 |
| 74 | Hg38_18_46958975_46962799_47102132_47108776_FF | OBD160.1093 |
| 75 | Hg38_3_126843341_126854251_126975387_126980610_FR | OBD160.1097 |
| 76 | Hg38_X_46451412_46455647_46498682_46508989_RF | OBD160.1101 |
| 77 | Hg38_6_47538465_47543792_47623265_47631675_FR | OBD160.1105 |
| 78 | Hg38_1_69357288_69362673_69431002_69439637_RF | OBD160.1109 |
| 79 | Hg38_2_3461060_3464337_3620203_3628632_RR | OBD160.1113 |
| 80 | Hg38_3_126715028_126724613_126843341_126854251_RF | OBD160.1117 |
| 81 | Hg38_X_104123664_104126241_104260928_104265364_RF | OBD160.1121 |
| 82 | Hg38_X_147757585_147771392_148009858_148011061_RF | OBD160.1125 |
| 83 | Hg38_X_46402474_46404815_46498682_46508989_RF | OBD160.1129 |
| 84 | Hg38_2_191868245_191877609_192052829_192065719_RR | OBD160.1133 |
| 85 | Hg38_20_19501430_19515696_19750261_19752596_RF | OBD160.1137 |
| 86 | Hg38_3_13325884_13327571_13490970_13499186_FF | OBD160.1141 |
| 87 | Hg38_18_46958975_46962799_47093730_47099291_FF | OBD160.1145 |
| 88 | Hg38_4_145441661_145450618_145487239_145494181_FR | OBD160.1149 |
| 89 | Hg38_X_104171401_104174188_104260928_104265364_RF | OBD160.1153 |
| 90 | Hg38_5_150272362_150278699_150355072_150357974_FR | OBD160.1157 |
| 91 | Hg38_7_148757046_148761950_148843816_148853209_RF | OBD160.1161 |
| 92 | Hg38_X_46462567_46466475_46498682_46508989_RF | OBD160.1165 |
| 93 | Hg38_1_211900971_211908280_212023996_212027825_FF | OBD160.1169 |
| 94 | Hg38_7_27236651_27239035_27302548_27312509_FF | OBD160.1173 |
| 95 | Hg38_18_46917333_46922591_46958975_46962799_RF | OBD160.1177 |
| 96 | Hg38_17_17926103_17934669_18063962_18066155_RF | OBD160.1181 |
| 97 | Hg38_17_44261859_44271957_44545451_44548502_RF | OBD160.1185 |
| 98 | Hg38_4_145441661_145450618_145529286_145534613_FF | OBD160.1189 |
| 99 | Hg38_3_52542416_52548124_52787566_52791869_FR | OBD160.1193 |
| 100 | Hg38_2_27492451_27495056_27719544_27726504_FF | OBD160.1197 |

TABLE 1B (part 2f)

| No. | Primer Sequence | Primer ID | Primer Sequence |
|---|---|---|---|
| 51 | CTCTGTTCCGCCTACCCAGGTCCACACC | OBD160.1003 | CCTTCTTCCTGTCTCCTTCTCCTCCATC |
| 52 | CAGGGCACTCAGGCTGGGTGAAGG | OBD160.1007 | AGCACTCGCCCAAACCAGCACTCCGACC |
| 53 | TTCCCAGTGGTTGACACAAAGTAGGTGC | OBD160.1011 | GGGATGGAGGGAAGAGTCACAGGAAAGT |
| 54 | TCACTTTGACATTCATCCCTGGG | OBD160.1015 | TGATGGAGAAGTTTATGATGAAGGAATC |
| 55 | GGCTCACCACTGTAACCCCAGCG | OBD160.1019 | CCAGGGATGCCCAAGAAGTCCTT |
| 56 | GCAGTGGCTGGGTGTGGGAGGCA | OBD160.1023 | GGGAGATGGTGGGTTCATTCTGCTTTT |
| 57 | GCAGTGGCTGGGTGTGGGAGGCA | OBD160.1027 | GGCACTGGCTGTGAACCTGAGGAGGCTG |
| 58 | TTTCTAAATGTGATTGCCAAGCATAGGC | OBD160.1031 | GATGAAGGAATCAGGCATAACACCT |
| 59 | GCTGGGTGCGGGTCCTACTGGAGTCC | OBD160.1035 | GGGATGGAGGGAAGAGTCACAGGAAAGT |
| 60 | GGCAATGGTCAAGGGCACGAACACAATG | OBD160.1039 | GGGATGGAGGGAAGAGTCACAGGAAAGT |
| 61 | CCGTAAAGTTGGACACTAAATGACATAG | OBD160.1043 | TTCAGCGTTTGACTTTTGAGAAGAGTGC |
| 62 | CCGTCGCAAGTTTCGTTCCCCAACCCGC | OBD160.1047 | CTCGGCACTGACTCCCAACCCGC |
| 63 | CTGCCCTCAGAGAGTAAAGAGACAGGAC | OBD160.1051 | AGGCTGGGTTTACAGTCACTCCGAGAAA |
| 64 | CAAGTCTACAGTAACCAAAACAGCAT | OBD160.1055 | TTTTGGAGACTGTTTGAATGTGACCA |
| 65 | CTCTGTTCCGCCTACCCAGGTCCACACC | OBD160.1059 | CCATACCCCAGCAACGGACAGCCAG |
| 66 | GCCAATAAGGGTCTCTCTCAGAA | OBD160.1063 | GATGAAGGAATCAGGCATAACACC |
| 67 | GGAAGACCTCACAGGGACTCTGC | OBD160.1067 | GACGACGGCTGTCTCCTCAAACA |
| 68 | CTCTGTTCCGCCTACCCAGGTCCACACC | OBD160.1071 | GTGGCAGCAGGTGTCCCATCGGC |
| 69 | AGTGCTGAGGTTGAGAAGCCCCAAGTTA | OBD160.1075 | TTGTGTAAGTCAGTCCATCCTAAGCCTT |
| 70 | AGATTTTGGGCTGAGACAATGGG | OBD160.1079 | GATGACACCTCACTTCTTTCCGAA |
| 71 | TATCCGTAAGGCAAGTCCTGAGT | OBD160.1083 | TGGCTACACAGTGAGACTCCGTC |
| 72 | TATCCGTAAGGCAAGTCCTGAGT | OBD160.1087 | GCCCAGGAGTTTGAGACCAGCCT |
| 73 | GCTCCATTTTCCTTGTCCCTCCTCAGGT | OBD160.1091 | ACCGATGTGGACAATGAAGACCTGTGGG |
| 74 | CCGTCGCAAGTTTCGTTCCCCAACCCGC | OBD160.1095 | CCCCACAGCCCCACCTAACCTGATG |
| 75 | GGCAGAAAGGAAAGAGCCTCGCAGCATT | OBD160.1099 | GTCATAGCAAGAACCAGAGAAATGTCAG |
| 76 | AGATTTTGGGCTGAGACAATGGG | OBD160.1103 | GAAAGTTGTTTATGAGGCACTGTTTACA |
| 77 | GGGAGGGCAATGGTGGGCTGATGTC | OBD160.1107 | ATGGCATCACCTTCCTCAAGAGTGC |
| 78 | TTGTGAGAAGGGTAAAATCTTAGGCAGC | OBD160.1111 | CTATGCCCCAAGCCAGAATGAAGCGGTT |
| 79 | CCGTGAGCACAGTTCTCCGCTGGTAAAT | OBD160.1115 | GCTGTAGCAAAGCGACAAAACTAAGAGT |
| 80 | GGCAGAAAGGAAAGAGCCTCGCAGCATT | OBD160.1119 | CCCCAGGGAGATGGACGGGCAGAGC |
| 81 | TAATCCCAGCAAGTTGAGAGGCT | OBD160.1123 | TAGAGGAGGGATAGCATTAGCAG |
| 82 | CACCTAAGGAGCCAGCAACTGACAAGAG | OBD160.1127 | GGGTCTGCTGTGCTCCACGGCTGAAGG |
| 83 | AGATTTTGGGCTGAGACAATGGG | OBD160.1131 | TAGGAGTTTTGCTTTGGGACTTGTTA |
| 84 | CACCTGTAATCCCAGCACTTTGG | OBD160.1135 | GGACAAGTTATTCACACGCCAGTA |
| 85 | GCCTGACATTCCTGCCTTCTTAT | OBD160.1139 | GCAAGTGTCAGATTTTGATGTCCCAT |
| 86 | GGTGCTCTGCGTCCCACACACGG | OBD160.1143 | GTTGGGTTGGGTGGGCACATCCTGGGTC |
| 87 | CCGTCGCAAGTTTCGTTCCCCAACCCGC | OBD160.1147 | CTTCCCTTCCTTCCCTCCCTCAGTTCTC |
| 88 | CTGCCCTCAGAGAGTAAAGAGACAGGAC | OBD160.1151 | GGTGGCTACAGCAGAGTGATGAAAATAC |
| 89 | TGGCTCATACCTGTAATCCCAGCAAGTT | OBD160.1155 | CAGATTATTCAAAAGGAAAACACCCAAC |
| 90 | GGGCAAGGGTGGGAAGCGATGGC | OBD160.1159 | GGGAGGGTGCTGGTGGCAGTGGAGTTGA |
| 91 | CCCAACAGATACCAAAATCCACGAAGAC | OBD160.1163 | CCAGGCAAACTGTGATGGTAGGGTCAGG |
| 92 | AGATTTTGGGCTGAGACAATGGG | OBD160.1167 | GAGCCTAAGTAACTAACCCAGGT |
| 93 | ATCTACTTTCAGGCAGTGGCTCACGCC | OBD160.1171 | CTCTCTTCCTTTTCTGTGCCTGGACTTA |
| 94 | GCAAGGAGGTGACAGGAGTGGTC | OBD160.1175 | CAACAGAGATGGGAGACTTGAAG |
| 95 | CGTCGCAAGTTTCGTTCCCCAACCCGCA | OBD160.1179 | TCGTTGGCGGCAGCGGGAGTGGGT |
| 96 | GTCCCTGCTGGGCTGCCTCAGTC | OBD160.1183 | CCGCATCTCCCCTTGTGCCAGTCCAGAC |
| 97 | CCCATCACCCCAAGGACCCCTCC | OBD160.1187 | CACTCAGGGCTCTGCCGTCACCTTGGAG |
| 98 | CTGCCCTCAGAGAGTAAAGAGACAGGAC | OBD160.1191 | ACACTGAAAAGGAAGAACTGTGAGAGGG |
| 99 | ATTATCCTCCTTTGTTCCTTCCCC | OBD160.1195 | GCCACTTACTCCTTCTGGGTCCC |
| 100 | TCCGCCCTGCCCACACCCACCTC | OBD160.1199 | GGTGTTGAGATTGAAGGAGACTGTCAGG |

TABLE 1B (part 2g)

| No. | probe | Markers |
|---|---|---|
| 51 | Hg38_21_44826248_44830834_44873351_44875256_RF | OBD160.1001.1003 |
| 52 | Hg38_6_3114019_3116008_3146778_3149848_RR | OBD160.1005.1007 |
| 53 | Hg38_5_132755645_132763810_132924803_132929300_RF | OBD160.1009.1011 |
| 54 | Hg38_6_2882362_2886326_3146778_3149848_FR | OBD160.1013.1015 |
| 55 | Hg38_12_68747488_68752550_68814505_68818951_RR | OBD160.1017.1019 |
| 56 | Hg38_18_46958975_46962799_47078769_47089265_FR | OBD160.1021.1023 |
| 57 | Hg38_18_46958975_46962799_47114979_47119139_FR | OBD160.1025.1027 |
| 58 | Hg38_19_34277524_34285717_34302513_34306297_RF | OBD160.1029.1031 |
| 59 | Hg38_5_132755645_132763810_132929300_132935025_RF | OBD160.1033.1035 |
| 60 | Hg38_5_132755645_132763810_132845490_132849804_RF | OBD160.1037.1039 |
| 61 | Hg38_12_89512516_89520005_89567382_89573321_FF | OBD160.1041.1043 |
| 62 | Hg38_18_46958975_46962799_47181285_47187993_FF | OBD160.1045.1047 |
| 63 | Hg38_4_145441661_145450618_145639668_145646739_FR | OBD160.1049.1051 |
| 64 | Hg38_8_65429758_65438145_65603423_65608237_FR | OBD160.1053.1055 |
| 65 | Hg38_21_44873351_44875256_45009774_45011942_FR | OBD160.1057.1059 |
| 66 | Hg38_7_27176915_27178105_27302548_27312509_FF | OBD160.1061.1063 |
| 67 | Hg38_8_143073684_143079751_143187205_143191965_RF | OBD160.1065.1067 |
| 68 | Hg38_21_44873351_44875256_44975178_44979847_FF | OBD160.1069.1071 |
| 69 | Hg38_X_104167549_104169666_104260928_104265364_RF | OBD160.1073.1075 |
| 70 | Hg38_X_46474993_46482135_46498682_46508989_RF | OBD160.1077.1079 |
| 71 | Hg38_18_46958975_46962799_47152390_47157459_FR | OBD160.1081.1083 |
| 72 | Hg38_18_46958975_46962799_47218671_47222248_FF | OBD160.1085.1087 |
| 73 | Hg38_12_8943814_8945391_9061693_9073885_RR | OBD160.1089.1091 |
| 74 | Hg38_18_46958975_46962799_47102132_47108776_FF | OBD160.1093.1095 |
| 75 | Hg38_3_126843341_126854251_126975387_126980610_FR | OBD160.1097.1099 |
| 76 | Hg38_X_46451412_46455647_46498682_46508989_RF | OBD160.1101.1103 |
| 77 | Hg38_6_47538465_47543792_47623265_47631675_FR | OBD160.1105.1107 |
| 78 | Hg38_1_69357288_69362673_69431002_69439637_RF | OBD160.1109.1111 |
| 79 | Hg38_2_3461060_3464337_3620203_3628632_RR | OBD160.1113.1115 |
| 80 | Hg38_3_126715028_126724613_126843341_126854251_RF | OBD160.1117.1119 |
| 81 | Hg38_X_104123664_104126241_104260928_104265364_RF | OBD160.1121.1123 |
| 82 | Hg38_X_147757585_147771392_148009858_148011061_RF | OBD160.1125.1127 |
| 83 | Hg38_X_46402474_46404815_46498682_46508989_RF | OBD160.1129.1131 |
| 84 | Hg38_2_191868245_191877609_192052829_192065719_RR | OBD160.1133.1135 |
| 85 | Hg38_20_19501430_19515696_19750261_19752596_RF | OBD160.1137.1139 |
| 86 | Hg38_3_13325884_13327571_13490970_13499186_FF | OBD160.1141.1143 |
| 87 | Hg38_18_46958975_46962799_47093730_47099291_FF | OBD160.1145.1147 |
| 88 | Hg38_4_145441661_145450618_145487239_145494181_FR | OBD160.1149.1151 |
| 89 | Hg38_X_104171401_104174188_104260928_104265364_RF | OBD160.1153.1155 |
| 90 | Hg38_5_150272362_150278699_150355072_150357974_FR | OBD160.1157.1159 |
| 91 | Hg38_7_148757046_148761950_148843816_148853209_RF | OBD160.1161.1163 |
| 92 | Hg38_X_46462567_46466475_46498682_46508989_RF | OBD160.1165.1167 |
| 93 | Hg38_1_211900971_211908280_212023996_212027825_FF | OBD160.1169.1171 |
| 94 | Hg38_7_27236651_27239035_27302548_27312509_FF | OBD160.1173.1175 |
| 95 | Hg38_18_46917333_46922591_46958975_46962799_RF | OBD160.1177.1179 |
| 96 | Hg38_17_17926103_17934669_18063962_18066155_RF | OBD160.1181.1183 |
| 97 | Hg38_17_44261859_44271957_44545451_44548502_RF | OBD160.1185.1187 |
| 98 | Hg38_4_145441661_145450618_145529286_145534613_FF | OBD160.1189.1191 |
| 99 | Hg38_3_52542416_52548124_52787566_52791869_FR | OBD160.1193.1195 |
| 100 | Hg38_2_27492451_27495056_27719544_27726504_FF | OBD160.1197.1199 |

TABLE 1B (part 3a)

| | | | | FC: (class1/ | |
|---|---|---|---|---|---|
| No. | probe | RP/Rsum | Rprank | class2) | pfp |
| 101 | Hg38_7_148843816_148853209_149049131_149053931_FF | 3724.759194 | 76 | −1.439 | 0 |
| 102 | Hg38_5_150134627_150137703_150272362_150278699_RF | 5047.498487 | 139 | −1.438 | 0 |
| 103 | Hg38_X_46398622_46399662_46498682_46508989_RF | 6153.043315 | 200 | −1.438 | 0 |
| 104 | Hg38_3_52542416_52548124_52688436_52689750_FR | 4290.852063 | 98 | −1.438 | 0 |
| 105 | Hg38_13_77929805_77933751_77995325_78000878_FR | 4895.079027 | 128 | −1.437 | 0 |
| 106 | Hg38_X_77891626_77893835_78036858_78043877_RR | 4737.136557 | 123 | −1.437 | 0 |
| 107 | Hg38_5_132755645_132763810_132995308_132998933_RF | 5427.156748 | 152 | −1.436 | 0 |
| 108 | Hg38_8_65429758_65438145_65628655_65630980_FF | 5313.744389 | 148 | −1.436 | 0 |
| 109 | Hg38_5_94505842_94514405_94641711_94646370_FR | 5118.072112 | 141 | −1.436 | 0 |
| 110 | Hg38_2_13845156_13854426_13990532_13998047_FF | 4209.708849 | 93 | −1.435 | 0 |
| 111 | Hg38_18_46958975_46962799_47121690_47124504_FR | 5139.553936 | 144 | −1.435 | 0 |
| 112 | Hg38_X_46498682_46508989_46603634_46604788_FF | 7085.42019 | 267 | −1.435 | 0 |
| 113 | Hg38_11_83253501_83261801_83434843_83442656_RF | 4953.072097 | 133 | −1.435 | 0 |
| 114 | Hg38_5_34366017_34372954_34527904_34530421_RR | 4740.961086 | 124 | −1.434 | 0 |
| 115 | Hg38_18_46958975_46962799_47058015_47062163_FR | 6552.392969 | 229 | −1.434 | 0 |
| 116 | Hg38_19_45210518_45212404_45410404_45414235_FF | 4098.241985 | 89 | −1.433 | 0 |
| 117 | Hg38_3_126755033_126764525_126843341_126854251_RF | 5104.936168 | 140 | −1.433 | 0 |
| 118 | Hg38_12_6595585_6598606_6681014_6683610_FF | 4353.353812 | 103 | −1.432 | 0 |
| 119 | Hg38_9_87096089_87101882_87143570_87148986_RR | 5684.666484 | 166 | −1.431 | 0 |
| 120 | Hg38_3_52542416_52548124_52699561_52701305_FF | 4512.767485 | 108 | −1.43 | 0 |
| 121 | Hg38_17_17926103_17934669_18141138_18142995_RF | 4946.982257 | 131 | −1.429 | 0 |
| 122 | Hg38_3_126604176_126607234_126843341_126854251_RF | 5936.323554 | 185 | −1.429 | 0 |
| 123 | Hg38_2_27492451_27495056_27657039_27659544_FR | 6695.897118 | 239 | −1.428 | 0 |
| 124 | Hg38_20_45581538_45584773_45643172_45649623_FR | 6225.863337 | 205 | −1.426 | 0 |
| 125 | Hg38_3_126627400_126630792_126843341_126854251_RF | 5809.854722 | 174 | −1.425 | 0 |
| 126 | Hg38_4_112653708_112659532_112867641_112872043_RR | 4539.896621 | 114 | −1.425 | 0 |
| 127 | Hg38_17_17840582_17849843_17926103_17934669_FR | 5118.63214 | 142 | −1.424 | 0 |
| 128 | Hg38_15_88873420_88883875_88904252_88906658_FF | 3854.072433 | 79 | −1.424 | 0 |
| 129 | Hg38_5_132755645_132763810_132790667_132795213_RF | 5630.548983 | 163 | −1.423 | 0 |
| 130 | Hg38_8_65429758_65438145_65633322_65635344_FR | 5572.230006 | 159 | −1.422 | 0 |
| 131 | Hg38_4_145339443_145344834_145441661_145450618_RF | 7036.536906 | 262 | −1.422 | 0 |
| 132 | Hg38_X_155860883_155870741_155953718_155958970_FR | 5391.94258 | 150 | −1.422 | 0 |
| 133 | Hg38_3_39518499_39534447_39571894_39578360_RF | 5227.376758 | 147 | −1.422 | 0 |
| 134 | Hg38_18_34889295_34890326_35083399_35092622_RR | 7546.609989 | 292 | −1.421 | 0 |
| 135 | Hg38_3_52500907_52509286_52542416_52548124_RF | 5037.934163 | 138 | −1.421 | 0 |
| 136 | Hg38_3_52542416_52548124_52570196_52574410_FF | 4864.647822 | 126 | −1.421 | 0 |
| 137 | Hg38_X_109485286_109494606_109613112_109617390_RR | 6064.912335 | 194 | −1.42 | 0 |
| 138 | Hg38_7_148843816_148853209_148905600_148909514_FF | 4627.431815 | 119 | −1.42 | 0 |
| 139 | Hg38_11_7448008_7457037_7491523_7495793_RR | 7829.755413 | 306 | −1.419 | 0 |
| 140 | Hg38_21_44873351_44875256_45059036_45065042_FF | 5784.979186 | 171 | −1.418 | 0 |
| 141 | Hg38_6_3041214_3044157_3146778_3149848_RR | 5786.571716 | 172 | −1.418 | 0 |
| 142 | Hg38_8_142901645_142903892_143073684_143079751_FR | 2855.548039 | 44 | −1.418 | 0 |
| 143 | Hg38_17_44261859_44271957_44488590_44490000_RF | 8062.138802 | 317 | −1.418 | 0 |
| 144 | Hg38_1_224692837_224704390_224807715_224812345_RF | 4525.738485 | 110 | −1.417 | 0 |
| 145 | Hg38_17_17728293_17735070_17926103_17934669_FR | 5426.454521 | 151 | −1.417 | 0 |
| 146 | Hg38_4_36078971_36105117_36290916_36293184_FR | 4482.618456 | 107 | −1.416 | 0 |
| 147 | Hg38_5_132679860_132681735_132755645_132763810_FR | 6289.515975 | 209 | −1.416 | 0 |
| 148 | Hg38_7_143128013_143133284_143335881_143341356_RF | 4633.985604 | 121 | −1.415 | 0 |
| 149 | Hg38_22_29467180_29469328_29549133_29556322_FR | 6305.561422 | 213 | −1.415 | 0 |
| 150 | Hg38_2_223382061_223395131_223446116_223450606_RR | 5584.38367 | 160 | −1.414 | 0 |

TABLE 1B (part 3b)

| | P. | | Probe sequence |
|---|---|---|---|
| No. | value | Type | 60 mer |
| 101 | 0 | Healthy Control | GGCAGGCAGATCACTTGAGGTCAGGAATTCGATGTCTTCTTCCGCCATGGTGCTGAATCC |
| 102 | 0 | Healthy Control | AGTGCCTGTGAAAGTGTTTCTGAATTCCTCGAAGGCTGTGGTGAGAATCCACTGGGAAGT |
| 103 | 0 | Healthy Control | ACAGAGCCCTCAGAAATAATGCCGCATATCGAGGCCCTTGCATTCGTTTTCTATTGTTAA |
| 104 | 0 | Healthy Control | TGTTGAGGGTGTCCCGGAGCATCAAATCTCGACTCTAGCTTATGCTGTTTAATGCCTTTC |
| 105 | 0 | Healthy Control | ATAATGGGCGATGTTTCTCAGGGCTGCTTCGATTCTTATGACATTAACTTTCATTTTTAT |
| 106 | 0 | Healthy Control | AGCCCAGCCATTTATTCTCTGATATTTATCGAGGTGAAACATAATGGTAGCTTGGACCAG |
| 107 | 0 | Healthy Control | TAAAAGCAATTTCATTTTTTTTTTCTTTTCGAATATTAGTAGTCCCGTGGCAAGCCTTTG |
| 108 | 0 | Healthy Control | ACAGAGCCCTCAGAAATAATGCCACATATCGAAAATACTGTACAATAGTCTCAGGATTTG |
| 109 | 0 | Healthy Control | TGATGTGGATTTGTACTCTATTACAGTTTCGAGGGTGTGGGTAGATATATTCTCAGTTTT |
| 110 | 0 | Healthy Control | GGCAGGCAGATCACTTGAGGTCACGAGTTCGATAGGTAAAGACTTACTGTTGTCATTTTG |
| 111 | 0 | Healthy Control | ATCGTCGGGAGAATCTTCCCTTGTAGTCTCGAAAGCCTTGCCCTATCTATCCCCAACCCC |
| 112 | 0 | Healthy Control | ACAGAGCCCTCAGAAATAATGCCGCATATCGACCATTTTGTAAAAACAGGAGTGGCAATT |
| 113 | 0 | Healthy Control | ATAATGGGCGATGTTTCTCAGGGCTGCTTCGACAAACCTGTTCTTTCAGGGCAAAAAAAT |
| 114 | 0 | Healthy Control | ACATATTTCAAATGAGCTTTATAAATGTTCGAGGCGGGCAGATCACTTGAGGCCAGAAGT |
| 115 | 0 | Healthy Control | ATCGTCGGGAGAATCTTCCCTTGTAGTCTCGAGGAGGCAATCTTAGAAGAACCCAGTGAG |
| 116 | 0 | Healthy Control | TTAAATAGAAACCTCTTTATTCTAAGTATCGAAAGTGCTGGGATTATTAGGCATGAGCCA |
| 117 | 0 | Healthy Control | GCATATGCAGGTGTGGGCTTTTTAAAAATCGAGTACCTGAAGAACGATGGCCTGGAAAAG |
| 118 | 0 | Healthy Control | GAGGCGGGCAGATCACGAGGTCAAGAGATCGACTTACTGCACTCAGGTGATTCTCCTGCC |
| 119 | 0 | Healthy Control | GAGCCAGCCAGCCCTGGAATTAACTTCCTCGAGATACCATCATGTAGAAAAGTCACAAAC |
| 120 | 0 | Healthy Control | TGTTGAGGGTGTCCCGGAGCATCAAATCTCGATTGGATTTTGTGAATTTTTGTAGCTTTT |
| 121 | 0 | Healthy Control | GCTCTTTAACTTTGGCCATTGTCTATCCTCGAGGCCGCAGTGAGTTGTGATCATACCACT |
| 122 | 0 | Healthy Control | GCATATGCAGGTGTGGGCTTTTTAAAAATCGACTCTGTCAAGGCTGAGCTCTTCCTTGCT |
| 123 | 0 | Healthy Control | GGGCGCGGGTCTGCGGAGCCCCCAGGGCTCGACTCTCAACAGGCACTCAATAAATACTTG |
| 124 | 0 | Healthy Control | ATAATGGGCGATGTTTCTCAGGGCTGCTTCGATAACATAAAAAACTATAAGATTTTTTGG |
| 125 | 0 | Healthy Control | GCATATGCAGGTGTGGGCTTTTTAAAAATCGATGACTGGGAAAAAGCACAGAGCGCTTTC |
| 126 | 0 | Healthy Control | GGCAGGCAGATCACCTGAGGTCCGGGGTTCGAATCATACAATACATGGCCTTTTAAGTCT |
| 127 | 0 | Healthy Control | TTGCTTCTGTGAGAGAAGCAATTTCTTTTCGAGGCCGCAGTGAGTTGTGATCATACCACT |
| 128 | 0 | Healthy Control | GGCGGGCAGATCACTTGAGGCCACGAGTTCGATTTCATCCATGATGTTAATAAAAAACAC |
| 129 | 0 | Healthy Control | ATAAATTAAAAGAGACTCAAGAGACATATCGAATATTAGTAGTCCCGTGGCAAGCCTTTG |
| 130 | 0 | Healthy Control | ACAGAGCCCTCAGAAATAATGCCACATATCGAAGAGTTAACTGTAACTTTGAGAAGTTAA |
| 131 | 0 | Healthy Control | TCCCAGCCCAGTGTACAGAGTAAAAACATCGATTTCTACCAAATGGCTTCTGTGGTGAAA |
| 132 | 0 | Healthy Control | TGACTTGTCCTGTTATGGTTGTTATTCCTCGAGGTTTATGGTAAGGGTTAGGGTTGGAAT |
| 133 | 0 | Healthy Control | ATAATGGGCGATGTTTCTCAGGGCTGCTTCGACATTAACCAAATCTACCCTTCATGCAGA |
| 134 | 0 | Healthy Control | CCTGAGGTGGGGCTTACAAATTAGTATTTCGAACTTGTTTTTCATTTGTAGTAAAAGCTT |
| 135 | 0 | Healthy Control | TGTTGAGGGTGTCCCGGAGCATCAAATCTCGATGTGACATTAACAACTTGTAACAGACAG |
| 136 | 0 | Healthy Control | TGTTGAGGGTGTCCCGGAGCATCAAATCTCGAAATTCATGACTTAAAGTGTAATCCCTAG |
| 137 | 0 | Healthy Control | AGGGTTAGGTGAAAAATCTAAAGGAAGGTCGATGATGAAAATGTTTTGGAGTTGATAGTG |
| 138 | 0 | Healthy Control | GGCAGGCAGATCACTTGAGGTCAGGAATTCGAAGACTAAATTGTACTACTTAAAAAGAAA |
| 139 | 0 | Healthy Control | TGGTGAGGGTGTTTTTCATTCAGCTTTTTCGATTTATTATTTTTAGAGGTTCTCTATCTA |
| 140 | 0 | Healthy Control | TTTGCAGGTGTGTGGTTAACACTATCCTTCGAGGAGAAAAGTCTCTGCCGTCCTGTGCGG |
| 141 | 0 | Healthy Control | TATAATTGAGGGTGTAATTGCAATGGGCTCGAGTGCTGGTTTGGGTCGGAGTGCTGGTTC |
| 142 | 0 | Healthy Control | CCATGCTAATGCTTACCAAAGTGTGACCTCGAGGAGACAGCCGTTGTCCCCAGGGCTCCT |
| 143 | 0 | Healthy Control | TAGATGATCATATATCATACAAACCACTTCGAGGACACCCGGGATTCAGCCAGGGAGGCC |
| 144 | 0 | Healthy Control | GACAGGCAGATCACTTGAGGTCAGGCATTCGAAACATGCTCCTTAAGGACCACCGCTACA |
| 145 | 0 | Healthy Control | CCAAATAGAAACTTACTTTTTTTTTTTTTCGAGGCCGCAGTGAGTTGTGATCATACCACT |
| 146 | 0 | Healthy Control | GGCAGGTGGATCACTTGAGGTCAGCAATTCGAACACCTACCAAATGTTATTTTTTTCACC |
| 147 | 0 | Healthy Control | TGTAGCCCAGAGTGGATGTTATCTAATATCGAATATTAGTAGTCCCGTGGCAAGCCTTTG |
| 148 | 0 | Healthy Control | GGCAGGCAGATCACTTGAGGCCAGGAATTCGACAATAGCAAGACCACCTTTGCTAGCCAG |
| 149 | 0 | Healthy Control | CAGGCAGGCAGAATGCACCATCAATGAATCGATTCTGATGACATGGTTGTTCCTCCTGTT |
| 150 | 0 | Healthy Control | GGCAGGTGGATCATTTGTGGTCAGCAGTTCGATCTCCTGGATTCAATTGATCCTCGCATC |

TABLE 1B (part 3c)

| | Probe Location | | | | |
|---|---|---|---|---|---|
| No. | Chr | Start1 | End1 | Start2 | End2 |
| 101 | 7 | 148853178 | 148853207 | 149053900 | 149053929 |
| 102 | 5 | 150134629 | 150134658 | 150278668 | 150278697 |
| 103 | X | 46398624 | 46398653 | 46508958 | 46508987 |
| 104 | 3 | 52548093 | 52548122 | 52688438 | 52688467 |
| 105 | 13 | 77933720 | 77933749 | 77995327 | 77995356 |
| 106 | X | 77891628 | 77891657 | 78036860 | 78036889 |
| 107 | 5 | 132755647 | 132755676 | 132998902 | 132998931 |
| 108 | 8 | 65438114 | 65438143 | 65630949 | 65630978 |
| 109 | 5 | 94514374 | 94514403 | 94641713 | 94641742 |
| 110 | 2 | 13854395 | 13854424 | 13998016 | 13998045 |
| 111 | 18 | 46962768 | 46962797 | 47121692 | 47121721 |
| 112 | X | 46508958 | 46508987 | 46604757 | 46604786 |
| 113 | 11 | 83253503 | 83253532 | 83442625 | 83442654 |
| 114 | 5 | 34366019 | 34366048 | 34527906 | 34527935 |
| 115 | 18 | 46962768 | 46962797 | 47058017 | 47058046 |
| 116 | 19 | 45212373 | 45212402 | 45414204 | 45414233 |
| 117 | 3 | 126755035 | 126755064 | 126854220 | 126854249 |
| 118 | 12 | 6598575 | 6598604 | 6683579 | 6683608 |
| 119 | 9 | 87096091 | 87096120 | 87143572 | 87143601 |
| 120 | 3 | 52548093 | 52548122 | 52701274 | 52701303 |
| 121 | 17 | 17926105 | 17926134 | 18142964 | 18142993 |
| 122 | 3 | 126604178 | 126604207 | 126854220 | 126854249 |
| 123 | 2 | 27495025 | 27495054 | 27657041 | 27657070 |
| 124 | 20 | 45584742 | 45584771 | 45643174 | 45643203 |
| 125 | 3 | 126627402 | 126627431 | 126854220 | 126854249 |
| 126 | 4 | 112653710 | 112653739 | 112867643 | 112867672 |
| 127 | 17 | 17849812 | 17849841 | 17926105 | 17926134 |
| 128 | 15 | 88883844 | 88883873 | 88906627 | 88906656 |
| 129 | 5 | 132755647 | 132755676 | 132795182 | 132795211 |
| 130 | 8 | 65438114 | 65438143 | 65633324 | 65633353 |
| 131 | 4 | 145339445 | 145339474 | 145450587 | 145450616 |
| 132 | X | 155870710 | 155870739 | 155953720 | 155953749 |
| 133 | 3 | 39518501 | 39518530 | 39578329 | 39578358 |
| 134 | 18 | 34889297 | 34889326 | 35083401 | 35083430 |
| 135 | 3 | 52500909 | 52500938 | 52548093 | 52548122 |
| 136 | 3 | 52548093 | 52548122 | 52574379 | 52574408 |
| 137 | X | 109485288 | 109485317 | 109613114 | 109613143 |
| 138 | 7 | 148853178 | 148853207 | 148909483 | 148909512 |
| 139 | 11 | 7448010 | 7448039 | 7491525 | 7491554 |
| 140 | 21 | 44875225 | 44875254 | 45065011 | 45065040 |
| 141 | 6 | 3041216 | 3041245 | 3146780 | 3146809 |
| 142 | 8 | 142903861 | 142903890 | 143073686 | 143073715 |
| 143 | 17 | 44261861 | 44261890 | 44489969 | 44489998 |
| 144 | 1 | 224692839 | 224692868 | 224812314 | 224812343 |
| 145 | 17 | 17735039 | 17735068 | 17926105 | 17926134 |
| 146 | 4 | 36105086 | 36105115 | 36290918 | 36290947 |
| 147 | 5 | 132681704 | 132681733 | 132755647 | 132755676 |
| 148 | 7 | 143128015 | 143128044 | 143341325 | 143341354 |
| 149 | 22 | 29469297 | 29469326 | 29549135 | 29549164 |
| 150 | 2 | 223382063 | 223382092 | 223446118 | 223446147 |

TABLE 1B (part 3d)

| | 4 kb Sequence Location | | | | |
|---|---|---|---|---|---|
| No. | Chr | Start1 | End1 | Start2 | End2 |
| 101 | 7 | 148849208 | 148853207 | 149049930 | 149053929 |
| 102 | 5 | 150134629 | 150138628 | 150274698 | 150278697 |
| 103 | X | 46398624 | 46402623 | 46504988 | 46508987 |
| 104 | 3 | 52544123 | 52548122 | 52688438 | 52692437 |
| 105 | 13 | 77929750 | 77933749 | 77995327 | 77999326 |
| 106 | X | 77891628 | 77895627 | 78036860 | 78040859 |
| 107 | 5 | 132755647 | 132759646 | 132994932 | 132998931 |
| 108 | 8 | 65434144 | 65438143 | 65626979 | 65630978 |
| 109 | 5 | 94510404 | 94514403 | 94641713 | 94645712 |
| 110 | 2 | 13850425 | 13854424 | 13994046 | 13998045 |
| 111 | 18 | 46958798 | 46962797 | 47121692 | 47125691 |
| 112 | X | 46504988 | 46508987 | 46600787 | 46604786 |
| 113 | 11 | 83253503 | 83257502 | 83438655 | 83442654 |
| 114 | 5 | 34366019 | 34370018 | 34527906 | 34531905 |
| 115 | 18 | 46958798 | 46962797 | 47058017 | 47062016 |
| 116 | 19 | 45208403 | 45212402 | 45410234 | 45414233 |
| 117 | 3 | 126755035 | 126759034 | 126850250 | 126854249 |
| 118 | 12 | 6594605 | 6598604 | 6679609 | 6683608 |
| 119 | 9 | 87096091 | 87100090 | 87143572 | 87147571 |
| 120 | 3 | 52544123 | 52548122 | 52697304 | 52701303 |
| 121 | 17 | 17926105 | 17930104 | 18138994 | 18142993 |
| 122 | 3 | 126604178 | 126608177 | 126850250 | 126854249 |
| 123 | 2 | 27491055 | 27495054 | 27657041 | 27661040 |
| 124 | 20 | 45580772 | 45584771 | 45643174 | 45647173 |
| 125 | 3 | 126627402 | 126631401 | 126850250 | 126854249 |
| 126 | 4 | 112653710 | 112657709 | 112867643 | 112871642 |
| 127 | 17 | 17845842 | 17849841 | 17926105 | 17930104 |
| 128 | 15 | 88879874 | 88883873 | 88902657 | 88906656 |
| 129 | 5 | 132755647 | 132759646 | 132791212 | 132795211 |
| 130 | 8 | 65434144 | 65438143 | 65633324 | 65637323 |
| 131 | 4 | 145339445 | 145343444 | 145446617 | 145450616 |
| 132 | X | 155866740 | 155870739 | 155953720 | 155957719 |
| 133 | 3 | 39518501 | 39522500 | 39574359 | 39578358 |
| 134 | 18 | 34889297 | 34893296 | 35083401 | 35087400 |
| 135 | 3 | 52500909 | 52504908 | 52544123 | 52548122 |
| 136 | 3 | 52544123 | 52548122 | 52570409 | 52574408 |
| 137 | X | 109485288 | 109489287 | 109613114 | 109617113 |
| 138 | 7 | 148849208 | 148853207 | 148905513 | 148909512 |
| 139 | 11 | 7448010 | 7452009 | 7491525 | 7495524 |
| 140 | 21 | 44871255 | 44875254 | 45061041 | 45065040 |
| 141 | 6 | 3041216 | 3045215 | 3146780 | 3150779 |
| 142 | 8 | 142899891 | 142903890 | 143073686 | 143077685 |
| 143 | 17 | 44261861 | 44265860 | 44485999 | 44489998 |
| 144 | 1 | 224692839 | 224696838 | 224808344 | 224812343 |
| 145 | 17 | 17731069 | 17735068 | 17926105 | 17930104 |
| 146 | 4 | 36101116 | 36105115 | 36290918 | 36294917 |
| 147 | 5 | 132677734 | 132681733 | 132755647 | 132759646 |
| 148 | 7 | 143128015 | 143132014 | 143337355 | 143341354 |
| 149 | 22 | 29465327 | 29469326 | 29549135 | 29553134 |
| 150 | 2 | 223382063 | 223386062 | 223446118 | 223450117 |

TABLE 1B (part 3e)

| No. | probe | Primer ID |
|---|---|---|
| 101 | Hg38_7_148843816_148853209_149049131_149053931_FF | OBD160.1201 |
| 102 | Hg38_5_150134627_150137703_150272362_150278699_RF | OBD160.1205 |
| 103 | Hg38_X_46398622_46399662_46498682_46508989_RF | OBD160.1209 |
| 104 | Hg38_3_52542416_52548124_52688436_52689750_FR | OBD160.1213 |
| 105 | Hg38_13_77929805_77933751_77995325_78000878_FR | OBD160.1217 |
| 106 | Hg38_X_77891626_77893835_78036858_78043877_RR | OBD160.1221 |
| 107 | Hg38_5_132755645_132763810_132995308_132998933_RF | OBD160.1225 |
| 108 | Hg38_8_65429758_65438145_65628655_65630980_FF | OBD160.1229 |
| 109 | Hg38_5_94505842_94514405_94641711_94646370_FR | OBD160.1233 |
| 110 | Hg38_2_13845156_13854426_13990532_13998047_FF | OBD160.1237 |
| 111 | Hg38_18_46958975_46962799_47121690_47124504_FR | OBD160.1241 |
| 112 | Hg38_X_46498682_46508989_46603634_46604788_FF | OBD160.1245 |
| 113 | Hg38_11_83253501_83261801_83434843_83442656_RF | OBD160.1249 |
| 114 | Hg38_5_34366017_34372954_34527904_34530421_RR | OBD160.1253 |
| 115 | Hg38_18_46958975_46962799_47058015_47062163_FR | OBD160.1257 |
| 116 | Hg38_19_45210518_45212404_45410404_45414235_FF | OBD160.1261 |
| 117 | Hg38_3_126755033_126764525_126843341_126854251_RF | OBD160.1265 |
| 118 | Hg38_12_6595585_6598606_6681014_6683610_FF | OBD160.1269 |
| 119 | Hg38_9_87096089_87101882_87143570_87148986_RR | OBD160.1273 |
| 120 | Hg38_3_52542416_52548124_52699561_52701305_FF | OBD160.1277 |
| 121 | Hg38_17_17926103_17934669_18141138_18142995_RF | OBD160.1281 |
| 122 | Hg38_3_126604176_126607234_126843341_126854251_RF | OBD160.1285 |
| 123 | Hg38_2_27492451_27495056_27657039_27659544_FR | OBD160.1289 |
| 124 | Hg38_20_45581538_45584773_45643172_45649623_FR | OBD160.1293 |
| 125 | Hg38_3_126627400_126630792_126843341_126854251_RF | OBD160.1297 |
| 126 | Hg38_4_112653708_112659532_112867641_112872043_RR | OBD160.1301 |
| 127 | Hg38_17_17840582_17849843_17926103_17934669_FR | OBD160.1305 |
| 128 | Hg38_15_88873420_88883875_88904252_88906658_FF | OBD160.1309 |
| 129 | Hg38_5_132755645_132763810_132790667_132795213_RF | OBD160.1313 |
| 130 | Hg38_8_65429758_65438145_65633322_65635344_FR | OBD160.1317 |
| 131 | Hg38_4_145339443_145344834_145441661_145450618_RF | OBD160.1321 |
| 132 | Hg38_X_155860883_155870741_155953718_155958970_FR | OBD160.1325 |
| 133 | Hg38_3_39518499_39534447_39571894_39578360_RF | OBD160.1329 |
| 134 | Hg38_18_34889295_34890326_35083399_35092622_RR | OBD160.1333 |
| 135 | Hg38_3_52500907_52509286_52542416_52548124_RF | OBD160.1337 |
| 136 | Hg38_3_52542416_52548124_52570196_52574410_FF | OBD160.1341 |
| 137 | Hg38_X_109485286_109494606_109613112_109617390_RR | OBD160.1345 |
| 138 | Hg38_7_148843816_148853209_148905600_148909514_FF | OBD160.1349 |
| 139 | Hg38_11_7448008_7457037_7491523_7495793_RR | OBD160.1353 |
| 140 | Hg38_21_44873351_44875256_45059036_45065042_FF | OBD160.1357 |
| 141 | Hg38_6_3041214_3044157_3146778_3149848_RR | OBD160.1361 |
| 142 | Hg38_8_142901645_142903892_143073684_143079751_FR | OBD160.1365 |
| 143 | Hg38_17_44261859_44271957_44488590_44490000_RF | OBD160.1369 |
| 144 | Hg38_1_224692837_224704390_224807715_224812345_RF | OBD160.1373 |
| 145 | Hg38_17_17728293_17735070_17926103_17934669_FR | OBD160.1377 |
| 146 | Hg38_4_36078971_36105117_36290916_36293184_FR | OBD160.1381 |
| 147 | Hg38_5_132679860_132681735_132755645_132763810_FR | OBD160.1385 |
| 148 | Hg38_7_143128013_143133284_143335881_143341356_RF | OBD160.1389 |
| 149 | Hg38_22_29467180_29469328_29549133_29556322_FR | OBD160.1393 |
| 150 | Hg38_2_223382061_223395131_223446116_223450606_RR | OBD160.1397 |

TABLE 1B (part 3f)

| No. | Primer Sequence | Primer ID | Primer Sequence |
|---|---|---|---|
| 101 | CATTAGTTAGATTCTCATAAGGATGGC | OBD160.1203 | GGAATCTCAGTCACTCAACTACAAAAT |
| 102 | ACAGGACAGGGCAGGAGAGGCAACGAG | OBD160.1207 | CGTGGGCTTCCTCCCTAATGATGCCGAG |
| 103 | AGATTTTGGGCTGAGACAATGGG | OBD160.1211 | GCTGCTACATTTGTGGTAACTTCTTA |
| 104 | ATTATCCTCCTTTGTTCCTTCCCC | OBD160.1215 | ATGCTTACAGGCAGAACCAGTTTTGTGC |
| 105 | GTGGTAAAGTGTGAGGATGGCA | OBD160.1219 | CTTGGGAGAAAGAGACAGATAGC |
| 106 | CTATTGCTAAATGGAATGCTACCCTGCT | OBD160.1223 | TTTTCCCAATACCGTCCCTCCTGTGG |
| 107 | GAGGTTGTGGAGGAGGCTGAGGATGAGG | OBD160.1227 | GGGATGGAGGGAAGAGTCACAGGAAAGT |
| 108 | CAAGTCTACAGTAACCAAAACAGCAT | OBD160.1231 | GCAGTTGGTGCTCTGTATCCACA |
| 109 | ATCTCAAACCTGGTAATAATCAGC | OBD160.1235 | AATGCTGAGAAAGCCCCAAACCCA |
| 110 | GCCTACCACTCTGCCTCACTTGCTCC | OBD160.1239 | GCACAAGGGAAAATGGAGGGAGAAAGGA |
| 111 | GGCAGTGGCTGGGTGTGGGAGGC | OBD160.1243 | AAAGGTAACTTGAGAGTGGAGATGCGGG |
| 112 | AGATTTTGGGCTGAGACAATGGG | OBD160.1247 | GGGATTACAGGCATAAGCCACTG |
| 113 | CCAGGATGAGCCAGAAGGAGTTTCACAA | OBD160.1251 | TCACTGAAGGTTCCAAATCTCCCAAT |
| 114 | GCACCTTACTTACTGTATCTCTTCAG | OBD160.1255 | CCTGCCTCAGCCTCCAAAGTAGC |
| 115 | CCGTCGCAAGTTTCGTTCCCCAACCCGC | OBD160.1259 | GTCCCCTGTCTTCCCCGCTGTGG |
| 116 | CTCCCTCTACCTCCACTACCCTGCCCAG | OBD160.1263 | AGGAGGGCAGGGCTGGGTCACAG |
| 117 | GGCAGAAAGGAAAGAGCCTCGCAGCATT | OBD160.1267 | CCAGCCAGGGATGTTACCCAGAGCCCC |
| 118 | TGCCTGAAATCCCAGCACCTTGG | OBD160.1271 | TGGCACACACCTGTAGTCCCAGC |
| 119 | GAGCCCTGAGCCCCTCCTGACCA | OBD160.1275 | GAAGCCAGGAGAAATGAAATGATGGGTG |
| 120 | ATTATCCTCCTTTGTTCCTTCCCC | OBD160.1279 | CACTTGACAAACCCACAGCACAGAAA |
| 121 | CTCCCCACTACCCCAACCCAGGC | OBD160.1283 | CCGCATCTCCCCTTGTGCCAGTCCAGAC |
| 122 | GGCAGAAAGGAAAGAGCCTCGCAGCATT | OBD160.1287 | GCAAACAAGGAAACCCCAAGCAGAGAAG |
| 123 | CCTATTTTCTCTACGCCCTCCCGCCCC | OBD160.1291 | GGTGAGGCGACCCTGTTGAAAAGTGGGC |
| 124 | GGTAAAGTGTTGGGAAGGCGAAA | OBD160.1295 | CAGAGATGAGGTTTTCTATGTTGGC |
| 125 | GTCATTCTCCCCTCTCATCTCTACCCTA | OBD160.1299 | CACACTCCTTTAGGTAACAGAACATTTC |
| 126 | TGTCTGTAATCCCAGCACTTTGG | OBD160.1303 | CCAAGAGAAATGAAAATGTTATGTCCAC |
| 127 | TCCCCTCCCTGTCTGGACCCTGG | OBD160.1307 | CCGCATCTCCCCTTGTGCCAGTCCAGAC |
| 128 | AGTGGCTCACGCCTGTAATCCTA | OBD160.1311 | TGGACCACAGCCCTTCTTTCGGG |
| 129 | CAAGGACAATCAATGGCTGCTCACATCA | OBD160.1315 | GGGATGGAGGGAAGAGTCACAGGAAAGT |
| 130 | CAAGTCTACAGTAACCAAAACAGCAT | OBD160.1319 | TGAGATAACTGAGGCTCTAAGAACTT |
| 131 | CTGCCCTCAGAGAGTAAAGAGACAGGAC | OBD160.1323 | CTGTTAGGCTCAAAGGTTGGGTATGGGC |
| 132 | TCTCCTACCTCTCTACACACTTTATTGA | OBD160.1327 | CAAGAGTCTAATCCCAAACCCCAACCAG |
| 133 | TGGTAAAGTGTTAGGACGGTGAAAAT | OBD160.1331 | GGAAGTATTTTCAGTGTATTTCAGAGAC |
| 134 | GTCAGGACTTGGCTGGGCACCTACC | OBD160.1335 | GCACTGGGAGCACAGGGCAGGAATGGTG |
| 135 | ATTATCCTCCTTTGTTCCTTCCCC | OBD160.1339 | TGCCACTTCCCATCTGCTCAGGG |
| 136 | ATTATCCTCCTTTGTTCCTTCCCC | OBD160.1343 | CCCATTCAGACAGAGCAGACCCATTT |
| 137 | GCTAACAGTGCCAGTTGAAAGGAGATGC | OBD160.1347 | CCCAGACAGGAGTGCCTTTGGAGTGGC |
| 138 | CATTAGTTAGATTCTCATAAGGATGGC | OBD160.1351 | GAATAGGACTGCCTGATAACTTGTAT |
| 139 | GCCTCCCCATTTCTCCTCAGTTTCACAA | OBD160.1355 | CTATGGCAAGGAGAGGCTGTAGGAGC |
| 140 | CTCTGTTCCGCCTACCCAGGTCCACACC | OBD160.1359 | GGCGGGAGACCCTTGCCCCTGAA |
| 141 | ACTTGTCTCCCATTCCTGGTGTGTTTGG | OBD160.1363 | CCGACCCAAACCAGCACTCCAGCCG |
| 142 | CAGAGAACAGAGAGGACTGAAGC | OBD160.1367 | GACGACGGCTGTCTCCTCAAACA |
| 143 | GCCTCCTAACCCACCTCCCCACC | OBD160.1371 | CACTCAGGGCTCTGCCGTCACCTTGGAG |
| 144 | CGTCTTCCACCCTGGGCTGTTCGTCCC | OBD160.1375 | CGGACCCATCAACAGTGAGCCAAGCATC |
| 145 | CCTGCCTGTGTGTCTGCGTGTGAGC | OBD160.1379 | CCGCATCTCCCCTTGTGCCAGTCCAGAC |
| 146 | GGCTCCCACCCTGTTAGAGATGTGACG | OBD160.1383 | CCTAACCAGTCTTCTTCCACAGCCCACA |
| 147 | GCTGGAGTGGGCTCACCGAGGATAGGG | OBD160.1387 | GGGATGGAGGGAAGAGTCACAGGAAAGT |
| 148 | TGTCCACAGTATGATTCTCGGGTTTTCA | OBD160.1391 | TACAGTGGTGCTACAGGTTATGGGAGGG |
| 149 | CCTCTTTCCCAAATGTCATCTTTTAGC | OBD160.1395 | GGAGGAAAATAGTTGAGCAGGAACAAT |
| 150 | CGATGGCTCATAGTGCTGTAATCCAAGC | OBD160.1399 | CACTTTGAGAGCCTGAAGCAGCAGCATC |

TABLE 1B (part 3g)

| No. | probe | Markers |
|---|---|---|
| 101 | Hg38_7_148843816_148853209_149049131_149053931_FF | OBD160.1201.1203 |
| 102 | Hg38_5_150134627_150137703_150272362_150278699_RF | OBD160.1205.1207 |
| 103 | Hg38_X_46398622_46399662_46498682_46508989_RF | OBD160.1209.1211 |
| 104 | Hg38_3_52542416_52548124_52688436_52689750_FR | OBD160.1213.1215 |
| 105 | Hg38_13_77929805_77933751_77995325_78000878_FR | OBD160.1217.1219 |
| 106 | Hg38_X_77891626_77893835_78036858_78043877_RR | OBD160.1221.1223 |
| 107 | Hg38_5_132755645_132763810_132995308_132998933_RF | OBD160.1225.1227 |
| 108 | Hg38_8_65429758_65438145_65628655_65630980_FF | OBD160.1229.1231 |
| 109 | Hg38_5_94505842_94514405_94641711_94646370_FR | OBD160.1233.1235 |
| 110 | Hg38_2_13845156_13854426_13990532_13998047_FF | OBD160.1237.1239 |
| 111 | Hg38_18_46958975_46962799_47121690_47124504_FR | OBD160.1241.1243 |
| 112 | Hg38_X_46498682_46508989_46603634_46604788_FF | OBD160.1245.1247 |
| 113 | Hg38_11_83253501_83261801_83434843_83442656_RF | OBD160.1249.1251 |
| 114 | Hg38_5_34366017_34372954_34527904_34530421_RR | OBD160.1253.1255 |
| 115 | Hg38_18_46958975_46962799_47058015_47062163_FR | OBD160.1257.1259 |
| 116 | Hg38_19_45210518_45212404_45410404_45414235_FF | OBD160.1261.1263 |
| 117 | Hg38_3_126755033_126764525_126843341_126854251_RF | OBD160.1265.1267 |
| 118 | Hg38_12_6595585_6598606_6681014_6683610_FF | OBD160.1269.1271 |
| 119 | Hg38_9_87096089_87101882_87143570_87148986_RR | OBD160.1273.1275 |
| 120 | Hg38_3_52542416_52548124_52699561_52701305_FF | OBD160.1277.1279 |
| 121 | Hg38_17_17926103_17934669_18141138_18142995_RF | OBD160.1281.1283 |
| 122 | Hg38_3_126604176_126607234_126843341_126854251_RF | OBD160.1285.1287 |
| 123 | Hg38_2_27492451_27495056_27657039_27659544_FR | OBD160.1289.1291 |
| 124 | Hg38_20_45581538_45584773_45643172_45649623_FR | OBD160.1293.1295 |
| 125 | Hg38_3_126627400_126630792_126843341_126854251_RF | OBD160.1297.1299 |
| 126 | Hg38_4_112653708_112659532_112867641_112872043_RR | OBD160.1301.1303 |
| 127 | Hg38_17_17840582_17849843_17926103_17934669_FR | OBD160.1305.1307 |
| 128 | Hg38_15_88873420_88883875_88904252_88906658_FF | OBD160.1309.1311 |
| 129 | Hg38_5_132755645_132763810_132790667_132795213_RF | OBD160.1313.1315 |
| 130 | Hg38_8_65429758_65438145_65633322_65635344_FR | OBD160.1317.1319 |
| 131 | Hg38_4_145339443_145344834_145441661_145450618_RF | OBD160.1321.1323 |
| 132 | Hg38_X_155860883_155870741_155953718_155958970_FR | OBD160.1325.1327 |
| 133 | Hg38_3_39518499_39534447_39571894_39578360_RF | OBD160.1329.1331 |
| 134 | Hg38_18_34889295_34890326_35083399_35092622_RR | OBD160.1333.1335 |
| 135 | Hg38_3_52500907_52509286_52542416_52548124_RF | OBD160.1337.1339 |
| 136 | Hg38_3_52542416_52548124_52570196_52574410_FF | OBD160.1341.1343 |
| 137 | Hg38_X_109485286_109494606_109613112_109617390_RR | OBD160.1345.1347 |
| 138 | Hg38_7_148843816_148853209_148905600_148909514_FF | OBD160.1349.1351 |
| 139 | Hg38_11_7448008_7457037_7491523_7495793_RR | OBD160.1353.1355 |
| 140 | Hg38_21_44873351_44875256_45059036_45065042_FF | OBD160.1357.1359 |
| 141 | Hg38_6_3041214_3044157_3146778_3149848_RR | OBD160.1361.1363 |
| 142 | Hg38_8_142901645_142903892_143073684_143079751_FR | OBD160.1365.1367 |
| 143 | Hg38_17_44261859_44271957_44488590_44490000_RF | OBD160.1369.1371 |
| 144 | Hg38_1_224692837_224704390_224807715_224812345_RF | OBD160.1373.1375 |
| 145 | Hg38_17_17728293_17735070_17926103_17934669_FR | OBD160.1377.1379 |
| 146 | Hg38_4_36078971_36105117_36290916_36293184_FR | OBD160.1381.1383 |
| 147 | Hg38_5_132679860_132681735_132755645_132763810_FR | OBD160.1385.1387 |
| 148 | Hg38_7_143128013_143133284_143335881_143341356_RF | OBD160.1389.1391 |
| 149 | Hg38_22_29467180_29469328_29549133_29556322_FR | OBD160.1393.1395 |
| 150 | Hg38_2_223382061_223395131_223446116_223450606_RR | OBD160.1397.1399 |

TABLE 1B (part 4a)

| | | | | FC: (class1/ | |
|---|---|---|---|---|---|
| No. | probe | RP/Rsum | Rprank | class2) | pfp |
| 151 | Hg38_3_52542416_52548124_52606123_52609751_FF | 5189.071535 | 145 | −1.414 | 0 |
| 152 | Hg38_8_143012831_143021167_143073684_143079751_FR | 3503.491132 | 63 | −1.414 | 0 |
| 153 | Hg38_1_212366521_212376213_212547138_212548374_RR | 4620.284986 | 118 | −1.413 | 0 |
| 154 | Hg38_X_46498682_46508989_46602210_46603634_FR | 6204.522114 | 202 | −1.412 | 0 |
| 155 | Hg38_21_44873351_44875256_44906478_44910319_FR | 6433.604863 | 223 | −1.412 | 0 |
| 156 | Hg38_9_78097129_78101713_78276332_78285196_FF | 6925.240863 | 254 | −1.412 | 0 |
| 157 | Hg38_3_126673758_126686020_126843341_126854251_RF | 6344.512786 | 216 | −1.411 | 0 |
| 158 | Hg38_17_68575027_68578789_68785810_68796098_RF | 5846.061342 | 177 | −1.411 | 0 |
| 159 | Hg38_5_24349034_24361029_24564285_24566267_FR | 5934.820934 | 184 | −1.411 | 0 |
| 160 | Hg38_11_7448008_7457037_7633311_7640164_RR | 8160.203972 | 325 | −1.411 | 0 |
| 161 | Hg38_7_27161846_27163700_27302548_27312509_FF | 4965.220225 | 134 | −1.411 | 0 |
| 162 | Hg38_13_80339373_80340696_80530340_80535867_RF | 6070.077564 | 196 | −1.409 | 0 |
| 163 | Hg38_9_4966910_4970390_5177459_5185304_FR | 5825.482506 | 175 | −1.409 | 0 |
| 164 | Hg38_3_126843341_126854251_127025761_127027674_FR | 6302.840977 | 212 | −1.409 | 0 |
| 165 | Hg38_2_13845156_13854426_13894360_13900303_FR | 4127.259421 | 90 | −1.409 | 0 |
| 166 | Hg38_3_126619219_126620249_126843341_126854251_RF | 6736.278815 | 243 | −1.409 | 0 |
| 167 | Hg38_11_19587032_19595878_19772073_19777708_RF | 5137.562086 | 143 | −1.408 | 0 |
| 168 | Hg38_3_107106310_107120834_107363175_107364266_RF | 4138.382317 | 91 | −1.408 | 0 |
| 169 | Hg38_2_172327203_172330194_172401667_172407636_FF | 7018.881201 | 259 | −1.408 | 0 |
| 170 | Hg38_2_27492451_27495056_27626184_27635183_FF | 7280.572764 | 284 | −1.407 | 0 |
| 171 | Hg38_6_108138075_108144989_108280425_108286846_RR | 5889.114204 | 182 | −1.407 | 0 |
| 172 | Hg38_12_95793441_95797455_95929302_95940423_FR | 4522.319776 | 109 | −1.407 | 0 |
| 173 | Hg38_17_34470224_34473967_34596926_34605729_RF | 181704.137 | 102782 | −1.406 | 0.203524936 |
| 174 | Hg38_3_52542416_52548124_52770196_52773754_FR | 5666.285584 | 164 | −1.405 | 0 |
| 175 | Hg38_1_186902459_186905074_187113187_187139713_RR | 6803.129009 | 247 | −1.405 | 0 |
| 176 | Hg38_X_46498682_46508989_46576307_46579924_FR | 5869.244424 | 180 | −1.405 | 0 |
| 177 | Hg38_6_3017794_3021910_3146778_3149848_RR | 6256.127058 | 207 | −1.403 | 0 |
| 178 | Hg38_1_32149028_32153363_32237144_32241139_RF | 6853.104399 | 250 | −1.402 | 0 |
| 179 | Hg38_11_316800_318015_481419_487611_FR | 7179.47109 | 277 | −1.402 | 0 |
| 180 | Hg38_15_53424923_53427397_53442412_53454683_FF | 7090.153633 | 268 | −1.401 | 0 |
| 181 | Hg38_17_68666853_68670189_68785810_68796098_RF | 6909.88586 | 253 | −1.401 | 0 |
| 182 | Hg38_11_7448008_7457037_7610329_7615891_RR | 9210.499261 | 399 | −1.401 | 0 |
| 183 | Hg38_17_77934896_77938750_78117632_78120001_RF | 6333.571605 | 214 | −1.4 | 0 |
| 184 | Hg38_5_43590188_43596508_43695191_43701837_RR | 5995.126969 | 190 | −1.4 | 0 |
| 185 | Hg38_1_60428956_60435934_60549270_60560051_FR | 6237.112636 | 206 | −1.399 | 0 |
| 186 | Hg38_5_150272362_150278699_150370575_150373999_FF | 6930.555298 | 255 | −1.398 | 0 |
| 187 | Hg38_9_130859900_130867193_130899377_130901466_RR | 6344.951249 | 217 | −1.398 | 0 |
| 188 | Hg38_1_91404825_91416231_91606200_91614477_FR | 3987.770204 | 84 | −1.397 | 0 |
| 189 | Hg38_14_91576230_91580637_91629033_91639298_RR | 5505.97469 | 156 | −1.397 | 0 |
| 190 | Hg38_2_27349601_27353190_27492451_27495056_RF | 7684.120774 | 302 | −1.396 | 0 |
| 191 | Hg38_5_150051979_150055894_150272362_150278699_RF | 6705.504005 | 240 | −1.396 | 0 |
| 192 | Hg38_1_185167238_185172841_185287397_185290374_FR | 6618.356808 | 234 | −1.395 | 0 |
| 193 | Hg38_3_112416500_112418982_112549606_112554897_FF | 6393.210055 | 220 | −1.395 | 0 |
| 194 | Hg38_6_132376734_132385044_132542354_132547678_FR | 6379.766204 | 219 | −1.395 | 0 |
| 195 | Hg38_1_189027657_189033909_189210801_189218947_FR | 5745.204415 | 170 | −1.395 | 0 |
| 196 | Hg38_2_233409972_233419808_233475741_233480083_FR | 8183.893064 | 330 | −1.395 | 0 |
| 197 | Hg38_17_35250006_35254814_35287155_35289074_RF | 7536.563459 | 291 | −1.395 | 0 |
| 198 | Hg38_5_140984243_140987011_141123576_141128793_FF | 5530.550379 | 157 | −1.394 | 0 |
| 199 | Hg38_13_40670004_40674221_40797038_40804771_RR | 6292.929057 | 210 | −1.394 | 0 |
| 200 | Hg38_15_34800440_34807454_35007401_35010342_RR | 5850.979126 | 179 | −1.393 | 0 |

TABLE 1B (part 4b)

| | P. | | Probe sequence |
|---|---|---|---|
| No. | value | Type | 60 mer |
| 151 | 0 | Healthy Control | TGTTGAGGGTGTCCCGGAGCATCAAATCTCGAAGTCACATGATGGCCAACAAGTACCAAG |
| 152 | 0 | Healthy Control | CTGTGTGTTTGAGTGTCGCTTGACCTGCTCGAGGAGACAGCCGTTGTCCCCAGGGCTCCT |
| 153 | 0 | Healthy Control | GAGGCAGGCGGATCACGAGGTCATAAGATCGAATTTCCAGTTTCTAAAAAGTTTAAGAAT |
| 154 | 0 | Healthy Control | ACAGAGCCCTCAGAAATAATGCCGCATATCGAAGGCTCCAAGGTTTTCGTGATCTTTTCA |
| 155 | 0 | Healthy Control | TTTGCAGGTGTGTGGTTAACACTATCCTTCGAAACAGGTGCTCTGGTCAAGTCAGTTTGG |
| 156 | 0 | Healthy Control | AGTGTGTGTGAGGAAATGCAGGGGTGAATCGAGCCCCTGCTTTCAATTCTTTTGGATATA |
| 157 | 0 | Healthy Control | GCATATGCAGGTGTGGGCTTTTTAAAAATCGAAATCCAGCTCCACTACTCACTAGTTGGT |
| 158 | 0 | Healthy Control | ATAATGGGCGATGTTTCTCAGGGCTGCTTCGATAGGTACAACTATTATGTATCAATATTT |
| 159 | 0 | Healthy Control | GGAGGGGAACATTACACACTGGGGCCTGTCGAATGCTTTATCTGGACCTACTTTTGTGAA |
| 160 | 0 | Healthy Control | TGGTGAGGGTGTTTTTCATTCAGCTTTTTCGAAGCTTTCTAGGGAAGGCCATGTCTTCTC |
| 161 | 0 | Healthy Control | TGACCTGCCTCTCGGTGAGGTTGAGCAGTCGAAGTAATGAAGAGGGAGGTGGTTTTCTTT |
| 162 | 0 | Healthy Control | CAGGTGTGTCCATGATTAAACTTTTAATTCGAGAGAAAAAAAAAAGATATCATATTTCTT |
| 163 | 0 | Healthy Control | GGCAGGCGGATCACTTGAGGCCAGGGATTCGACACACAGAGCTGTGTGGAGTCTCAGCAG |
| 164 | 0 | Healthy Control | GCATATGCAGGTGTGGGCTTTTTAAAAATCGACAGATGAATGAATAAGCAAAATGTGGTC |
| 165 | 0 | Healthy Control | GGCAGGCAGATCACTTGAGGTCACGAGTTCGATCATTTCAAGAACCTACCTTCAAGTTCA |
| 166 | 0 | Healthy Control | GCATATGCAGGTGTGGGCTTTTTAAAAATCGAAAGGAAAGAATCAGTGTATCAAAAAGAT |
| 167 | 0 | Healthy Control | GGAGGTGTGTGTGTTTAAACCAGGAAAGTCGAGCTTCACGGCCGCTTTGTTTACCTACTC |
| 168 | 0 | Healthy Control | GGCGGGCAGATCACTTGAGGTCAGAACTTCGAACTCTGCTTTCTATTTCAGCAGCATTTG |
| 169 | 0 | Healthy Control | AGGGCAGGGGCACTAGGATGAAAGGCCGTCGATCAATGGATAAAAACAAATATAAGAGCT |
| 170 | 0 | Healthy Control | GGGCGCGGGTCTGCGGAGCCCCCAGGGCTCGATGTGTCCATTACATAGATGACAACTGTT |
| 171 | 0 | Healthy Control | AAGGCAGGCGGATCACGAGGTCAGTAGATCGAAGATTACTCTGTGATTTTCTCCAATTAT |
| 172 | 0 | Healthy Control | GGCAGGCAGATCACTTGAGGTCAGGAATTCGAGGTGCCATCTGTCACCCCTTTCTTTGAC |
| 173 | 0. | Healthy Control | GAAACAGTGAGTGTTTATTTCTCGTGAGTCGAAGCCTTTATCAAAGTCTCAAAGGGGTCT |
| | 022523305 | | |
| 174 | 0 | Healthy Control | TGTTGAGGGTGTCCCGGAGCATCAAATCTCGAGTTTTAAGTGAATCCAGCAGTCTACAGT |
| 175 | 0 | Healthy Control | ATAATGGGCGATGTTTCTCAGGGCTGCTTCGATACAAAGAGATGGCCAGATTTCATCTGA |
| 176 | 0 | Healthy Control | ACAGAGCCCTCAGAAATAATGCCGCATATCGACAAGACCTCAGGGTTGGGTGTCCCCCAC |
| 177 | 0 | Healthy Control | CCCCTCAAGAACGAGCTCTCCCCCTGTCTCGAGTGCTGGTTTGGGTCGGAGTGCTGGTTC |
| 178 | 0 | Healthy Control | AAGGGCTCGGGAGCTCCCTCGGCACACCTCGATTCTGTTCATTTTTCTTCATTCTTTATT |
| 179 | 0 | Healthy Control | GAGCTGGGTTAGGGTTGGGAAGACAGCTTCGAATCTGCAAGTCTGTTTTGGGAGTAGTGT |
| 180 | 0 | Healthy Control | AGAATGGGCGATGTTTCTCAGGGCTGCTTCGAGCATATGCTACACAGTGGAGCAACCTTC |
| 181 | 0 | Healthy Control | ATAATGGGCGATGTTTCTCAGGGCTGCTTCGAATGGTAGTTCTGCGTTTAGCTCTTTGAG |
| 182 | 0 | Healthy Control | TGGTGAGGGTGTTTTTCATTCAGCTTTTTCGATCTGATGACTGAAGTGTCTGAGCTGAAG |
| 183 | 0 | Healthy Control | TTTTTATTATTATTAAATTCATATATTCTCGAAGGTGTGTGTGGATTTTTCTTGTTTGCT |
| 184 | 0 | Healthy Control | AGCAGGCAGATCACTTGAGGTCAGGAGTTCGAAAACAATTTCTCACTTGTCTGGGCAAAA |
| 185 | 0 | Healthy Control | AGGGACAGCCAGCCAGCCACAGATGATGTCGATCAGACTAGATATCTGTTTTGTGAGCTA |
| 186 | 0 | Healthy Control | AGTGCCTGTGAAAGTGTTTCTGAATTCCTCGATCAGCTTCACACACTCTCACTCCTGGAG |
| 187 | 0 | Healthy Control | AAGGAGGGCAGATCACGAGGTCAGGCGTTCGATAAATACTGGATTTTATTAAATAAACTC |
| 188 | 0 | Healthy Control | ATGTGTTTGCTTATTTATCATTAATAATTCGAACATCTTAGAGACCAAGACCAAAAGGGG |
| 189 | 0 | Healthy Control | GGCAGGAGGATCACTTGAGGCCAGGCATTCGAAAACTTATATCTATACAAAACCTGCAAA |
| 190 | 0 | Healthy Control | GGGCGCGGGTCTGCGGAGCCCCCAGGGCTCGAATTTAGGGGGAAAACATTAAATAGGAAT |
| 191 | 0 | Healthy Control | AGTGCCTGTGAAAGTGTTTCTGAATTCCTCGACAATGCCACTCACTATTACCTCTACAAC |
| 192 | 0 | Healthy Control | GCCGAGGTGGGCAGATCACGAGGTCAAATCGAAAGGATCAGGAATTGGAATAATTCCTCT |
| 193 | 0 | Healthy Control | AGAATGGGCGATGTTTCTCAGGGCTGCTTCGAAGTAAAAGAACAGAAACATAAAGTTTCT |
| 194 | 0 | Healthy Control | GAGAGGTGGGCAGGTGTTAGATTTAGTTTCGATTAATCTTTTATATTTAAAAACTTGGCC |
| 195 | 0 | Healthy Control | GGCAGGCAGATCACTTGAGGTCAGCAGTTCGACACAGACACCCTCCCAAGACTAAACCAG |
| 196 | 0 | Healthy Control | GGGTGTGGGTGGATTTTCACCTTGTCTGTCGAAGGCAAAGCTTTTCAGCGATTTCATTAA |
| 197 | 0 | Healthy Control | ATTGTTTAAGCCAGCCAGCCTATGGTATTCGAACCTACAGTTGAAGGCCTTATATGCAAA |
| 198 | 0 | Healthy Control | GGCAGGCAGATCACTTGAGGTCATGAGTTCGAAAAAAAAAAAAAAAGTATTTCTTTTAGT |
| 199 | 0 | Healthy Control | TCGCCTAGGCTGGAGCAGTGGTGTGATCTCGATCTTTGAGAACCTACTAGGTGTCAGTCT |
| 200 | 0 | Healthy Control | GGCGGGCAGATCACTTGAGGCCAGGAGCTCGATCAGTGATGTTAAATTAAATGAGCAGGA |

TABLE 1B (part 4c)

| | Probe Location | | | | |
|---|---|---|---|---|---|
| No. | Chr | Start1 | End1 | Start2 | End2 |
| 151 | 3 | 52548093 | 52548122 | 52609720 | 52609749 |
| 152 | 8 | 143021136 | 143021165 | 143073686 | 143073715 |
| 153 | 1 | 212366523 | 212366552 | 212547140 | 212547169 |
| 154 | X | 46508958 | 46508987 | 46602212 | 46602241 |
| 155 | 21 | 44875225 | 44875254 | 44906480 | 44906509 |
| 156 | 9 | 78101682 | 78101711 | 78285165 | 78285194 |
| 157 | 3 | 126673760 | 126673789 | 126854220 | 126854249 |
| 158 | 17 | 68575029 | 68575058 | 68796067 | 68796096 |
| 159 | 5 | 24360998 | 24361027 | 24564287 | 24564316 |
| 160 | 11 | 7448010 | 7448039 | 7633313 | 7633342 |
| 161 | 7 | 27163669 | 27163698 | 27312478 | 27312507 |
| 162 | 13 | 80339375 | 80339404 | 80535836 | 80535865 |
| 163 | 9 | 4970359 | 4970388 | 5177461 | 5177490 |
| 164 | 3 | 126854220 | 126854249 | 127025763 | 127025792 |
| 165 | 2 | 13854395 | 13854424 | 13894362 | 13894391 |
| 166 | 3 | 126619221 | 126619250 | 126854220 | 126854249 |
| 167 | 11 | 19587034 | 19587063 | 19777677 | 19777706 |
| 168 | 3 | 107106312 | 107106341 | 107364235 | 107364264 |
| 169 | 2 | 172330163 | 172330192 | 172407605 | 172407634 |
| 170 | 2 | 27495025 | 27495054 | 27635152 | 27635181 |
| 171 | 6 | 108138077 | 108138106 | 108280427 | 108280456 |
| 172 | 12 | 95797424 | 95797453 | 95929304 | 95929333 |
| 173 | 17 | 34470226 | 34470255 | 34605698 | 34605727 |
| 174 | 3 | 52548093 | 52548122 | 52770198 | 52770227 |
| 175 | 1 | 186902461 | 186902490 | 187113189 | 187113218 |
| 176 | X | 46508958 | 46508987 | 46576309 | 46576338 |
| 177 | 6 | 3017796 | 3017825 | 3146780 | 3146809 |
| 178 | 1 | 32149030 | 32149059 | 32241108 | 32241137 |
| 179 | 11 | 317984 | 318013 | 481421 | 481450 |
| 180 | 15 | 53427366 | 53427395 | 53454652 | 53454681 |
| 181 | 17 | 68666855 | 68666884 | 68796067 | 68796096 |
| 182 | 11 | 7448010 | 7448039 | 7610331 | 7610360 |
| 183 | 17 | 77934898 | 77934927 | 78119970 | 78119999 |
| 184 | 5 | 43590190 | 43590219 | 43695193 | 43695222 |
| 185 | 1 | 60435903 | 60435932 | 60549272 | 60549301 |
| 186 | 5 | 150278668 | 150278697 | 150373968 | 150373997 |
| 187 | 9 | 130859902 | 130859931 | 130899379 | 130899408 |
| 188 | 1 | 91416200 | 91416229 | 91606202 | 91606231 |
| 189 | 14 | 91576232 | 91576261 | 91629035 | 91629064 |
| 190 | 2 | 27349603 | 27349632 | 27495025 | 27495054 |
| 191 | 5 | 150051981 | 150052010 | 150278668 | 150278697 |
| 192 | 1 | 185172810 | 185172839 | 185287399 | 185287428 |
| 193 | 3 | 112418951 | 112418980 | 112554866 | 112554895 |
| 194 | 6 | 132385013 | 132385042 | 132542356 | 132542385 |
| 195 | 1 | 189033878 | 189033907 | 189210803 | 189210832 |
| 196 | 2 | 233419777 | 233419806 | 233475743 | 233475772 |
| 197 | 17 | 35250008 | 35250037 | 35289043 | 35289072 |
| 198 | 5 | 140986980 | 140987009 | 141128762 | 141128791 |
| 199 | 13 | 40670006 | 40670035 | 40797040 | 40797069 |
| 200 | 15 | 34800442 | 34800471 | 35007403 | 35007432 |

TABLE 1B (part 4d)

| | 4 kb Sequence Location | | | | |
|---|---|---|---|---|---|
| No. | Chr | Start1 | End1 | Start2 | End2 |
| 151 | 3 | 52544123 | 52548122 | 52605750 | 52609749 |
| 152 | 8 | 143017166 | 143021165 | 143073686 | 143077685 |
| 153 | 1 | 212366523 | 212370522 | 212547140 | 212551139 |
| 154 | X | 46504988 | 46508987 | 46602212 | 46606211 |
| 155 | 21 | 44871255 | 44875254 | 44906480 | 44910479 |
| 156 | 9 | 78097712 | 78101711 | 78281195 | 78285194 |
| 157 | 3 | 126673760 | 126677759 | 126850250 | 126854249 |
| 158 | 17 | 68575029 | 68579028 | 68792097 | 68796096 |
| 159 | 5 | 24357028 | 24361027 | 24564287 | 24568286 |
| 160 | 11 | 7448010 | 7452009 | 7633313 | 7637312 |
| 161 | 7 | 27159699 | 27163698 | 27308508 | 27312507 |
| 162 | 13 | 80339375 | 80343374 | 80531866 | 80535865 |
| 163 | 9 | 4966389 | 4970388 | 5177461 | 5181460 |
| 164 | 3 | 126850250 | 126854249 | 127025763 | 127029762 |
| 165 | 2 | 13850425 | 13854424 | 13894362 | 13898361 |
| 166 | 3 | 126619221 | 126623220 | 126850250 | 126854249 |
| 167 | 11 | 19587034 | 19591033 | 19773707 | 19777706 |
| 168 | 3 | 107106312 | 107110311 | 107360265 | 107364264 |
| 169 | 2 | 172326193 | 172330192 | 172403635 | 172407634 |
| 170 | 2 | 27491055 | 27495054 | 27631182 | 27635181 |
| 171 | 6 | 108138077 | 108142076 | 108280427 | 108284426 |
| 172 | 12 | 95793454 | 95797453 | 95929304 | 95933303 |
| 173 | 17 | 34470226 | 34474225 | 34601728 | 34605727 |
| 174 | 3 | 52544123 | 52548122 | 52770198 | 52774197 |
| 175 | 1 | 186902461 | 186906460 | 187113189 | 187117188 |
| 176 | X | 46504988 | 46508987 | 46576309 | 46580308 |
| 177 | 6 | 3017796 | 3021795 | 3146780 | 3150779 |
| 178 | 1 | 32149030 | 32153029 | 32237138 | 32241137 |
| 179 | 11 | 314014 | 318013 | 481421 | 485420 |
| 180 | 15 | 53423396 | 53427395 | 53450682 | 53454681 |
| 181 | 17 | 68666855 | 68670854 | 68792097 | 68796096 |
| 182 | 11 | 7448010 | 7452009 | 7610331 | 7614330 |
| 183 | 17 | 77934898 | 77938897 | 78116000 | 78119999 |
| 184 | 5 | 43590190 | 43594189 | 43695193 | 43699192 |
| 185 | 1 | 60431933 | 60435932 | 60549272 | 60553271 |
| 186 | 5 | 150274698 | 150278697 | 150369998 | 150373997 |
| 187 | 9 | 130859902 | 130863901 | 130899379 | 130903378 |
| 188 | 1 | 91412230 | 91416229 | 91606202 | 91610201 |
| 189 | 14 | 91576232 | 91580231 | 91629035 | 91633034 |
| 190 | 2 | 27349603 | 27353602 | 27491055 | 27495054 |
| 191 | 5 | 150051981 | 150055980 | 150274698 | 150278697 |
| 192 | 1 | 185168840 | 185172839 | 185287399 | 185291398 |
| 193 | 3 | 112414981 | 112418980 | 112550896 | 112554895 |
| 194 | 6 | 132381043 | 132385042 | 132542356 | 132546355 |
| 195 | 1 | 189029908 | 189033907 | 189210803 | 189214802 |
| 196 | 2 | 233415807 | 233419806 | 233475743 | 233479742 |
| 197 | 17 | 35250008 | 35254007 | 35285073 | 35289072 |
| 198 | 5 | 140983010 | 140987009 | 141124792 | 141128791 |
| 199 | 13 | 40670006 | 40674005 | 40797040 | 40801039 |
| 200 | 15 | 34800442 | 34804441 | 35007403 | 35011402 |

TABLE 1B (part 4e)

| No. | probe | Primer ID |
|---|---|---|
| 151 | Hg38_3_52542416_52548124_52606123_52609751_FF | OBD160.1401 |
| 152 | Hg38_8_143012831_143021167_143073684_143079751_FR | OBD160.1405 |
| 153 | Hg38_1_212366521_212376213_212547138_212548374_RR | OBD160.1409 |
| 154 | Hg38_X_46498682_46508989_46602210_46603634_FR | OBD160.1413 |
| 155 | Hg38_21_44873351_44875256_44906478_44910319_FR | OBD160.1417 |
| 156 | Hg38_9_78097129_78101713_78276332_78285196_FF | OBD160.1421 |
| 157 | Hg38_3_126673758_126686020_126843341_126854251_RF | OBD160.1425 |
| 158 | Hg38_17_68575027_68578789_68785810_68796098_RF | OBD160.1429 |
| 159 | Hg38_5_24349034_24361029_24564285_24566267_FR | OBD160.1433 |
| 160 | Hg38_11_7448008_7457037_7633311_7640164_RR | OBD160.1437 |
| 161 | Hg38_7_27161846_27163700_27302548_27312509_FF | OBD160.1441 |
| 162 | Hg38_13_80339373_80340696_80530340_80535867_RF | OBD160.1445 |
| 163 | Hg38_9_4966910_4970390_5177459_5185304_FR | OBD160.1449 |
| 164 | Hg38_3_126843341_126854251_127025761_127027674_FR | OBD160.1453 |
| 165 | Hg38_2_13845156_13854426_13894360_13900303_FR | OBD160.1457 |
| 166 | Hg38_3_126619219_126620249_126843341_126854251_RF | OBD160.1461 |
| 167 | Hg38_11_19587032_19595878_19772073_19777708_RF | OBD160.1465 |
| 168 | Hg38_3_107106310_107120834_107363175_107364266_RF | OBD160.1469 |
| 169 | Hg38_2_172327203_172330194_172401667_172407636_FF | OBD160.1473 |
| 170 | Hg38_2_27492451_27495056_27626184_27635183_FF | OBD160.1477 |
| 171 | Hg38_6_108138075_108144989_108280425_108286846_RR | OBD160.1481 |
| 172 | Hg38_12_95793441_95797455_95929302_95940423_FR | OBD160.1485 |
| 173 | Hg38_17_34470224_34473967_34596926_34605729_RF | OBD160.1489 |
| 174 | Hg38_3_52542416_52548124_52770196_52773754_FR | OBD160.1493 |
| 175 | Hg38_1_186902459_186905074_187113187_187139713_RR | OBD160.1497 |
| 176 | Hg38_X_46498682_46508989_46576307_46579924_FR | OBD160.1501 |
| 177 | Hg38_6_3017794_3021910_3146778_3149848_RR | OBD160.1505 |
| 178 | Hg38_1_32149028_32153363_32237144_32241139_RF | OBD160.1509 |
| 179 | Hg38_11_316800_318015_481419_487611_FR | OBD160.1513 |
| 180 | Hg38_15_53424923_53427397_53442412_53454683_FF | OBD160.1517 |
| 181 | Hg38_17_68666853_68670189_68785810_68796098_RF | OBD160.1521 |
| 182 | Hg38_11_7448008_7457037_7610329_7615891_RR | OBD160.1525 |
| 183 | Hg38_17_77934896_77938750_78117632_78120001_RF | OBD160.1529 |
| 184 | Hg38_5_43590188_43596508_43695191_43701837_RR | OBD160.1533 |
| 185 | Hg38_1_60428956_60435934_60549270_60560051_FR | OBD160.1537 |
| 186 | Hg38_5_150272362_150278699_150370575_150373999_FF | OBD160.1541 |
| 187 | Hg38_9_130859900_130867193_130899377_130901466_RR | OBD160.1545 |
| 188 | Hg38_1_91404825_91416231_91606200_91614477_FR | OBD160.1549 |
| 189 | Hg38_14_91576230_91580637_91629033_91639298_RR | OBD160.1553 |
| 190 | Hg38_2_27349601_27353190_27492451_27495056_RF | OBD160.1557 |
| 191 | Hg38_5_150051979_150055894_150272362_150278699_RF | OBD160.1561 |
| 192 | Hg38_1_185167238_185172841_185287397_185290374_FR | OBD160.1565 |
| 193 | Hg38_3_112416500_112418982_112549606_112554897_FF | OBD160.1569 |
| 194 | Hg38_6_132376734_132385044_132542354_132547678_FR | OBD160.1573 |
| 195 | Hg38_1_189027657_189033909_189210801_189218947_FR | OBD160.1577 |
| 196 | Hg38_2_233409972_233419808_233475741_233480083_FR | OBD160.1581 |
| 197 | Hg38_17_35250006_35254814_35287155_35289074_RF | OBD160.1585 |
| 198 | Hg38_5_140984243_140987011_141123576_141128793_FF | OBD160.1589 |
| 199 | Hg38_13_40670004_40674221_40797038_40804771_RR | OBD160.1593 |
| 200 | Hg38_15_34800440_34807454_35007401_35010342_RR | OBD160.1597 |

TABLE 1B (part 4f)

| No. | Primer Sequence | Primer ID | Primer Sequence |
|---|---|---|---|
| 151 | ATTATCCTCCTTTGTTCCTTCCCC | OBD160.1403 | CCCTCCAGGTCTCTGCGTGTTTC |
| 152 | GCTCCCAGCAGAGGGCTCACCCG | OBD160.1407 | ATCCCGAGGACGACGGCTGTCTC |
| 153 | CTTCACAGCAGTCAAGTGGTGGTATCAC | OBD160.1411 | CTCCTTGTTTGACTGACTTAGTTTCTCA |
| 154 | AGATTTTGGGCTGAGACAATGGG | OBD160.1415 | CAGAACTCAAGAACATACTCCATAAAGT |
| 155 | CTCTGTTCCGCCTACCCAGGTCCACACC | OBD160.1419 | CCCAGCCCTTACCATCGGTGCCC |
| 156 | GCTCTAACTGATGCTTTCTCCAAG | OBD160.1423 | TGAGTAACAAAGTGAGACCTTATGTC |
| 157 | GGCAGAAAGGAAAGAGCCTCGCAGCATT | OBD160.1427 | TCCTCATCCTTGCTTTCCTCATCTGTGA |
| 158 | CTGGCTATTTTCACTTCTTTTGTGGA | OBD160.1431 | GCAAGGCTGTGTCCCCTCCTGAG |
| 159 | GTGGGAGTTGAACAATGAGAACAC | OBD160.1435 | AATGCCACCACCAGTAATGTTTCTAT |
| 160 | GCCTCCCCATTTCTCCTCAGTTTCACAA | OBD160.1439 | GGCTCTGGAGACTCAGCACCATTCTGGG |
| 161 | CATTTTCATCCTGCGGTTCTGGA | OBD160.1443 | CAACAGAGATGGGAGACTTGAAG |
| 162 | CGTGGCTCCTACAGAACTCCATA | OBD160.1447 | AAAGCAGTTTCTGTGTCCGTTTGCCCCA |
| 163 | GCCTCTCTCCCATCAACATCCTTCAGAC | OBD160.1451 | ATGGCGGCTGCCCCTCCTCCCAGG |
| 164 | GGCAGAAAGGAAAGAGCCTCGCAGCATT | OBD160.1455 | CTCAGACTCCTCCCAGCCCCACTCAGG |
| 165 | AATAGGGAACAGGTGTGTGACCCATAGC | OBD160.1459 | GAAATGCCAGAGAAGAAAGACAACAACT |
| 166 | GGCAGAAAGGAAAGAGCCTCGCAGCATT | OBD160.1463 | CTCCCCATCCTCCCCTTTACCCTACCC |
| 167 | CTTGGTATTTCCCTTATTGTTTTGAAGC | OBD160.1467 | CCACCATTGCTGAGGCTTGAGTA |
| 168 | CCACATCTTCCACCCCTGACACC | OBD160.1471 | GGTGGAGGAGAACGGTTTGATGC |
| 169 | CTCCCCAAGCCTCCCCAAGCAGG | OBD160.1475 | GCTGGTGTTGGATTGAATAGGAGGAAGA |
| 170 | TCCGCCCTGCCCACACCCACCTCC | OBD160.1479 | GCACACCCTGCCTAAAGTGGTTTTCCCT |
| 171 | CAGTGTCTCTGCTACAGGCTACAACATC | OBD160.1483 | CAAAGAATAAACAAGGAGACAAGGGCTT |
| 172 | GTGCGGTGGCTCACACTTGTAAT | OBD160.1487 | AGCCGAAGCAGGGCAAGGCATTG |
| 173 | CCACCCTGCTGTCCTGCTTGGGC | OBD160.1491 | GAGTTGGGAGGAATCTAAAGGGTTACCT |
| 174 | ATTATCCTCCTTTGTTCCTTCCCC | OBD160.1495 | CTCGGATGGCGTTTCCTCAGGGA |
| 175 | AGGAGAAGGAATGTCACAAGGTAAGGTC | OBD160.1499 | GTAGAGAAGGACAATGGTATGGGTTCAG |
| 176 | AGATTTTGGGCTGAGACAATGGG | OBD160.1503 | AAGGCTCCATCTGTCAAACGGAT |
| 177 | TAGGAGGAGAAAACAGCAAATGGTCGGG | OBD160.1507 | CCGACCCAAACCAGCACTCCAGCCG |
| 178 | GCCCTTTCCAGCCCCTCAGTCCTGTAGC | OBD160.1511 | CCATCCCTCAGGAAGCCCAGAGACTG |
| 179 | CCAGGAATGTCAGGCGACCCCTCAGGTG | OBD160.1515 | GCCATCCCATCTTCAAGGACTGACGGAG |
| 180 | TGAAGTCTTGAGGCGGCGAAAAT | OBD160.1519 | GGTAACCAACATCTTGTATTGTATTAGC |
| 181 | CTGGCTATTTTCACTTCTTTTGTGGA | OBD160.1523 | GTTGTGGAAAGCAGTATCGTGATTC |
| 182 | GCCTCCCCATTTCTCCTCAGTTTCACAA | OBD160.1527 | CTGCTTCTCCTCCTGCTCTCTCTGCTCC |
| 183 | GGGCTGGAGCACAGTGACGCAATCGTGG | OBD160.1531 | GACTCCCTTTGTTCTGCCTGGCTTCTGA |
| 184 | CTCTGCCTCCTCTGCCCTGTTCCTACTG | OBD160.1535 | GCCACCACAACACACTGCTGCCTCACAC |
| 185 | CACACAGATAAAGATGAGCAGGAGGGAC | OBD160.1539 | GCTCTCCTGGACTTGAGTGTGGAAGGG |
| 186 | GGGCAAGGGTGGGAAGCGATGGC | OBD160.1543 | AGGGAGCCAGAGACAGAGGAGGCAGTGG |
| 187 | AGGTGCTCAGTCAGGGCTGCTGCCTCTC | OBD160.1547 | TCCTGTGGGTCCCTGTGGCTGGG |
| 188 | GTAACTACATCGCTCCCAACTTTCAATG | OBD160.1551 | CTGCTCCTTTGCCCTTCCAGAGAGACAA |
| 189 | ACATTTCTTGCCCACTGTTCAGCCGAT | OBD160.1555 | CCCTCACCCCTGGCAACCACCGAT |
| 190 | CCTGCCCACACCCACCTCCGCCG | OBD160.1559 | GGCAACAGAGAGAGGCTCCGTCT |
| 191 | ACAGGACAGGGCAGGAGAGGCAACGAGG | OBD160.1563 | CCCAAGGCGGTAGAGGGTGTCACG |
| 192 | ACCACTATGACCTCTTTCCCAAATG | OBD160.1567 | CTGTGGCTATTACTGTGTTACAAT |
| 193 | GCCTGACATTCCTGCCGTCTTAT | OBD160.1571 | GTGACACCCACATACTTGCCAAAT |
| 194 | CAGTGCCCAAAGAGGTCCCTGAGGTC | OBD160.1575 | GTGGCTTTACAACCTCACTCTGAACCTT |
| 195 | CTGGGCTATTCTAAATGCCAACTAAT | OBD160.1579 | ATTCAGAGATTCAACTTCTTCCTGGT |
| 196 | GGCAGCAGGTAAGAGCCCCAGGG | OBD160.1583 | CCGCTCAGGAGACTCGCACTGGGTGTAG |
| 197 | TCTACTAACCAGAAAGACAGCCCTCACC | OBD160.1587 | GATGGTGTGTGGGTTAGGCACTACTGGG |
| 198 | ATTCTCAGGATGGAGGCTGGGTGTGGT | OBD160.1591 | GGAAAGCATCTCAAGACCAAAGTCAAAG |
| 199 | GTCACCCTTCAGAGATGGCAACATTTAC | OBD160.1595 | TAGCAACAACTGTCTCCCGTCCTCTGC |
| 200 | CCTCTTTCCCAAATGTCATCTTTTAG | OBD160.1599 | GGAGGAAAATAGTTGAGCAGGAACA |

TABLE 1B (part 4g)

| No. | Probe | Markers |
|---|---|---|
| 151 | Hg38_3_52542416_52548124_52606123_52609751_FF | OBD160.1401.1403 |
| 152 | Hg38_8_143012831_143021167_143073684_143079751_FR | OBD160.1405.1407 |
| 153 | Hg38_1_212366521_212376213_212547138_212548374_RR | OBD160.1409.1411 |
| 154 | Hg38_X_46498682_46508989_46602210_46603634_FR | OBD160.1413.1415 |
| 155 | Hg38_21_44873351_44875256_44906478_44910319_FR | OBD160.1417.1419 |
| 156 | Hg38_9_78097129_78101713_78276332_78285196_FF | OBD160.1421.1423 |
| 157 | Hg38_3_126673758_126686020_126843341_126854251_RF | OBD160.1425.1427 |
| 158 | Hg38_17_68575027_68578789_68785810_68796098_RF | OBD160.1429.1431 |
| 159 | Hg38_5_24349034_24361029_24564285_24566267_FR | OBD160.1433.1435 |
| 160 | Hg38_11_7448008_7457037_7633311_7640164_RR | OBD160.1437.1439 |
| 161 | Hg38_7_27161846_27163700_27302548_27312509_FF | OBD160.1441.1443 |
| 162 | Hg38_13_80339373_80340696_80530340_80535867_RF | OBD160.1445.1447 |
| 163 | Hg38_9_4966910_4970390_5177459_5185304_FR | OBD160.1449.1451 |
| 164 | Hg38_3_126843341_126854251_127025761_127027674_FR | OBD160.1453.1455 |
| 165 | Hg38_2_13845156_13854426_13894360_13900303_FR | OBD160.1457.1459 |
| 166 | Hg38_3_126619219_126620249_126843341_126854251_RF | OBD160.1461.1463 |
| 167 | Hg38_11_19587032_19595878_19772073_19777708_RF | OBD160.1465.1467 |
| 168 | Hg38_3_107106310_107120834_107363175_107364266_RF | OBD160.1469.1471 |
| 169 | Hg38_2_172327203_172330194_172401667_172407636_FF | OBD160.1473.1475 |
| 170 | Hg38_2_27492451_27495056_27626184_27635183_FF | OBD160.1477.1479 |
| 171 | Hg38_6_108138075_108144989_108280425_108286846_RR | OBD160.1481.1483 |
| 172 | Hg38_12_95793441_95797455_95929302_95940423_FR | OBD160.1485.1487 |
| 173 | Hg38_17_34470224_34473967_34596926_34605729_RF | OBD160.1489.1491 |
| 174 | Hg38_3_52542416_52548124_52770196_52773754_FR | OBD160.1493.1495 |
| 175 | Hg38_1_186902459_186905074_187113187_187139713_RR | OBD160.1497.1499 |
| 176 | Hg38_X_46498682_46508989_46576307_46579924_FR | OBD160.1501.1503 |
| 177 | Hg38_6_3017794_3021910_3146778_3149848_RR | OBD160.1505.1507 |
| 178 | Hg38_1_32149028_32153363_32237144_32241139_RF | OBD160.1509.1511 |
| 179 | Hg38_11_316800_318015_481419_487611_FR | OBD160.1513.1515 |
| 180 | Hg38_15_53424923_53427397_53442412_53454683_FF | OBD160.1517.1519 |
| 181 | Hg38_17_68666853_68670189_68785810_68796098_RF | OBD160.1521.1523 |
| 182 | Hg38_11_7448008_7457037_7610329_7615891_RR | OBD160.1525.1527 |
| 183 | Hg38_17_77934896_77938750_78117632_78120001_RF | OBD160.1529.1531 |
| 184 | Hg38_5_43590188_43596508_43695191_43701837_RR | OBD160.1533.1535 |
| 185 | Hg38_1_60428956_60435934_60549270_60560051_FR | OBD160.1537.1539 |
| 186 | Hg38_5_150272362_150278699_150370575_150373999_FF | OBD160.1541.1543 |
| 187 | Hg38_9_130859900_130867193_130899377_130901466_RR | OBD160.1545.1547 |
| 188 | Hg38_1_91404825_91416231_91606200_91614477_FR | OBD160.1549.1551 |
| 189 | Hg38_14_91576230_91580637_91629033_91639298_RR | OBD160.1553.1555 |
| 190 | Hg38_2_27349601_27353190_27492451_27495056_RF | OBD160.1557.1559 |
| 191 | Hg38_5_150051979_150055894_150272362_150278699_RF | OBD160.1561.1563 |
| 192 | Hg38_1_185167238_185172841_185287397_185290374_FR | OBD160.1565.1567 |
| 193 | Hg38_3_112416500_112418982_112549606_112554897_FF | OBD160.1569.1571 |
| 194 | Hg38_6_132376734_132385044_132542354_132547678_FR | OBD160.1573.1575 |
| 195 | Hg38_1_189027657_189033909_189210801_189218947_FR | OBD160.1577.1579 |
| 196 | Hg38_2_233409972_233419808_233475741_233480083_FR | OBD160.1581.1583 |
| 197 | Hg38_17_35250006_35254814_35287155_35289074_RF | OBD160.1585.1587 |
| 198 | Hg38_5_140984243_140987011_141123576_141128793_FF | OBD160.1589.1591 |
| 199 | Hg38_13_40670004_40674221_40797038_40804771_RR | OBD160.1593.1595 |
| 200 | Hg38_15_34800440_34807454_35007401_35010342_RR | OBD160.1597.1599 |

1. A method of detecting the muscular atrophy status in an individual, comprising determining the presence or absence of one or more chromosome interactions represented by the probes shown in Table 1, to thereby detect muscular atrophy in the individual.

2. A method according to claim 1 wherein:
(i) the presence or absence of at least 5 chromosome interactions from Table 1A is determined, and/or
(ii) the presence or absence of at least 5 chromosome interactions from Table 1B is determined.

3. A method according to claim 1 wherein the presence or absence of one or more chromosome interactions is determined:
in a sample from an individual, and/or
by detecting the presence or absence of a DNA loop at the site of the chromosome interactions, and/or
detecting the presence or absence of distal regions of a chromosome being brought together in a chromosome conformation, and/or
by detecting the presence of a ligated nucleic acid which is generated during said typing and whose sequence comprises two regions each corresponding to the regions of the chromosome which come together in the chromosome interaction, and/or
by a process which detects the proximity of the chromosome regions which have come together in the chromosome interaction.

4. A method according to claim 1 wherein said determining of the presence or absence of the chromosome interactions is by a process comprising:
(i) in vitro crosslinking of epigenetic chromosomal interactions which are present;
(ii) optionally isolating the cross-linked DNA;
(iii) subjecting said cross-linked DNA to cleaving;
(iv) ligating said cross-linked cleaved DNA ends to form ligated DNA; and
(v) identifying the presence or absence of said ligated DNA;
to thereby determine the presence or absence of the chromosome interaction.

5. A method according to claim 3 wherein said ligated DNA is detected by PCR or by use of a probe.

6. A method according to claim 5 wherein:
(i) detection is by use of a probe, wherein said probe has at least 70% identity to any of the probes shown in Table 1, or
(ii) detection is by use of PCR, wherein the PCR uses a primer pair that has at least 70% identity to any of the primer pairs shown in Table 1.

7. A method according to claim 1 wherein:
(i) the method is carried out to select an individual for receiving therapy or a treatment for muscular atrophy, and/or
(ii) the method is carried out on individual that has been preselected based on a physical characteristic, risk factor or the presence of a symptom, and/or
(iii) the method is carried out to diagnose muscular atrophy or to determine prognosis for muscular atrophy, and preferably to determine severity of muscular atrophy.

8. A method according to claim 7(ii) wherein the individual is preselected for one or more of the following characteristics:
(a) being male, and/or
(b) being aged 30 to 60, and/or
(c) having gynecomastia, and/or
(d) having testicular atrophy, and/or
(e) having androgen insensitivity, and/or
(e) having reduced fertility, preferably as a result of androgen insensitivity.

9. A method according to claim 1 wherein the presence or absence of at least 5 chromosome interactions is determined which are selected from:
(i) interaction numbers 1 to 40 from Table 1A, or
(ii) interaction numbers 1 to 40 from Table 1B.

10. A method according to claim 1, wherein determining the presence or absence of one or more chromosome interactions comprises specific detection of a ligated product by quantitative PCR (qPCR) which uses primers capable of amplifying the ligated product and a probe which binds the ligation site during the PCR reaction, wherein said probe comprises sequence which is complementary to sequence from each of the chromosome regions that have come together in the chromosome interaction, wherein preferably said probe comprises:
an oligonucleotide which specifically binds to said ligated product, and/or
a fluorophore covalently attached to the 5′ end of the oligonucleotide, and/or
a quencher covalently attached to the 3′ end of the oligonucleotide, and
optionally
said fluorophore is selected from HEX, Texas Red and FAM; and/or
said probe comprises a nucleic acid sequence of length 10 to 40 nucleotide bases, preferably a length of −20 to 30 nucleotide bases.

11. A method of treating muscular atrophy comprising carrying out the method of claim 1 on an individual and administering an anti-muscular atrophy therapeutic agent to an individual that has been identified as having muscular atrophy by the method of claim 1.


SEQUENCE LISTING (1200 sequences)

SEQ ID NO: 1 - DNA, 60 bp, Homo sapiens
aatgttgataagtattagacgactggtttcgattttcttttaatcactgctaaatctgca

SEQ ID NO: 2 - DNA, 60 bp, Homo sapiens
tacttacactcaacaggagggatatatttcgaatcattttgggggcataaacataaaacc

SEQ ID NO: 3 - DNA, 60 bp, Homo sapiens
gtaagagaaactgttctttctgtcagcttcgaactacattgaaaatctcttgtttgtgta

SEQ ID NO: 4 - DNA, 60 bp, Homo sapiens
tttgcatttatttgatatattcccattctcgaccattttttcaattgagttattagtttt

SEQ ID NO: 5 - DNA, 60 bp, Homo sapiens
caaacatgtctaactcatgcaattttcttcgagtctccatttgaaaaaagaggttttttg

SEQ ID NO: 6 - DNA, 60 bp, Homo sapiens
gactgaaaaagttaagaaaactttctactcgattaacacctattttatttgtgctgcatt

SEQ ID NO: 7 - DNA, 60 bp, Homo sapiens
cttcagaacagtaaaaaaaaaaaaaagttcgactcctatttcttcatcaagctttccatt

SEQ ID NO: 8 - DNA, 60 bp, Homo sapiens
gctgacttgattttgctggataagcttttcgacttccttatactagtaagagacttaagc

SEQ ID NO: 9 - DNA, 60 bp, Homo sapiens
cagagagggacattatataatgaaatagtcgaggtctgatagcaaccatttaccaaactt

SEQ ID NO: 10 - DNA, 60 bp, Homo sapiens
gcttcaaaaggtcctagaaataatccactcgaatccttggcttttatttttcttcaggga

SEQ ID NO: 11 - DNA, 60 bp, Homo sapiens
aggaaagaaacataagaatattattatttcgaagtcattgaagattcacagcaggagaca

SEQ ID NO: 12 - DNA, 60 bp, Homo sapiens
ccaggccttgaagagataatacaggaattcgaaccaatgtacatgttacacatattgatt

SEQ ID NO: 13 - DNA, 60 bp, Homo sapiens
acaatctctgttctttctttcttttttttcgagaatcatggctttaaagacacagagatg

SEQ ID NO: 14 - DNA, 60 bp, Homo sapiens
aaatttaaagtttcttcttacaaagcattcgaagtgtaagccgtcatgatttgtttcata

SEQ ID NO: 15 - DNA, 60 bp, Homo sapiens
agtactgaaatattgtattgctactatttcgacacacacttgtattgaataaaaggcgta

SEQ ID NO: 16 - DNA, 60 bp, Homo sapiens
gtctttttagagatgaacaaaaataaattcgatctgtctggtcaacattctgggacaatg

SEQ ID NO: 17 - DNA, 60 bp, Homo sapiens
aagtgttagagaaagaagattaagcaattcgacattcccaccaataaggtataagtattt

SEQ ID NO: 18 - DNA, 60 bp, Homo sapiens
tcaagtcactggcaggaagaacaagctctcgaaaaaatacagtgacatctttatttgtct

SEQ ID NO: 19 - DNA, 60 bp, Homo sapiens
tatccatttccagaaatttttcattatctcgatgtatggattaactgtgtgatattaaca

SEQ ID NO: 20 - DNA, 60 bp, Homo sapiens
atattgggattactatgcagattacatttcgagtgttttaggaagtcatataatgaacaa

SEQ ID NO: 21 - DNA, 60 bp, Homo sapiens
ataaaaatggagtagaaatcagtgaaattcgaatcattcatgaaaggaaaatctgagaag

SEQ ID NO: 22 - DNA, 60 bp, Homo sapiens
gtgtctgttactgatccttatagcttcttcgaagaaacaaaactatcttcaaaaataagt

SEQ ID NO: 23 - DNA, 60 bp, Homo sapiens
agtttggtaaagtttcaggataaaaaaatcgaattaggagtcttgtaattccatgttcat

SEQ ID NO: 24 - DNA, 60 bp, Homo sapiens
tgtattggcataaagatagacaaatagatcgagaagaacaggattttggaattgagatag

SEQ ID NO: 25 - DNA, 60 bp, Homo sapiens
tgtgaaacaaagcccgctattcacagtctcgactttgttcagagttcagaatcaatatct

SEQ ID NO: 26 - DNA, 60 bp, Homo sapiens
caaatctttcccaaagttaaagtccaagtcgaacacagctactacattaaatataacaat

SEQ ID NO: 27 - DNA, 60 bp, Homo sapiens
ctcatcagaattgttataataaaaaagatcgaacaagactctctttctctagtaagtcct

SEQ ID NO: 28 - DNA, 60 bp, Homo sapiens
agaaataaaagaaggagtcaaaataccttcgatttgagcagaaagtcctgaaggcacaca

SEQ ID NO: 29 - DNA, 60 bp, Homo sapiens
gattgtattacttcaaaataaaacttgatcgaagaagtacaagtcaaaggcagacagcac

SEQ ID NO: 30 - DNA, 60 bp, Homo sapiens
gaaaaagagagaaacaaactattacttatcgagttttagattctaaaatgttgtttagta

SEQ ID NO: 31 - DNA, 60 bp, Homo sapiens
aatcaaatgatagaaacatagaccaaagtcgagtttaaggcaaaatgcagtctatgaaaa

SEQ ID NO: 32 - DNA, 60 bp, Homo sapiens
taatcttttacattctgctttaagcacatcgatttttaagattctctcagttggaagaaa

SEQ ID NO: 33 - DNA, 60 bp, Homo sapiens
ttgaacacagacaaggtactaggtaatatcgattttagcttttttttgactttttctcca

SEQ ID NO: 34 - DNA, 60 bp, Homo sapiens
ttcttggtatgcctggtttttttttaattcgattttagccctaatcccattgtgctgtaa

SEQ ID NO: 35 - DNA, 60 bp, Homo sapiens
tgacttctgctaggatacaccagaaaattcgagtatgacaaagagcttaagccaaggtag

SEQ ID NO: 36 - DNA, 60 bp, Homo sapiens
tacatggctttattgaaataacattccatcgagttttcttttcaaaggagatggatccaa

SEQ ID NO: 37 - DNA, 60 bp, Homo sapiens
cgtgactgtatatcatgtcctttgttaatcgagactggttaatttaaaaagaaaagaggt

SEQ ID NO: 38 - DNA, 60 bp, Homo sapiens
tagaaacaaaagttacagaaataatccctcgataacattgttttaattttctgagaaaaa

SEQ ID NO: 39 - DNA, 60 bp, Homo sapiens
caagcaaaacaatggcttaaattgtttctcgaacagctgaaatccctgatgtatgcaaag

SEQ ID NO: 40 - DNA, 60 bp, Homo sapiens
tcctacaagaaatgctaaagagagttattcgatgaattaatacatgcattttgaaattga

SEQ ID NO: 41 - DNA, 60 bp, Homo sapiens
tcttaggtttgcaaacagaccaagtaagtcgatattggttgggaactggttagtaattaa

SEQ ID NO: 42 - DNA, 60 bp, Homo sapiens
ctaaaagaaaacataggggaaaaagtcctcgattttcataacatttatcttagcctcaaa

SEQ ID NO: 43 - DNA, 60 bp, Homo sapiens
ttaccctttaagtcaatgcctcaaaagttcgaaaactgcctttgataataccagtgtctt

SEQ ID NO: 44 - DNA, 60 bp, Homo sapiens
aatgtacacaaccagtcataaccaagtatcgacttttctcttcccaagggagagtttact

SEQ ID NO: 45 - DNA, 60 bp, Homo sapiens
tataaactggaactttcaaatttcatgctcgattacttaaggcatgctattataaataaa

SEQ ID NO: 46 - DNA, 60 bp, Homo sapiens
ttgtgtactagtaaactgcaggcatccatcgaatgctttgcagtcaagagaaggcaatga

SEQ ID NO: 47 - DNA, 60 bp, Homo sapiens
taaacacgagctttctcagctgtgctgctcgagattttttaattaataggatagatggat

SEQ ID NO: 48 - DNA, 60 bp, Homo sapiens
catggtcaccaaatatggcatgacagaatcgacagagaaagctacgacgcaataggaggt

SEQ ID NO: 49 - DNA, 60 bp, Homo sapiens
atattattcttttgatttttccaaccattcgacgctgtccagtgaaaatataacacgagc

SEQ ID NO: 50 - DNA, 60 bp, Homo sapiens
atctgtgagatttgcagcttctttttgctcgaattgagtttatttcaaagatattgataa

SEQ ID NO: 51 - DNA, 26 bp, Homo sapiens
cacactgagactgatggtgcgtgagt

SEQ ID NO: 52 - DNA, 25 bp, Homo sapiens
cagtgtctgaagcaaatcctctgac

SEQ ID NO: 53 - DNA, 23 bp, Homo sapiens
aaacttcttagtggaggaggctc

SEQ ID NO: 54 - DNA, 23 bp, Homo sapiens
agcctgggtgacagagtgagact

SEQ ID NO: 55 - DNA, 23 bp, Homo sapiens
agtgtctggagttggaagagcag

SEQ ID NO: 56 - DNA, 23 bp, Homo sapiens
acagcaagttatcccgaagatgc

SEQ ID NO: 57 - DNA, 28 bp, Homo sapiens
cacctcctgtggtttctgctttatgtgt

SEQ ID NO: 58 - DNA, 28 bp, Homo sapiens
gcttcaagacattagttttggcaatagt

SEQ ID NO: 59 - DNA, 26 bp, Homo sapiens
ccctgtgagataggtgaagtgggtgt

SEQ ID NO: 60 - DNA, 26 bp, Homo sapiens
atggctgagtctgaaaatggaatggg

SEQ ID NO: 61 - DNA, 26 bp, Homo sapiens
gcaaaagcaatagccagcaccataga

SEQ ID NO: 62 - DNA, 26 bp, Homo sapiens
accaacctctcttccttctcagcctg

SEQ ID NO: 63 - DNA, 23 bp, Homo sapiens
gagcccacaaagcctaaaatacg

SEQ ID NO: 64 - DNA, 23 bp, Homo sapiens
gcccacactgttcttacacttgc

SEQ ID NO: 65 - DNA, 23 bp, Homo sapiens
tgcgtatgttgaaccagtcttgc

SEQ ID NO: 66 - DNA, 23 bp, Homo sapiens
gcctggaaatgaggaaccctggt

SEQ ID NO: 67 - DNA, 26 bp, Homo sapiens
gataatgccatcgtcaggttgaaatc

SEQ ID NO: 68 - DNA, 23 bp, Homo sapiens
gccaaagcggaaccaggtaacta

SEQ ID NO: 69 - DNA, 23 bp, Homo sapiens
gccctcctccataccaactcgct

SEQ ID NO: 70 - DNA, 23 bp, Homo sapiens
ccctttgccaccacagacttcag

SEQ ID NO: 71 - DNA, 26 bp, Homo sapiens
aggcaatgtggatgctgtagtcaact

SEQ ID NO: 72 - DNA, 26 bp, Homo sapiens
cttcccctgctgccatctaacatcct

SEQ ID NO: 73 - DNA, 26 bp, Homo sapiens
ccctgagtgaaaatccaaaatccttg

SEQ ID NO: 74 - DNA, 25 bp, Homo sapiens
catttgcccaggatggtcggtggta

SEQ ID NO: 75 - DNA, 23 bp, Homo sapiens
accccaaccagtagcagtatctg

SEQ ID NO: 76 - DNA, 25 bp, Homo sapiens
gttggctatccactctttctcatct

SEQ ID NO: 77 - DNA, 23 bp, Homo sapiens
ctaaaggagtgaactgaagcagc

SEQ ID NO: 78 - DNA, 25 bp, Homo sapiens
gtttgctgagaatgatggtttccag

SEQ ID NO: 79 - DNA, 26 bp, Homo sapiens
cgttcagactttccatcaatgtaaca

SEQ ID NO: 80 - DNA, 26 bp, Homo sapiens
gctgtggtttacctctctgctatctt

SEQ ID NO: 81 - DNA, 28 bp, Homo sapiens
gggctggaagaaaatgaagagtgactac

SEQ ID NO: 82 - DNA, 28 bp, Homo sapiens
gctatttgccaagaccacagtcagacca

SEQ ID NO: 83 - DNA, 28 bp, Homo sapiens
cataactgactttacttttgggatgatg

SEQ ID NO: 84 - DNA, 28 bp, Homo sapiens
gtagacagcatccagattccttgagtgt

SEQ ID NO: 85 - DNA, 26 bp, Homo sapiens
ttgatgcccaggtgcttgtttgatgc

SEQ ID NO: 86 - DNA, 24 bp, Homo sapiens
gctgagagcattgtggaagaatct

SEQ ID NO: 87 - DNA, 23 bp, Homo sapiens
gcagcccttaggtagcaagatgc

SEQ ID NO: 88 - DNA, 26 bp, Homo sapiens
gaagttatcaagttcaacctcctatc

SEQ ID NO: 89 - DNA, 26 bp, Homo sapiens
tgacatcaggaagagactcagtgtat

SEQ ID NO: 90 - DNA, 26 bp, Homo sapiens
ctgactgatgtatcctaccaataagc

SEQ ID NO: 91 - DNA, 24 bp, Homo sapiens
acacagttggtaagtggcaaagcc

SEQ ID NO: 92 - DNA, 24 bp, Homo sapiens
aactattctaagggtcttggaaat

SEQ ID NO: 93 - DNA, 26 bp, Homo sapiens
tctgtgattttccttctcaagttgtg

SEQ ID NO: 94 - DNA, 26 bp, Homo sapiens
cattttggtgccttcccacacataaa

SEQ ID NO: 95 - DNA, 23 bp, Homo sapiens
gccaaataggacagacccacagc

SEQ ID NO: 96 - DNA, 28 bp, Homo sapiens
gtcattcgttccccagtcacctgcttca

SEQ ID NO: 97 - DNA, 26 bp, Homo sapiens
aacacaatctcaccccaaatcccagc

SEQ ID NO: 98 - DNA, 26 bp, Homo sapiens
cttacccatctgactgtgttcttagc

SEQ ID NO: 99 - DNA, 26 bp, Homo sapiens
ctactctcaatgtgacgactggaagg

SEQ ID NO: 100 - DNA, 26 bp, Homo sapiens
gtagggaggcagaaatggtgtgtttg

SEQ ID NO: 101 - DNA, 26 bp, Homo sapiens
tatgagtccccagaggatagaagaat

SEQ ID NO: 102 - DNA, 26 bp, Homo sapiens
tggcactacataaaatcacccattgt

SEQ ID NO: 103 - DNA, 26 bp, Homo sapiens
cagcaagatgcctgaacagactttac

SEQ ID NO: 104 - DNA, 26 bp, Homo sapiens
tatccacaacgaggtgaggactctcc

SEQ ID NO: 105 - DNA, 28 bp, Homo sapiens
ccagaaaagcagcagttacacctacagg

SEQ ID NO: 106 - DNA, 28 bp, Homo sapiens
cttgagagtaaaggtggtaacataacac

SEQ ID NO: 107 - DNA, 26 bp, Homo sapiens
atgaagggcaccaaaagatgctacct

SEQ ID NO: 108 - DNA, 26 bp, Homo sapiens
tggcagcaaactctcaccttctggag

SEQ ID NO: 109 - DNA, 26 bp, Homo sapiens
cgggaggctcaaaagagggagaaaga

SEQ ID NO: 110 - DNA, 26 bp, Homo sapiens
ttggctaacacagtctcatccagtga

SEQ ID NO: 111 - DNA, 26 bp, Homo sapiens
gaggtgtttcagtgagagtagagagg

SEQ ID NO: 112 - DNA, 26 bp, Homo sapiens
agagccacattcaatagacgacccgt

SEQ ID NO: 113 - DNA, 26 bp, Homo sapiens
tgtagtcaccaataccgtaaaacccc

SEQ ID NO: 114 - DNA, 26 bp, Homo sapiens
caaagactactgggtaactacagacg

SEQ ID NO: 115 - DNA, 28 bp, Homo sapiens
tttccttatcttggtgctgttacatagc

SEQ ID NO: 116 - DNA, 28 bp, Homo sapiens
ggcttttctgtgatgctctggttggcaa

SEQ ID NO: 117 - DNA, 26 bp, Homo sapiens
tttctcctcaagatgggtcctatttt

SEQ ID NO: 118 - DNA, 26 bp, Homo sapiens
ctgccacctcctctgtatggttctta

SEQ ID NO: 119 - DNA, 26 bp, Homo sapiens
gacacacacagaggtagtagagattc

SEQ ID NO: 120 - DNA, 26 bp, Homo sapiens
gctctctcagcccaggtgcttagttt

SEQ ID NO: 121 - DNA, 26 bp, Homo sapiens
cgaaggattgactctgataggcttac

SEQ ID NO: 122 - DNA, 26 bp, Homo sapiens
tgcttggctggctctaaaggcttgga

SEQ ID NO: 123 - DNA, 28 bp, Homo sapiens
acttttattccactggcagcaaggtaat

SEQ ID NO: 124 - DNA, 28 bp, Homo sapiens
gaagacagatgacagaagacaggtggct

SEQ ID NO: 125 - DNA, 26 bp, Homo sapiens
aaggagttgttctcaaggtatgttac

SEQ ID NO: 126 - DNA, 26 bp, Homo sapiens
ctgactgattgctcctattggtaaat

SEQ ID NO: 127 - DNA, 26 bp, Homo sapiens
cagcccaaagcgagggtggaaaacat

SEQ ID NO: 128 - DNA, 26 bp, Homo sapiens
gtgaactactctttcttgctgcttcc

SEQ ID NO: 129 - DNA, 21 bp, Homo sapiens
acctgtcctacaagaaatgct

SEQ ID NO: 130 - DNA, 24 bp, Homo sapiens
acatgcattttgaaattgatagtt

SEQ ID NO: 131 - DNA, 26 bp, Homo sapiens
cagtaactaatcttctctggtgacaa

SEQ ID NO: 132 - DNA, 26 bp, Homo sapiens
actctacagattcccaggacctactg

SEQ ID NO: 133 - DNA, 26 bp, Homo sapiens
caagaccaccaaaatacagaatggag

SEQ ID NO: 134 - DNA, 26 bp, Homo sapiens
gagaaacgattcctaacccctaccat

SEQ ID NO: 135 - DNA, 26 bp, Homo sapiens
ccttttcctcaggtttctcttttccc

SEQ ID NO: 136 - DNA, 26 bp, Homo sapiens
gaagccaagcgaaggcatctgactca

SEQ ID NO: 137 - DNA, 28 bp, Homo sapiens
gaggagcgaaagaaagggttttagactc

SEQ ID NO: 138 - DNA, 28 bp, Homo sapiens
cactaaaagtcctaaagaagaagccaaa

SEQ ID NO: 139 - DNA, 26 bp, Homo sapiens
ggaatcagaagaacttgtgtgaccca

SEQ ID NO: 140 - DNA, 26 bp, Homo sapiens
cagtgagagagagcaatggaggaagc

SEQ ID NO: 141 - DNA, 28 bp, Homo sapiens
gttggtagaatgaatcaatggtagaatg

SEQ ID NO: 142 - DNA, 23 bp, Homo sapiens
gcctgggtgacagagggagactc

SEQ ID NO: 143 - DNA, 26 bp, Homo sapiens
gcttcaaacagacaaaggtatttcct

SEQ ID NO: 144 - DNA, 24 bp, Homo sapiens
gcccagagactatgctttgagaat

SEQ ID NO: 145 - DNA, 23 bp, Homo sapiens
ccacccagcagcaaaagcagcag

SEQ ID NO: 146 - DNA, 23 bp, Homo sapiens
ccttctgcttctccaccacccat

SEQ ID NO: 147 - DNA, 28 bp, Homo sapiens
cccttgtagttggaaagcagccgcagcc

SEQ ID NO: 148 - DNA, 28 bp, Homo sapiens
aatgtagccagtgtcacaggcagaagtg

SEQ ID NO: 149 - DNA, 26 bp, Homo sapiens
gtgcctctccttacactcatctctca

SEQ ID NO: 150 - DNA, 26 bp, Homo sapiens
gcgaaagactctgtaactgtgttggc

SEQ ID NO: 151 - DNA, 60 bp, Homo sapiens
atacaggtatcaaatcatctagttgtactcgatgtataaaaatcacaagcattcctatac

SEQ ID NO: 152 - DNA, 60 bp, Homo sapiens
tttagggtcttttctttatccttgatcttcgagaagatgttgagttttatcagatgcttt

SEQ ID NO: 153 - DNA, 60 bp, Homo sapiens
tatttgttatctattacttcctaacaaatcgaggacctgaattacttatcacccttctca

SEQ ID NO: 154 - DNA, 60 bp, Homo sapiens
tctgtttagcaaataaacacaaaaagtatcgacagattgaccagtttttagttgtagcac

SEQ ID NO: 155 - DNA, 60 bp, Homo sapiens
actgaagtcccagtagaacagaagagaatcgaaagattctgaatggggaaaaacttactc

SEQ ID NO: 156 - DNA, 60 bp, Homo sapiens
cacttaagagccagctatttgactgaattcgaaaagcttctaccaaaaaaatccttaaaa

SEQ ID NO: 157 - DNA, 60 bp, Homo sapiens
tttccactgttttcttacaactttcccttcgatttgtcttagcagacattttaagttaca

SEQ ID NO: 158 - DNA, 60 bp, Homo sapiens
aatggaaaaattcctggattaagttatgtcgatacaatcaaattttttaagcatttaaaa

SEQ ID NO: 159 - DNA, 60 bp, Homo sapiens
cccatgagtaaatgtagacctatgtctatcgacgtataccctatggagaggatattccct

SEQ ID NO: 160 - DNA, 60 bp, Homo sapiens
cacatctaacacaattcacagtacaaaatcgaatcaaagctgtgtcacaaactatgtaac

SEQ ID NO: 161 - DNA, 60 bp, Homo sapiens
taaaaatgtaaacggcctgtttttaaactcgatgtttgatcatttaaatgcattatgtct

SEQ ID NO: 162 - DNA, 60 bp, Homo sapiens
ataatttccacatgttctattggtccaatcgactgaagcaccaaaattaaaaatattttt

SEQ ID NO: 163 - DNA, 60 bp, Homo sapiens
cttgatctactgaacatatcttgagttttcgactggtttttctcctcattatattttcct

SEQ ID NO: 164 - DNA, 60 bp, Homo sapiens
agcctgtgatggcaagacttgccaaaactcgagttgcccaggtatgcatctttagaaagg

SEQ ID NO: 165 - DNA, 60 bp, Homo sapiens
aaaatcttttccagaagttgtttccagatcgagacttggactagaattaagtctgtgaac

SEQ ID NO: 166 - DNA, 60 bp, Homo sapiens
tgccttgatgattggaagagatataacttcgaaagtataaattctctaaattctaatttt

SEQ ID NO: 167 - DNA, 60 bp, Homo sapiens
ctgccttttaatcctatgtggaaattggtcgagttatttttgacaattgaactttttggt

SEQ ID NO: 168 - DNA, 60 bp, Homo sapiens
acccagaagaaagactgccaaatcttcctcgaaccaaagaacatgggaatggttcagagg

SEQ ID NO: 169 - DNA, 60 bp, Homo sapiens
tagtctatttctgttctagtatttattgtcgaacacatttctcaatatcatctatttggt

SEQ ID NO: 170 - DNA, 60 bp, Homo sapiens
cactcagaatgttaatgtatgtaattattcgatcatgtctctttccacatgtcttcttct

SEQ ID NO: 171 - DNA, 60 bp, Homo sapiens
aacaaactagacattgaagaaatgtacctcgatactattctaatctcccatttagtaggc

SEQ ID NO: 172 - DNA, 60 bp, Homo sapiens
agatcccttgtacactgtatactgtttgtcgaaatacctacgagtcctaaaatatagttc

SEQ ID NO: 173 - DNA, 60 bp, Homo sapiens
gatattggacaggatcaacaaaaaggaatcgactcgtgtatatgtttgatatatcccaca

SEQ ID NO: 174 - DNA, 60 bp, Homo sapiens
cactttcgtaatttgtcatcagataacttcgatgaacttggcaccaaggtcagtatgaga

SEQ ID NO: 175 - DNA, 60 bp, Homo sapiens
ctatatgttttgttccctatcatgcctttcgattactctaagatttaaagtcatgtggag

SEQ ID NO: 176 - DNA, 60 bp, Homo sapiens
ggaatcacatgggcaagagagctgagactcgaatgtcccccaccatcttacacatcattg

SEQ ID NO: 177 - DNA, 60 bp, Homo sapiens
cattacctgaccaggtcaatccagaatttcgacaaaaacattgtaggatgaaaagaggag

SEQ ID NO: 178 - DNA, 60 bp, Homo sapiens
tccaaagtttcaacttacaatcacatcttcgactcctttgcatgttttcttgcatccact

SEQ ID NO: 179 - DNA, 60 bp, Homo sapiens
ccttttcaaaaaaaaacaactctcattctcgatgacatcaatggttttgcagtattgagc

SEQ ID NO: 180 - DNA, 60 bp, Homo sapiens
ggaatcttcaaagaataagtgtttttcatcgaacatcataatgttctacttgcaaaacag

SEQ ID NO: 181 - DNA, 60 bp, Homo sapiens
ttaactccaaatttccaagtcagttaactcgacctttttctgtttcagtaaccaacccag

SEQ ID NO: 182 - DNA, 60 bp, Homo sapiens
actgcagatctgtacccaagtgatatgatcgacattccagtaagacttgtcttttattgg

SEQ ID NO: 183 - DNA, 60 bp, Homo sapiens
ggaacacttctctaagaacctgaattattcgagcttcaaatacaaaacatttgggctcaa

SEQ ID NO: 184 - DNA, 60 bp, Homo sapiens
taatccctttcaatttcgttaagtttcctcgattaagccatgcattgatacctgacccaa

SEQ ID NO: 185 - DNA, 60 bp, Homo sapiens
atgcagttcatctttccctcagtagatatcgagactaagaaacaaacacttgtcaaaata

SEQ ID NO: 186 - DNA, 60 bp, Homo sapiens
ttataaacatcttttggagttctgtacttcgagttttggaatctggcagtggaacttcag

SEQ ID NO: 187 - DNA, 60 bp, Homo sapiens
ttttcttcctaattttggttagccagtatcgagtcttagaagtattcaatttgcaatttg

SEQ ID NO: 188 - DNA, 60 bp, Homo sapiens
aaattgaaatgtaacaagtctgcatttgtcgaaacaaatatttgtctactgcattgttaa

SEQ ID NO: 189 - DNA, 60 bp, Homo sapiens
catcacttaaaaaccttgttaacaatcttcgaggttaccataacccattattttaagctg

SEQ ID NO: 190 - DNA, 60 bp, Homo sapiens
ttttaaagaatgcagaaaaaaaacaaaatcgacaaacagttcacacaataacactatgtg

SEQ ID NO: 191 - DNA, 60 bp, Homo sapiens
tttttaaggttatgtgtttgagtttgaatcgaaaagaagtattacactattttgctcccc

SEQ ID NO: 192 - DNA, 60 bp, Homo sapiens
acgatgaagataatgttacatttatcattcgaagttaaagtttactttcacaggacataa

SEQ ID NO: 193 - DNA, 60 bp, Homo sapiens
ttgtctatctcttttccttagaagactctcgaaaacaaaaacaaacaaaaaaataaaaat

SEQ ID NO: 194 - DNA, 60 bp, Homo sapiens
ctatgatttaactttacttcattaggtatcgaattacaacaaaaaaacccccaaaatcct

SEQ ID NO: 195 - DNA, 60 bp, Homo sapiens
cttggaaggttcaatgtgaaagaaagagtcgattttgaaatattatatagattcaggaga

SEQ ID NO: 196 - DNA, 60 bp, Homo sapiens
aatttatctcatttcagcattgtatcagtcgaataatcccacaggaaaaccaagttggcc

SEQ ID NO: 197 - DNA, 60 bp, Homo sapiens
catcataggtaccttaagaggtagactttcgaaatacatagtcagttcccagtttgtata

SEQ ID NO: 198 - DNA, 60 bp, Homo sapiens
tttgtatatgggctcagtaaaatgtgaatcgaacttctgcttttaaaatactctactctc

SEQ ID NO: 199 - DNA, 60 bp, Homo sapiens
caaagaaatgttgttcatgaaacttatatcgacataaagagcgttagggcaaattataag

SEQ ID NO: 200 - DNA, 60 bp, Homo sapiens
aaaaaaaaagaaaacagaaatttgcaactcgaaaaaggaaccttgattctctgagtgaat

SEQ ID NO: 201 - DNA, 26 bp, Homo sapiens
ggaatagagggaagaaaacccataaa

SEQ ID NO: 202 - DNA, 27 bp, Homo sapiens
ctctctgtttgtctgttattggtgtat

SEQ ID NO: 203 - DNA, 26 bp, Homo sapiens
agtctgtaaggtttcctatgagaagc

SEQ ID NO: 204 - DNA, 26 bp, Homo sapiens
gaaacgaatacccctgatgaagatag

SEQ ID NO: 205 - DNA, 26 bp, Homo sapiens
ctcagatttctcacctgtagagcagt

SEQ ID NO: 206 - DNA, 26 bp, Homo sapiens
ggaaaagtgaagaataagcacccatc

SEQ ID NO: 207 - DNA, 26 bp, Homo sapiens
ttctggtagcagtggtgggagtgtca

SEQ ID NO: 208 - DNA, 26 bp, Homo sapiens
aatgccatactcatactcaccgtgct

SEQ ID NO: 209 - DNA, 26 bp, Homo sapiens
gccttgagaaccttagaacaatgcca

SEQ ID NO: 210 - DNA, 26 bp, Homo sapiens
ggagaaatcccagttacacagttggc

SEQ ID NO: 211 - DNA, 23 bp, Homo sapiens
taagtccaggagattgaggctgc

SEQ ID NO: 212 - DNA, 26 bp, Homo sapiens
agtaactaagtctctaccttctttgc

SEQ ID NO: 213 - DNA, 26 bp, Homo sapiens
caatgtgtctaagtaacgcctgtgtg

SEQ ID NO: 214 - DNA, 26 bp, Homo sapiens
gaagaacataactgcccctgctccag

SEQ ID NO: 215 - DNA, 23 bp, Homo sapiens
tgctggctgggattacaggcgta

SEQ ID NO: 216 - DNA, 25 bp, Homo sapiens
ccacagacggtcaacaacaaaatgt

SEQ ID NO: 217 - DNA, 28 bp, Homo sapiens
gattggtagaggaaagagaagcagagaa

SEQ ID NO: 218 - DNA, 28 bp, Homo sapiens
gtgccagaaagggttatgctgttagtga

SEQ ID NO: 219 - DNA, 24 bp, Homo sapiens
ctaagaagacaacacaacctcctc

SEQ ID NO: 220 - DNA, 26 bp, Homo sapiens
gtatgctgcttacaccagataactta

SEQ ID NO: 221 - DNA, 26 bp, Homo sapiens
gaggaactcagaagactggctttagg

SEQ ID NO: 222 - DNA, 26 bp, Homo sapiens
cactggaaaagaggcaggtttgagac

SEQ ID NO: 223 - DNA, 23 bp, Homo sapiens
gccaccttcccagaaagtgaggc

SEQ ID NO: 224 - DNA, 23 bp, Homo sapiens
gaccccaaagcactctggacatc

SEQ ID NO: 225 - DNA, 24 bp, Homo sapiens
gctctactctttccctacaggctg

SEQ ID NO: 226 - DNA, 26 bp, Homo sapiens
aggaatagaaaagtgtagccacccta

SEQ ID NO: 227 - DNA, 26 bp, Homo sapiens
ttgtgctgtctggagttgtaggaggg

SEQ ID NO: 228 - DNA, 26 bp, Homo sapiens
ccctacttcatctcctctacacacaa

SEQ ID NO: 229 - DNA, 26 bp, Homo sapiens
tcattgtgtggctctgacttcccagt

SEQ ID NO: 230 - DNA, 26 bp, Homo sapiens
ccgaatagccaaggcagaacagcata

SEQ ID NO: 231 - DNA, 26 bp, Homo sapiens
tacttcctcttacttttctccccaga

SEQ ID NO: 232 - DNA, 26 bp, Homo sapiens
gtcctctggtgctcttgatacctacg

SEQ ID NO: 233 - DNA, 26 bp, Homo sapiens
ccatctttcatccactcatccctcca

SEQ ID NO: 234 - DNA, 26 bp, Homo sapiens
gaatccatcataatgctgactctgaa

SEQ ID NO: 235 - DNA, 23 bp, Homo sapiens
ccctgggtccctctcttatcatc

SEQ ID NO: 236 - DNA, 23 bp, Homo sapiens
gcctccactccacatctcccatc

SEQ ID NO: 237 - DNA, 23 bp, Homo sapiens
agagccgagttgtcccagcagag

SEQ ID NO: 238 - DNA, 23 bp, Homo sapiens
gcaggtctctgagtaggcacaac

SEQ ID NO: 239 - DNA, 26 bp, Homo sapiens
gaaaatgagagagggagaagggaggg

SEQ ID NO: 240 - DNA, 26 bp, Homo sapiens
ccaatacgactggtgttccttgtaag

SEQ ID NO: 241 - DNA, 23 bp, Homo sapiens
ctcagtagacgcagaaaacgctt

SEQ ID NO: 242 - DNA, 26 bp, Homo sapiens
atctctctattcctatttccagccta

SEQ ID NO: 243 - DNA, 23 bp, Homo sapiens
gctggcaggttcggtgtcttgtg

SEQ ID NO: 244 - DNA, 23 bp, Homo sapiens
ggaaaagtctggctgggagatgc

SEQ ID NO: 245 - DNA, 26 bp, Homo sapiens
cagttcacaggtcagtatcctttctg

SEQ ID NO: 246 - DNA, 26 bp, Homo sapiens
ctttgccaacttttgtccttattgcc

SEQ ID NO: 247 - DNA, 26 bp, Homo sapiens
gtaatcaccaccagtctgtaaatagc

SEQ ID NO: 248 - DNA, 24 bp, Homo sapiens
ggtaggacccaagagtcagattta

SEQ ID NO: 249 - DNA, 26 bp, Homo sapiens
ctattgaggatttctaaaaggaggtc

SEQ ID NO: 250 - DNA, 26 bp, Homo sapiens
tgagcaatgagcccctaaataccagg

SEQ ID NO: 251 - DNA, 26 bp, Homo sapiens
gtgtcagcagatgtagccagtagcca

SEQ ID NO: 252 - DNA, 26 bp, Homo sapiens
tgccagatgacaaaatgggaagcgta

SEQ ID NO: 253 - DNA, 23 bp, Homo sapiens
gattggaacctgtgagccaccag

SEQ ID NO: 254 - DNA, 25 bp, Homo sapiens
ctcacaggcaagatacttccacggg

SEQ ID NO: 255 - DNA, 26 bp, Homo sapiens
ggctaaacttccttccttcagaatgc

SEQ ID NO: 256 - DNA, 26 bp, Homo sapiens
ctcaagttgggagggctgatggaaac

SEQ ID NO: 257 - DNA, 26 bp, Homo sapiens
tgggtcttctattttcttagtctggc

SEQ ID NO: 258 - DNA, 26 bp, Homo sapiens
acacccacagaaactaagcagccagt

SEQ ID NO: 259 - DNA, 26 bp, Homo sapiens
cttgtttctctgctcactggctactc

SEQ ID NO: 260 - DNA, 26 bp, Homo sapiens
ggcttttggttttgcttttggtgtcc

SEQ ID NO: 261 - DNA, 25 bp, Homo sapiens
gcacttcactgctccccttgccagc

SEQ ID NO: 262 - DNA, 28 bp, Homo sapiens
ctgttctcccaagtctctaactgtaggc

SEQ ID NO: 263 - DNA, 23 bp, Homo sapiens
ccccagcctcgtgagagagtcta

SEQ ID NO: 264 - DNA, 23 bp, Homo sapiens
cgaagagccccaacccaacacat

SEQ ID NO: 265 - DNA, 26 bp, Homo sapiens
cctgtgcttctgaaatgtgctctgct

SEQ ID NO: 266 - DNA, 26 bp, Homo sapiens
gctatttaccctcagagtagtatcat

SEQ ID NO: 267 - DNA, 26 bp, Homo sapiens
tagccgactcttggcatttatgaggg

SEQ ID NO: 268 - DNA, 26 bp, Homo sapiens
gcacccttgtattacttatctatggc

SEQ ID NO: 269 - DNA, 23 bp, Homo sapiens
gcctgggtgacagagcaagactc

SEQ ID NO: 270 - DNA, 23 bp, Homo sapiens
cagcaaataccccaaatcctgga

SEQ ID NO: 271 - DNA, 23 bp, Homo sapiens
ttccaacctcagtgacagagtga

SEQ ID NO: 272 - DNA, 26 bp, Homo sapiens
acacagttagtaaacaccacaggtat

SEQ ID NO: 273 - DNA, 26 bp, Homo sapiens
ggaagttcaggaatgtggtgtgctct

SEQ ID NO: 274 - DNA, 25 bp, Homo sapiens
caactctggaaaatgcttcttgtgc

SEQ ID NO: 275 - DNA, 26 bp, Homo sapiens
gggattgtgctctcctttgttgacca

SEQ ID NO: 276 - DNA, 26 bp, Homo sapiens
cagaactttcacttgaggtcccatca

SEQ ID NO: 277 - DNA, 27 bp, Homo sapiens
cctgggacactcactcacaaaagcata

SEQ ID NO: 278 - DNA, 28 bp, Homo sapiens
gtagaaagactaaaagacaaacctctca

SEQ ID NO: 279 - DNA, 26 bp, Homo sapiens
gctggaacaatgtatcactttaggga

SEQ ID NO: 280 - DNA, 26 bp, Homo sapiens
tattgaagtttccctgtccgcccagc

SEQ ID NO: 281 - DNA, 24 bp, Homo sapiens
ccttagaaaacataaacacaatac

SEQ ID NO: 282 - DNA, 24 bp, Homo sapiens
gacaagagtagaggtgagtgggta

SEQ ID NO: 283 - DNA, 26 bp, Homo sapiens
gggaccttgtctgctttactcactgc

SEQ ID NO: 284 - DNA, 26 bp, Homo sapiens
cagtgtggagacttatgggaaaacag

SEQ ID NO: 285 - DNA, 25 bp, Homo sapiens
tatcctctgcccaccactgtcactt

SEQ ID NO: 286 - DNA, 26 bp, Homo sapiens
gagagttattcctcaagtgctcccac

SEQ ID NO: 287 - DNA, 26 bp, Homo sapiens
gcagaggtaaaacaggtagaaaggtc

SEQ ID NO: 288 - DNA, 26 bp, Homo sapiens
gtctcctaaagccaaaacaccaccat

SEQ ID NO: 289 - DNA, 26 bp, Homo sapiens
ctaaagtcagggaaggcaaagtaatc

SEQ ID NO: 290 - DNA, 26 bp, Homo sapiens
cagtatgaacgaggtcacagagggta

SEQ ID NO: 291 - DNA, 28 bp, Homo sapiens
tttcttagagatacaatcattctggcag

SEQ ID NO: 292 - DNA, 28 bp, Homo sapiens
cctcataggctgcttcttttgtttctcc

SEQ ID NO: 293 - DNA, 28 bp, Homo sapiens
gcctcggtcagatgttcaatggcaggaa

SEQ ID NO: 294 - DNA, 27 bp, Homo sapiens
tactggctgagggattttcttcaccat

SEQ ID NO: 295 - DNA, 26 bp, Homo sapiens
tcagtttcagagacctcaccacatta

SEQ ID NO: 296 - DNA, 26 bp, Homo sapiens
gtcctcagagaaaatcgcagggagtc

SEQ ID NO: 297 - DNA, 28 bp, Homo sapiens
gggacttacacagccctatgcttccttg

SEQ ID NO: 298 - DNA, 28 bp, Homo sapiens
gccgagtgggctcttctgcctacacaac

SEQ ID NO: 299 - DNA, 23 bp, Homo sapiens
ggcaggagaatcacttgaacccg

SEQ ID NO: 300 - DNA, 23 bp, Homo sapiens
cttggtgttacagccccagaaat

SEQ ID NO: 301 - DNA, 60 bp, Homo sapiens
aataaaatgatggtgtcttaagcataaatcgattgatgtaggagtattagtagtaattga

SEQ ID NO: 302 - DNA, 60 bp, Homo sapiens
tcatgctatttctcatggagagatggcatcgaagatgaaattaattttaaatgattgtct

SEQ ID NO: 303 - DNA, 60 bp, Homo sapiens
atttattttctcagttctggagactcagtcgaaggctagagttatcagaaaatgttaatt

SEQ ID NO: 304 - DNA, 60 bp, Homo sapiens
agttaatatagaaacaacattcttcttttcgacgttttattccatgttctagtctcatcc

SEQ ID NO: 305 - DNA, 60 bp, Homo sapiens
ttgtttgtttactttgctattgagttcttcgatttccagacatttccatacgtcctctga

SEQ ID NO: 306 - DNA, 60 bp, Homo sapiens
acatctgatttaaccaaatctttgccaatcgattgtatcttctataattagcaatagcta

SEQ ID NO: 307 - DNA, 60 bp, Homo sapiens
atatatttaatgaaataaaattactatatcgattacactctgggctagaggttcctaaat

SEQ ID NO: 308 - DNA, 60 bp, Homo sapiens
ccataatttttagcacataaaacttttctcgacagaagtgaagaaacaatcctgaaaaaa

SEQ ID NO: 309 - DNA, 60 bp, Homo sapiens
tgacatgatctaagataatctccttatctcgaggtgagaatttaacacattattaaatta

SEQ ID NO: 310 - DNA, 60 bp, Homo sapiens
atgtcaataaaattgtacaggattacattcgaaaaaaccaaaaaccaaacaaacaaaaaa

SEQ ID NO: 311 - DNA, 60 bp, Homo sapiens
ataaaacagatgaatagactgtaaataatcgacagtaaatttatagcagctgagtagcct

SEQ ID NO: 312 - DNA, 60 bp, Homo sapiens
actgagtttatacaaccttttaaaattttcgagaataactcaggataaaatagatacaag

SEQ ID NO: 313 - DNA, 60 bp, Homo sapiens
tattgtggggtttttttattgtttgttttcgagtaaaaaacagtttttgcaatatcaagt

SEQ ID NO: 314 - DNA, 60 bp, Homo sapiens
gttctttcataaaagggtgaatttttaatcgatgtttaccattttgactgaagatataat

SEQ ID NO: 315 - DNA, 60 bp, Homo sapiens
aaaatgtactatcttgttcataaagttctcgatgtagcctttcttaatctttgggccttt

SEQ ID NO: 316 - DNA, 60 bp, Homo sapiens
atttttccctctattctttaaaaacttttcgagattcttagttgcagaaaggaattagca

SEQ ID NO: 317 - DNA, 60 bp, Homo sapiens
tctgcatttaatgttgcaacatgaggaatcgatgtataatgttgttcagttaaactatgt

SEQ ID NO: 318 - DNA, 60 bp, Homo sapiens
tgactttttttctcctttacagttgtgttcgagcttgttattttctctttcttacctagt

SEQ ID NO: 319 - DNA, 60 bp, Homo sapiens
acacaactatgatgcataagaaaaaaaatcgaatttaaaaacaaaaaaagcccctaatat

SEQ ID NO: 320 - DNA, 60 bp, Homo sapiens
aataactgcagaatatacatttttcccatcgagtctgattgattcagactctgaaatcaa

SEQ ID NO: 321 - DNA, 60 bp, Homo sapiens
tatcatcaccactttacaaatgagaaaatcgaaaatagaaacttacgttatcaggttcaa

SEQ ID NO: 322 - DNA, 60 bp, Homo sapiens
actatattatgacctgtatcaagatgtatcgaaatacaagaatctagtctgattagatta

SEQ ID NO: 323 - DNA, 60 bp, Homo sapiens
atgggatctcagaatagaaaaagaacattcgacctctacatatcatctaaagaaattaac

SEQ ID NO: 324 - DNA, 60 bp, Homo sapiens
atttttaaaatgaaaaaaatgacataattcgacctctcttttcttgctggaaaatatgct

SEQ ID NO: 325 - DNA, 60 bp, Homo sapiens
cagcttaaaataatgggttatggtaacctcgaggtagatacataggaggtgcaaacacaa

SEQ ID NO: 326 - DNA, 60 bp, Homo sapiens
aaacattcaaatttttttcttttactaatcgagggatatgatgttctcagttggattttt

SEQ ID NO: 327 - DNA, 60 bp, Homo sapiens
cagtgtgattttatcaaactcaagaatctcgagtatatgcaagctatgtaagctacttgt

SEQ ID NO: 328 - DNA, 60 bp, Homo sapiens
attttataaagccagaataaccctgatatcgaaccctataaatactgttttttcctataa

SEQ ID NO: 329 - DNA, 60 bp, Homo sapiens
aatgttatgaaatgaagacatatgtgactcgaaaccatctttcataccattaaatatttt

SEQ ID NO: 330 - DNA, 60 bp, Homo sapiens
gctattttaaacaaaacatcatttaatatcgattttggaagggacttaatttcagctacc

SEQ ID NO: 331 - DNA, 60 bp, Homo sapiens
tttgtttctgagcattcattaattctattcgattactgatagatatactgtaggtacatt

SEQ ID NO: 332 - DNA, 60 bp, Homo sapiens
ggttaagaacataagtatacttaaccagtcgatatctgttgagtcctcactatctgactg

SEQ ID NO: 333 - DNA, 60 bp, Homo sapiens
tttattacttgttgaaatggtaacgttttcgagaaccactggattaaatgatctcagctt

SEQ ID NO: 334 - DNA, 60 bp, Homo sapiens
aaatattgtagtacaaatgtctacactctcgatttgtggttgttgccaggaatggaggca

SEQ ID NO: 335 - DNA, 60 bp, Homo sapiens
acagaaatgagaaaaacaatagagaatatcgactctaaagtctatgtagagaagcaaagg

SEQ ID NO: 336 - DNA, 60 bp, Homo sapiens
accaataaaagggaaagagtagagatactcgagaaaatgaaggaaatgattgccataaaa

SEQ ID NO: 337 - DNA, 60 bp, Homo sapiens
aaacagcttaagtttattttgtgtatgatcgataagctacccttccttaggataacaaat

SEQ ID NO: 338 - DNA, 60 bp, Homo sapiens
ccggataattttttttatttcttacttttcgatcctacctatttccccaaccattaatat

SEQ ID NO: 339 - DNA, 60 bp, Homo sapiens
tttgattcctatagttttgcagtataattcgaagatcaaaacatgcaaatactagcagtc

SEQ ID NO: 340 - DNA, 60 bp, Homo sapiens
caggactgtaaaagagccttttcttctttcgattggaacctttatgtctggactctgaaa

SEQ ID NO: 341 - DNA, 60 bp, Homo sapiens
accaaaagtcagaggaacaaataatttgtcgattcttcagaacttccttgatattttaaa

SEQ ID NO: 342 - DNA, 60 bp, Homo sapiens
attctctatctccatgaatttaattgtttcgaccttcagaactttctgtttaggtagaat

SEQ ID NO: 343 - DNA, 60 bp, Homo sapiens
ctgcctttttggcctttatatacacacttcgaaatcctttgttggattcagatttctaaa

SEQ ID NO: 344 - DNA, 60 bp, Homo sapiens
aaacatcatttctaccagcaagtttatttcgaacaatgtcatttgttttcattacacttt

SEQ ID NO: 345 - DNA, 60 bp, Homo sapiens
aaacagaacccggatttttacagactgatcgaagttaactgcacatccttttttttatct

SEQ ID NO: 346 - DNA, 60 bp, Homo sapiens
gcctttaataaatgcttgttgtgataattcgagtcatttaggttgaaagggaccagttgt

SEQ ID NO: 347 - DNA, 60 bp, Homo sapiens
gaatgcacaatgtaaaactattaacctttcgaacttatctctatttttactgtactgtgg

SEQ ID NO: 348 - DNA, 60 bp, Homo sapiens
aaacagaacccggatttttacagactgatcgactcaaaaattactgtatgtgatatggga

SEQ ID NO: 349 - DNA, 60 bp, Homo sapiens
tgtcccagtaaatcgctggggaatagaatcgataagaactatgaaaaattttaatttaaa

SEQ ID NO: 350 - DNA, 60 bp, Homo sapiens
atcactttcagaaaagaatagaggtttttcgatattcaagctcacaaaataagctcaaac

SEQ ID NO: 351 - DNA, 26 bp, Homo sapiens
gtggactttcctgtaacatcctttta

SEQ ID NO: 352 - DNA, 25 bp, Homo sapiens
ggctgggttctaaagatgaagactt

SEQ ID NO: 353 - DNA, 23 bp, Homo sapiens
gtgggcacttggctctgagatgc

SEQ ID NO: 354 - DNA, 23 bp, Homo sapiens
gccctcaacggtggagtaggata

SEQ ID NO: 355 - DNA, 23 bp, Homo sapiens
ggtgggagagacactgaggctgt

SEQ ID NO: 356 - DNA, 23 bp, Homo sapiens
caccacctacctgccaagttgct

SEQ ID NO: 357 - DNA, 24 bp, Homo sapiens
gatgtcccactaccttgcctgagg

SEQ ID NO: 358 - DNA, 23 bp, Homo sapiens
gcagggagacttggacctattgg

SEQ ID NO: 359 - DNA, 28 bp, Homo sapiens
ccagcagtggatgagggctcgcctgtct

SEQ ID NO: 360 - DNA, 28 bp, Homo sapiens
cacagcagcctctttcatcacagcccag

SEQ ID NO: 361 - DNA, 28 bp, Homo sapiens
ggaatgatagagatagggagaaatggaa

SEQ ID NO: 362 - DNA, 28 bp, Homo sapiens
gagaagcagacggaaactgtgactgaca

SEQ ID NO: 363 - DNA, 28 bp, Homo sapiens
ctcaagcaactcagcagcataaaacaca

SEQ ID NO: 364 - DNA, 28 bp, Homo sapiens
cttccccactcatttcttgtgattctat

SEQ ID NO: 365 - DNA, 26 bp, Homo sapiens
tttatttgtcaagtgccaaggaatgc

SEQ ID NO: 366 - DNA, 26 bp, Homo sapiens
taggaaacaccagtgagtccaaggct

SEQ ID NO: 367 - DNA, 28 bp, Homo sapiens
cacagtcccctcttactacagcgtgaaa

SEQ ID NO: 368 - DNA, 28 bp, Homo sapiens
acactatttcattgtagaggttaccacg

SEQ ID NO: 369 - DNA, 26 bp, Homo sapiens
cttggaaatgtcagacccttgcccat

SEQ ID NO: 370 - DNA, 25 bp, Homo sapiens
attgagccgtgtgtttggtgcttgc

SEQ ID NO: 371 - DNA, 26 bp, Homo sapiens
ggctggatggcagtagaatgctgttc

SEQ ID NO: 372 - DNA, 26 bp, Homo sapiens
ctccattcatcgcactctactttagc

SEQ ID NO: 373 - DNA, 23 bp, Homo sapiens
cctgcctcaacctcccaagtagc

SEQ ID NO: 374 - DNA, 24 bp, Homo sapiens
ctgaggtaaacggagtaaagggta

SEQ ID NO: 375 - DNA, 24 bp, Homo sapiens
ctgttggtagttcctaacctcata

SEQ ID NO: 376 - DNA, 25 bp, Homo sapiens
caataagaagtaaacaaagcataac

SEQ ID NO: 377 - DNA, 26 bp, Homo sapiens
gccccaagggaataaaactaagacta

SEQ ID NO: 378 - DNA, 26 bp, Homo sapiens
cttcttctctcttcatcacaacagtc

SEQ ID NO: 379 - DNA, 26 bp, Homo sapiens
cttcttggttgtctatcctttcagat

SEQ ID NO: 380 - DNA, 26 bp, Homo sapiens
caagttggcaccgagtggtttcctaa

SEQ ID NO: 381 - DNA, 26 bp, Homo sapiens
accaaaagttctcactggaggcacca

SEQ ID NO: 382 - DNA, 26 bp, Homo sapiens
ctcgtccacaatgtccagatttaccc

SEQ ID NO: 383 - DNA, 23 bp, Homo sapiens
ccatccccaggaccccactgtgc

SEQ ID NO: 384 - DNA, 28 bp, Homo sapiens
cagacaaaggtgacttcagagcaagaac

SEQ ID NO: 385 - DNA, 28 bp, Homo sapiens
gtgactcccatctttcccatagaaccag

SEQ ID NO: 386 - DNA, 25 bp, Homo sapiens
cactcttgtgtcgctcctgaggcta

SEQ ID NO: 387 - DNA, 26 bp, Homo sapiens
cgggattcataaaggagaaagcaaga

SEQ ID NO: 388 - DNA, 26 bp, Homo sapiens
gtgccaaccccacagagcaacttatg

SEQ ID NO: 389 - DNA, 28 bp, Homo sapiens
ggaaaaggaaggacgcaaatggtgtcac

SEQ ID NO: 390 - DNA, 28 bp, Homo sapiens
gtcttgtgccagttttcaaagggagagc

SEQ ID NO: 391 - DNA, 26 bp, Homo sapiens
taagattattgagcactctctctgtg

SEQ ID NO: 392 - DNA, 23 bp, Homo sapiens
gggtgtagggcacagattcttac

SEQ ID NO: 393 - DNA, 25 bp, Homo sapiens
tccagcaaaagtatccttcaacagg

SEQ ID NO: 394 - DNA, 25 bp, Homo sapiens
ctgctgtatgctcaggctttgctcc

SEQ ID NO: 395 - DNA, 26 bp, Homo sapiens
tacagcaatagaaaggagtgcggtat

SEQ ID NO: 396 - DNA, 26 bp, Homo sapiens
gtcaaagccagtgtccagaagggtat

SEQ ID NO: 397 - DNA, 26 bp, Homo sapiens
gcctcattaggaaagtgtagaagaga

SEQ ID NO: 398 - DNA, 26 bp, Homo sapiens
gactatcctggagactctggaaaact

SEQ ID NO: 399 - DNA, 27 bp, Homo sapiens
gtagaaagactaaaagacaaacctctc

SEQ ID NO: 400 - DNA, 23 bp, Homo sapiens
cacgcacagcctccaagaaacta

SEQ ID NO: 401 - DNA, 26 bp, Homo sapiens
cttgacctttatttactatgcttggc

SEQ ID NO: 402 - DNA, 26 bp, Homo sapiens
caagggacctctcaaagcagaagcaa

SEQ ID NO: 403 - DNA, 24 bp, Homo sapiens
attttggtgacagactgggcttct

SEQ ID NO: 404 - DNA, 28 bp, Homo sapiens
catctctctcttcttcattacagtcccc

SEQ ID NO: 405 - DNA, 28 bp, Homo sapiens
aaggattggtgaactcaaagacaggtta

SEQ ID NO: 406 - DNA, 28 bp, Homo sapiens
ggtattttgagagagagtccacattgac

SEQ ID NO: 407 - DNA, 26 bp, Homo sapiens
cggaactttagcatctggaagaagaa

SEQ ID NO: 408 - DNA, 26 bp, Homo sapiens
caacatcagagaggtgagaaacaacc

SEQ ID NO: 409 - DNA, 26 bp, Homo sapiens
cagtcttttggtagagcaatcaggat

SEQ ID NO: 410 - DNA, 26 bp, Homo sapiens
gaacatctccttgacaaccctcccct

SEQ ID NO: 411 - DNA, 26 bp, Homo sapiens
gttggcgacgctcttcaataagtttt

SEQ ID NO: 412 - DNA, 26 bp, Homo sapiens
ggagcaacacaagccaaacacagcag

SEQ ID NO: 413 - DNA, 26 bp, Homo sapiens
ccatccagagaagtaaccactatccc

SEQ ID NO: 414 - DNA, 26 bp, Homo sapiens
gactgcttggtcattttgtgcctcag

SEQ ID NO: 415 - DNA, 26 bp, Homo sapiens
cctgtgaggtccagtttggtagcccc

SEQ ID NO: 416 - DNA, 28 bp, Homo sapiens
ccttcagtttccttgtgtgctgtagaca

SEQ ID NO: 417 - DNA, 23 bp, Homo sapiens
actcactccttcccttcagcagc

SEQ ID NO: 418 - DNA, 23 bp, Homo sapiens
actgcctcaggagttggctgcct

SEQ ID NO: 419 - DNA, 23 bp, Homo sapiens
atgaaacagaaatgagaaaaaca

SEQ ID NO: 420 - DNA, 25 bp, Homo sapiens
tctatgtagagaagcaaagggccaa

SEQ ID NO: 421 - DNA, 26 bp, Homo sapiens
gtgatgtgtgtcttagaggagtaacc

SEQ ID NO: 422 - DNA, 26 bp, Homo sapiens
agccctctttgtcccctatccagttg

SEQ ID NO: 423 - DNA, 28 bp, Homo sapiens
ttgggtgggttttctgctctggaggtat

SEQ ID NO: 424 - DNA, 28 bp, Homo sapiens
atcaggagaagagcaagagttgaaatca

SEQ ID NO: 425 - DNA, 23 bp, Homo sapiens
ctcacctcagtctcccaagtagc

SEQ ID NO: 426 - DNA, 23 bp, Homo sapiens
gtgtgccactccaatcactgtat

SEQ ID NO: 427 - DNA, 26 bp, Homo sapiens
ttattgaagagactgtcctttcccta

SEQ ID NO: 428 - DNA, 26 bp, Homo sapiens
gtgaatagtgccgcaataaacatacg

SEQ ID NO: 429 - DNA, 26 bp, Homo sapiens
attatgcctcctacattccctccccg

SEQ ID NO: 430 - DNA, 26 bp, Homo sapiens
cctccctgtcttccctgttgataaaa

SEQ ID NO: 431 - DNA, 26 bp, Homo sapiens
gaatgagtgaatacagccagggaatg

SEQ ID NO: 432 - DNA, 26 bp, Homo sapiens
tggctatggagatgtcacctgaaggg

SEQ ID NO: 433 - DNA, 28 bp, Homo sapiens
acccatccccaagcatttatcctttgtg

SEQ ID NO: 434 - DNA, 28 bp, Homo sapiens
gttgctgctgttttgctgtcttgtgtcc

SEQ ID NO: 435 - DNA, 23 bp, Homo sapiens
ggcgaataggagaggtgatgacg

SEQ ID NO: 436 - DNA, 23 bp, Homo sapiens
gccctgtttcccacgaggtagga

SEQ ID NO: 437 - DNA, 28 bp, Homo sapiens
gtcatctaaactacctccccaggcaagt

SEQ ID NO: 438 - DNA, 28 bp, Homo sapiens
ggtttagaatggatgatttgcgtaatgg

SEQ ID NO: 439 - DNA, 23 bp, Homo sapiens
cagcggcaaacacaggcagatgt

SEQ ID NO: 440 - DNA, 23 bp, Homo sapiens
ccccagagccccatcctgtcaat

SEQ ID NO: 441 - DNA, 26 bp, Homo sapiens
cctctgaatggttatgaatggttgtg

SEQ ID NO: 442 - DNA, 26 bp, Homo sapiens
cctgcctggtcaacccacagaatcat

SEQ ID NO: 443 - DNA, 24 bp, Homo sapiens
attcagtgtaatccctatcaaaat

SEQ ID NO: 444 - DNA, 24 bp, Homo sapiens
tctgccataaagtcattctcaaaa

SEQ ID NO: 445 - DNA, 26 bp, Homo sapiens
ccattgaacagtagagacagggtccc

SEQ ID NO: 446 - DNA, 26 bp, Homo sapiens
ggcactctttctgctctattttgtgg

SEQ ID NO: 447 - DNA, 26 bp, Homo sapiens
gttattactggttgatgactccacat

SEQ ID NO: 448 - DNA, 28 bp, Homo sapiens
gcaatctcagaaatcaacactaaagaac

SEQ ID NO: 449 - DNA, 26 bp, Homo sapiens
gatgtctgagaatgtggagtgtgcta

SEQ ID NO: 450 - DNA, 26 bp, Homo sapiens
tttaccgccttttcctcccacctctc

SEQ ID NO: 451 - DNA, 60 bp, Homo sapiens
tgagctaataaactatttctggttttgctcgagttccacattctataccatgtttctttt

SEQ ID NO: 452 - DNA, 60 bp, Homo sapiens
tgtcttctggattttattattttttctgtcgaaatgatcatataattttactccttcagt

SEQ ID NO: 453 - DNA, 60 bp, Homo sapiens
tttttttttttttcagactttcctgtggtcgacaaggaggtaagattagatacatacatc

SEQ ID NO: 454 - DNA, 60 bp, Homo sapiens
tacattcggtcactgaaaacattagttttcgatgagggtgtcttttctccagtttatgtt

SEQ ID NO: 455 - DNA, 60 bp, Homo sapiens
tttgagtgttcttttcttctgtggaatatcgattgctcacatgtggttgttaataaaact

SEQ ID NO: 456 - DNA, 60 bp, Homo sapiens
gcatgtcaggtaatgaaaacagacattatcgaaagacatttatacacgactttaaccgtt

SEQ ID NO: 457 - DNA, 60 bp, Homo sapiens
tgaataatttttaatttacataaatggatcgattctttttaaacatatagcaccaggcag

SEQ ID NO: 458 - DNA, 60 bp, Homo sapiens
aaacttcagtttgctttactgtaaaatgtcgacttactgttgctgaagagagagataaga

SEQ ID NO: 459 - DNA, 60 bp, Homo sapiens
gaattgggtttcttctttcttttgaatgtcgactttaatttatcctttctttgaaagcca

SEQ ID NO: 460 - DNA, 60 bp, Homo sapiens
ttacactcattgattttcagatattaaatcgaattcttgtacatgtaagtggcagataag

SEQ ID NO: 461 - DNA, 60 bp, Homo sapiens
cagaagattggagttatcaaatgaattatcgaagagaagcttcagtgcagatggatttga

SEQ ID NO: 462 - DNA, 60 bp, Homo sapiens
gccaaggcaaaactccaattattagttttcgaaagtttatatgtttaggtttttttgttt

SEQ ID NO: 463 - DNA, 60 bp, Homo sapiens
aagggactagaagaagaaatcattaagttcgacaatttttaaataactgcctgaagataa

SEQ ID NO: 464 - DNA, 60 bp, Homo sapiens
cttaaaaatcaaccaaagtacagtattttcgaactgcagacttagagctgtcaagcggtt

SEQ ID NO: 465 - DNA, 60 bp, Homo sapiens
tagctttgcatgaactaaatgtcaaatttcgaaaaattatactgcgagaaaattcatacc

SEQ ID NO: 466 - DNA, 60 bp, Homo sapiens
ggcaaatctcgtatagatttgtatttcatcgatataattttaatgagtgctcaattacag

SEQ ID NO: 467 - DNA, 60 bp, Homo sapiens
ttagaggcaaaatataacacagatgagatcgacatattgcaaaatttatactgtggtcag

SEQ ID NO: 468 - DNA, 60 bp, Homo sapiens
aactataaaaaggatttagatgagcccttcgagatttaaaacacacacacacacatattt

SEQ ID NO: 469 - DNA, 60 bp, Homo sapiens
tctaaacttggagtcagaagaactgagttcgagctagaattccaagcttctctcctattg

SEQ ID NO: 470 - DNA, 60 bp, Homo sapiens
ttgctattctttcttcattcatcactcttcgatttgtaaacttacaaaacctaaacattc

SEQ ID NO: 471 - DNA, 60 bp, Homo sapiens
ccatttgtatttcctgtctgtgaatttttcgaaatgtatgaatcagaataattcagagga

SEQ ID NO: 472 - DNA, 60 bp, Homo sapiens
ttcactcaaagcctgaatgattttagggtcgaaagaaaccagaaagtaactatgcaagta

SEQ ID NO: 473 - DNA, 60 bp, Homo sapiens
ctgaaatagaaggaaagatattaaggactcgagtgaaaaaaggaaggaagctataactta

SEQ ID NO: 474 - DNA, 60 bp, Homo sapiens
ttgcttgttgttctgtgatttgttttaatcgacagaacatcttctggtgttctgttcacc

SEQ ID NO: 475 - DNA, 60 bp, Homo sapiens
atttcagcagagtttacagactgtaatttcgaggctcagacctcatgtctctcttgttgc

SEQ ID NO: 476 - DNA, 60 bp, Homo sapiens
agtaaggcagaagtgatattgtatgacttcgaacttttttttttctactgcatacatata

SEQ ID NO: 477 - DNA, 60 bp, Homo sapiens
tatgtaagctttgctggagaagttttagtcgagtttgaggcactaaggtaaataaaacat

SEQ ID NO: 478 - DNA, 60 bp, Homo sapiens
gcagattatagctggaattcacaatatttcgaaaatctgagtttaaaaaaagagcacaga

SEQ ID NO: 479 - DNA, 60 bp, Homo sapiens
attttcaggtaggatcataaaggacttatcgaaaagaaaaaaaagaattatttaacttaa

SEQ ID NO: 480 - DNA, 60 bp, Homo sapiens
cttcaagtagtcacagagaatgacagaatcgaagtactgttttgtttattgctgtatccc

SEQ ID NO: 481 - DNA, 60 bp, Homo sapiens
agtcttcatagagatcattcacatgttttcgaaagtattaagttcttctttttttttttt

SEQ ID NO: 482 - DNA, 60 bp, Homo sapiens
ccatctgattacagttacatgtatcatatcgaatgacctcccttctattcctgttaataa

SEQ ID NO: 483 - DNA, 60 bp, Homo sapiens
acttaacttccttctaaaacaacctttttcgactttagttggtgacatcatcatccccct

SEQ ID NO: 484 - DNA, 60 bp, Homo sapiens
cagctattgactgtgtatctttagaaaatcgatctctaaataaactgcagaggaggaggt

SEQ ID NO: 485 - DNA, 60 bp, Homo sapiens
ccttaaaagatgtgaaacagagaacagctcgaattatttttgtaatttatatttttctag

SEQ ID NO: 486 - DNA, 60 bp, Homo sapiens
agtttttccacctatgacaaataatctatcgattagtaaaagaaaaaaatttgaatgttt

SEQ ID NO: 487 - DNA, 60 bp, Homo sapiens
ctcctcttcatcttccacaatagtcaagtcgacagaatttttgtattcttttgtaaatag

SEQ ID NO: 488 - DNA, 60 bp, Homo sapiens
gtagttgatttatttgctacagttgtaatcgaaagattagttacttcatgaatctttgtt

SEQ ID NO: 489 - DNA, 60 bp, Homo sapiens
aatattagaatgggctactcgcctttcctcgagacgtgggcaaatgtatttaaaagtcat

SEQ ID NO: 490 - DNA, 60 bp, Homo sapiens
aagttcaacacagttgcagaatataggatcgaaatgtttcttcagaatcatgttttatat

SEQ ID NO: 491 - DNA, 60 bp, Homo sapiens
cccctatgtggaattatgtagttatctctcgaaacagtatgttctggcataaaaacagac

SEQ ID NO: 492 - DNA, 60 bp, Homo sapiens
ctgtacttagggtatcataaggctgaaatcgatttcctgtcttacccacacattcaggct

SEQ ID NO: 493 - DNA, 60 bp, Homo sapiens
gaatattttatttgccatttcaacaaaatcgaaatgcggctgcttttcaaggtaagtgga

SEQ ID NO: 494 - DNA, 60 bp, Homo sapiens
ccaccaccaccagtggtcctcccagtattcgatggaatagtgaaaaccttcaccaattgc

SEQ ID NO: 495 - DNA, 60 bp, Homo sapiens
cttcaactcaatataaataagatctctatcgaggtaactaggtctttacttgcctgaatc

SEQ ID NO: 496 - DNA, 60 bp, Homo sapiens
gtgtcctttacatcctaagtacagagattcgatggagaaagtggtgtgcacaccagtaca

SEQ ID NO: 497 - DNA, 60 bp, Homo sapiens
agaaacaagtaagagattctaaaagttctcgattttgaaggtaatttgcacttttctact

SEQ ID NO: 498 - DNA, 60 bp, Homo sapiens
atttcaccacttttattcaacataatactcgacagaatgaagaacaaaaacgatttgatc

SEQ ID NO: 499 - DNA, 60 bp, Homo sapiens
ttaaaatgatttagcagggcacaaaatgtcgataaataatacgacatttgcatcactacc

SEQ ID NO: 500 - DNA, 60 bp, Homo sapiens
atttgtttttcagaatagaaatgtactgtcgacctatcttcagactcactgtttctttcc

SEQ ID NO: 501 - DNA, 23 bp, Homo sapiens
tacgcctgccatccactctgaat

SEQ ID NO: 502 - DNA, 23 bp, Homo sapiens
ggattacagatgtgagccaccac

SEQ ID NO: 503 - DNA, 28 bp, Homo sapiens
atgtgttgctctactggtttgtaagtta

SEQ ID NO: 504 - DNA, 28 bp, Homo sapiens
agacacatccaataaagaaactgcgggc

SEQ ID NO: 505 - DNA, 26 bp, Homo sapiens
ggtctatgtgatgctgaagttttggg

SEQ ID NO: 506 - DNA, 26 bp, Homo sapiens
gccctggttttctgtaggcaatcagc

SEQ ID NO: 507 - DNA, 26 bp, Homo sapiens
atgaagggaaaacattggagtggaat

SEQ ID NO: 508 - DNA, 26 bp, Homo sapiens
cacacacctacagtgaactcattttc

SEQ ID NO: 509 - DNA, 27 bp, Homo sapiens
atagtggtcaaagtgaaaagtctgagc

SEQ ID NO: 510 - DNA, 27 bp, Homo sapiens
gcaccttgagcagcaccataggagttt

SEQ ID NO: 511 - DNA, 23 bp, Homo sapiens
gctcacagtctcctggctgaacc

SEQ ID NO: 512 - DNA, 23 bp, Homo sapiens
gagggttctgcggtatctgccta

SEQ ID NO: 513 - DNA, 25 bp, Homo sapiens
aagcatccttccagtctgtgtttgc

SEQ ID NO: 514 - DNA, 25 bp, Homo sapiens
taatcatttagaggcggctcaaagc

SEQ ID NO: 515 - DNA, 26 bp, Homo sapiens
ccagaggttgatggtggtttcttgtg

SEQ ID NO: 516 - DNA, 26 bp, Homo sapiens
ggtttatttactggagggtcagagga

SEQ ID NO: 517 - DNA, 26 bp, Homo sapiens
cctcctcttggttgattttgctgtta

SEQ ID NO: 518 - DNA, 26 bp, Homo sapiens
cacctttggttggaagtttagggagg

SEQ ID NO: 519 - DNA, 26 bp, Homo sapiens
ctacaaccagtgaaagatggctccaa

SEQ ID NO: 520 - DNA, 26 bp, Homo sapiens
ctgccatccccactaatgtcacctct

SEQ ID NO: 521 - DNA, 20 bp, Homo sapiens
gaatgcagaagattggagtt

SEQ ID NO: 522 - DNA, 20 bp, Homo sapiens
tgcagatggatttgagatgc

SEQ ID NO: 523 - DNA, 25 bp, Homo sapiens
gcacactctgaggattttagacttc

SEQ ID NO: 524 - DNA, 28 bp, Homo sapiens
cacaaatagccatcaaaatgtcaacaag

SEQ ID NO: 525 - DNA, 26 bp, Homo sapiens
gaacaactacctgtgcctcaaaagac

SEQ ID NO: 526 - DNA, 26 bp, Homo sapiens
caactctgaagcctactgtgtttatc

SEQ ID NO: 527 - DNA, 23 bp, Homo sapiens
caacagttcttcagcacccaagt

SEQ ID NO: 528 - DNA, 25 bp, Homo sapiens
gaaggctgacttaggtttccacata

SEQ ID NO: 529 - DNA, 26 bp, Homo sapiens
gggtgtcactgttctttccagatagc

SEQ ID NO: 530 - DNA, 26 bp, Homo sapiens
tcttctcattctggaacaaccgattg

SEQ ID NO: 531 - DNA, 26 bp, Homo sapiens
tcacaactcctgtttggaatcgccct

SEQ ID NO: 532 - DNA, 26 bp, Homo sapiens
cttcctcaaatggtccttcaataaca

SEQ ID NO: 533 - DNA, 26 bp, Homo sapiens
acccatagattccaatacgctctttg

SEQ ID NO: 534 - DNA, 26 bp, Homo sapiens
gtagttccaagttcaggagatacacc

SEQ ID NO: 535 - DNA, 23 bp, Homo sapiens
actgtagtttccaggacctcagg

SEQ ID NO: 536 - DNA, 26 bp, Homo sapiens
agcattgacttttgtccctcttgggc

SEQ ID NO: 537 - DNA, 23 bp, Homo sapiens
cctacccctctcttagcctcact

SEQ ID NO: 538 - DNA, 23 bp, Homo sapiens
cctggaggtagtgatggctgagg

SEQ ID NO: 539 - DNA, 26 bp, Homo sapiens
cggtttctccaatgccctttattcta

SEQ ID NO: 540 - DNA, 26 bp, Homo sapiens
gtgagaagggtaaaatcttaggcagc

SEQ ID NO: 541 - DNA, 23 bp, Homo sapiens
ctggcactggagagcagcaccaa

SEQ ID NO: 542 - DNA, 23 bp, Homo sapiens
gcgaacactgccttgaatctgcc

SEQ ID NO: 543 - DNA, 28 bp, Homo sapiens
ttgtaaggcattgacctgttctaccata

SEQ ID NO: 544 - DNA, 26 bp, Homo sapiens
aaaagcaagtccataactccagaaca

SEQ ID NO: 545 - DNA, 23 bp, Homo sapiens
ccttggtgccgctggctgtctgc

SEQ ID NO: 546 - DNA, 26 bp, Homo sapiens
ccgttttccccttcattgttgtgtgc

SEQ ID NO: 547 - DNA, 26 bp, Homo sapiens
ttcccacagcaaggcataggtttcta

SEQ ID NO: 548 - DNA, 26 bp, Homo sapiens
gtgaagagatactcatttgtggattg

SEQ ID NO: 549 - DNA, 28 bp, Homo sapiens
tcaacgaggtttcaccaaagccaccaat

SEQ ID NO: 550 - DNA, 28 bp, Homo sapiens
cagagttgctgaatgagaggagaggacg

SEQ ID NO: 551 - DNA, 26 bp, Homo sapiens
gctggcaggaaaagtgtagactgttt

SEQ ID NO: 552 - DNA, 26 bp, Homo sapiens
gaggcaaagtaataagagaggaaaac

SEQ ID NO: 553 - DNA, 26 bp, Homo sapiens
cgcacactcaccctgactttgttatc

SEQ ID NO: 554 - DNA, 26 bp, Homo sapiens
ccatttgtaaactgctgacttcattg

SEQ ID NO: 555 - DNA, 26 bp, Homo sapiens
ttgctcagccccaggaaataacttgg

SEQ ID NO: 556 - DNA, 26 bp, Homo sapiens
gtggataggcagaatcaataatactg

SEQ ID NO: 557 - DNA, 26 bp, Homo sapiens
cagataatgaaacaaccaccatcggt

SEQ ID NO: 558 - DNA, 26 bp, Homo sapiens
gacttcctattcctgctgcctatggt

SEQ ID NO: 559 - DNA, 28 bp, Homo sapiens
tcataacacctccctcctctgtggaaca

SEQ ID NO: 560 - DNA, 28 bp, Homo sapiens
ccttcgggagatgtatttaggagttgcg

SEQ ID NO: 561 - DNA, 24 bp, Homo sapiens
accaaggtatctcctgaaagaacc

SEQ ID NO: 562 - DNA, 26 bp, Homo sapiens
taatacttaggctgggagcgatggct

SEQ ID NO: 563 - DNA, 23 bp, Homo sapiens
ggaatgaagcccagttgagttgc

SEQ ID NO: 564 - DNA, 23 bp, Homo sapiens
gcccaaacccgtgtggtccaaca

SEQ ID NO: 565 - DNA, 24 bp, Homo sapiens
tttccttttgacttttcccctaat

SEQ ID NO: 566 - DNA, 24 bp, Homo sapiens
attggcaggaggaataaggaagag

SEQ ID NO: 567 - DNA, 26 bp, Homo sapiens
attgagaggacaccaggtttgaatcc

SEQ ID NO: 568 - DNA, 26 bp, Homo sapiens
ggtgattaggttatgagggctccgtc

SEQ ID NO: 569 - DNA, 26 bp, Homo sapiens
atttgagttacttgtttagggctcct

SEQ ID NO: 570 - DNA, 28 bp, Homo sapiens
caatggttgaacaatgactaaactgcca

SEQ ID NO: 571 - DNA, 26 bp, Homo sapiens
tatgcctctgggtaatgggctaacag

SEQ ID NO: 572 - DNA, 26 bp, Homo sapiens
cttgacctttatttactatgcttggc

SEQ ID NO: 573 - DNA, 23 bp, Homo sapiens
aggatggcacggtggctctaagc

SEQ ID NO: 574 - DNA, 24 bp, Homo sapiens
atcaagccctctctatccaccata

SEQ ID NO: 575 - DNA, 23 bp, Homo sapiens
cagccttttccttgtcctctcaa

SEQ ID NO: 576 - DNA, 23 bp, Homo sapiens
gcccaggagttcaagaccagcct

SEQ ID NO: 577 - DNA, 23 bp, Homo sapiens
cagagacggtttccacggttgtc

SEQ ID NO: 578 - DNA, 23 bp, Homo sapiens
acacctgggactggctccaactc

SEQ ID NO: 579 - DNA, 26 bp, Homo sapiens
cacttccgttcaacttttactggagg

SEQ ID NO: 580 - DNA, 26 bp, Homo sapiens
atgttgcctccacagaaaccagaaaa

SEQ ID NO: 581 - DNA, 23 bp, Homo sapiens
taatgtcctctgggctcatccat

SEQ ID NO: 582 - DNA, 24 bp, Homo sapiens
ctgctctattggtctaagtgtctg

SEQ ID NO: 583 - DNA, 27 bp, Homo sapiens
gttagaaatctggtatgaaatgactgg

SEQ ID NO: 584 - DNA, 26 bp, Homo sapiens
tttattaggaatgctggagaactcag

SEQ ID NO: 585 - DNA, 28 bp, Homo sapiens
gcctaaactcctcaaataatgctaatga

SEQ ID NO: 586 - DNA, 28 bp, Homo sapiens
aatgttggcagacctggaagaaccctat

SEQ ID NO: 587 - DNA, 26 bp, Homo sapiens
gctcaactctgccatcttactgtgaa

SEQ ID NO: 588 - DNA, 26 bp, Homo sapiens
ctcaccttcctctcaccttttgaccc

SEQ ID NO: 589 - DNA, 28 bp, Homo sapiens
ggctcaaagagaatgtgtaagagagaaa

SEQ ID NO: 590 - DNA, 28 bp, Homo sapiens
gtccctgaacttgattgcctgtggtcta

SEQ ID NO: 591 - DNA, 26 bp, Homo sapiens
ctgtttactcccttggcatctgctgc

SEQ ID NO: 592 - DNA, 26 bp, Homo sapiens
ggctatttgctcttgctacttctttc

SEQ ID NO: 593 - DNA, 26 bp, Homo sapiens
gctgccttcctgttttgataccctgc

SEQ ID NO: 594 - DNA, 26 bp, Homo sapiens
aatactaccaaatgacagcccagcat

SEQ ID NO: 595 - DNA, 24 bp, Homo sapiens
gacttccagtttcaaaatggtggc

SEQ ID NO: 596 - DNA, 26 bp, Homo sapiens
attgtgttgaggtatgttccttccat

SEQ ID NO: 597 - DNA, 28 bp, Homo sapiens
gacatctctatctttacagactgcccct

SEQ ID NO: 598 - DNA, 28 bp, Homo sapiens
gcctcccaaagcactgagattatgacat

SEQ ID NO: 599 - DNA, 24 bp, Homo sapiens
tttcttggtatctttcataggaat

SEQ ID NO: 600 - DNA, 24 bp, Homo sapiens
aataacagcagtgaagaatacctt

SEQ ID NO: 601 - DNA, 60 bp, Homo sapiens
cccgatttgcttaatctcggcatagatttcgaattatatttgatttgtcttatctcctca

SEQ ID NO: 602 - DNA, 60 bp, Homo sapiens
cccgatttgcttaatctcggcatagatttcgaatcaaatagtttatttagagaacctcca

SEQ ID NO: 603 - DNA, 60 bp, Homo sapiens
agtgcctgtgaaagtgtttctgaattcctcgatcagcttctcagaaaaatgaaattgact

SEQ ID NO: 604 - DNA, 60 bp, Homo sapiens
cccgatttgcttaatctcggcatagatttcgaattacagattttatccaaaatatctgtc

SEQ ID NO: 605 - DNA, 60 bp, Homo sapiens
agtgcctgtgaaagtgtttctgaattcctcgacttcttgcttttgatttattgaagaatc

SEQ ID NO: 606 - DNA, 60 bp, Homo sapiens
agtgcctgtgaaagtgtttctgaattcctcgacaaaaatcagagggggtttaatcacctt

SEQ ID NO: 607 - DNA, 60 bp, Homo sapiens
agttattttatagtttagtacatagttgtcgagtgtgtgaggaaaaattattctgaaaat

SEQ ID NO: 608 - DNA, 60 bp, Homo sapiens
catgttttggagtcttttatctagtttatcgaggagacagccgttgtccccagggctcct

SEQ ID NO: 609 - DNA, 60 bp, Homo sapiens
aagggctcgggagctccctcggcacacctcgaggagtgccaggcatctactgctctgtcc

SEQ ID NO: 610 - DNA, 60 bp, Homo sapiens
acagagccctcagaaataatgccgcatatcgatgagaagtctgattcttgctttttggag

SEQ ID NO: 611 - DNA, 60 bp, Homo sapiens
tggggtgattgggttcatttatgtattatcgagagacataacaatatttataaagtttgg

SEQ ID NO: 612 - DNA, 60 bp, Homo sapiens
aaacttggtatgtgtctaataaacagcttcgagtgctggtttgggtcggagtgctggttc

SEQ ID NO: 613 - DNA, 60 bp, Homo sapiens
ctttaagtcaacaaatacactgaagacttcgaagtaatgaagagggaggtggttttcttt

SEQ ID NO: 614 - DNA, 60 bp, Homo sapiens
agccagccattgttaacatagttcaacttcgaaggaatgattcttttttcttaaccatgc

SEQ ID NO: 615 - DNA, 60 bp, Homo sapiens
cctccagtcctatttctttttttttttttcgagtgctggtttgggtcggagtgctggttc

SEQ ID NO: 616 - DNA, 60 bp, Homo sapiens
agtgcctgtgaaagtgtttctgaattcctcgaattattatgagtctccgtccacgcagtg

SEQ ID NO: 617 - DNA, 60 bp, Homo sapiens
tcgtctatgcatggaatattctccaaaatcgaggagacagccgttgtccccagggctcct

SEQ ID NO: 618 - DNA, 60 bp, Homo sapiens
agtgcctgtgaaagtgtttctgaattcctcgagcgtacaccaatggaggaggtatgggcc

SEQ ID NO: 619 - DNA, 60 bp, Homo sapiens
cccgatttgcttaatctcggcatagatttcgaaaggcctgagaaaaaagggagctggtgg

SEQ ID NO: 620 - DNA, 60 bp, Homo sapiens
cagccagccagtagatcttcataggagctcgaaattttgctataaatgtgagctttgaaa

SEQ ID NO: 621 - DNA, 60 bp, Homo sapiens
atgctttttggaataaatatattatttttcgaagtaatgaagagggaggtggttttcttt

SEQ ID NO: 622 - DNA, 60 bp, Homo sapiens
cccgatttgcttaatctcggcatagatttcgaagaactccttttagcatttcttgtaggt

SEQ ID NO: 623 - DNA, 60 bp, Homo sapiens
ctcttcaggggtgtgtggagtaaatagctcgaggaaacctaaaagtgtcatgttatccat

SEQ ID NO: 624 - DNA, 60 bp, Homo sapiens
acagagccctcagaaataatgccgcatatcgaggatctatctcctttctcacctgtctgg

SEQ ID NO: 625 - DNA, 60 bp, Homo sapiens
agccagccattgttaacatagttcaacttcgatatttttacatcataattttgttttatt

SEQ ID NO: 626 - DNA, 60 bp, Homo sapiens
gccccattctgagaactcctgattggattcgagtgctggtttgggtcggagtgctggttc

SEQ ID NO: 627 - DNA, 60 bp, Homo sapiens
tgcccagcccattattgatttctttatgtcgaacttgccaacggaaattgaggaaaattg

SEQ ID NO: 628 - DNA, 60 bp, Homo sapiens
tgggtgttagggtttcaatatatgaatttcgaaggcctcttccaattctgaagtaaacag

SEQ ID NO: 629 - DNA, 60 bp, Homo sapiens
agtgcctgtgaaagtgtttctgaattcctcgagtccctcatgagattttccagcgtagtg

SEQ ID NO: 630 - DNA, 60 bp, Homo sapiens
tgaggatggtgaaagatacctaagtcactcgagtgctggtttgggtcggagtgctggttc

SEQ ID NO: 631 - DNA, 60 bp, Homo sapiens
aaggcgggcagatcacgaggtcaggcaatcgaaaaaaaaaaaaaagaaagaaaaaaaaag

SEQ ID NO: 632 - DNA, 60 bp, Homo sapiens
tgatcactccaaacacccaaaggtgacttcgagtgctggtttgggtcggagtgctggttc

SEQ ID NO: 633 - DNA, 60 bp, Homo sapiens
cagccagccagtagatcttcataggagctcgaattcctatccagcactggtcattagggg

SEQ ID NO: 634 - DNA, 60 bp, Homo sapiens
agtggtgtaacctcagttgactgcagcctcgagtgctggtttgggtcggagtgctggttc

SEQ ID NO: 635 - DNA, 60 bp, Homo sapiens
ttgtgaaagtcatatccttgcgtcttcatcgaagtaatgaagagggaggtggttttcttt

SEQ ID NO: 636 - DNA, 60 bp, Homo sapiens
ggcaggggcagtattctcaggtggttcctcgaagaacgggcctggcccaaggcactttgg

SEQ ID NO: 637 - DNA, 60 bp, Homo sapiens
tgtcttgaggcttactagtgcactcccgtcgatgtattcactaataaaaaaggaagattc

SEQ ID NO: 638 - DNA, 60 bp, Homo sapiens
gtcagggtcagatattgatttaacgaagtcgaagaactaaaaacttttttctagtttcac

SEQ ID NO: 639 - DNA, 60 bp, Homo sapiens
tggggtgattgggttcatttatgtattatcgactcatagatccaggtacaacgtataatt

SEQ ID NO: 640 - DNA, 60 bp, Homo sapiens
aagcagtcttggggaaaaagaaaaaagctcgaggagacagccgttgtccccagggctcct

SEQ ID NO: 641 - DNA, 60 bp, Homo sapiens
tcaaagtccaagttagaattctgagaactcgagtgctggtttgggtcggagtgctggttc

SEQ ID NO: 642 - DNA, 60 bp, Homo sapiens
aaggcgggcagatcacgaggtcaacagatcgacatttcatctgttctcttttagatcaaa

SEQ ID NO: 643 - DNA, 60 bp, Homo sapiens
actatggtggatttaaaaataaatgccctcgagtgctggtttgggtcggagtgctggttc

SEQ ID NO: 644 - DNA, 60 bp, Homo sapiens
ggcaggggcagtattctcaggtggttcctcgatacacttctcataaaaaatggcactgaa

SEQ ID NO: 645 - DNA, 60 bp, Homo sapiens
acagagccctcagaaataatgccgcatatcgataatttgctagaatgactcacagaacac

SEQ ID NO: 646 - DNA, 60 bp, Homo sapiens
agtgcctgtgaaagtgtttctgaattcctcgatgtgttttggggactgaaaagtcctgca

SEQ ID NO: 647 - DNA, 60 bp, Homo sapiens
gtcagggtcagatattgatttaacgaagtcgacatgcgatttgaacagggacacaaatcc

SEQ ID NO: 648 - DNA, 60 bp, Homo sapiens
acagagccctcagaaataatgccgcatatcgaggctaatttagagtataacagtttggga

SEQ ID NO: 649 - DNA, 60 bp, Homo sapiens
aaaaggaaaaagggaaaccaggttttaatcgaagtaatgaagagggaggtggttttcttt

SEQ ID NO: 650 - DNA, 60 bp, Homo sapiens
tctttttattgaagagcgtgttttaaaatcgaatattagtagtcccgtggcaagcctttg

SEQ ID NO: 651 - DNA, 27 bp, Homo sapiens
gcaggtaggacaggtgccatcattccc

SEQ ID NO: 652 - DNA, 28 bp, Homo sapiens
cccaacctgaagaacagagagaagactg

SEQ ID NO: 653 - DNA, 28 bp, Homo sapiens
ggacacgagccaggatgctaaacagtga

SEQ ID NO: 654 - DNA, 28 bp, Homo sapiens
gctgtgtaatgaggactgaataagactg

SEQ ID NO: 655 - DNA, 23 bp, Homo sapiens
gggcaagggtgggaagcgatggc

SEQ ID NO: 656 - DNA, 28 bp, Homo sapiens
ttccctgccaccattctccagacgggcg

SEQ ID NO: 657 - DNA, 27 bp, Homo sapiens
gcaggtaggacaggtgccatcattccc

SEQ ID NO: 658 - DNA, 27 bp, Homo sapiens
cctcccacatctgatactcctcacccc

SEQ ID NO: 659 - DNA, 28 bp, Homo sapiens
agcccagaaaccttggcaaacagaagag

SEQ ID NO: 660 - DNA, 28 bp, Homo sapiens
gcacccaataaggaaaacagaaaagttc

SEQ ID NO: 661 - DNA, 23 bp, Homo sapiens
gggcaagggtgggaagcgatggc

SEQ ID NO: 662 - DNA, 28 bp, Homo sapiens
gagggtcaggtgggctctgggagttgtg

SEQ ID NO: 663 - DNA, 26 bp, Homo sapiens
tagttcccttcagtcactctttgtat

SEQ ID NO: 664 - DNA, 23 bp, Homo sapiens
tgagccctggaggtggaggttgc

SEQ ID NO: 665 - DNA, 23 bp, Homo sapiens
ccaaccaactgatttatgccagg

SEQ ID NO: 666 - DNA, 23 bp, Homo sapiens
gacgacggctgtctcctcaaaca

SEQ ID NO: 667 - DNA, 23 bp, Homo sapiens
cgccctttccagcccctcagtcc

SEQ ID NO: 668 - DNA, 28 bp, Homo sapiens
ctccctccttgctgcctcttcccaccag

SEQ ID NO: 669 - DNA, 23 bp, Homo sapiens
agattttgggctgagacaatggg

SEQ ID NO: 670 - DNA, 26 bp, Homo sapiens
gggcattccataactacaagagaaaa

SEQ ID NO: 671 - DNA, 24 bp, Homo sapiens
agagcagagggaagaaggaagtct

SEQ ID NO: 672 - DNA, 24 bp, Homo sapiens
tattattatccattctgccaaagc

SEQ ID NO: 673 - DNA, 26 bp, Homo sapiens
gaaggtctctgtgtaatgatgaaccc

SEQ ID NO: 674 - DNA, 24 bp, Homo sapiens
aaccagcactcgcccaaaccagca

SEQ ID NO: 675 - DNA, 24 bp, Homo sapiens
caacaaaacaggaaaccaggactg

SEQ ID NO: 676 - DNA, 23 bp, Homo sapiens
caacagagatgggagacttgaag

SEQ ID NO: 677 - DNA, 28 bp, Homo sapiens
gccctatccttactccctgaaccttggg

SEQ ID NO: 678 - DNA, 28 bp, Homo sapiens
gaactggcatctcacgaatcctccctgc

SEQ ID NO: 679 - DNA, 28 bp, Homo sapiens
atccatcaaacagagacaaccacccttg

SEQ ID NO: 680 - DNA, 23 bp, Homo sapiens
cgcccaaaccagcactccgaccc

SEQ ID NO: 681 - DNA, 24 bp, Homo sapiens
cagccagcagatgggtgggtgggc

SEQ ID NO: 682 - DNA, 28 bp, Homo sapiens
ataatgggctgctacagagaaggcactg

SEQ ID NO: 683 - DNA, 27 bp, Homo sapiens
caacacctcattgacaccactggacag

SEQ ID NO: 684 - DNA, 28 bp, Homo sapiens
ctcaaacagggaccacgaggatgatggc

SEQ ID NO: 685 - DNA, 23 bp, Homo sapiens
gggcaagggtgggaagcgatggc

SEQ ID NO: 686 - DNA, 28 bp, Homo sapiens
accccagacaccctccgacaggacctca

SEQ ID NO: 687 - DNA, 27 bp, Homo sapiens
gcaggtaggacaggtgccatcattccc

SEQ ID NO: 688 - DNA, 28 bp, Homo sapiens
cactcactaaggtgggtggaggtaatcc

SEQ ID NO: 689 - DNA, 28 bp, Homo sapiens
gagcagcaagtgagccagcattaccgcc

SEQ ID NO: 690 - DNA, 27 bp, Homo sapiens
gcgtgtctctcagggaaggcaggatgc

SEQ ID NO: 691 - DNA, 23 bp, Homo sapiens
gccagagaaggagggattgattc

SEQ ID NO: 692 - DNA, 23 bp, Homo sapiens
caacagagatgggagacttgaag

SEQ ID NO: 693 - DNA, 27 bp, Homo sapiens
gcaggtaggacaggtgccatcattccc

SEQ ID NO: 694 - DNA, 28 bp, Homo sapiens
gggatttcatcaacaccagccctgacct

SEQ ID NO: 695 - DNA, 27 bp, Homo sapiens
cattagttagattctcataaggatggc

SEQ ID NO: 696 - DNA, 23 bp, Homo sapiens
tcacggcgaacccaatgtctatt

SEQ ID NO: 697 - DNA, 23 bp, Homo sapiens
agattttgggctgagacaatggg

SEQ ID NO: 698 - DNA, 23 bp, Homo sapiens
aaacaagcctgctgaccgccaga

SEQ ID NO: 699 - DNA, 28 bp, Homo sapiens
gccctatccttactccctgaaccttggg

SEQ ID NO: 700 - DNA, 27 bp, Homo sapiens
gtctggagaacacggcagtttggcagg

SEQ ID NO: 701 - DNA, 28 bp, Homo sapiens
gcatcctgtgagggcacctgtgtcattt

SEQ ID NO: 702 - DNA, 27 bp, Homo sapiens
ctccgacccaaaccagcactccagccg

SEQ ID NO: 703 - DNA, 27 bp, Homo sapiens
aggttacagccactgagcctggatgtg

SEQ ID NO: 704 - DNA, 28 bp, Homo sapiens
gggaaatgagtaagtctgagggaaacaa

SEQ ID NO: 705 - DNA, 28 bp, Homo sapiens
gggagggcaatggtgggctgatgtctct

SEQ ID NO: 706 - DNA, 27 bp, Homo sapiens
aatcagagggctgccaccaggcgagtg

SEQ ID NO: 707 - DNA, 28 bp, Homo sapiens
acaggacagggcaggagaggcaacgagg

SEQ ID NO: 708 - DNA, 28 bp, Homo sapiens
gccttgctgaatgctggtcacatcgcag

SEQ ID NO: 709 - DNA, 28 bp, Homo sapiens
gtagatggcattgacaagaaccagcctg

SEQ ID NO: 710 - DNA, 25 bp, Homo sapiens
ccgacccaaaccagcactccagccg

SEQ ID NO: 711 - DNA, 28 bp, Homo sapiens
atgcccactttcaccactgctattggac

SEQ ID NO: 712 - DNA, 26 bp, Homo sapiens
cgtccacctacatctcccttacctat

SEQ ID NO: 713 - DNA, 23 bp, Homo sapiens
ggtgggtgctgaggagtgggctc

SEQ ID NO: 714 - DNA, 28 bp, Homo sapiens
actccgacccaaaccagcactccagccg

SEQ ID NO: 715 - DNA, 28 bp, Homo sapiens
gagcagcaagtgagccagcattaccgcc

SEQ ID NO: 716 - DNA, 27 bp, Homo sapiens
tgggtgaggtggagccgagtttgggtc

SEQ ID NO: 717 - DNA, 23 bp, Homo sapiens
ccttcctccctccctccccgctt

SEQ ID NO: 718 - DNA, 28 bp, Homo sapiens
actccgacccaaaccagcactccagccg

SEQ ID NO: 719 - DNA, 24 bp, Homo sapiens
tagttagattctcataaggatggc

SEQ ID NO: 720 - DNA, 24 bp, Homo sapiens
tttcgtccttcgtcacattcacgg

SEQ ID NO: 721 - DNA, 28 bp, Homo sapiens
ctggctcagaggaggtggtgattatgga

SEQ ID NO: 722 - DNA, 26 bp, Homo sapiens
gccgaggtctccagtaacacccttgc

SEQ ID NO: 723 - DNA, 28 bp, Homo sapiens
cagtcttgtgatagaggttggtgagtgt

SEQ ID NO: 724 - DNA, 27 bp, Homo sapiens
gatttcagaagcacctccctccccagc

SEQ ID NO: 725 - DNA, 28 bp, Homo sapiens
ctttgtcagtgcctgttgccaccttggt

SEQ ID NO: 726 - DNA, 28 bp, Homo sapiens
ctgtggaggaaaatagttgagcaggaac

SEQ ID NO: 727 - DNA, 25 bp, Homo sapiens
gctcttgggtaggcagccagcagcc

SEQ ID NO: 728 - DNA, 28 bp, Homo sapiens
cccacttactctctgacctatcacccag

SEQ ID NO: 729 - DNA, 28 bp, Homo sapiens
gtgttttgacaagttggtgccgaatgct

SEQ ID NO: 730 - DNA, 28 bp, Homo sapiens
ctcaaacagggaccacgaggatgatggc

SEQ ID NO: 731 - DNA, 23 bp, Homo sapiens
cagggagggagaaacccagacag

SEQ ID NO: 732 - DNA, 23 bp, Homo sapiens
caaaccagcactccgacccaaac

SEQ ID NO: 733 - DNA, 26 bp, Homo sapiens
aacagcaagccagccaggtgtggtgg

SEQ ID NO: 734 - DNA, 27 bp, Homo sapiens
cactctcctcctcaccttcagcacccg

SEQ ID NO: 735 - DNA, 23 bp, Homo sapiens
gtgagcaggaggcttgggcaggc

SEQ ID NO: 736 - DNA, 28 bp, Homo sapiens
actccgacccaaaccagcactccagccg

SEQ ID NO: 737 - DNA, 28 bp, Homo sapiens
ctggctcagaggaggtggtgattatgga

SEQ ID NO: 738 - DNA, 27 bp, Homo sapiens
ccaaccccagccacacacagcagaccc

SEQ ID NO: 739 - DNA, 23 bp, Homo sapiens
agattttgggctgagacaatggg

SEQ ID NO: 740 - DNA, 26 bp, Homo sapiens
ggctatttccgagttgtatcctttat

SEQ ID NO: 741 - DNA, 23 bp, Homo sapiens
gggcaagggtgggaagcgatggc

SEQ ID NO: 742 - DNA, 28 bp, Homo sapiens
aggttggctggctgctcctgctcccttg

SEQ ID NO: 743 - DNA, 27 bp, Homo sapiens
tttgtcagtgcctgttgccaccttggt

SEQ ID NO: 744 - DNA, 28 bp, Homo sapiens
gttttctgaggctgatttcttgttgaga

SEQ ID NO: 745 - DNA, 23 bp, Homo sapiens
agattttgggctgagacaatggg

SEQ ID NO: 746 - DNA, 26 bp, Homo sapiens
agagccattatctttcctttggtgaa

SEQ ID NO: 747 - DNA, 26 bp, Homo sapiens
caggcattagttagattctcataagg

SEQ ID NO: 748 - DNA, 23 bp, Homo sapiens
tcacggcgaacccaatgtctatt

SEQ ID NO: 749 - DNA, 28 bp, Homo sapiens
caaccacttcaccaaggagccagagttc

SEQ ID NO: 750 - DNA, 28 bp, Homo sapiens
gggatggagggaagagtcacaggaaagt

SEQ ID NO: 751 - DNA, 60 bp, Homo sapiens
tttgcaggtgtgtggttaacactatccttcgatgagttctttgagagttctgtattgttt

SEQ ID NO: 752 - DNA, 60 bp, Homo sapiens
aatataaattcaattcattaaaaaataatcgagtgctggtttgggtcggagtgctggttc

SEQ ID NO: 753 - DNA, 60 bp, Homo sapiens
aaaatttgatttaattctccaattatcatcgaatattagtagtcccgtggcaagcctttg

SEQ ID NO: 754 - DNA, 60 bp, Homo sapiens
caaataaagccagaactcactcattacctcgagtgctggtttgggtcggagtgctggttc

SEQ ID NO: 755 - DNA, 60 bp, Homo sapiens
aggcgggcagatcacctgaggtcagagttcgaaggaaaagctaaattctagttagaagtt

SEQ ID NO: 756 - DNA, 60 bp, Homo sapiens
atcgtcgggagaatcttcccttgtagtctcgattttcctaagcccaaataaaatggttca

SEQ ID NO: 757 - DNA, 60 bp, Homo sapiens
atcgtcgggagaatcttcccttgtagtctcgacaggaaacctcattttataggtagccat

SEQ ID NO: 758 - DNA, 60 bp, Homo sapiens
aggactgtgggtgagaaaagctctggattcgattagattttgattcagtattttaggcaa

SEQ ID NO: 759 - DNA, 60 bp, Homo sapiens
atataattctgaactaattttccaaacttcgaatattagtagtcccgtggcaagcctttg

SEQ ID NO: 760 - DNA, 60 bp, Homo sapiens
agtttttaatgtcaaatatgatatacagtcgaatattagtagtcccgtggcaagcctttg

SEQ ID NO: 761 - DNA, 60 bp, Homo sapiens
gccaaagagagctttggtaccaataaattcgagtttgcgtgggcaggaaatttattcttt

SEQ ID NO: 762 - DNA, 60 bp, Homo sapiens
atcgtcgggagaatcttcccttgtagtctcgatagtttaatttcctaagaggaaagtact

SEQ ID NO: 763 - DNA, 60 bp, Homo sapiens
tcccagcccagtgtacagagtaaaaacatcgaatggcgacttttttcacaatatttggat

SEQ ID NO: 764 - DNA, 60 bp, Homo sapiens
acagagccctcagaaataatgccacatatcgagcaattcatgttttaaagttggactcta

SEQ ID NO: 765 - DNA, 60 bp, Homo sapiens
tttgcaggtgtgtggttaacactatccttcgaaaaaaaagccttttgagacttcagttgg

SEQ ID NO: 766 - DNA, 60 bp, Homo sapiens
cctaatgaaaagataccaggtcctgagatcgaagtaatgaagagggaggtggttttcttt

SEQ ID NO: 767 - DNA, 60 bp, Homo sapiens
gatcggtcagacattgcccatctgttgatcgaggagacagccgttgtccccagggctcct

SEQ ID NO: 768 - DNA, 60 bp, Homo sapiens
tttgcaggtgtgtggttaacactatccttcgattaccgagggattcatgttcctgctcct

SEQ ID NO: 769 - DNA, 60 bp, Homo sapiens
ggcaggcagatcacttgaggtcagtagttcgattctctctttttttcctcctcctttatt

SEQ ID NO: 770 - DNA, 60 bp, Homo sapiens
acagagccctcagaaataatgccgcatatcgaaaccatgaagctagcaaacactggaatg

SEQ ID NO: 771 - DNA, 60 bp, Homo sapiens
atcgtcgggagaatcttcccttgtagtctcgaatgttttcacaatatttgacttctaatg

SEQ ID NO: 772 - DNA, 60 bp, Homo sapiens
atcgtcgggagaatcttcccttgtagtctcgacttcccaggtgattctcctacctcagcc

SEQ ID NO: 773 - DNA, 60 bp, Homo sapiens
gtcgtgcagtggtgatgatctcctgcaatcgatcatgatggcaggcaatcaaagaatctt

SEQ ID NO: 774 - DNA, 60 bp, Homo sapiens
atcgtcgggagaatcttcccttgtagtctcgagcatcagatgaagaaaaaattaatccac

SEQ ID NO: 775 - DNA, 60 bp, Homo sapiens
gcatatgcaggtgtgggctttttaaaaatcgattaagtttttattctgattattgtatgt

SEQ ID NO: 776 - DNA, 60 bp, Homo sapiens
acagagccctcagaaataatgccgcatatcgatatataaagattgttttaatttctatct

SEQ ID NO: 777 - DNA, 60 bp, Homo sapiens
tgggtgttagggtttcaatatatgaatttcgaagctagccaaagcatgtattgtttatgt

SEQ ID NO: 778 - DNA, 60 bp, Homo sapiens
gaatgtttaggttttgtaagtttacaaatcgagtgcccagttacagaattttctggagtt

SEQ ID NO: 779 - DNA, 60 bp, Homo sapiens
atgggtgtgggagttaaggtcgctgtcttcgattggagatttttgactacagaatcagtc

SEQ ID NO: 780 - DNA, 60 bp, Homo sapiens
gcatatgcaggtgtgggctttttaaaaatcgactagtttttaaagtaggttgtctgtttt

SEQ ID NO: 781 - DNA, 60 bp, Homo sapiens
ggcaggcagatcacttgaggtcagtagttcgacctgtcatctacattaggtatttctgct

SEQ ID NO: 782 - DNA, 60 bp, Homo sapiens
aaggcgggcagatcacgaggtcaggagttcgatctcatatagatataaaccaagtctgtg

SEQ ID NO: 783 - DNA, 60 bp, Homo sapiens
acagagccctcagaaataatgccgcatatcgacaagcacttttctgctagaatgtgatat

SEQ ID NO: 784 - DNA, 60 bp, Homo sapiens
ggcgggcagatcacttgaggtcagggattcgaagtccatccttaatgaaagatacctcta

SEQ ID NO: 785 - DNA, 60 bp, Homo sapiens
agaatgggcgatgtttctcagggctgcttcgaaaagcatttattaaacacctaccttgct

SEQ ID NO: 786 - DNA, 60 bp, Homo sapiens
tgactgaggtcccaggtgtggccccaactcgaaaaagaaaaaaagagcctggtgaaaaag

SEQ ID NO: 787 - DNA, 60 bp, Homo sapiens
atcgtcgggagaatcttcccttgtagtctcgaccagaatcctcttctccaggcggcgtaa

SEQ ID NO: 788 - DNA, 60 bp, Homo sapiens
tcccagcccagtgtacagagtaaaaacatcgaaaacaaatcacattcctaaccaaaagct

SEQ ID NO: 789 - DNA, 60 bp, Homo sapiens
ggcaggcagatcacttgaggtcagtagttcgaagtgttaggtaactttctcttagtggaa

SEQ ID NO: 790 - DNA, 60 bp, Homo sapiens
agtgcctgtgaaagtgtttctgaattcctcgacaacttctcatcttcatctctagcccag

SEQ ID NO: 791 - DNA, 60 bp, Homo sapiens
ggcaggcagatcacttgaggtcaggaattcgattttcaagcaaagtgctgaatggaattt

SEQ ID NO: 792 - DNA, 60 bp, Homo sapiens
acagagccctcagaaataatgccgcatatcgactcccctcattcctggctgtgtgacctg

SEQ ID NO: 793 - DNA, 60 bp, Homo sapiens
ggcaggcagatcacttgaggtcaggagctcgatgccaaccttttcaaagtagttaaacag

SEQ ID NO: 794 - DNA, 60 bp, Homo sapiens
ctcatctacctgcaagtgtgagatccactcgaagtaatgaagagggaggtggttttcttt

SEQ ID NO: 795 - DNA, 60 bp, Homo sapiens
atcgtcgggagaatcttcccttgtagtctcgaaatccgccattttataccacccgcgggc

SEQ ID NO: 796 - DNA, 60 bp, Homo sapiens
agcaaatgttgacttagtaatttttatatcgaggccgcagtgagttgtgatcataccact

SEQ ID NO: 797 - DNA, 60 bp, Homo sapiens
gttctagaaggttctggaagacaaaatatcgaggacacccgggattcagccagggaggcc

SEQ ID NO: 798 - DNA, 60 bp, Homo sapiens
tcccagcccagtgtacagagtaaaaacatcgagtcttttagacatttaattccactacat

SEQ ID NO: 799 - DNA, 60 bp, Homo sapiens
tgttgagggtgtcccggagcatcaaatctcgataatatgtccttgtcttctacagagatg

SEQ ID NO: 800 - DNA, 60 bp, Homo sapiens
gggcgcgggtctgcggagcccccagggctcgagaagacttattgttttgtctgcccagaa

SEQ ID NO: 801 - DNA, 28 bp, Homo sapiens
ctctgttccgcctacccaggtccacacc

SEQ ID NO: 802 - DNA, 28 bp, Homo sapiens
ccttcttcctgtctccttctcctccatc

SEQ ID NO: 803 - DNA, 24 bp, Homo sapiens
cagggcactcaggctgggtgaagg

SEQ ID NO: 804 - DNA, 28 bp, Homo sapiens
agcactcgcccaaaccagcactccgacc

SEQ ID NO: 805 - DNA, 28 bp, Homo sapiens
ttcccagtggttgacacaaagtaggtgc

SEQ ID NO: 806 - DNA, 28 bp, Homo sapiens
gggatggagggaagagtcacaggaaagt

SEQ ID NO: 807 - DNA, 23 bp, Homo sapiens
tcactttgacattcatccctggg

SEQ ID NO: 808 - DNA, 28 bp, Homo sapiens
tgatggagaagtttatgatgaaggaatc

SEQ ID NO: 809 - DNA, 23 bp, Homo sapiens
ggctcaccactgtaaccccagcg

SEQ ID NO: 810 - DNA, 23 bp, Homo sapiens
ccagggatgcccaagaagtcctt

SEQ ID NO: 811 - DNA, 23 bp, Homo sapiens
gcagtggctgggtgtgggaggca

SEQ ID NO: 812 - DNA, 27 bp, Homo sapiens
gggagatggtgggttcattctgctttt

SEQ ID NO: 813 - DNA, 23 bp, Homo sapiens
gcagtggctgggtgtgggaggca

SEQ ID NO: 814 - DNA, 28 bp, Homo sapiens
ggcactggctgtgaacctgaggaggctg

SEQ ID NO: 815 - DNA, 28 bp, Homo sapiens
tttctaaatgtgattgccaagcataggc

SEQ ID NO: 816 - DNA, 25 bp, Homo sapiens
gatgaaggaatcaggcataacacct

SEQ ID NO: 817 - DNA, 26 bp, Homo sapiens
gctgggtgcgggtcctactggagtcc

SEQ ID NO: 818 - DNA, 28 bp, Homo sapiens
gggatggagggaagagtcacaggaaagt

SEQ ID NO: 819 - DNA, 28 bp, Homo sapiens
ggcaatggtcaagggcacgaacacaatg

SEQ ID NO: 820 - DNA, 28 bp, Homo sapiens
gggatggagggaagagtcacaggaaagt

SEQ ID NO: 821 - DNA, 28 bp, Homo sapiens
ccgtaaagttggacactaaatgacatag

SEQ ID NO: 822 - DNA, 28 bp, Homo sapiens
ttcagcgtttgacttttgagaagagtgc

SEQ ID NO: 823 - DNA, 28 bp, Homo sapiens
ccgtcgcaagtttcgttccccaacccgc

SEQ ID NO: 824 - DNA, 23 bp, Homo sapiens
ctcggcactgactcccaacccgc

SEQ ID NO: 825 - DNA, 28 bp, Homo sapiens
ctgccctcagagagtaaagagacaggac

SEQ ID NO: 826 - DNA, 28 bp, Homo sapiens
aggctgggtttacagtcactccgagaaa

SEQ ID NO: 827 - DNA, 26 bp, Homo sapiens
caagtctacagtaaccaaaacagcat

SEQ ID NO: 828 - DNA, 26 bp, Homo sapiens
ttttggagactgtttgaatgtgacca

SEQ ID NO: 829 - DNA, 28 bp, Homo sapiens
ctctgttccgcctacccaggtccacacc

SEQ ID NO: 830 - DNA, 25 bp, Homo sapiens
ccataccccagcaacggacagccag

SEQ ID NO: 831 - DNA, 23 bp, Homo sapiens
gccaataagggtctctctcagaa

SEQ ID NO: 832 - DNA, 24 bp, Homo sapiens
gatgaaggaatcaggcataacacc

SEQ ID NO: 833 - DNA, 23 bp, Homo sapiens
ggaagacctcacagggactctgc

SEQ ID NO: 834 - DNA, 23 bp, Homo sapiens
gacgacggctgtctcctcaaaca

SEQ ID NO: 835 - DNA, 28 bp, Homo sapiens
ctctgttccgcctacccaggtccacacc

SEQ ID NO: 836 - DNA, 23 bp, Homo sapiens
gtggcagcaggtgtcccatcggc

SEQ ID NO: 837 - DNA, 28 bp, Homo sapiens
agtgctgaggttgagaagccccaagtta

SEQ ID NO: 838 - DNA, 28 bp, Homo sapiens
ttgtgtaagtcagtccatcctaagcctt

SEQ ID NO: 839 - DNA, 23 bp, Homo sapiens
agattttgggctgagacaatggg

SEQ ID NO: 840 - DNA, 24 bp, Homo sapiens
gatgacacctcacttctttccgaa

SEQ ID NO: 841 - DNA, 23 bp, Homo sapiens
tatccgtaaggcaagtcctgagt

SEQ ID NO: 842 - DNA, 23 bp, Homo sapiens
tggctacacagtgagactccgtc

SEQ ID NO: 843 - DNA, 23 bp, Homo sapiens
tatccgtaaggcaagtcctgagt

SEQ ID NO: 844 - DNA, 23 bp, Homo sapiens
gcccaggagtttgagaccagcct

SEQ ID NO: 845 - DNA, 28 bp, Homo sapiens
gctccattttccttgtccctcctcaggt

SEQ ID NO: 846 - DNA, 28 bp, Homo sapiens
accgatgtggacaatgaagacctgtggg

SEQ ID NO: 847 - DNA, 28 bp, Homo sapiens
ccgtcgcaagtttcgttccccaacccgc

SEQ ID NO: 848 - DNA, 25 bp, Homo sapiens
ccccacagccccacctaacctgatg

SEQ ID NO: 849 - DNA, 28 bp, Homo sapiens
ggcagaaaggaaagagcctcgcagcatt

SEQ ID NO: 850 - DNA, 28 bp, Homo sapiens
gtcatagcaagaaccagagaaatgtcag

SEQ ID NO: 851 - DNA, 23 bp, Homo sapiens
agattttgggctgagacaatggg

SEQ ID NO: 852 - DNA, 28 bp, Homo sapiens
gaaagttgtttatgaggcactgtttaca

SEQ ID NO: 853 - DNA, 25 bp, Homo sapiens
gggagggcaatggtgggctgatgtc

SEQ ID NO: 854 - DNA, 25 bp, Homo sapiens
atggcatcaccttcctcaagagtgc

SEQ ID NO: 855 - DNA, 28 bp, Homo sapiens
ttgtgagaagggtaaaatcttaggcagc

SEQ ID NO: 856 - DNA, 28 bp, Homo sapiens
ctatgccccaagccagaatgaagcggtt

SEQ ID NO: 857 - DNA, 28 bp, Homo sapiens
ccgtgagcacagttctccgctggtaaat

SEQ ID NO: 858 - DNA, 28 bp, Homo sapiens
gctgtagcaaagcgacaaaactaagagt

SEQ ID NO: 859 - DNA, 28 bp, Homo sapiens
ggcagaaaggaaagagcctcgcagcatt

SEQ ID NO: 860 - DNA, 25 bp, Homo sapiens
ccccagggagatggacgggcagagc

SEQ ID NO: 861 - DNA, 23 bp, Homo sapiens
taatcccagcaagttgagaggct

SEQ ID NO: 862 - DNA, 23 bp, Homo sapiens
tagaggagggatagcattagcag

SEQ ID NO: 863 - DNA, 28 bp, Homo sapiens
cacctaaggagccagcaactgacaagag

SEQ ID NO: 864 - DNA, 27 bp, Homo sapiens
gggtctgctgtgctccacggctgaagg

SEQ ID NO: 865 - DNA, 23 bp, Homo sapiens
agattttgggctgagacaatggg

SEQ ID NO: 866 - DNA, 26 bp, Homo sapiens
taggagttttgctttgggacttgtta

SEQ ID NO: 867 - DNA, 23 bp, Homo sapiens
cacctgtaatcccagcactttgg

SEQ ID NO: 868 - DNA, 24 bp, Homo sapiens
ggacaagttattcacacgccagta

SEQ ID NO: 869 - DNA, 23 bp, Homo sapiens
gcctgacattcctgccttcttat

SEQ ID NO: 870 - DNA, 26 bp, Homo sapiens
gcaagtgtcagattttgatgtcccat

SEQ ID NO: 871 - DNA, 23 bp, Homo sapiens
ggtgctctgcgtcccacacacgg

SEQ ID NO: 872 - DNA, 28 bp, Homo sapiens
gttgggttgggtgggcacatcctgggtc

SEQ ID NO: 873 - DNA, 28 bp, Homo sapiens
ccgtcgcaagtttcgttccccaacccgc

SEQ ID NO: 874 - DNA, 28 bp, Homo sapiens
cttcccttccttccctccctcagttctc

SEQ ID NO: 875 - DNA, 28 bp, Homo sapiens
ctgccctcagagagtaaagagacaggac

SEQ ID NO: 876 - DNA, 28 bp, Homo sapiens
ggtggctacagcagagtgatgaaaatac

SEQ ID NO: 877 - DNA, 28 bp, Homo sapiens
tggctcatacctgtaatcccagcaagtt

SEQ ID NO: 878 - DNA, 28 bp, Homo sapiens
cagattattcaaaaggaaaacacccaac

SEQ ID NO: 879 - DNA, 23 bp, Homo sapiens
gggcaagggtgggaagcgatggc

SEQ ID NO: 880 - DNA, 28 bp, Homo sapiens
gggagggtgctggtggcagtggagttga

SEQ ID NO: 881 - DNA, 28 bp, Homo sapiens
cccaacagataccaaaatccacgaagac

SEQ ID NO: 882 - DNA, 28 bp, Homo sapiens
ccaggcaaactgtgatggtagggtcagg

SEQ ID NO: 883 - DNA, 23 bp, Homo sapiens
agattttgggctgagacaatggg

SEQ ID NO: 884 - DNA, 23 bp, Homo sapiens
gagcctaagtaactaacccaggt

SEQ ID NO: 885 - DNA, 27 bp, Homo sapiens
atctactttcaggcagtggctcacgcc

SEQ ID NO: 886 - DNA, 28 bp, Homo sapiens
ctctcttccttttctgtgcctggactta

SEQ ID NO: 887 - DNA, 23 bp, Homo sapiens
gcaaggaggtgacaggagtggtc

SEQ ID NO: 888 - DNA, 23 bp, Homo sapiens
caacagagatgggagacttgaag

SEQ ID NO: 889 - DNA, 28 bp, Homo sapiens
cgtcgcaagtttcgttccccaacccgca

SEQ ID NO: 890 - DNA, 24 bp, Homo sapiens
tcgttggcggcagcgggagtgggt

SEQ ID NO: 891 - DNA, 23 bp, Homo sapiens
gtccctgctgggctgcctcagtc

SEQ ID NO: 892 - DNA, 28 bp, Homo sapiens
ccgcatctccccttgtgccagtccagac

SEQ ID NO: 893 - DNA, 23 bp, Homo sapiens
cccatcaccccaaggacccctcc

SEQ ID NO: 894 - DNA, 28 bp, Homo sapiens
cactcagggctctgccgtcaccttggag

SEQ ID NO: 895 - DNA, 28 bp, Homo sapiens
ctgccctcagagagtaaagagacaggac

SEQ ID NO: 896 - DNA, 28 bp, Homo sapiens
acactgaaaaggaagaactgtgagaggg

SEQ ID NO: 897 - DNA, 24 bp, Homo sapiens
attatcctcctttgttccttcccc

SEQ ID NO: 898 - DNA, 23 bp, Homo sapiens
gccacttactccttctgggtccc

SEQ ID NO: 899 - DNA, 23 bp, Homo sapiens
tccgccctgcccacacccacctc

SEQ ID NO: 900 - DNA, 28 bp, Homo sapiens
ggtgttgagattgaaggagactgtcagg

SEQ ID NO: 901 - DNA, 60 bp, Homo sapiens
ggcaggcagatcacttgaggtcaggaattcgatgtcttcttccgccatggtgctgaatcc

SEQ ID NO: 902 - DNA, 60 bp, Homo sapiens
agtgcctgtgaaagtgtttctgaattcctcgaaggctgtggtgagaatccactgggaagt

SEQ ID NO: 903 - DNA, 60 bp, Homo sapiens
acagagccctcagaaataatgccgcatatcgaggcccttgcattcgttttctattgttaa

SEQ ID NO: 904 - DNA, 60 bp, Homo sapiens
tgttgagggtgtcccggagcatcaaatctcgactctagcttatgctgtttaatgcctttc

SEQ ID NO: 905 - DNA, 60 bp, Homo sapiens
ataatgggcgatgtttctcagggctgcttcgattcttatgacattaactttcatttttat

SEQ ID NO: 906 - DNA, 60 bp, Homo sapiens
agcccagccatttattctctgatatttatcgaggtgaaacataatggtagcttggaccag

SEQ ID NO: 907 - DNA, 60 bp, Homo sapiens
taaaagcaatttcattttttttttcttttcgaatattagtagtcccgtggcaagcctttg

SEQ ID NO: 908 - DNA, 60 bp, Homo sapiens
acagagccctcagaaataatgccacatatcgaaaatactgtacaatagtctcaggatttg

SEQ ID NO: 909 - DNA, 60 bp, Homo sapiens
tgatgtggatttgtactctattacagtttcgagggtgtgggtagatatattctcagtttt

SEQ ID NO: 910 - DNA, 60 bp, Homo sapiens
ggcaggcagatcacttgaggtcacgagttcgataggtaaagacttactgttgtcattttg

SEQ ID NO: 911 - DNA, 60 bp, Homo sapiens
atcgtcgggagaatcttcccttgtagtctcgaaagccttgccctatctatccccaacccc

SEQ ID NO: 912 - DNA, 60 bp, Homo sapiens
acagagccctcagaaataatgccgcatatcgaccattttgtaaaaacaggagtggcaatt

SEQ ID NO: 913 - DNA, 60 bp, Homo sapiens
ataatgggcgatgtttctcagggctgcttcgacaaacctgttctttcagggcaaaaaaat

SEQ ID NO: 914 - DNA, 60 bp, Homo sapiens
acatatttcaaatgagctttataaatgttcgaggcgggcagatcacttgaggccagaagt

SEQ ID NO: 915 - DNA, 60 bp, Homo sapiens
atcgtcgggagaatcttcccttgtagtctcgaggaggcaatcttagaagaacccagtgag

SEQ ID NO: 916 - DNA, 60 bp, Homo sapiens
ttaaatagaaacctctttattctaagtatcgaaagtgctgggattattaggcatgagcca

SEQ ID NO: 917 - DNA, 60 bp, Homo sapiens
gcatatgcaggtgtgggctttttaaaaatcgagtacctgaagaacgatggcctggaaaag

SEQ ID NO: 918 - DNA, 60 bp, Homo sapiens
gaggcgggcagatcacgaggtcaagagatcgacttactgcactcaggtgattctcctgcc

SEQ ID NO: 919 - DNA, 60 bp, Homo sapiens
gagccagccagccctggaattaacttcctcgagataccatcatgtagaaaagtcacaaac

SEQ ID NO: 920 - DNA, 60 bp, Homo sapiens
tgttgagggtgtcccggagcatcaaatctcgattggattttgtgaatttttgtagctttt

SEQ ID NO: 921 - DNA, 60 bp, Homo sapiens
gctctttaactttggccattgtctatcctcgaggccgcagtgagttgtgatcataccact

SEQ ID NO: 922 - DNA, 60 bp, Homo sapiens
gcatatgcaggtgtgggctttttaaaaatcgactctgtcaaggctgagctcttccttgct

SEQ ID NO: 923 - DNA, 60 bp, Homo sapiens
gggcgcgggtctgcggagcccccagggctcgactctcaacaggcactcaataaatacttg

SEQ ID NO: 924 - DNA, 60 bp, Homo sapiens
ataatgggcgatgtttctcagggctgcttcgataacataaaaaactataagattttttgg

SEQ ID NO: 925 - DNA, 60 bp, Homo sapiens
gcatatgcaggtgtgggctttttaaaaatcgatgactgggaaaaagcacagagcgctttc

SEQ ID NO: 926 - DNA, 60 bp, Homo sapiens
ggcaggcagatcacctgaggtccggggttcgaatcatacaatacatggccttttaagtct

SEQ ID NO: 927 - DNA, 60 bp, Homo sapiens
ttgcttctgtgagagaagcaatttcttttcgaggccgcagtgagttgtgatcataccact

SEQ ID NO: 928 - DNA, 60 bp, Homo sapiens
ggcgggcagatcacttgaggccacgagttcgatttcatccatgatgttaataaaaaacac

SEQ ID NO: 929 - DNA, 60 bp, Homo sapiens
ataaattaaaagagactcaagagacatatcgaatattagtagtcccgtggcaagcctttg

SEQ ID NO: 930 - DNA, 60 bp, Homo sapiens
acagagccctcagaaataatgccacatatcgaagagttaactgtaactttgagaagttaa

SEQ ID NO: 931 - DNA, 60 bp, Homo sapiens
tcccagcccagtgtacagagtaaaaacatcgatttctaccaaatggcttctgtggtgaaa

SEQ ID NO: 932 - DNA, 60 bp, Homo sapiens
tgacttgtcctgttatggttgttattcctcgaggtttatggtaagggttagggttggaat

SEQ ID NO: 933 - DNA, 60 bp, Homo sapiens
ataatgggcgatgtttctcagggctgcttcgacattaaccaaatctacccttcatgcaga

SEQ ID NO: 934 - DNA, 60 bp, Homo sapiens
cctgaggtggggcttacaaattagtatttcgaacttgtttttcatttgtagtaaaagctt

SEQ ID NO: 935 - DNA, 60 bp, Homo sapiens
tgttgagggtgtcccggagcatcaaatctcgatgtgacattaacaacttgtaacagacag

SEQ ID NO: 936 - DNA, 60 bp, Homo sapiens
tgttgagggtgtcccggagcatcaaatctcgaaattcatgacttaaagtgtaatccctag

SEQ ID NO: 937 - DNA, 60 bp, Homo sapiens
agggttaggtgaaaaatctaaaggaaggtcgatgatgaaaatgttttggagttgatagtg

SEQ ID NO: 938 - DNA, 60 bp, Homo sapiens
ggcaggcagatcacttgaggtcaggaattcgaagactaaattgtactacttaaaaagaaa

SEQ ID NO: 939 - DNA, 60 bp, Homo sapiens
tggtgagggtgtttttcattcagctttttcgatttattatttttagaggttctctatcta

SEQ ID NO: 940 - DNA, 60 bp, Homo sapiens
tttgcaggtgtgtggttaacactatccttcgaggagaaaagtctctgccgtcctgtgcgg

SEQ ID NO: 941 - DNA, 60 bp, Homo sapiens
tataattgagggtgtaattgcaatgggctcgagtgctggtttgggtcggagtgctggttc

SEQ ID NO: 942 - DNA, 60 bp, Homo sapiens
ccatgctaatgcttaccaaagtgtgacctcgaggagacagccgttgtccccagggctcct

SEQ ID NO: 943 - DNA, 60 bp, Homo sapiens
tagatgatcatatatcatacaaaccacttcgaggacacccgggattcagccagggaggcc

SEQ ID NO: 944 - DNA, 60 bp, Homo sapiens
gacaggcagatcacttgaggtcaggcattcgaaacatgctccttaaggaccaccgctaca

SEQ ID NO: 945 - DNA, 60 bp, Homo sapiens
ccaaatagaaacttactttttttttttttcgaggccgcagtgagttgtgatcataccact

SEQ ID NO: 946 - DNA, 60 bp, Homo sapiens
ggcaggtggatcacttgaggtcagcaattcgaacacctaccaaatgttatttttttcacc

SEQ ID NO: 947 - DNA, 60 bp, Homo sapiens
tgtagcccagagtggatgttatctaatatcgaatattagtagtcccgtggcaagcctttg

SEQ ID NO: 948 - DNA, 60 bp, Homo sapiens
ggcaggcagatcacttgaggccaggaattcgacaatagcaagaccacctttgctagccag

SEQ ID NO: 949 - DNA, 60 bp, Homo sapiens
caggcaggcagaatgcaccatcaatgaatcgattctgatgacatggttgttcctcctgtt

SEQ ID NO: 950 - DNA, 60 bp, Homo sapiens
ggcaggtggatcatttgtggtcagcagttcgatctcctggattcaattgatcctcgcatc

SEQ ID NO: 951 - DNA, 27 bp, Homo sapiens
cattagttagattctcataaggatggc

SEQ ID NO: 952 - DNA, 27 bp, Homo sapiens
ggaatctcagtcactcaactacaaaat

SEQ ID NO: 953 - DNA, 27 bp, Homo sapiens
acaggacagggcaggagaggcaacgag

SEQ ID NO: 954 - DNA, 28 bp, Homo sapiens
cgtgggcttcctccctaatgatgccgag

SEQ ID NO: 955 - DNA, 23 bp, Homo sapiens
agattttgggctgagacaatggg

SEQ ID NO: 956 - DNA, 26 bp, Homo sapiens
gctgctacatttgtggtaacttctta

SEQ ID NO: 957 - DNA, 24 bp, Homo sapiens
attatcctcctttgttccttcccc

SEQ ID NO: 958 - DNA, 28 bp, Homo sapiens
atgcttacaggcagaaccagttttgtgc

SEQ ID NO: 959 - DNA, 22 bp, Homo sapiens
gtggtaaagtgtgaggatggca

SEQ ID NO: 960 - DNA, 23 bp, Homo sapiens
cttgggagaaagagacagatagc

SEQ ID NO: 961 - DNA, 28 bp, Homo sapiens
ctattgctaaatggaatgctaccctgct

SEQ ID NO: 962 - DNA, 26 bp, Homo sapiens
ttttcccaataccgtccctcctgtgg

SEQ ID NO: 963 - DNA, 28 bp, Homo sapiens
gaggttgtggaggaggctgaggatgagg

SEQ ID NO: 964 - DNA, 28 bp, Homo sapiens
gggatggagggaagagtcacaggaaagt

SEQ ID NO: 965 - DNA, 26 bp, Homo sapiens
caagtctacagtaaccaaaacagcat

SEQ ID NO: 966 - DNA, 23 bp, Homo sapiens
gcagttggtgctctgtatccaca

SEQ ID NO: 967 - DNA, 24 bp, Homo sapiens
atctcaaacctggtaataatcagc

SEQ ID NO: 968 - DNA, 24 bp, Homo sapiens
aatgctgagaaagccccaaaccca

SEQ ID NO: 969 - DNA, 26 bp, Homo sapiens
gcctaccactctgcctcacttgctcc

SEQ ID NO: 970 - DNA, 28 bp, Homo sapiens
gcacaagggaaaatggagggagaaagga

SEQ ID NO: 971 - DNA, 23 bp, Homo sapiens
ggcagtggctgggtgtgggaggc

SEQ ID NO: 972 - DNA, 28 bp, Homo sapiens
aaaggtaacttgagagtggagatgcggg

SEQ ID NO: 973 - DNA, 23 bp, Homo sapiens
agattttgggctgagacaatggg

SEQ ID NO: 974 - DNA, 23 bp, Homo sapiens
gggattacaggcataagccactg

SEQ ID NO: 975 - DNA, 28 bp, Homo sapiens
ccaggatgagccagaaggagtttcacaa

SEQ ID NO: 976 - DNA, 26 bp, Homo sapiens
tcactgaaggttccaaatctcccaat

SEQ ID NO: 977 - DNA, 26 bp, Homo sapiens
gcaccttacttactgtatctcttcag

SEQ ID NO: 978 - DNA, 23 bp, Homo sapiens
cctgcctcagcctccaaagtagc

SEQ ID NO: 979 - DNA, 28 bp, Homo sapiens
ccgtcgcaagtttcgttccccaacccgc

SEQ ID NO: 980 - DNA, 23 bp, Homo sapiens
gtcccctgtcttccccgctgtgg

SEQ ID NO: 981 - DNA, 28 bp, Homo sapiens
ctccctctacctccactaccctgcccag

SEQ ID NO: 982 - DNA, 23 bp, Homo sapiens
aggagggcagggctgggtcacag

SEQ ID NO: 983 - DNA, 28 bp, Homo sapiens
ggcagaaaggaaagagcctcgcagcatt

SEQ ID NO: 984 - DNA, 27 bp, Homo sapiens
ccagccagggatgttacccagagcccc

SEQ ID NO: 985 - DNA, 23 bp, Homo sapiens
tgcctgaaatcccagcaccttgg

SEQ ID NO: 986 - DNA, 23 bp, Homo sapiens
tggcacacacctgtagtcccagc

SEQ ID NO: 987 - DNA, 23 bp, Homo sapiens
gagccctgagcccctcctgacca

SEQ ID NO: 988 - DNA, 28 bp, Homo sapiens
gaagccaggagaaatgaaatgatgggtg

SEQ ID NO: 989 - DNA, 24 bp, Homo sapiens
attatcctcctttgttccttcccc

SEQ ID NO: 990 - DNA, 26 bp, Homo sapiens
cacttgacaaacccacagcacagaaa

SEQ ID NO: 991 - DNA, 23 bp, Homo sapiens
ctccccactaccccaacccaggc

SEQ ID NO: 992 - DNA, 28 bp, Homo sapiens
ccgcatctccccttgtgccagtccagac

SEQ ID NO: 993 - DNA, 28 bp, Homo sapiens
ggcagaaaggaaagagcctcgcagcatt

SEQ ID NO: 994 - DNA, 28 bp, Homo sapiens
gcaaacaaggaaaccccaagcagagaag

SEQ ID NO: 995 - DNA, 27 bp, Homo sapiens
cctattttctctacgccctcccgcccc

SEQ ID NO: 996 - DNA, 28 bp, Homo sapiens
ggtgaggcgaccctgttgaaaagtgggc

SEQ ID NO: 997 - DNA, 23 bp, Homo sapiens
ggtaaagtgttgggaaggcgaaa

SEQ ID NO: 998 - DNA, 25 bp, Homo sapiens
cagagatgaggttttctatgttggc

SEQ ID NO: 999 - DNA, 28 bp, Homo sapiens
gtcattctcccctctcatctctacccta

SEQ ID NO: 1000 - DNA, 28 bp, Homo sapiens
cacactcctttaggtaacagaacatttc

SEQ ID NO: 1001 - DNA, 23 bp, Homo sapiens
tgtctgtaatcccagcactttgg

SEQ ID NO: 1002 - DNA, 28 bp, Homo sapiens
ccaagagaaatgaaaatgttatgtccac

SEQ ID NO: 1003 - DNA, 23 bp, Homo sapiens
tcccctccctgtctggaccctgg

SEQ ID NO: 1004 - DNA, 28 bp, Homo sapiens
ccgcatctccccttgtgccagtccagac

SEQ ID NO: 1005 - DNA, 23 bp, Homo sapiens
agtggctcacgcctgtaatccta

SEQ ID NO: 1006 - DNA, 23 bp, Homo sapiens
tggaccacagcccttctttcggg

SEQ ID NO: 1007 - DNA, 28 bp, Homo sapiens
caaggacaatcaatggctgctcacatca

SEQ ID NO: 1008 - DNA, 28 bp, Homo sapiens
gggatggagggaagagtcacaggaaagt

SEQ ID NO: 1009 - DNA, 26 bp, Homo sapiens
caagtctacagtaaccaaaacagcat

SEQ ID NO: 1010 - DNA, 26 bp, Homo sapiens
tgagataactgaggctctaagaactt

SEQ ID NO: 1011 - DNA, 28 bp, Homo sapiens
ctgccctcagagagtaaagagacaggac

SEQ ID NO: 1012 - DNA, 28 bp, Homo sapiens
ctgttaggctcaaaggttgggtatgggc

SEQ ID NO: 1013 - DNA, 28 bp, Homo sapiens
tctcctacctctctacacactttattga

SEQ ID NO: 1014 - DNA, 28 bp, Homo sapiens
caagagtctaatcccaaaccccaaccag

SEQ ID NO: 1015 - DNA, 26 bp, Homo sapiens
tggtaaagtgttaggacggtgaaaat

SEQ ID NO: 1016 - DNA, 28 bp, Homo sapiens
ggaagtattttcagtgtatttcagagac

SEQ ID NO: 1017 - DNA, 25 bp, Homo sapiens
gtcaggacttggctgggcacctacc

SEQ ID NO: 1018 - DNA, 28 bp, Homo sapiens
gcactgggagcacagggcaggaatggtg

SEQ ID NO: 1019 - DNA, 24 bp, Homo sapiens
attatcctcctttgttccttcccc

SEQ ID NO: 1020 - DNA, 23 bp, Homo sapiens
tgccacttcccatctgctcaggg

SEQ ID NO: 1021 - DNA, 24 bp, Homo sapiens
attatcctcctttgttccttcccc

SEQ ID NO: 1022 - DNA, 26 bp, Homo sapiens
cccattcagacagagcagacccattt

SEQ ID NO: 1023 - DNA, 28 bp, Homo sapiens
gctaacagtgccagttgaaaggagatgc

SEQ ID NO: 1024 - DNA, 27 bp, Homo sapiens
cccagacaggagtgcctttggagtggc

SEQ ID NO: 1025 - DNA, 27 bp, Homo sapiens
cattagttagattctcataaggatggc

SEQ ID NO: 1026 - DNA, 26 bp, Homo sapiens
gaataggactgcctgataacttgtat

SEQ ID NO: 1027 - DNA, 28 bp, Homo sapiens
gcctccccatttctcctcagtttcacaa

SEQ ID NO: 1028 - DNA, 26 bp, Homo sapiens
ctatggcaaggagaggctgtaggagc

SEQ ID NO: 1029 - DNA, 28 bp, Homo sapiens
ctctgttccgcctacccaggtccacacc

SEQ ID NO: 1030 - DNA, 23 bp, Homo sapiens
ggcgggagacccttgcccctgaa

SEQ ID NO: 1031 - DNA, 28 bp, Homo sapiens
acttgtctcccattcctggtgtgtttgg

SEQ ID NO: 1032 - DNA, 25 bp, Homo sapiens
ccgacccaaaccagcactccagccg

SEQ ID NO: 1033 - DNA, 23 bp, Homo sapiens
cagagaacagagaggactgaagc

SEQ ID NO: 1034 - DNA, 23 bp, Homo sapiens
gacgacggctgtctcctcaaaca

SEQ ID NO: 1035 - DNA, 23 bp, Homo sapiens
gcctcctaacccacctccccacc

SEQ ID NO: 1036 - DNA, 28 bp, Homo sapiens
cactcagggctctgccgtcaccttggag

SEQ ID NO: 1037 - DNA, 27 bp, Homo sapiens
cgtcttccaccctgggctgttcgtccc

SEQ ID NO: 1038 - DNA, 28 bp, Homo sapiens
cggacccatcaacagtgagccaagcatc

SEQ ID NO: 1039 - DNA, 25 bp, Homo sapiens
cctgcctgtgtgtctgcgtgtgagc

SEQ ID NO: 1040 - DNA, 28 bp, Homo sapiens
ccgcatctccccttgtgccagtccagac

SEQ ID NO: 1041 - DNA, 27 bp, Homo sapiens
ggctcccaccctgttagagatgtgacg

SEQ ID NO: 1042 - DNA, 28 bp, Homo sapiens
cctaaccagtcttcttccacagcccaca

SEQ ID NO: 1043 - DNA, 27 bp, Homo sapiens
gctggagtgggctcaccgaggataggg

SEQ ID NO: 1044 - DNA, 28 bp, Homo sapiens
gggatggagggaagagtcacaggaaagt

SEQ ID NO: 1045 - DNA, 28 bp, Homo sapiens
tgtccacagtatgattctcgggttttca

SEQ ID NO: 1046 - DNA, 28 bp, Homo sapiens
tacagtggtgctacaggttatgggaggg

SEQ ID NO: 1047 - DNA, 27 bp, Homo sapiens
cctctttcccaaatgtcatcttttagc

SEQ ID NO: 1048 - DNA, 27 bp, Homo sapiens
ggaggaaaatagttgagcaggaacaat

SEQ ID NO: 1049 - DNA, 28 bp, Homo sapiens
cgatggctcatagtgctgtaatccaagc

SEQ ID NO: 1050 - DNA, 28 bp, Homo sapiens
cactttgagagcctgaagcagcagcatc

SEQ ID NO: 1051 - DNA, 60 bp, Homo sapiens
tgttgagggtgtcccggagcatcaaatctcgaagtcacatgatggccaacaagtaccaag

SEQ ID NO: 1052 - DNA, 60 bp, Homo sapiens
ctgtgtgtttgagtgtcgcttgacctgctcgaggagacagccgttgtccccagggctcct

SEQ ID NO: 1053 - DNA, 60 bp, Homo sapiens
gaggcaggcggatcacgaggtcataagatcgaatttccagtttctaaaaagtttaagaat

SEQ ID NO: 1054 - DNA, 60 bp, Homo sapiens
acagagccctcagaaataatgccgcatatcgaaggctccaaggttttcgtgatcttttca

SEQ ID NO: 1055 - DNA, 60 bp, Homo sapiens
tttgcaggtgtgtggttaacactatccttcgaaacaggtgctctggtcaagtcagtttgg

SEQ ID NO: 1056 - DNA, 60 bp, Homo sapiens
agtgtgtgtgaggaaatgcaggggtgaatcgagcccctgctttcaattcttttggatata

SEQ ID NO: 1057 - DNA, 60 bp, Homo sapiens
gcatatgcaggtgtgggctttttaaaaatcgaaatccagctccactactcactagttggt

SEQ ID NO: 1058 - DNA, 60 bp, Homo sapiens
ataatgggcgatgtttctcagggctgcttcgataggtacaactattatgtatcaatattt

SEQ ID NO: 1059 - DNA, 60 bp, Homo sapiens
ggaggggaacattacacactggggcctgtcgaatgctttatctggacctacttttgtgaa

SEQ ID NO: 1060 - DNA, 60 bp, Homo sapiens
tggtgagggtgtttttcattcagctttttcgaagctttctagggaaggccatgtcttctc

SEQ ID NO: 1061 - DNA, 60 bp, Homo sapiens
tgacctgcctctcggtgaggttgagcagtcgaagtaatgaagagggaggtggttttcttt

SEQ ID NO: 1062 - DNA, 60 bp, Homo sapiens
caggtgtgtccatgattaaacttttaattcgagagaaaaaaaaaagatatcatatttctt

SEQ ID NO: 1063 - DNA, 60 bp, Homo sapiens
ggcaggcggatcacttgaggccagggattcgacacacagagctgtgtggagtctcagcag

SEQ ID NO: 1064 - DNA, 60 bp, Homo sapiens
gcatatgcaggtgtgggctttttaaaaatcgacagatgaatgaataagcaaaatgtggtc

SEQ ID NO: 1065 - DNA, 60 bp, Homo sapiens
ggcaggcagatcacttgaggtcacgagttcgatcatttcaagaacctaccttcaagttca

SEQ ID NO: 1066 - DNA, 60 bp, Homo sapiens
gcatatgcaggtgtgggctttttaaaaatcgaaaggaaagaatcagtgtatcaaaaagat

SEQ ID NO: 1067 - DNA, 60 bp, Homo sapiens
ggaggtgtgtgtgtttaaaccaggaaagtcgagcttcacggccgctttgtttacctactc

SEQ ID NO: 1068 - DNA, 60 bp, Homo sapiens
ggcgggcagatcacttgaggtcagaacttcgaactctgctttctatttcagcagcatttg

SEQ ID NO: 1069 - DNA, 60 bp, Homo sapiens
agggcaggggcactaggatgaaaggccgtcgatcaatggataaaaacaaatataagagct

SEQ ID NO: 1070 - DNA, 60 bp, Homo sapiens
gggcgcgggtctgcggagcccccagggctcgatgtgtccattacatagatgacaactgtt

SEQ ID NO: 1071 - DNA, 60 bp, Homo sapiens
aaggcaggcggatcacgaggtcagtagatcgaagattactctgtgattttctccaattat

SEQ ID NO: 1072 - DNA, 60 bp, Homo sapiens
ggcaggcagatcacttgaggtcaggaattcgaggtgccatctgtcacccctttctttgac

SEQ ID NO: 1073 - DNA, 60 bp, Homo sapiens
gaaacagtgagtgtttatttctcgtgagtcgaagcctttatcaaagtctcaaaggggtct

SEQ ID NO: 1074 - DNA, 60 bp, Homo sapiens
tgttgagggtgtcccggagcatcaaatctcgagttttaagtgaatccagcagtctacagt

SEQ ID NO: 1075 - DNA, 60 bp, Homo sapiens
ataatgggcgatgtttctcagggctgcttcgatacaaagagatggccagatttcatctga

SEQ ID NO: 1076 - DNA, 60 bp, Homo sapiens
acagagccctcagaaataatgccgcatatcgacaagacctcagggttgggtgtcccccac

SEQ ID NO: 1077 - DNA, 60 bp, Homo sapiens
cccctcaagaacgagctctccccctgtctcgagtgctggtttgggtcggagtgctggttc

SEQ ID NO: 1078 - DNA, 60 bp, Homo sapiens
aagggctcgggagctccctcggcacacctcgattctgttcatttttcttcattctttatt

SEQ ID NO: 1079 - DNA, 60 bp, Homo sapiens
gagctgggttagggttgggaagacagcttcgaatctgcaagtctgttttgggagtagtgt

SEQ ID NO: 1080 - DNA, 60 bp, Homo sapiens
agaatgggcgatgtttctcagggctgcttcgagcatatgctacacagtggagcaaccttc

SEQ ID NO: 1081 - DNA, 60 bp, Homo sapiens
ataatgggcgatgtttctcagggctgcttcgaatggtagttctgcgtttagctctttgag

SEQ ID NO: 1082 - DNA, 60 bp, Homo sapiens
tggtgagggtgtttttcattcagctttttcgatctgatgactgaagtgtctgagctgaag

SEQ ID NO: 1083 - DNA, 60 bp, Homo sapiens
tttttattattattaaattcatatattctcgaaggtgtgtgtggatttttcttgtttgct

SEQ ID NO: 1084 - DNA, 60 bp, Homo sapiens
agcaggcagatcacttgaggtcaggagttcgaaaacaatttctcacttgtctgggcaaaa

SEQ ID NO: 1085 - DNA, 60 bp, Homo sapiens
agggacagccagccagccacagatgatgtcgatcagactagatatctgttttgtgagcta

SEQ ID NO: 1086 - DNA, 60 bp, Homo sapiens
agtgcctgtgaaagtgtttctgaattcctcgatcagcttcacacactctcactcctggag

SEQ ID NO: 1087 - DNA, 60 bp, Homo sapiens
aaggagggcagatcacgaggtcaggcgttcgataaatactggattttattaaataaactc

SEQ ID NO: 1088 - DNA, 60 bp, Homo sapiens
atgtgtttgcttatttatcattaataattcgaacatcttagagaccaagaccaaaagggg

SEQ ID NO: 1089 - DNA, 60 bp, Homo sapiens
ggcaggaggatcacttgaggccaggcattcgaaaacttatatctatacaaaacctgcaaa

SEQ ID NO: 1090 - DNA, 60 bp, Homo sapiens
gggcgcgggtctgcggagcccccagggctcgaatttagggggaaaacattaaataggaat

SEQ ID NO: 1091 - DNA, 60 bp, Homo sapiens
agtgcctgtgaaagtgtttctgaattcctcgacaatgccactcactattacctctacaac

SEQ ID NO: 1092 - DNA, 60 bp, Homo sapiens
gccgaggtgggcagatcacgaggtcaaatcgaaaggatcaggaattggaataattcctct

SEQ ID NO: 1093 - DNA, 60 bp, Homo sapiens
agaatgggcgatgtttctcagggctgcttcgaagtaaaagaacagaaacataaagtttct

SEQ ID NO: 1094 - DNA, 60 bp, Homo sapiens
gagaggtgggcaggtgttagatttagtttcgattaatcttttatatttaaaaacttggcc

SEQ ID NO: 1095 - DNA, 60 bp, Homo sapiens
ggcaggcagatcacttgaggtcagcagttcgacacagacaccctcccaagactaaaccag

SEQ ID NO: 1096 - DNA, 60 bp, Homo sapiens
gggtgtgggtggattttcaccttgtctgtcgaaggcaaagcttttcagcgatttcattaa

SEQ ID NO: 1097 - DNA, 60 bp, Homo sapiens
attgtttaagccagccagcctatggtattcgaacctacagttgaaggccttatatgcaaa

SEQ ID NO: 1098 - DNA, 60 bp, Homo sapiens
ggcaggcagatcacttgaggtcatgagttcgaaaaaaaaaaaaaaagtatttcttttagt

SEQ ID NO: 1099 - DNA, 60 bp, Homo sapiens
tcgcctaggctggagcagtggtgtgatctcgatctttgagaacctactaggtgtcagtct

SEQ ID NO: 1100 - DNA, 60 bp, Homo sapiens
ggcgggcagatcacttgaggccaggagctcgatcagtgatgttaaattaaatgagcagga

SEQ ID NO: 1101 - DNA, 24 bp, Homo sapiens
attatcctcctttgttccttcccc

SEQ ID NO: 1102 - DNA, 23 bp, Homo sapiens
ccctccaggtctctgcgtgtttc

SEQ ID NO: 1103 - DNA, 23 bp, Homo sapiens
gctcccagcagagggctcacccg

SEQ ID NO: 1104 - DNA, 23 bp, Homo sapiens
atcccgaggacgacggctgtctc

SEQ ID NO: 1105 - DNA, 28 bp, Homo sapiens
cttcacagcagtcaagtggtggtatcac

SEQ ID NO: 1106 - DNA, 28 bp, Homo sapiens
ctccttgtttgactgacttagtttctca

SEQ ID NO: 1107 - DNA, 23 bp, Homo sapiens
agattttgggctgagacaatggg

SEQ ID NO: 1108 - DNA, 28 bp, Homo sapiens
cagaactcaagaacatactccataaagt

SEQ ID NO: 1109 - DNA, 28 bp, Homo sapiens
ctctgttccgcctacccaggtccacacc

SEQ ID NO: 1110 - DNA, 23 bp, Homo sapiens
cccagcccttaccatcggtgccc

SEQ ID NO: 1111 - DNA, 24 bp, Homo sapiens
gctctaactgatgctttctccaag

SEQ ID NO: 1112 - DNA, 26 bp, Homo sapiens
tgagtaacaaagtgagaccttatgtc

SEQ ID NO: 1113 - DNA, 28 bp, Homo sapiens
ggcagaaaggaaagagcctcgcagcatt

SEQ ID NO: 1114 - DNA, 28 bp, Homo sapiens
tcctcatccttgctttcctcatctgtga

SEQ ID NO: 1115 - DNA, 26 bp, Homo sapiens
ctggctattttcacttcttttgtgga

SEQ ID NO: 1116 - DNA, 23 bp, Homo sapiens
gcaaggctgtgtcccctcctgag

SEQ ID NO: 1117 - DNA, 24 bp, Homo sapiens
gtgggagttgaacaatgagaacac

SEQ ID NO: 1118 - DNA, 26 bp, Homo sapiens
aatgccaccaccagtaatgtttctat

SEQ ID NO: 1119 - DNA, 28 bp, Homo sapiens
gcctccccatttctcctcagtttcacaa

SEQ ID NO: 1120 - DNA, 28 bp, Homo sapiens
ggctctggagactcagcaccattctggg

SEQ ID NO: 1121 - DNA, 23 bp, Homo sapiens
cattttcatcctgcggttctgga

SEQ ID NO: 1122 - DNA, 23 bp, Homo sapiens
caacagagatgggagacttgaag

SEQ ID NO: 1123 - DNA, 23 bp, Homo sapiens
cgtggctcctacagaactccata

SEQ ID NO: 1124 - DNA, 28 bp, Homo sapiens
aaagcagtttctgtgtccgtttgcccca

SEQ ID NO: 1125 - DNA, 28 bp, Homo sapiens
gcctctctcccatcaacatccttcagac

SEQ ID NO: 1126 - DNA, 24 bp, Homo sapiens
atggcggctgcccctcctcccagg

SEQ ID NO: 1127 - DNA, 28 bp, Homo sapiens
ggcagaaaggaaagagcctcgcagcatt

SEQ ID NO: 1128 - DNA, 27 bp, Homo sapiens
ctcagactcctcccagccccactcagg

SEQ ID NO: 1129 - DNA, 28 bp, Homo sapiens
aatagggaacaggtgtgtgacccatagc

SEQ ID NO: 1130 - DNA, 28 bp, Homo sapiens
gaaatgccagagaagaaagacaacaact

SEQ ID NO: 1131 - DNA, 28 bp, Homo sapiens
ggcagaaaggaaagagcctcgcagcatt

SEQ ID NO: 1132 - DNA, 27 bp, Homo sapiens
ctccccatcctcccctttaccctaccc

SEQ ID NO: 1133 - DNA, 28 bp, Homo sapiens
cttggtatttcccttattgttttgaagc

SEQ ID NO: 1134 - DNA, 23 bp, Homo sapiens
ccaccattgctgaggcttgagta

SEQ ID NO: 1135 - DNA, 23 bp, Homo sapiens
ccacatcttccacccctgacacc

SEQ ID NO: 1136 - DNA, 23 bp, Homo sapiens
ggtggaggagaacggtttgatgc

SEQ ID NO: 1137 - DNA, 23 bp, Homo sapiens
ctccccaagcctccccaagcagg

SEQ ID NO: 1138 - DNA, 28 bp, Homo sapiens
gctggtgttggattgaataggaggaaga

SEQ ID NO: 1139 - DNA, 24 bp, Homo sapiens
tccgccctgcccacacccacctcc

SEQ ID NO: 1140 - DNA, 28 bp, Homo sapiens
gcacaccctgcctaaagtggttttccct

SEQ ID NO: 1141 - DNA, 28 bp, Homo sapiens
cagtgtctctgctacaggctacaacatc

SEQ ID NO: 1142 - DNA, 28 bp, Homo sapiens
caaagaataaacaaggagacaagggctt

SEQ ID NO: 1143 - DNA, 23 bp, Homo sapiens
gtgcggtggctcacacttgtaat

SEQ ID NO: 1144 - DNA, 23 bp, Homo sapiens
agccgaagcagggcaaggcattg

SEQ ID NO: 1145 - DNA, 23 bp, Homo sapiens
ccaccctgctgtcctgcttgggc

SEQ ID NO: 1146 - DNA, 28 bp, Homo sapiens
gagttgggaggaatctaaagggttacct

SEQ ID NO: 1147 - DNA, 24 bp, Homo sapiens
attatcctcctttgttccttcccc

SEQ ID NO: 1148 - DNA, 23 bp, Homo sapiens
ctcggatggcgtttcctcaggga

SEQ ID NO: 1149 - DNA, 28 bp, Homo sapiens
aggagaaggaatgtcacaaggtaaggtc

SEQ ID NO: 1150 - DNA, 28 bp, Homo sapiens
gtagagaaggacaatggtatgggttcag

SEQ ID NO: 1151 - DNA, 23 bp, Homo sapiens
agattttgggctgagacaatggg

SEQ ID NO: 1152 - DNA, 23 bp, Homo sapiens
aaggctccatctgtcaaacggat

SEQ ID NO: 1153 - DNA, 28 bp, Homo sapiens
taggaggagaaaacagcaaatggtcggg

SEQ ID NO: 1154 - DNA, 25 bp, Homo sapiens
ccgacccaaaccagcactccagccg

SEQ ID NO: 1155 - DNA, 28 bp, Homo sapiens
gccctttccagcccctcagtcctgtagc

SEQ ID NO: 1156 - DNA, 26 bp, Homo sapiens
ccatccctcaggaagcccagagactg

SEQ ID NO: 1157 - DNA, 28 bp, Homo sapiens
ccaggaatgtcaggcgacccctcaggtg

SEQ ID NO: 1158 - DNA, 28 bp, Homo sapiens
gccatcccatcttcaaggactgacggag

SEQ ID NO: 1159 - DNA, 23 bp, Homo sapiens
tgaagtcttgaggcggcgaaaat

SEQ ID NO: 1160 - DNA, 28 bp, Homo sapiens
ggtaaccaacatcttgtattgtattagc

SEQ ID NO: 1161 - DNA, 26 bp, Homo sapiens
ctggctattttcacttcttttgtgga

SEQ ID NO: 1162 - DNA, 25 bp, Homo sapiens
gttgtggaaagcagtatcgtgattc

SEQ ID NO: 1163 - DNA, 28 bp, Homo sapiens
gcctccccatttctcctcagtttcacaa

SEQ ID NO: 1164 - DNA, 28 bp, Homo sapiens
ctgcttctcctcctgctctctctgctcc

SEQ ID NO: 1165 - DNA, 28 bp, Homo sapiens
gggctggagcacagtgacgcaatcgtgg

SEQ ID NO: 1166 - DNA, 28 bp, Homo sapiens
gactccctttgttctgcctggcttctga

SEQ ID NO: 1167 - DNA, 28 bp, Homo sapiens
ctctgcctcctctgccctgttcctactg

SEQ ID NO: 1168 - DNA, 28 bp, Homo sapiens
gccaccacaacacactgctgcctcacac

SEQ ID NO: 1169 - DNA, 28 bp, Homo sapiens
cacacagataaagatgagcaggagggac

SEQ ID NO: 1170 - DNA, 27 bp, Homo sapiens
gctctcctggacttgagtgtggaaggg

SEQ ID NO: 1171 - DNA, 23 bp, Homo sapiens
gggcaagggtgggaagcgatggc

SEQ ID NO: 1172 - DNA, 28 bp, Homo sapiens
agggagccagagacagaggaggcagtgg

SEQ ID NO: 1173 - DNA, 28 bp, Homo sapiens
aggtgctcagtcagggctgctgcctctc

SEQ ID NO: 1174 - DNA, 23 bp, Homo sapiens
tcctgtgggtccctgtggctggg

SEQ ID NO: 1175 - DNA, 28 bp, Homo sapiens
gtaactacatcgctcccaactttcaatg

SEQ ID NO: 1176 - DNA, 28 bp, Homo sapiens
ctgctcctttgcccttccagagagacaa

SEQ ID NO: 1177 - DNA, 27 bp, Homo sapiens
acatttcttgcccactgttcagccgat

SEQ ID NO: 1178 - DNA, 24 bp, Homo sapiens
ccctcacccctggcaaccaccgat

SEQ ID NO: 1179 - DNA, 23 bp, Homo sapiens
cctgcccacacccacctccgccg

SEQ ID NO: 1180 - DNA, 23 bp, Homo sapiens
ggcaacagagagaggctccgtct

SEQ ID NO: 1181 - DNA, 28 bp, Homo sapiens
acaggacagggcaggagaggcaacgagg

SEQ ID NO: 1182 - DNA, 24 bp, Homo sapiens
cccaaggcggtagagggtgtcacg

SEQ ID NO: 1183 - DNA, 25 bp, Homo sapiens
accactatgacctctttcccaaatg

SEQ ID NO: 1184 - DNA, 24 bp, Homo sapiens
ctgtggctattactgtgttacaat

SEQ ID NO: 1185 - DNA, 23 bp, Homo sapiens
gcctgacattcctgccgtcttat

SEQ ID NO: 1186 - DNA, 24 bp, Homo sapiens
gtgacacccacatacttgccaaat

SEQ ID NO: 1187 - DNA, 26 bp, Homo sapiens
cagtgcccaaagaggtccctgaggtc

SEQ ID NO: 1188 - DNA, 28 bp, Homo sapiens
gtggctttacaacctcactctgaacctt

SEQ ID NO: 1189 - DNA, 26 bp, Homo sapiens
ctgggctattctaaatgccaactaat

SEQ ID NO: 1190 - DNA, 26 bp, Homo sapiens
attcagagattcaacttcttcctggt

SEQ ID NO: 1191 - DNA, 23 bp, Homo sapiens
ggcagcaggtaagagccccaggg

SEQ ID NO: 1192 - DNA, 28 bp, Homo sapiens
ccgctcaggagactcgcactgggtgtag

SEQ ID NO: 1193 - DNA, 28 bp, Homo sapiens
tctactaaccagaaagacagccctcacc

SEQ ID NO: 1194 - DNA, 28 bp, Homo sapiens
gatggtgtgtgggttaggcactactggg

SEQ ID NO: 1195 - DNA, 27 bp, Homo sapiens
attctcaggatggaggctgggtgtggt

SEQ ID NO: 1196 - DNA, 28 bp, Homo sapiens
ggaaagcatctcaagaccaaagtcaaag

SEQ ID NO: 1197 - DNA, 28 bp, Homo sapiens
gtcacccttcagagatggcaacatttac

SEQ ID NO: 1198 - DNA, 27 bp, Homo sapiens
tagcaacaactgtctcccgtcctctgc

SEQ ID NO: 1199 - DNA, 26 bp, Homo sapiens
cctctttcccaaatgtcatcttttag

SEQ ID NO: 1200 - DNA, 25 bp, Homo sapiens
ggaggaaaatagttgagcaggaaca